(19) United States
(12) Patent Application Publication
(10) Pub. No.: US 2023/0313224 A1
(43) Pub. Date: Oct. 5, 2023

(54) INTEGRATION OF LARGE ADENOVIRUS PAYLOADS

(71) Applicants: Fred Hutchinson Cancer Center, Seattle, WA (US); University of Washington, Seattle, WA (US)

(72) Inventors: Andre Lieber, Seattle, WA (US); Hans-Peter Kiem, Seattle, WA (US); Hongjie Wang, Seattle (CN)

(73) Assignees: Fred Hutchinson Cancer Center, Seattle, WA (US); University of Washington, Seattle, WA (US)

(21) Appl. No.: 17/995,671

(22) PCT Filed: Apr. 12, 2021
(86) PCT No.: PCT/US2021/026880

§ 371 Date: Oct. 6, 2022

Related U.S. Application Data
(60) Provisional application No. 63/009,298, filed on Apr. 13, 2020.

(51) Int. Cl. C12N 15/86; C07K 14/075; C07K 14/755; C12N 5/0789; A61P 7/00

(52) U.S. Cl. CPC C12N 15/86; A61P 7/00; C07K 14/075; C07K 14/755; C12N 5/0647; C12N 2710/10343; C12N 2800/90

(57) ABSTRACT

The current disclosure provides recombinant adenoviral vectors and adenoviral genomes that can accommodate or that contain a large transposon payload, for instance a transposon payload of up to 40 kb. The adenoviral vectors and genomes can deliver the large transposon payload into a target genome, for instance for gene therapy.

### CROSS REFERENCE TO RELATED APPLICATIONS

This is the 371 National Phase of co-pending international application No. PCT/US2021/026880, filed Apr. 12, 2021, which claims priority to and the benefit of the earlier filing date of U.S. Provisional Application No. 63/009,298, filed on Apr. 13, 2020, which is incorporated by reference herein in its entirety.

### STATEMENT REGARDING FEDERALLY SPONSORED RESEARCH OR DEVELOPMENT

This invention was made with government support under grant Numbers HL128288 and HL136135, awarded by the National Institutes of Health. The government has certain rights in the invention.

### FIELD OF THE DISCLOSURE

The current disclosure provides, among other things, recombinant adenoviral vectors and adenoviral genomes that can accommodate or that contain a large transposon payload, for instance a transposon payload of up to 40 kb. Certain of the adenoviral vectors and genomes can deliver the large transposon payload into a target genome, for instance for gene therapy.

### BACKGROUND OF THE DISCLOSURE

Gene therapy presents many challenges. Viral vectors are one means of gene therapy. Various challenges in the development of viral vectors for gene therapy include, in some instances, vector payload capacity, efficiency of transgene integration into target cell genomes, cell type specificity of transgene expression, level of transgene expression, and positional effects of integration. Various methods of gene therapy using viral vectors require resource-consuming steps of removing cells from a subject and engineering and/or expanding the cells ex vivo before administering them to a subject. For at least these reasons, and particularly in view of the growing number of therapies that utilize viral vectors, there is a great need for improved viral vector designs.

Hemoglobinopathies are one of the most prevalent genetic disorders worldwide, notably with a significantly reduced survival rate for patients born in underdeveloped countries. Examples of hemoglobinopathies include sickle-cell disease and thalassemia. Patient-specific blood stem/progenitor cell (HSPC) gene therapy has great potential to treat hemoglobinopathies.

Further, more than 80 primary immune deficiency diseases are recognized by the World Health Organization. These diseases are characterized by an intrinsic defect in the immune system in which, in some cases, the body is unable to produce any or enough antibodies against infection. In other cases, cellular defenses to fight infection fail to work properly. Typically, primary immune deficiencies are inherited disorders.

Secondary, or acquired, immune deficiencies are not the result of inherited genetic abnormalities, but rather occur in individuals in which the immune system is compromised by factors outside the immune system. Examples include trauma, viruses, chemotherapy, toxins, and pollution. Acquired immunodeficiency syndrome (AIDS) is an example of a secondary immune deficiency disorder caused by a virus, the human immunodeficiency virus (HIV), in which a depletion of T lymphocytes renders the body unable to fight infection.

X-linked severe combined immunodeficiency (SCID-X1) is both a cellular and humoral immune depletion caused by mutations in the common gamma chain gene (γC), which result in the absence of T and natural killer (NK) lymphocytes and the presence of nonfunctional B lymphocytes. SCID-X1 is fatal in the first two years of life unless the immune system is reconstituted, for example, through bone marrow transplant (BMT) or gene therapy.

Because most individuals lack a matched donor for BMT or non-autologous gene therapy, haploidentical parental bone marrow depleted of mature T cells is often used; however, complications include graft versus host disease (GVHD), failure to make adequate antibodies hence requiring long-term immunoglobulin replacement, late loss of T cells due to failure to engraft hematopoietic stem and progenitor cells (HSPCs), chronic warts, and lymphocyte dysregulation.

Fanconi anemia (FA) is an inherited blood disorder that leads to bone marrow failure. It is characterized, in part, by a deficient DNA-repair mechanism. At least 20% of patients with FA develop cancers such as acute myeloid leukemias, and cancers of the skin, liver, gastrointestinal tract, and gynecological systems. The skin and gastrointestinal tumors are usually squamous cell carcinomas. The average age of patients who develop cancer is 15 years for leukemia, 16 years for liver tumors, and 23 years for other tumors.

Treatments using in vivo gene therapy, which includes the direct delivery of a viral vector to a patient, has been explored. In vivo gene therapy is a simple and attractive approach because it may not require any genotoxic conditioning (or could require less genotoxic conditioning) nor ex vivo cell processing and thus could be adopted at many institutions worldwide, including those in developing countries, as the therapy could be administered through an injection, similar to what is already done worldwide for the delivery of vaccines.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a S′-tripartite leader (TPL) sequence which makes them preferred mRNAs for translation.

For successful gene therapy, without position effects of integration and transcriptional silencing, the transferred gene must be expressed at high levels in desired tissues or cells. A locus control region (LCR) is particularly suitable for this task as an LCR is characterized by its ability to enhance the expression of linked genes to physiological levels in a tissue-specific and copy number-dependent manner at ectopic chromatic sites. The components of an LCR commonly colocalize to sites of DNAse I hypersensitivity (HS) in the chromatin of expressing cells. The core determinants at individual HSs are composed of arrays of multiple ubiquitous and lineage-specific transcription factor-binding sites.

### SUMMARY

The present disclosure includes, among other things, adenoviral vectors and adenoviral genomes, systems including two or more adenoviral vectors and/or adenoviral genomes of the present disclosure, and uses of such adenoviral vectors, adenoviral genomes, and systems. In certain embodiments, the present invention includes adenoviral vectors and/or adenoviral genomes that include a transposon payload of, e.g., 1 kb to 40 kb. In certain embodiments of the present disclosure, a transposase can cause integration of a transposon payload of, e.g., up to 40 kb into the genome of a target cell. Thus, the present disclosure includes, among other things vectors, genomes, and systems that enable integration of a payload of up to 40 kb present in an adenoviral donor vector into a target cell genome. As those of skill in the art will appreciate, vector integration capacity, in and of itself, is one critically important feature a gene therapy system, at least in part because integration capacity limits the length and/or complexity of therapeutic payloads.

Certain examples of long and/or complex nucleic acid payloads recognized in the present disclosure include payloads that include a Long Locus Control Region. Due to their length, Long Locus Control Regions have been historically unsuitable for inclusion in adenoviral payloads, but long and/or complex nucleic acid payloads including without limitation long and/or complex nucleic acid payloads including Long Locus Control Regions, can be integrated into target cell genomes in accordance with vectors, genomes, and systems disclosed herein.

Thus there is provided in one embodiment an adenoviral donor vector including: (a) an adenoviral capsid; and (b) a linear, double-stranded DNA genome including: (i) a transposon payload of at least 10 kb; (ii) transposon inverted repeats (IRs) that flank the transposon payload; and (iii) recombinase direct repeats (DRs) that flank the transposon inverted repeats.

Another embodiment is an adenoviral donor genome including: (a) a transposon payload of at least 10 kb; (b) transposon inverted repeats (IRs) that flank the transposon payload; and (c) recombinase direct repeats (DRs) that flank the transposon inverted repeats.

Also provided is an adenoviral transposition system including: (a) an adenoviral donor vector as described herein; and (b) an adenoviral support vector including (i) the adenoviral capsid; and (ii) an adenoviral support genome including a nucleic acid sequence encoding a transposase.

Yet another embodiment is an adenoviral transposition system including: (a) an adenoviral donor genome as described herein; and (b) an adenoviral support genome including a nucleic acid sequence encoding a transposase.

Further, there is provided an adenoviral production system including: (a) a nucleic acid including an adenoviral donor genome as described herein; and (b) a nucleic acid including an adenoviral helper genome including a conditional packaging element.

Further embodiments are cells (for instance, a hematopoietic stem cell) that include a vector, genome, or system according to any one of the various embodiments described herein.

Also described are cell(s) (for instance, a hematopoietic stem cell) including in its genome the transposon payload of any embodiment described herein, wherein the transposon payload present in the genome of the cell is flanked by the transposon inverted repeats.

Yet another embodiment is an adenovirus-producing cell including an adenoviral production system according to any one of the embodiments described herein, optionally wherein the cell is a HEK293 cell.

A method of modifying a cell, the method including contacting the cell with a vector, genome, or system according to any one of the embodiments described herein.

Another embodiment is a method of modifying a cell of a subject, the method including administering to the subject a vector, genome, or system according to any one of the embodiments described herein.

Another embodiment is a method of modifying a cell of a subject without isolation of the cell from the subject, the method including administering to the subject a vector, genome, or system according to any one of the embodiments described herein.

Also provided are methods of treating a disease or condition in a subject in need thereof, the method including administering to the subject a vector, genome, or system according to any one of the embodiments described herein.

In at least one aspect, the present disclosure provides an adenoviral donor vector including: (a) an adenoviral capsid; and (b) a linear, double-stranded DNA genome including: (i) a transposon payload of at least 10 kb; (ii) transposon inverted repeats (IRs) that flank the transposon payload; and (iii) recombinase direct repeats (DRs) that flank the transposon inverted repeats.

In at least one aspect, the present disclosure provides an adenoviral donor genome including: (a) a transposon payload of at least 10 kb; (b) transposon inverted repeats (IRs) that flank the transposon payload; and (c) recombinase direct repeats (DRs) that flank the transposon inverted repeats.

In at least one aspect, the present disclosure provides an adenoviral transposition system including: (a) the adenoviral donor vector of embodiment 1; and (b) an adenoviral support vector including (i) the adenoviral capsid; and (ii) an adenoviral support genome including a nucleic acid sequence encoding a transposase.

In at least one aspect, the present disclosure provides an adenoviral transposition system including: (a) the adenoviral donor genome of embodiment 2; and (b) an adenoviral support genome including a nucleic acid sequence encoding a transposase.

In at least one aspect, the present disclosure provides an adenoviral production system including: (a) a nucleic acid including the adenoviral donor genome of embodiment 2; and (b) a nucleic acid including an adenoviral helper genome including a conditional packaging element.

In various embodiments, the transposon payload includes a Long LCR, optionally where the Long LCR is a β-globin Long LCR including β-globin LCR HS1 to HS5. In various embodiments, the Long LCR has a length of at least 27 kb. In various embodiments, the transposon payload includes an LCR set forth in Table 1. In various embodiments, the transposon payload has a length of at least 15 kb, at least 16 kb, at least 17 kb, at least 18 kb, at least 19 kb, at least 20 kb, at least 21 kb, at least 22 kb, at least 23 kb, at least 24 kb, at least 25 kb, at least 30 kb, at least 35 kb, at least 38 kb, or at least 40 kb. In various embodiments, the transposon payload has a length of 10 kb-35 kb, 10 kb-30 kb, 15 kb-35 kb, 15 kb-30 kb, 20 kb-35 kb, or 20 kb-30 kb. In various embodiments, the transposon payload has a length of 10 kb-32.4 kb, 15 kb-32.4 kb, or 20 kb-32.4 kb.

In various embodiments, the transposon payload includes a nucleic acid sequence that encodes a protein, optionally where the protein is a therapeutic protein. In various embodiments, the protein is selected from the group consisting of a β globin replacement protein and a γ-globin replacement protein. In various embodiments, the protein is a Factor VIII replacement protein. In various embodiments, the nucleic acid sequence that encodes the protein is operably linked with a promoter, optionally where the promoter is a β globin promoter.

In various embodiments, the transposon inverted repeats are Sleeping Beauty (SB) inverted repeats, optionally where the SB inverted repeats are pT4 inverted repeats. In various embodiments, the transposase is a Sleeping Beauty (SB) transposase, optionally where the transposase is Sleeping Beauty 100x (SB1 00x). In various embodiments, the recombinase direct repeats are FRT sites. In various embodiments, the adenoviral support genome includes a nucleic acid encoding a recombinase. In various embodiments, the recombinase is a FLP recombinase. In various embodiments, the transposon payload includes a β-globin long LCR, the transposon payload includes a nucleic acid sequence that encodes β-globin operably linked with a β-globin promoter, the inverted repeats are SB inverted repeats, and the recombinase direct repeats are FRT sites.

In various embodiments, the transposon payload includes a selection cassette, optionally where the selection cassette includes a nucleic acid sequence that encodes mgmt^{P140K}.

In various embodiments, the adenoviral capsid is modified for increased affinity to CD46, optionally where the adenoviral capsid is an Ad35++ capsid.

In various embodiments, the adenoviral helper genome conditional packaging element includes a packaging sequence flanked by recombinase direct repeats.

In various embodiments, the recombinase direct repeats that flank the packaging sequence of the conditional packaging element are LoxP sites.

In various embodiments, the present disclosure provides a cell including a vector, genome, or system according to the present disclosure.

In various embodiments, the present disclosure provides a cell including in its genome the transposon payload according to the present disclosure, where the transposon payload present in the genome of the cell is flanked by the transposon inverted repeats.

In various embodiments, the cell is a hematopoietic stem cell.

In various embodiments, the present disclosure provides an adenovirus-producing cell including an adenoviral production system according to the present disclosure, optionally where the cell is a HEK293 cell.

In various embodiments, the present disclosure provides a method of modifying a cell, the method including contacting the cell with a vector, genome, or system according to the present disclosure.

In various embodiments, the present disclosure provides a method of modifying a cell of a subject, the method including administering to the subject a vector, genome, or system according to the present disclosure.

In various embodiments, the present disclosure provides a method of modifying a cell of a subject without isolation of the cell from the subject, the method including administering to the subject a vector, genome, or system according to the present disclosure.

In various embodiments, the present disclosure provides a method of treating a disease or condition in a subject in need thereof, the method including administering to the subject a vector, genome, or system according to the present disclosure.

In various embodiments, the adenoviral donor vector is administered to the subject intravenously.

In various embodiments, the method includes administering to the subject a mobilization agent, optionally where the mobilization agent includes one or more of granulocyte-colony stimulating factor (G-CSF), a CXCR4 antagonist, and a CXCR2 agonist. In various embodiments, the CXCR4 antagonist is AMD3100. In various embodiments, the CXCR2 agonist is GRO-β.

In various embodiments, the transposon payload includes a selection cassette and the method includes administering a selection agent to the subject. In various embodiments, the selection cassette encodes mgmt^{P140K} and the selection agent is O⁶BG/BCNU.

In various embodiments, the method causes integration and/or expression of at least one copy of the transposon payload in at least 20%, 30%, 40%, 50%, 60%, 70%, 80%, 90%, or 95% of cells expressing CD46. In various embodiments, the method causes integration and/or expression of at least one copy of the transposon payload in at least 20%, 30%, 40%, 50%, 60%, 70%, 80%, 90%, or 95% of hematopoietic stem cells and/or erythroid Ter119⁺ cells. In various embodiments, the method causes integration of an average of at least 2 copies of the transposon payload in the genomes of cells including at least 1 copy of the transposon payload. In various embodiments, the method causes integration of an average of at least 2.5 copies of the transposon payload in the genomes of cells including at least 1 copy of the transposon payload. In various embodiments, the method causes expression of a protein encoded by the transposon payload at a level that is at least about 20% of the level of reference, optionally where the reference is expression of an endogenous reference protein in the subject or in a reference population. In various embodiments, the method causes expression of a protein encoded by the transposon payload at a level that is at least about 25% of the level of reference, optionally where the reference is expression of an endogenous reference protein in the subject or in a reference population.

In various embodiments, the subject is a subject suffering from thalassemia intermedia, where the transposase payload includes a β-globin Long LCR including β-globin LCR HS1 to HS5 and a nucleic acid sequence encoding a β globin replacement protein and/or γ-globin replacement protein operably linked with a β globin promoter. In various embodiments, the subject is a subject suffering from hemophilia, where the transposase payload includes a β-globin Long LCR including β-globin LCR HS1 to HS5 and a nucleic acid sequence encoding a Factor VIII replacement protein operably linked with a β globin promoter. In various embodiments, expression of the protein in the subject reduces at least one symptom of thalassemia intermedia and/or treats thalassemia intermedia.

### DEFINITIONS

A, An, The: As used herein, “a”, “an”, and “the” refer to one or to more than one (i.e., to at least one) of the grammatical object of the article. By way of example, “an element” discloses embodiments of exactly one element and embodiments including more than one element.

About: As used herein, term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system to achieve delivery of an agent that is, or is included in, the composition.

Adoptive cell therapy: As used herein, “adoptive cell therapy” or “ACT” involves transfer of cells with a therapeutic activity into a subject, e.g., a subject in need of treatment for a condition, disorder, or disease. In some embodiments, ACT includes transfer into a subject of cells after ex vivo and/or in vitro engineering and/or expansion of the cells.

Affinity: As used herein, “affinity” refers to the strength of the sum total of non-covalent interactions between a particular binding agent (e.g., a viral vector), and/or a binding moiety thereof, with a binding target (e.g., a cell). Unless indicated otherwise, as used herein, “binding affinity” refers to a 1:1 interaction between a binding agent and a binding target thereof (e.g., a viral vector with a target cell of the viral vector). Those of skill in the art appreciate that a change in affinity can be described by comparison to a reference (e.g., increased or decreased relative to a reference), or can be described numerically. Affinity can be measured and/or expressed in a number of ways known in the art, including, but not limited to, equilibrium dissociation constant (K_{D}) and/or equilibrium association constant (K_{A}). K_{D} is the quotient of k_{off}/kₒₙ, whereas K_{A} is the quotient of kₒₙ/kₒf_{f}, where kₒₙ refers to the association rate constant of, e.g., viral vector with target cell, and k_{off} refers to the dissociation of, e.g., viral vector from target cell. The kₒₙ and k_{off} can be determined by techniques known to those of skill in the art.

Agent: As used herein, the term “agent” may refer to any chemical entity, including without limitation any of one or more of an atom, molecule, compound, amino acid, polypeptide, nucleotide, nucleic acid, protein, protein complex, liquid, solution, saccharide, polysaccharide, lipid, or combination or complex thereof.

Allogeneic: As used herein, term “allogeneic” refers to any material derived from one subject which is then introduced to another subject, e.g., allogeneic T cell transplantation.

Between or From: As used herein, the term “between” refers to content that falls between indicated upper and lower, or first and second, boundaries, inclusive of the boundaries. Similarly, the term “from”, when used in the context of a range of values, indicates that the range includes content that falls between indicated upper and lower, or first and second, boundaries, inclusive of the boundaries.

Binding: As used herein, the term “binding” refers to a non-covalent association between or among two or more agents. “Direct” binding involves physical contact between agents; indirect binding involves physical interaction by way of physical contact with one or more intermediate agents. Binding between two or more agents can occur and/or be assessed in any of a variety of contexts, including where interacting agents are studied in isolation or in the context of more complex systems (e.g., while covalently or otherwise associated with a carrier agents and/or in a biological system or cell).

Cancer: As used herein, the term “cancer” refers to a condition, disorder, or disease in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer can include one or more tumors. In some embodiments, a cancer can be or include cells that are precancerous (e.g., benign), malignant, pre-metastatic, metastatic, and/or non-metastatic. In some embodiments, a cancer can be or include a solid tumor. In some embodiments, a cancer can be or include a hematologic tumor.

Chimeric antigen receptor: As used herein, “Chimeric antigen receptor” or “CAR” refers to an engineered protein that includes (i) an extracellular domain that includes a moiety that binds a target antigen; (ii) a transmembrane domain; and (iii) an intracellular signaling domain that sends activating signals when the CAR is stimulated by binding of the extracellular binding moiety with a target antigen. A T cell that has been genetically engineered to express a chimeric antigen receptor may be referred to as a CAR T cell. Thus, for example, when certain CARs are expressed by a T cell, binding of the CAR extracellular binding moiety with a target antigen can activate the T cell. CARs are also known as chimeric T cell receptors or chimeric immunoreceptors.

Combination therapy: As used herein, the term “combination therapy” refers to administration to a subject of to two or more agents or regimens such that the two or more agents or regimens together treat a condition, disorder, or disease of the subject. In some embodiments, the two or more therapeutic agents or regimens can be administered simultaneously, sequentially, or in overlapping dosing regimens. Those of skill in the art will appreciate that combination therapy includes but does not require that the two agents or regimens be administered together in a single composition, nor at the same time.

Control expression or activity: As used herein, a first element (e.g., a protein, such as a transcription factor, or a nucleic acid sequence, such as promoter) “controls” or “drives” expression or activity of a second element (e.g., a protein or a nucleic acid encoding an agent such as a protein) if the expression or activity of the second element is wholly or partially dependent upon status (e.g., presence, absence, conformation, chemical modification, interaction, or other activity) of the first under at least one set of conditions. Control of expression or activity can be substantial control or activity, e.g., in that a change in status of the first element can, under at least one set of conditions, result in a change in expression or activity of the second element of at least 10% (e.g., at least 20%, 30%, 40%, 50%, 60%, 70%, 80%, 90%, 100%, 2-fold, 3-fold, 4-fold, 5-fold, 10-fold, 20-fold, 30-fold, 40-fold, 50-fold, 100-fold) as compared to a reference control.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of skill in the art appreciate that residues in a provided polypeptide or polynucleotide sequence are often designated (e.g., numbered or labeled) according to the scheme of a related reference sequence (even if, e.g., such designation does not reflect literal numbering of the provided sequence). By way of illustration, if a reference sequence includes a particular amino acid motif at positions 100-110, and a second related sequence includes the same motif at positions 110-120, the motif positions of the second related sequence can be said to “correspond to” positions 100-110 of the reference sequence. Those of skill in the art appreciate that corresponding positions can be readily identified, e.g., by alignment of sequences, and that such alignment is commonly accomplished by any of a variety of known tools, strategies, and/or algorithms, including without limitation software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE.

Dosing regimen: As used herein, the term “dosing regimen” can refer to a set of one or more same or different unit doses administered to a subject, typically including a plurality of unit doses administration of each of which is separated from administration of the others by a period of time. In various embodiments, one or more or all unit doses of a dosing regimen may be the same or can vary (e.g., increase over time, decrease over time, or be adjusted in accordance with the subject and/or with a medical practitioner’s determination). In various embodiments, one or more or all of the periods of time between each dose may be the same or can vary (e.g., increase over time, decrease over time, or be adjusted in accordance with the subject and/or with a medical practitioner’s determination). In some embodiments, a given therapeutic agent has a recommended dosing regimen, which can involve one or more doses. Typically, at least one recommended dosing regimen of a marketed drug is known to those of skill in the art. In some embodiments, a dosing regimen is correlated with a desired or beneficial outcome when administered across a relevant population (i.e., is a therapeutic dosing regimen).

Downstream and Upstream: As used herein, the term” downstream” means that a first DNA region is closer, relative to a second DNA region, to the C-terminus of a nucleic acid that includes the first DNA region and the second DNA region. As used herein, the term “upstream” means a first DNA region is closer, relative to a second DNA region, to the N-terminus of a nucleic acid that includes the first DNA region and the second DNA region.

Engineered: As used herein, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence. In some embodiments, an engineered polynucleotide includes a coding sequence and/or a regulatory sequence that is found in nature operably linked with a first sequence but is not found in nature operably linked with a second sequence, which is in the engineered polynucleotide operably linked in with the second sequence by the hand of man. In some embodiments, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

Excipient: As used herein, “excipient” refers to a non-therapeutic agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or the like.

Expression: As used herein, “expression” refers individually and/or cumulatively to one or more biological process that result in production from a nucleic acid sequence of an encoded agent, such as a protein. Expression specifically includes either or both of transcription and translation.

Fragment: As used herein, “fragment” refers a structure that includes and/or consists of a discrete portion of a reference agent (sometimes referred to as the “parent” agent). In some embodiments, a fragment lacks one or more moieties found in the reference agent. In some embodiments, a fragment includes or consists of one or more moieties found in the reference agent. In some embodiments, the reference agent is a polymer such as a polynucleotide or polypeptide. In some embodiments, a fragment of a polymer includes or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) of the reference polymer. In some embodiments, a fragment of a polymer includes or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the reference polymer. A fragment of a reference polymer is not necessarily identical to a corresponding portion of the reference polymer. For example, a fragment of a reference polymer can be a polymer having a sequence of residues having at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more identity to the reference polymer. A fragment may, or may not, be generated by physical fragmentation of a reference agent. In some instances, a fragment is generated by physical fragmentation of a reference agent. In some instances, a fragment is not generated by physical fragmentation of a reference agent and can be instead, for example, produced by de novo synthesis or other means.

Gene, Transgene: As used herein, the term “gene” refers to a DNA sequence that is or includes coding sequence (i.e., a DNA sequence that encodes an expression product, such as an RNA product and/or a polypeptide product), optionally together with some or all of regulatory sequences that control expression of the coding sequence. In some embodiments, a gene includes non-coding sequence such as, without limitation, introns. In some embodiments, a gene may include both coding (e.g., exonic) and non-coding (e.g., intronic) sequences. In some embodiments, a gene includes a regulatory sequence that is a promoter. In some embodiments, a gene includes one or both of a (i) DNA nucleotides extending a predetermined number of nucleotides upstream of the coding sequence in a reference context, such as a source genome, and (ii) DNA nucleotides extending a predetermined number of nucleotides downstream of the coding sequence in a reference context, such as a source genome. In various embodiments, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb. As used herein, a “transgene” refers to a gene that is not endogenous or native to a reference context in which the gene is present or into which the gene may be placed by engineering.

Gene product or expression product: As used herein, the term “gene product” or “expression product” generally refers to an RNA transcribed from the gene (pre-and/or post-processing) or a polypeptide (pre- and/or post-modification) encoded by an RNA transcribed from the gene.

Host cell, target cell: As used herein, “host cell” refers to a cell into which exogenous DNA (recombinant or otherwise), such as a transgene, has been introduced. Those of skill in the art appreciate that a “host cell” can be the cell into which the exogenous DNA was initially introduced and/or progeny or copies, perfect or imperfect, thereof. In some embodiments, a host cell includes one or more viral genes or transgenes. In some embodiments, an intended or potential host cell can be referred to as a target cell.

Identity: As used herein, the term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided sequences are known in the art. Calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally accounting for the number of gaps, and the length of each gap, which may need to be introduced for optimal alignment of the two sequences. The comparison of sequences and determination of percent identity between two sequences can be accomplished using a computational algorithm, such as BLAST (basic local alignment search tool).

“Improve,” “increase,” “inhibit,” or “reduce”: As used herein, the terms “improve”, “increase”, “inhibit”, and “reduce”, and grammatical equivalents thereof, indicate qualitative or quantitative difference from a reference.

Isolated: As used herein, “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Operably linked: As used herein, “operably linked” refers to the association of at least a first element and a second element such that the component elements are in a relationship permitting them to function in their intended manner. For example, a nucleic acid regulatory sequence is “operably linked” to a nucleic acid coding sequence if the regulatory sequence and coding sequence are associated in a manner that permits control of expression of the coding sequence by the regulatory sequence. In some embodiments, an “operably linked” regulatory sequence is directly or indirectly covalently associated with a coding sequence (e.g., in a single nucleic acid). In some embodiments, a regulatory sequence controls expression of a coding sequence in trans and inclusion of the regulatory sequence in the same nucleic acid as the coding sequence is not a requirement of operable linkage.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation of an agent (e.g., a pharmaceutical agent), modifies bioavailability of an agent, or facilitates transport of an agent from one organ or portion of a subject to another. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer’s solution; ethyl alcohol; pH buffered solutions; polyesters, polycarbonates and/or polyanhydrides; and other non-toxic compatible substances employed in pharmaceutical formulations.

Pharmaceutical composition: As used herein, the term “pharmaceutical composition” refers to a composition in which an active agent is formulated together with one or more pharmaceutically acceptable carriers.

Promoter: As used herein, a “promoter” or “promoter sequence” can be a DNA regulatory region that directly or indirectly (e.g., through promoter-bound proteins or substances) participates in initiation and/or processivity of transcription of a coding sequence. A promoter may, under suitable conditions, initiate transcription of a coding sequence upon binding of one or more transcription factors and/or regulatory moieties with the promoter. A promoter that participates in initiation of transcription of a coding sequence can be “operably linked” to the coding sequence. In certain instances, a promoter can be or include a DNA regulatory region that extends from a transcription initiation site (at its 3′ terminus) to an upstream (5′ direction) position such that the sequence so designated includes one or both of a minimum number of bases or elements necessary to initiate a transcription event. A promoter may be, include, or be operably associated with or operably linked to, expression control sequences such as enhancer and repressor sequences. In some embodiments, a promoter may be inducible. In some embodiments, a promoter may be a constitutive promoter. In some embodiments, a conditional (e.g., inducible) promoter may be unidirectional or bi-directional. A promoter may be or include a sequence identical to a sequence known to occur in the genome of particular species. In some embodiments, a promoter can be or include a hybrid promoter, in which a sequence containing a transcriptional regulatory region can be obtained from one source and a sequence containing a transcription initiation region can be obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, NY, 1989).

Reference: As used herein, “reference” refers to a standard or control relative to which a comparison is performed. For example, in some embodiments, an agent, sample, sequence, subject, animal, or individual, or population thereof, or a measure or characteristic representative thereof, is compared with a reference, an agent, sample, sequence, subject, animal, or individual, or population thereof, or a measure or characteristic representative thereof. In some embodiments, a reference is a measured value. In some embodiments, a reference is an established standard or expected value. In some embodiments, a reference is a historical reference. A reference can be quantitative of qualitative. Typically, as would be understood by those of skill in the art, a reference and the value to which it is compared represents measure under comparable conditions. Those of skill in the art will appreciate when sufficient similarities are present to justify reliance on and/or comparison. In some embodiments, an appropriate reference may be an agent, sample, sequence, subject, animal, or individual, or population thereof, under conditions those of skill in the art will recognize as comparable, e.g., for the purpose of assessing one or more particular variables (e.g., presence or absence of an agent or condition), or a measure or characteristic representative thereof.

Regulatory Sequence: As used herein in the context of expression of a nucleic acid coding sequence, a regulatory sequence is a nucleic acid sequence that controls expression of a coding sequence. In some embodiments, a regulatory sequence can control or impact one or more aspects of gene expression (e.g., cell-type-specific expression, inducible expression, etc.).

Subject: As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. In some instances, a human subject can be interchangeably referred to as a “patient” or “individual.”

Therapeutic agent: As used herein, the term “therapeutic agent” refers to any agent that elicits a desired pharmacological effect when administered to a subject. In some embodiments, an agent is considered to be a therapeutic agent if it demonstrates a statistically significant effect across an appropriate population. In some embodiments, the appropriate population can be a population of model organisms or a human population. In some embodiments, an appropriate population can be defined by various criteria, such as a certain age group, gender, genetic background, preexisting clinical conditions, etc. In some embodiments, a therapeutic agent is a substance that can be used for treatment of a disease, disorder, or condition. In some embodiments, a therapeutic agent is an agent that has been or is required to be approved by a government agency before it can be marketed for administration to humans. In some embodiments, a therapeutic agent is an agent for which a medical prescription is required for administration to humans.

Therapeutically effective amount: As used herein, “therapeutically effective amount” refers to an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, or condition.

Unit dose: As used herein, the term “unit dose” refers to an amount administered as a single dose and/or in a physically discrete unit of a pharmaceutical composition. In many embodiments, a unit dose contains a predetermined quantity of an active agent, for instance a predetermined viral titer (the number of viruses, virions, or viral particles in a given volume). In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic moiety(s). For example, acceptable carriers (e.g., pharmaceutically acceptable carriers), diluents, stabilizers, buffers, preservatives, etc., can be included. It will be appreciated by those skilled in the art, in many embodiments, a total appropriate daily dosage of a particular therapeutic agent can include a portion, or a plurality, of unit doses, and can be decided, for example, by a medical practitioner within the scope of sound medical judgment. In some embodiments, the specific effective dose level for any particular subject or organism can depend upon a variety of factors including the disorder being treated and the severity of the disorder; activity of specific active compound employed; specific composition employed; age, body weight, general health, sex, and diet of the subject; time of administration, and rate of excretion of the specific active compound employed; duration of the treatment; drugs and/or additional therapies used in combination or coincidental with specific compound(s) employed, and like factors well known in the medical arts.

### BRIEF DESCRIPTION OF THE FIGURES

One or more of the figures submitted herein are better understood in color. Applicant considers the color versions of the drawings as part of the original submission and reserve the right to present color images of the drawings in later proceedings.

FIGS. 1A-1D. Ex vivo HSPC transduction study with HDAd-long-LCR. (FIG. 1A) Vector structure. The γ-globin gene under the control of a 21.5 kb β-globin LCR, a 1.6 kb β-globin promoter and a 3′HS1 region also derived from the β-globin locus. For RNA stabilization in erythroid cells a β-globin gene UTR was linked to the 3′ end of the γ-globin gene. The vector also contains an expression cassette for mgmt^{P140K} allowing for in vivo selection of transduced HSPCs and HSPC progeny. The γ-globin and mgmt expression cassettes are separated by a chicken globin HS4 insulator. The 32.4 kb LCR-γ-globin/mgtm transposon is flanked by inverted repeats (IRs) that are recognized by SB1 00x and by ftr sites that allow for circularization of the transposon by Flpe recombinase. (FIG. 1B) Experimental regimen. Bone marrow Lin⁻ cells from CD46-transgenic mice were transduced with HDAd-long-LCR and HDAd-SB at a total MOI of 500 vp/cell. After one day in culture, 1×10⁶ transduced cells/mouse were transplanted into lethally irradiated C57Bl/6 mice. At week 4, O⁶BG/BCNU treatment was started and repeated four times every two weeks. With each cycle, the BCNU concentration was increased from 5 mg/kg, to 7.5 mg/kg, to 10 mg/kg (twice). At week 20, mice were sacrificed. (FIG. 1C) Percentage of human γ-globin-positive peripheral red blood cells (RBC) measured by flow cytometry. Each symbol is an individual animal. (FIG. 1D) Representative flow cytometry data showing human γ-globin-expression in erythroid (Ter119⁺) bone marrow cells (lower panel) at week 20 after transplantation. The top panel shows a mouse transplanted with mock-transduced cells.

FIGS. 2A-2C. iPCR analysis of vector/chromosome junctions in bone marrow cells from animals at week 20 after transplantation. (FIG. 2A) Schematic of iPCR analysis. Five micrograms of genomic DNAs were digested with Sacl, re-ligated, and subjected to nested, inverse PCR with the indicated primers (see Materials and Methods). (FIG. 2B) Agarose gel electrophoresis of cloned plasmids containing integration junctions. Indicated bands were excised and sequenced. The chromosomal integration sites are shown below the gel. (FIG. 2C) Examples of junction sequences: 5′ end vector sequence, Sleeping beauty IR/DR sequence, integration junction (chr15, 6805206) SEQ ID NO: 1; 5′ end vector sequence, Sleeping beauty IR/DR sequence, integration junction (chrX, 16897322) SEQ ID NO: 2; 3′ end vector sequence, Sleeping beauty IR/DR sequence, integration junction (chr4, 10207667) SEQ ID NO: 3. The vector body and IR/DR sequences are designated in plain text and underlining, respectively. The chromosomal sequence is designated in bold text. The TA dinucleotides used by SB100x at the junction of the IR and chromosomal DNA are bracketed.

FIGS. 3A-3E. In vivo HSPC transduction with HDAd-long-LCR containing the 32.4 kb transposon and HDAd-short-LCR containing an 11.8 kb transposon. (FIG. 3A) Instead of the 21.5 kb HS1-HS5 LCR and 3′HS1 (FIG. 1A HDAd-short-LCR), this vector contains a 4.3 kb mini-LCR including the core regions of DNase hypersensitivity sites (HS) 1 to 4. (FIG. 3B) Treatment regimen. hCD46tg mice were mobilized and IV injected with the either HDAd-short-LCR + HDAd-SB or HDAd-long-LCR +HDAd-SB (2 times each 4x10¹⁰ vp of a 1:1 mixture of both viruses). Five weeks later, O⁶BG/BCNU treatment was started. With each cycle, the BCNU concentration was increased from 2.5 mg/kg, to 7.5 mg/kg, and 10 mg/kg. The O⁶BG concentration was 30 mg/kg in all three treatments. Mice were followed until week 20 when animals were sacrificed for analysis and Lin⁻ cell transplantation into secondary recipients. Secondary recipients were then followed for 16 weeks. In vivo HSPC transduced animals received immunosuppressive (IS) drugs to prevent immune responses against the human γ-globin and mgtm proteins. (FIG. 3C) Percentage of human γ-globin-positive cells in peripheral red blood cells (RBCs) measured by flow cytometry. Each symbol is an individual animal. In mice that were mock-transduced, less than 0.1% of cells were γ-globin-positive. (FIG. 3D) γ-globin protein chain levels measured by HPLC in RBCs at week 20 after in vivo HSPC transduction. Shown are the percentages of human γ-globin to mouse α-globin protein chains. (FIG. 3E) γ-globin mRNA levels measured by qRT-PCR in total blood at week 20 after in vivo HSPC transduction. Shown are the percentages of human γ-globin mRNA to mouse α-globin mRNA.

FIG. 4. Vector copy number per cell in bone marrow MNCs harvested at week 20 after in vivo HSPC transduction. The difference between the two groups is not significant.

FIGS. 5A-5D. Hematological parameters at week 20 after in vivo HSPC transduction. (FIG. 5A) White blood cells (WBC), neutrophils (NE), leukocytes (LY), monocytes (MO), eosinophils (EO), and basophils (BA). (FIG. 5B) Erythropoietic parameters. RBC: red blood cells, Hb: hemoglobin, MCV: mean corpuscular volume, MCH: mean corpuscular hemoglobin, MCHC: mean corpuscular hemoglobin concentration, RDW: red cell distribution width. The differences between the three groups were not significant. (FIG. 5C) Cellular bone marrow composition. (FIG. 5D) Colony-forming potential of bone marrow Lin⁻ cells. The differences between the groups were not significant in FIGS. 5A-5D. Data in panels of FIG. 5 show that in vivo HSPC transduction with HDAd short-LCR and/or long-LCR vectors do not affect hematopoiesis and cellular distribution in bone marrow.

FIG. 6. The localization of Nhel and Kpnl sites in the HDAd-globin vectors in relation to the Sleeping Beauty inverted repeated (IRs) is indicated. These enzymes cut close, but outside of the SB IR/DR and are used to decrease the background of unintegrated vectors. Remaining genomic DNA from bone marrow Lin⁻ cells was digested with Nhel and Kpnl, and after heat inactivation further digested with Nlalll. Nlalll is a 4-cutter and will create small DNA fragments. Digested DNA was then ligated with double stranded oligos with known sequence and compatible ends to the digested Nlalll fragments. Following heat-inactivation and clean-up, the linker-ligated product was used for linear amplification, which creates a single stranded (ss) DNA population primed from the SB left arm. The primer is biotinylated, so the ssDNAs can be collected with streptavidin beads. After extensive washing, ssDNA was eluted from the beads and subjected to further amplification by two rounds of nested PCR. PCR amplicons were gel purified, cloned, sequenced and mapped to the mouse genome sequences to mark the integration sites.

FIGS. 7A-7D. Analysis of vector integration sites in HSPCs. Genomic DNA isolated from bone marrow Lin- cells harvested at week 20 after in vivo transduction with HDAd-long-LCR +HDAd-SB. (FIG. 7A, on two pages) Chromosomal distribution of integration sites. Genome-wide Sleeping Beauty integrations. The integration sites are marked by vertical lines. (FIG. 7B) Examples of junction sequences: Sleeping beauty IR/DR sequence, integration junction (chr7, 79796094) SEQ ID NO: 4; Sleeping beauty IR/DR sequence, Integration junction (repeat region) SEQ ID NO: 5. IR/DR sequences are designated by underlining and bold text. The chromosomal sequence is designated in plain text. The TA dinucleotides used by SB100x at the junction of the IR and chromosomal DNA are bolded. (FIG. 7C) Genome-wide Sleeping Beauty integrations in relation to RefSeq annotation. Integration sites were mapped to the mouse genome and their location with respect to genes was analyzed. Shown is the percentage of integration events that occurred 1 kb upstream transcription start sites, 3′UTR of exons, protein coding sequences, introns, 3′UTRs, 1 kb downstream from 3′UTR, and intergenic. (FIG. 7D) Sleeping Beauty integration pattern compared to randomized control. Integration pattern in mouse genomic windows. The number of integrations overlapping with continuous genomic windows and randomized mouse genomic windows and size was compared. This shows that the pattern of integration is similar in continuous and random windows. Maximum number of integrations in any given window was not more than 3; with one integration per window having the higher incidence. Values represent means ± s.d. Data in panels of FIG. 7 shows a near-random integration pattern without a preference for genes.

FIGS. 8A-8E. Analysis of secondary recipients. Bone marrow Lin⁻ cells harvested at week 20 from in vivo transduced CD46tg mice were transplanted into lethally irradiated C57BI/6 mice. Secondary recipients were followed for 16 weeks. (FIG. 8A) Engraftment rates based on the percentage of CD46-positive PBMCs. The differences between the two groups were not significant. (FIG. 8B) Percentage of γ-globin-expressing peripheral blood RBCs measured by flow cytometry. The differences between the two groups are not significant. (FIG. 8C) Analysis of human γ-globin chains by HPLC in RBCs of secondary recipients. Shown is the percentage of human γ-globin to adult mouse α globin at weeks 4, 8, 12, and 16 after transplantation. * p<0.0001. Statistical analysis was performed using two-way ANOVA. (FIG. 8D) γ-globin mRNA levels in total blood cells. Shown are percentages of human γ-globin mRNA to mouse α and β-major globin mRNA. (FIG. 8E) γ-globin mRNA levels bone marrow MNCs at week 16 p.t. Shown are percentages of human γ-globin m-RNA to mouse α and β-major globin mRNA. The panels of FIGS. 8 and 9, individually or together, show that integration of the “32.4” kb transposon occurred in long-term repopulating cells; that the level of γ-globin expression from vector with long LCR increased over time compared to vector with short LCR, and that vector with long LCR provided a more stringent erythroid specificity of γ-globin expression.

FIGS. 9A-9C. Erythroid specificity of γ-globin expression in bone marrow of secondary recipients (week 16 after transplantation) (FIG. 9A) Percentage of γ-globin expressing erythroid (Ter119⁺ cells) in all bone marrow MNCs. (FIG. 9B) Erythroid specificity. Percentage of γ-globin+ cells in erythroid (Ter119⁺) and non-erythroid (Ter119⁻) cells. (FIG. 9C) Vector copy number (VCN) per cell in bone marrow MNCs harvested at week 20 after in vivo HSPC transduction. The difference between the two groups is not significant.

FIGS. 10A-10D. Hematological parameters in secondary recipients at week 16 after transplantation. (FIG. 10A) White blood cells. (FIG. 10B) Erythropoietic parameters. RBC: red blood cells, Hb: hemoglobin, MCV: mean corpuscular volume, MCH: mean corpuscular hemoglobin, MCHC: mean corpuscular hemoglobin concentration, RDW: red cell distribution width. The differences between the three groups were not significant. (FIG. 10C) Cellular bone marrow composition. (FIG. 10D) Colony-forming potential of bone marrow Lin⁻ cells.

FIGS. 11A-11C. In vitro studies with human CD34+ cells. (FIG. 11A) Schematic of the experiment. CD34+ cells were transduced with HDAd-long-LCR + HD-SB or HDAd-short-LCR + HDAd-SB and subjected to erythroid differentiation (ED). In vitro selection with O⁶BG-BCNU was started at day 5 of ED. At day 18 cells were analyzed by flow cytometry (FIG. 11B) and HPLC (FIG. 11C). Panels of FIG. 11 show in a human cell system that HDAd long-LCR vectors provide higher γ-globin expression after erythroid differentiation of transduced human HSCs/CD34+ cells.

FIGS. 12A-12B. In vivo HSC transduction in vector hCD46tg in mice: “long” vs “short” vectors LCR. (FIG. 12A) HDAd-long-LCR-y-globin/mgmt. vector and HDAd-short-LCR-y-globin/mgmt. vector. (FIG. 12B) In vivo transduction of vector Hbb^{th3}/CD46 in mice. Group 1 shows the in vivo transduction of HDAd-long-LCR-y-globin/mgmt plus HDAd-SB/Flpe in 7 mice. Group 2 shows the in vivo transduction of HDAd-short-LCRy-globin/mgmt plus HDAd-SB/Flpe in 3 mice. Only three selection cycles were needed for O⁶BG, BCNU.

FIG. 13. Thbb mice test (W6). The graphical results show no difference and almost no human γ-globin expression among the mice when transduced with Long LCR vectors verses Short LCR vectors. On two pages.

FIG. 14. Thbb mice test (W8). The graphical results show a difference among the mice when transduced with Long LCR vectors verses Short LCR vectors, however, it is unclear if Short LCR virus were dead in the mice. On two pages.

FIG. 15. Graphic depiction showing the percentage of human γ-globin expressing RBC in mice. The graph illustrates 100% marking after only three cycles of in vivo selection.

FIG. 16. Graphic depiction of HPLC showing the relative human γ-globin to mouse HBA (week 10). The graph shows significantly higher γ-globin levels for long LCR compared to short LCR.

FIG. 17. Graphical depiction of example Week 10 blood HPLC of mouse #57 containing a Long LCR vector.

FIGS. 18A-18D. Human γ-globin expression after in vivo HSC gene therapy of Hbb^{th3}/CD46 mice with HDAd-short-LCR and HDAd-long-LCR. (FIG. 18A) Treatment regimen. In contrast to FIGS. 3A-3E, FIGS. 18A-18D show results within thalassemic Hbb^{th3}/CD46 mice. (FIG. 18B) Percentage of human γ -globin-positive cells in peripheral red blood cells (RBCs) measured by flow cytometry. Each symbol is an individual animal. (FIG. 18C) γ-globin protein chain levels measured by HPLC in RBCs at week 18 after in vivo HSPC transduction. Shown are the percentages of human γ-globin to mouse α -globin protein chains. (FIG. 18D) Representative chromatograms of an untreated Hbb^{th3}/CD46 mouse (left panel) and a mouse at week 21 after treatment. Mouse α- and β-chains as well the added human γ-globin are indicated. Data in panels of FIG. 18 show that with long-LCR HDAd vectors 100% GRP marking can be achieved with less intense and/or fewer rounds and/or lower doses of in vivo selection. The γ-globin expression levels are in a range expected to provide effective therapy (at or above 20%).

FIG. 19. Micrographs showing the normalized erythrocyte morphology of C57BL6 (Normal mice) and the Townes SCA mice, before treatment and at week 10 after treatment-long LCR.

FIG. 20. Micrographs showing the normalized erythropoiesis (reticulocyte count) for Townes mice, before treatment, and Townes mice at week 10, after treatment (long LCR).

FIGS. 21A-21C. Phenotypic correction. (FIGS. 21A, 21B) Blood cell morphology with left panel displaying blood smears stained with Giemsa stain and right panels displaying blood smears stained with May-Grünwald stain. Remnants of nuclei and cytoplasm in reticulocytes results in purple staining. (FIG. 21A) Comparison before and at week 14. (FIG. 21B) Comparison of Giemsa stain and reticulocytes for CD46tg, Hbb^{th3}/CD46 mice before, Hbb^{th3}/CD46 mice with HDAd-long-LCR at week 18, and Hbb^{th3}/CD46 mice with HDAd-long-LCR at week 21. (FIG. 21C) Bone marrow cytospins. Visible is a bac k-shift in erythropoiesis with pro-erythroblast predominance in treated. The scale bar is 20 µm. Data in panels of FIG. 21 show that blood cell morphology is normalized after in vivo HSC gene therapy with HDAd long-LCR vectors.

FIG. 22. Hematological parameters before and after in vivo HSC gene therapy of Hbb^{th3}/CD46⁺ mice. Hbb^{th3}/CD46⁺ mice display a thalassemia intermedia phenotype. Mice were treated with adenoviral donor vectors including a γ-globin nucleic acid sequence operably linked to, among other things, either a long LCR or a short LCR. At weeks 1 and 10 after treatment, mice were sampled. FIG. 22 shows a graphical depiction of normalized erythrocyte parameters of WBC, RBC, Hb, HCT, MCV, MCH, MCHC, and RDW from samples from mice treated with long LCR vectors, mice treated with short LCR vectors, and control CD46tg, at Week 1 (top panel) and Week 10 (bottom panel).

FIGS. 23A, 23B. Hematological parameters before and after in vivo HSC gene therapy of Hbb^{th3}/CD46⁺ mice. Hbb^{th3}/CD46⁺ mice display a thalassemia intermedia phenotype. Mice were treated with adenoviral donor vectors including a γ-globin nucleic acid sequence operably linked to, among other things, either a long LCR or a short LCR. At week 18 after treatment, mice were sacrificed and sampled. Percentage of reticulocytes was counted on blood smears (FIG. 23A; Reticulocyte counts). Hematological parameters at week 18 post in vivo transduction were indistinguishable from their control CD46tg counterparts, suggesting complete phenotypic correction, including a normalization in white and red blood cell counts as well as erythroid cell features (Hb, HCT, MHCH, and RDW) (FIG. 23B; Hematological parameters).

FIGS. 24A, 24B. Phenotypic correction of extramedullary hematopoiesis in spleen and liver. (FIG. 24A) Spleen size at sacrifice (wk21) The top two panels show representative spleen images. The bottom panel is a dot plot summarizing those results. Each symbol represents an individual animal. Data are presented as means ± standard error of mean (SEM). * p ≤ 0.05. Statistical analysis was performed using one-way ANOVA. (FIG. 24B). Extramedullary hemopoiesis by hematoxylin/eosin staining in liver and spleen sections. Clusters of erythroblasts in the liver and megakaryocytes in the spleen of Hbb^{th3}/CD46 mice are indicated by black arrows. The scale bars are 20 µm.

FIG. 25. Phenotypic correction of hemosiderosis in spleen and liver. Iron deposition is shown by Perl’s staining as cytoplasmic blue pigments of hemosiderin in spleen and liver sections. The scale bars are 20 µm. (Exp: 2.24 ms, gain: 4.1x, saturation: 1.50, gamma: 0.60).

FIGS. 26A-26C. Analysis of bone marrow at sacrifice (week 21). Bone marrow was harvested at week 21 after in vivo HSC transduction of Hbb^{th3}/CD46tg mice. (FIG. 26A) Vector copy number per cell in bone marrow MNCs. The difference between the two groups is not significant but could become significant if analyzed with greater sample size. (FIGS. 26B, 26C) Erythroid specificity of γ-globin expression. (FIG. 26B) Percentage of γ-globin expressing erythroid (Ter119⁺) and non-erythroid (Ter119⁻) cells. *p<0.05. Statistical analyses were performed using two-way ANOVA.

FIG. 27. Extramedullary hemopoiesis by hematoxylin/eosin staining in liver and spleen sections from CD46tg and CD46^{+/+}/Hbb^{th-3} mice prior to administration of an adenoviral donor vector. Iron deposition is shown by Perl’s staining as cytoplasmic blue pigments of hemosiderin in spleen.

FIG. 28. Schematic of experimental design for comparison of integration SB100x transposase efficacy using different inverted repeats (IR). Three plasmids were used in which the mgmt./GFP transposon payload is flanked by (i) pT0 ITRs; (ii) pT2 ITRs; or (iii) pT4 ITRs, which plasmids were otherwise identical. 293 cells were transfected with the three plasmids including the mgmt./GFP transposon payload, with or without a support plasmid encoding pSB1 00x. Cells were cultured for 17 days with or without selection. Culture samples were drawn on days 3, 12, and 17 for cells not under selection, and on day 17 for cells under selection by a single addition of 50 µM O⁶BG/BCNU on day 3.

FIG. 29. Percentage of GFP-expressing 293 cells on days 12 and 17 of culture for cells cultured with or without SB1 00x plasmid for each of the T0, T2, and T4 plasmids.

FIG. 30. Percentage of GFP-expressing 293 cells on day 17 of culture for cells under selection with O⁶BG/BCNU for cells cultured with or without SB100x plasmid for each of T0, T2, and T4 plasmids.

FIG. 31. Schematic of a nucleic acid (pWEAd5-PT4-LCR-globin-mgmt) that includes a 31.776 kb transposon payload (integration cassette). The schematic is divided into two overlapping portions for ease of presentation, the relationship of which portions will be evident to those of skill in the art. The schematic provides the transposon payload in a circularized plasmid context. Those of skill in the art will appreciate that the transposon payload can be readily utilized, using techniques of molecular biology, in other contexts, e.g., in a viral vector genome. The transposon payload is flanked by transposon IRs (in particular Sleeping Beauty IRs), which are in turn flanked by recombinase direct repeats (DRs, in particular FRT DRs). The transposon includes: (i) a gamma-globin coding sequence operably linked with a beta promoter, a long LCR including HS1-HS5, and a 3′HS1 and (ii) an MGMT^{P140K} selection cassette in which an MGMT^{P140K} coding sequence is operably linked with an Ef1 a promoter.

FIG. 32. Schematic of a nucleic acid (HDAd5-PT4-long LCR globin-rhMGMT) that includes a 31.772 kb transposon payload (integration cassette). The schematic is divided into two overlapping portions for ease of presentation, the relationship of which portions will be evident to those of skill in the art. The schematic provides the transposon payload in a circularized plasmid context. Those of skill in the art will appreciate that the transposon payload can be readily utilized, using techniques of molecular biology, in other contexts, e.g., in a viral vector genome. The transposon payload is flanked by transposon IRs (in particular Sleeping Beauty IRs), which are in turn flanked by recombinase DRs (in particular FRT DRs). The transposon includes: (i) a gamma-globin coding sequence operably linked with a beta promoter, a long LCR including HS1 -HS5, and a 3′HS1 and (ii) an MGMT^{P140K} selection cassette in which an MGMT^{P140K} coding sequence is operably linked with an Ef1a promoter.

FIG. 33. Schematic of a nucleic acid (HDAd-Ad5-PT4-LCR-hACE2/mgmt) that includes a 13.173 kb transposon payload (integration cassette). The schematic is divided into two overlapping portions for ease of presentation, the relationship of which portions will be evident to those of skill in the art. The schematic provides the transposon payload in a circularized plasmid context. Those of skill in the art will appreciate that the transposon payload can be readily utilized, using techniques of molecular biology, in other contexts, e.g., in a viral vector genome. The transposon payload is flanked by transposon IRs (in particular Sleeping Beauty IRs), which are in turn flanked by recombinase DRs (in particular FRT DRs). The transposon includes: (i) a recombinant human ACE2 coding sequence operably linked with a beta promoter, and a long LCR including HS1-HS4 and (ii) an MGMT^{P140K} selection cassette in which an MGMT^{P140K} coding sequence is operably linked with an Ef1a promoter.

FIG. 34. Schematic of a nucleic acid (pWEHCB-microLCR-globin/mgmt) that includes a 12.169 kb transposon payload (integration cassette). The schematic provides the transposon payload in a circularized plasmid context. Those of skill in the art will appreciate that the transposon payload can be readily utilized, using techniques of molecular biology, in other contexts, e.g., in a viral vector genome. The transposon payload is flanked by transposon IRs (in particular Sleeping Beauty IRs), which are in turn flanked by recombinase DRs (in particular FRT DRs). The transposon includes: (i) a gamma globin coding sequence operably linked with a beta promoter, and a long LCR including HS1-HS4 and (ii) an MGMT^{P140K} selection cassette in which an MGMT^{P140K} coding sequence is operably linked with an Ef1a promoter.

FIG. 35. Schematic of a nucleic acid (pWEHCA-Faconi-GFP) that includes a 9.382 kb transposon payload (integration cassette). The schematic provides the transposon payload in a circularized plasmid context. Those of skill in the art will appreciate that the transposon payload can be readily utilized, using techniques of molecular biology, in other contexts, e.g., in a viral vector genome. The transposon payload is flanked by transposon IRs (in particular Sleeping Beauty IRs), which are in turn flanked by recombinase DRs (in particular FRT DRs). The transposon includes: (i) a FancA coding sequence operably linked with a pgk promoter and (ii) a GFP coding sequence operably linked with an Ef1a promoter.

FIG. 36. Schematic of a nucleic acid (pHCA-T4-rhMGMT-GFP) that includes a 5.490 kb transposon payload (integration cassette). The schematic provides the transposon payload in a circularized plasmid context. Those of skill in the art will appreciate that the transposon payload can be readily utilized, using techniques of molecular biology, in other contexts, e.g., in a viral vector genome. The transposon payload is flanked by transposon inverted repeats (IRs, in particular Sleeping Beauty IRs), which are in turn flanked by recombinase direct repeats (DRs, in particular FRT DRs). The transposon includes: (i) a GFP coding sequence operably linked with a PGK promoter and (ii) an MGMT^{P140K} selection cassette in which an MGMT^{P140K} coding sequence is operably linked with an EF1 a promoter.

FIG. 37. Schematic of a nucleic acid that includes a 3.797 kb transposon payload (integration cassette). The schematic provides the transposon payload in a circularized plasmid context. Those of skill in the art will appreciate that the transposon payload can be readily utilized, using techniques of molecular biology, in other contexts, e.g., in a viral vector genome. The transposon payload is flanked by transposon inverted repeats (IRs, in particular Sleeping Beauty IRs), which are in turn flanked by recombinase direct repeats (DRs, in particular FRT DRs). The transposon includes: (i) a GFP coding sequence and (ii) an MGMT^{P140K} coding sequence, operably linked with an EF1 a promoter.

FIG. 38. Schematic of a nucleic acid (pBHCA-PT0-EF1a-mgmt/GFP) that includes a 3.709 kb transposon payload (integration cassette). The schematic is divided into two overlapping portions for ease of presentation, the relationship of which portions will be evident to those of skill in the art. The schematic provides the transposon payload in a circularized plasmid context. Those of skill in the art will appreciate that the transposon payload can be readily utilized, using techniques of molecular biology, in other contexts, e.g., in a viral vector genome. The transposon payload is flanked by transposon inverted repeats (IRs, in particular Sleeping Beauty IRs), which are in turn flanked by recombinase direct repeats (DRs, in particular FRT DRs). The transposon includes: (i) an eGFP coding sequence and (ii) an MGMT^{P140K} coding sequence, operably linked with an EF1a promoter.

FIG. 39. Schematic of a nucleic acid (pHCA(Ad35)-PT4-EF1a-mgmt/GFP) that includes a 3.547 kb transposon payload (integration cassette). The schematic is divided into two overlapping portions for ease of presentation, the relationship of which portions will be evident to those of skill in the art. The schematic provides the transposon payload in a circularized plasmid context. Those of skill in the art will appreciate that the transposon payload can be readily utilized, using techniques of molecular biology, in other contexts, e.g., in a viral vector genome. The transposon payload is flanked by transposon inverted repeats (IRs, in particular Sleeping Beauty IRs), which are in turn flanked by recombinase direct repeats (DRs, in particular FRT DRs). The transposon includes: (i) a GFP coding sequence and (ii) an MGMT^{P140K} coding sequence, operably linked with an EF1a promoter.

FIG. 40. Schematic of a nucleic acid (pHCA-Ad5-PT4-Ef1a-mgmt/GFP) that includes a 3.543 kb transposon payload (integration cassette). The schematic is divided into two overlapping portions for ease of presentation, the relationship of which portions will be evident to those of skill in the art. The schematic provides the transposon payload in a circularized plasmid context. Those of skill in the art will appreciate that the transposon payload can be readily utilized, using techniques of molecular biology, in other contexts, e.g., in a viral vector genome. The transposon payload is flanked by transposon inverted repeats (IRs, in particular Sleeping Beauty IRs), which are in turn flanked by recombinase direct repeats (DRs, in particular FRT DRs). The transposon includes: (i) a GFP coding sequence and (ii) an MGMT^{P140K} coding sequence, operably linked with an EF1a promoter.

FIG. 41. Schematic of a nucleic acid (pHCA(Ad35)-PT4-EF1a-mgmt) that includes a 2.781 kb transposon payload (integration cassette). The schematic is divided into two overlapping portions for ease of presentation, the relationship of which portions will be evident to those of skill in the art. The schematic provides the transposon payload in a circularized plasmid context. Those of skill in the art will appreciate that the transposon payload can be readily utilized, using techniques of molecular biology, in other contexts, e.g., in a viral vector genome. The transposon payload is flanked by transposon inverted repeats (IRs, in particular Sleeping Beauty IRs), which are in turn flanked by recombinase direct repeats (DRs, in particular FRT DRs). The transposon includes: an MGMT^{P140K} selection cassette in which an MGMT^{P140K} coding sequence is operably linked with an EF1a promoter.

FIG. 42. Schematic of a nucleic acid (pHCA-T4-Ef1a-rhMGMT) that includes a 2.777 kb transposon payload (integration cassette). The schematic provides the transposon payload in a circularized plasmid context. Those of skill in the art will appreciate that the transposon payload can be readily utilized, using techniques of molecular biology, in other contexts, e.g., in a viral vector genome. The transposon payload is flanked by transposon inverted repeats (IRs, in particular Sleeping Beauty IRs), which are in turn flanked by recombinase direct repeats (DRs, in particular FRT DRs). The transposon includes: an MGMT^{P140K} selection cassette in which an MGMT^{P140K} coding sequence is operably linked with an EF1 a promoter.

FIG. 43. Schematic of a nucleic acid (pHCA-Ad5-PT4-Ef1a-mgmt) that includes a 2.751 kb transposon payload (integration cassette). The schematic is divided into two overlapping portions for ease of presentation, the relationship of which portions will be evident to those of skill in the art. The schematic provides the transposon payload in a circularized plasmid context. Those of skill in the art will appreciate that the transposon payload can be readily utilized, using techniques of molecular biology, in other contexts, e.g., in a viral vector genome. The transposon payload is flanked by transposon inverted repeats (IRs, in particular Sleeping Beauty IRs), which are in turn flanked by recombinase direct repeats (DRs, in particular FRT DRs). The transposon includes: an MGMT^{P140K} selection cassette in which an MGMT^{P140K} coding sequence is operably linked with an EF1 a promoter.

### DETAILED DESCRIPTION

The present disclosure includes, among other things, adenoviral vectors, adenoviral vector genomes, and combinations and uses thereof. Adenoviral vectors and adenoviral vector genomes of the present disclosure can include transposon payload of up to, e.g., 20, 25, 30, or even more than 30 kb, and moreover in various embodiments successfully integrate such large transposon payloads into the genomes of host cells. As those of skill in the art will appreciate, vector integration capacity, in and of itself, is one critically important feature a gene therapy system, at least in part because integration capacity limits the length and/or complexity of therapeutic payloads. Accordingly, the methods and compositions provided herein provide, among other things, a platform for effective gene therapy using adenoviral vectors that permits transpositional integration of nucleic acid payloads of e.g., 20, 25, 30, or even more than 30 kb, into host cell genomes. As those of skill in the art will appreciate from the present disclosure, and as is exemplified by various embodiments herein, such integration capacity permits engineering of therapeutic payloads with a greater complexity and diversity than possible with various previous systems.

The methods and compositions of the present disclosure overcome certain previously understood constrains on integration capacity. Certain such constraints are associated with viral vector type. For instance, lentiviral vector payload capacity is about 9 kb, retroviral payload capacity is about 8 kb, and adeno-associated virus (AAV) payload capacity is about 5 kb. Other such constraints were previously understood to be inherent to transposition. For instance, studies had shown that integration of transposons was length dependent-- as length increases, ability to transpose rapidly declines, which phenomenon is sometimes referred to in the art as “length-dependence.” In view of these extant expectations, the discovery that compositions and methods disclosed herein break the previously understood limits of adenoviral transpositional integration capacity was a surprising result revealed by the present disclosure and the Examples provided herein. To the knowledge of the present inventors, this work represents the first demonstration that methods and compositions as provided herein can integrate transposon payloads of various certain sizes disclosed herein. This discovery is exemplified, for instance, by integration of transposon payloads including large regulatory regions (locus control regions, or “LCRs”) for improved transgene expression. However, for the avoidance of any doubt, those of skill in the art will appreciate that such exemplification is representative of the more general discovery of the high transpositional integration capacity of adenoviral compositions and methods provided herein, and the significance thereof in various fields including in particular the field of gene therapy.

Aspects of the current disclosure are now described in more supporting detail as follows: (I) Viral Vector Payload Integration into Target Cell Genomes; (II) Types of Large Payloads; (III) Long LCRs; (IV) Coding Sequences Operably linked with Long LCR; (V) Transposases; (VI) Regulatory Components; (VII) Vectors; (VIII) Formulations; (IX) Applications; (X) Exemplary Embodiments; (XI) Experimental Example(s); and (XII) Closing Paragraphs.

### (I) Viral Vector Payload Integration Into Target Cell Genomes

Gene therapy often requires integration of a desired nucleic acid payload into the genome of a target cell. In view of the diversity of conditions that may be treated by various gene therapies, many strategies for design of nucleic acid payloads have been conceived. However, in practice, delivery of therapeutic payloads has been limited in many contexts by the difficulty of integrating large payloads into target cell genomes. For instance, the lentiviral vector payload capacity is about 9 kb, the retroviral payload capacity is about 8 kb, and the adeno-associated virus (AAV) payload capacity is about 5 kb. Considering existing interest in payloads capable of expressing large genes, utilizing large human regulatory sequences, and/or expressing multiple genes, these are substantial limitations. Moreover, as is well appreciated by those of skill in the art, each viral platform is associated with a diversity of different characteristics that render each uniquely more or less suitable for various uses, which factors can include, without limitation, recipient immune responses (e.g., inflammation and/or interaction with pre-existing antibodies), difficulty of vector production, efficacy of cell transduction, efficacy of payload integration, transgene expression characteristics, cell types targeted, risk of genotoxicity (e.g., oncogenesis), and others, any or all of which may be uniquely weighed by researchers and medical practitioners in various contexts. The present disclosure recognizes that efficiency of transposon payload integration using certain known compositions and methods in one or more systems is dependent on one or more of target cell type, plasmid backbone, and/or transposon length, and that certain such dependencies are reduced or eliminated in at least certain compositions and methods of the present disclosure, e.g., compositions and methods including an adenoviral genome including a transposon payload flanked by SB inverted repeats (e.g., for transposition by an SB100x transposase or another SB transposase, e.g., in human subject cells, e.g., hematopoietic stem cells and/or in an in vivo therapy).

Adenoviral vectors are among the most commonly utilized gene therapy vectors. For example, according to at least some reports, adenoviral vectors are the most commonly employed vector for cancer gene therapy. Indeed, more than 400 gene therapy trials have been initiated and/or completed using human Ad vectors, e.g., for vaccine use, therapeutic transgene introduction, and/or cancer treatment. Various advantages of adenoviral vectors that contribute to, and/or are at least in part responsible for, the prevalence of adenoviral vectors in gene therapy are known in the art. Nevertheless, even with commonly used vectors, gene therapy remains a difficult challenge, at least in part because long-term phenotypic correction requires sufficiently efficient and sufficiently stable integration and expression of therapeutic transgenes.

Although some adenoviral vectors are known to have a high cloning capacity of up to about 36-37 kb, the ability to physically generate a vector carrying a large payload does not reflect the ability of that vector to efficiently mediate integration of the payload into a target cell genome. In fact, adenoviral vector genomes, which typically are linear, double-stranded DNA genomes of 26-45 kb (e.g., about 36 kb for Ad5), do not typically naturally integrate into host cell genomes. To the contrary, adenoviral vectors are characterized by episomal maintenance of viral genomes in host cells. While episomal maintenance minimizes risk of insertional effects, episomal genomes are often insufficiently retained by target cells and target cell progeny, among other difficulties known to those of skill in the art. For at least these reasons, efforts have been made to produce adenoviral vectors that, unlike their natural counterparts, are engineered for integration into host cell genomes. These approaches, too, have not been without challenges. For instance, one problem with certain integrating adenoviral vectors has been integration site preferences characterized by genotoxic effects.

One means of engineering adenoviral vectors that integrate a payload into a host cell genome has been to produce integrating viral hybrid vectors. Integrating viral hybrid vectors combine genetic elements of a vector that efficiently transduces target cells with genetic elements of a vector that stably integrates its vector payload. Integration elements of interest, e.g., for use in combination with adenoviral vectors, have included those of bacteriophage integrase PHiC31, retrotransposons, retrovirus (e.g., LTR-mediated or retrovirus integrate-mediated), zinc-finger nuclease, DNA-binding domain-retroviral integrase fusion proteins, AAV (e.g., AAV-ITR or AAV-Rep protein-mediated), and Sleeping Beauty (SB) transposase.

Like the vectors themselves, the integration systems of integrating viral hybrid vectors are subject to their own unique advantages and disadvantages, including characteristic positional integration patterns and payload capacities. Studies had shown, for example, that integration of transposons was length dependent; as length increases, ability to transpose rapidly declines, which phenomenon is sometimes referred to in the art as “length-dependence.” In the case of SB transposase, studies had shown that SB transposon efficacy decreased by 30% for each added 1 kb of transposon (payload) length and was lost entirely above about 9 kb. While some studies indicated that a small fraction of SB transposon integration was retained up to at least about 10 kb, evidence demonstrated that larger SB transposons would not efficiently integrate relative to smaller counterparts. Certain SB systems modified to enhance integration efficacy also suffered from significant length-dependent effects with substantially reduced transposon integration levels (Turchiano et al., PLOS One, 9: e112712, 2014).

The present disclosure provides, among other things, the present inventors surprising discovery that transposon payloads of up to at least about 30 kb to about 35 kb could be integrated into host cell genomes with sufficient efficacy for therapeutic use. In various embodiments, the present disclosure provides vectors, genomes, and systems for integration of a large payload (e.g., up to at last about 30 kb to about 35 kb) that include an adenoviral genome including a transposon payload flanked by SB inverted repeats, which are in turn flanked by FRT recombination sites, such that the genome or a portion thereof including the transposon payload is circularized in the presence of recombinase, which the present inventors have discovered can integrate the large transposon payload into a target cell genome in the presence of an SB transposase. The present disclosure further provides that such compositions are sufficiently efficient, e.g., for integration and transgene expression, to achieve in vivo therapy. These remarkable findings, which contrasts sharply with prior notions of length dependence and integration efficacy, open the door to therapeutic and research uses of adenoviral vectors previously thought unachievable.

### (II) Types of Large Payloads

In particular embodiments, the invention disclosed herein facilitates the delivery and integration of large transposon payloads. The large payloads include coding sequences linked to long LCR, including for instance those that are described herein. In particular embodiments, payloads are at least 10 kb. In particular embodiments, payloads are at least 10 kb, 15 kb, 20 kb, 25 kb, 30 kb, 35 kb, 40 kb, or more. In particular embodiments, the payload has a length of 10 kb-35 kb, 10 kb-30 kb, 15 kb-35 kb, 15 kb-30 kb, 20 kb-35 kb, or 20 kb-30 kb. In particular embodiments, the payload has a length of 10 kb-32.4 kb, 15 kb-32.4 kb, or 20 kb-32.4 kb. In particular embodiments, payloads encode a single long (large) protein. In particular embodiments, payloads encode multiple proteins; for instance, two or more proteins, such as two, three, four, or five proteins or more. In embodiments wherein the payload encodes multiple proteins, any individual protein so encoded need not be independently considered “large” or “long”; rather, it is understood that the entire payload carried by the adenoviral vector is “large”, even if it contains a number of smaller individual protein encoding sequences. In particular embodiments, payloads include long LCR.

### (III) Long LCRs

The ability to integrate large payloads into host cell genomes opens the door to integration of constructs previously thought too large for effective therapeutic use. Beyond the immediately evident general utility of being able to integrate large payloads, one category of large payloads includes payloads that include a Long Locus Control Region (or Long LCR). In some instances, regulatory regions larger than those accommodated by at least certain existing vector systems for gene therapy, such as lentiviral and AAV systems, are useful for achieving therapeutically effective transgene expression from a payload and/or increase the level of expression (e.g., in the number or frequency of production of mRNAs encoding a transgene expression product and/or of a transgene expression product encoded by the transgene) and/or specificity of expression (e.g., in the timing and/or cell or tissue specificity of expression of expression).

Without wishing to be bound by any particular scientific theory, the human genome is organized three dimensional structures that include long-range direct and/or indirect interactions between regulatory regions (such as transcription factor binding sites and the coding regions they control expression of), e.g., through loop forming. In many instances, these long-range interactions occur in the context of topologically associating domains (TADs). TADs are considered functional units of chromosome organization that can facilitate the interaction of enhancers with other regulatory regions to control transcription. TADs are demarcated by boundaries, which boundaries are thought to restrict the search space of enhancers and promoters and to prevent unwanted regulatory contacts to be formed. TAD boundaries, at both side of these domains, are conserved between different mammalian cell types and even across species.

Because of their important role in the genome, and particularly their role in organizing nucleic acid sequences and proteins that contribute to gene and transgene expression, TADs can be used to increase the safety and/or efficacy of gene therapy. TADs themselves are too large for inclusion in any existing viral vectors. The median size of TAD is 880 kb. However, certain functional elements present within TADs that capture some or all of the gene or transgene expression effects of TADs have been identified and are of sizes suitable for inclusion in adenoviral vectors disclosed herein, though in many instances remain too large for inclusion in certain other vectors such as lentiviral and AAV vectors. In some instances, a regulatory sequence including one or more nucleic acid sequences of a TAD can be referred to as an LCR. LCRs have been engineered to have various length, e.g., in some instances to have a relatively short length for inclusion in vectors with relatively small payload capacities such as lentiviral or AAV vectors. However, without wishing to be bound by any particular theory, those of skill in the art appreciate that longer sequences have a greater capacity to confer to associated genes or transgenes the advantageous expression effects of endogenous sequences from which, in whole or in part, they are derived or upon which, in whole or in part, their sequences are based. Thus, some LCRs have been engineered to have a relatively short length, e.g., of 5 kb or less, 6 kb or less, 7 kb or less, 8 kb or less, or 9 kb or less. By contrast, the present disclosure recognizes that Long LCRs (e.g., regulatory sequences of 9 kb or more, 10 kb or more, 11 kb or more, 12 kb or more, 13 kb or more, 14 kb or more 15 kb or more, 20 kb or more, 25 kb or more, or 30 kb or more) can be integrated into host cell genomes using vectors, genomes, and methods provided herein. In various embodiments, the Long LCRs include regulatory sequences with range of lengths having a lower bound selected from any of 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, and 30 kb, and an upper bound selected from any of 30 kb, 31 kb, 32 kb, 33 kb, 34 kb, 35 kb, 36 kb, 37 kb, 38 kb, 39 kb, and 40 kb. Long LCRs can also have any length of any LCR provided herein, which such length can be regarded in various embodiments as a lower bound or upper bound.

Examples of LCRs include those shown in Table 1. Except as otherwise indicated or as would be clear to those of skill in the art, the reference genome is a GRCh38 reference genome such as GRCH38/hg38 or GRCh38.p13.

TABLE 1:

| | |
|---|---|
| LCR | Exemplary Tissue Expression |
| β-Globin LCR | Erythrocytes |
| Immunoglobulin Heavy Chain LCR | B cells |
| T Cell Receptor a/δ LCR | T cells |
| Adenosine Deaminase LCR | Enriched in blood, intestine, and lymphoid tissue |
| Apolipoprotein E/C-1 LCR | Adrenal gland, liver |
| Th2 Cytokine LCR | Th2 cells |
| CD2 LCR | T cells |
| S100β LCR | Brain astrocytes |
| Growth Hormone LCR | Pituitary gland |
| Apolipoprotein B LCR | Intestine, liver |
| β Myosin Heavy Chain LCR | Heart muscle, skeletal muscle |
| MHC Class I HLA-B7 LCR | All cells |
| Keratin 18 LCR | Epithelial cells |
| MHC Class I HLA G LCR | All cells |
| Complement Component C4A/B LCR | Liver |
| Red and Green Visual Pigment LCR (OPSIN LCR) | Cone photoreceptors |
| CD4 LCR | Cd4+ t cells |
| α-Lactalbumin LCR | Mammary glands |
| Desmin LCR | Heart muscle, skeletal muscle, smooth muscle |
| CYP19/aromatase LCR | Multiple tissues |
| C-fes Proto-Oncogene LCR | Myeloid cells including macrophages and neutrophils |
| α-globin locus control region | Erythrocytes |
| nuclear factor, erythroid 2 like 1 (NFE2L1) LCR | Erythrocytes |

The β-globin LCR is exemplary of at least some LCRs in at least several respects. For example, like many other LCRs, the β-globin LCR enhances expression (e.g., increased transcription, increased translation, and/or increased cell or tissue specificity) of operably linked genes or transgenes and includes DNAse hypersensitive (HS) regions understood by those of skill in the art to mediate the expression effects of the LCR. In addition, like many other LCRs, the β-globin LCR can be utilized in whole or in part, e.g., in that it can be utilized in nucleic acids that include a β-globin LCR sequence that includes all of the β-globin LCR HS regions (HS1-HS5) or includes a subset of the β-globin LCR HS regions (e.g., HS1-HS4).

An exemplary nucleic acid sequence for the Homo sapiens β-globin region on chromosome 11 is provided at GenBank Accession Number NG_000007. A β-globin long LCR can, in some instances, be or include a sequence located 6 to 22 kb 5′ to the first (embryonic) globin gene in the locus. A β-globin long LCR can include 5 DNAse I hypersensitive sites, 5′HSs 1 to 5. Li et al., Blood, 100(9):3077-3086, 2002. NG_000007 provides the location of the restriction sites that delineate the DNAse I hypersensitive sites HS1, HS2, HS3, and HS4 within the Locus Control Region (e.g., the SnaBl and BstXl restriction sites of HS2, the Hindlll and BamHl restriction sites of HS3, and the BamHl and Banll restriction sites of HS4), and is incorporated herein by reference in its entirety and particularly with respect to hyper sensitive site positions. The sequence and position of HS1 is described, for example, by Pasceri et al., Ann NY Acad. Sci. 1998; 850:377-381; Pasceri et al., Blood. 92:653-663, 1998; and Milot et al., Cell. 87:105-114, 1996. In particular embodiments, the HS2 region extends from position 16,671 to 17,058 of the Locus Control Region. The SnaBl and BstXl restriction sites of HS2 are located at positions 17,093 and 16,240, respectively. The HS3 region extends from position 12,459 to 13,097 of the Locus Control Region. The BamHl and Hindlll restriction sites of HS3 are located at positions 12,065 and 13,360, respectively. The HS4 region extends from position 9,048 to 9,713 of the Locus Control Region. The BamHl and Banll restriction sites of HS4 are located at positions 8,496 and 9,576 respectively.

Particular embodiments disclosed herein utilize mini-portions of the β-globin LCR. Mini-portions include less than all 5 HS regions, such as HS1, HS2, HS3, HS4, and/or HS5, so long as the LCR does not include all 5 segments of the β-globin LCR. The 4.3 kb HS1-HS4 LCR utilized in Example 1 of the disclosure provides one example of a mini-LCR. Other mini-LCR can include, for example, HS1, HS2, and HS3; HS2, HS3, and HS4; HS3, HS4, and HS5; HS1, HS3, and HS5; HS1, HS2, and HS5; and HS1, HS4, and HS5. For additional examples of mini-LCR, see Sadelain et al., Proc. Nat. Acad. Sci. (USA) 92: 6728-6732, 1995; and Lebouich et al., EMBO J. 13: 3065-3076, 1994. Particular embodiments can utilize a mini-β-globin LCR in combination with a β-globin promoter. In particular embodiments, this combination yields a 5.9 kb LCR-promoter combination. In relation to LCR, “mini” and “micro” are used interchangeably herein.

Particular embodiments disclosed herein utilize long portions of the locus control region (LCR). A long β-globin LCR can include HS1, HS2, HS3, HS4, and HS5. In particular embodiments, a long LCR includes an approximately 21.5 kb sequence including HS1, HS2, HS3, HS4, and HS5 of the β-globin LCR. A long β-globin LCR can be coupled with the β-globin promoter to drive high protein expression levels.

Particular embodiments can include as a long β-globin LCR positions 5292319-5270789 (21,531 bp) of human chromosome 11 (SEQ ID NO: 6) as enumerated in GRCH38/hg38. In various embodiments, a long LCR can have a total length equal to or greater than, 18 kb, 18.5 kb, 19 kb, 19.5 kb, 20 kb, 20.5 kb, 21 kb, 21.5 kb, or 21.531 kb. In various embodiments, a long LCR can have a total length equal to or greater than 70%, 75%, 80%, 85%, 90%, 91%, 92%, 93%, 94%, 95%, 96%, 97%, 98%, or 99% of the length of SEQ ID NO: 6. In various embodiments, a long LCR can include at least 18 kb, 18.5 kb, 19 kb, 19.5 kb, 20 kb, 20.5 kb, 21 kb, or 21.5 kb of SEQ ID NO: 6. In any of the various embodiments provided herein, a long LCR can be or include a nucleic acid having at least 70%, 75%, 80%, 85%, 90%, 91%, 92%, 93%, 94%, 95%, 96%, 97%, 98%, or 99% identity with a corresponding contiguous portion of SEQ ID NO: 6. In various embodiments, a long LCR can differ from a natural genomic sequence in that it includes one or more restriction sites, such as a Xhol restriction site (see, e.g. SEQ ID NO: 98, in which an exemplary Xhol site (italicized) is provided at positions 10655-10661). In any of the various embodiments provided herein, a long LCR can include HS1, HS2, HS3, HS4, and HS5.

In various embodiments, an Ad35 vector system can include, e.g., a transposable transgene insert that includes positions 5228631-5227018 (1614 bp) of human chromosome 11 (SEQ ID NO: 7) as enumerated in GRCh38 as a β-globin promoter. In various embodiments, a β-globin promoter can have a total length equal to or greater than, e.g., 1.0 kb, 1.1. kb, 1.2 kb, 1.3 kb, 1.4 kb, 1.5 kb, 1.6 kb, or 1.609 kb. In various embodiments, a β-globin promoter can include at least 1.0 kb, 1.1 kb, 1.2 kb, 1.3 kb, 1.4 kb, 1.5 kb, 1.6 kb, or 1.609 kb of SEQ ID NO: 7. In various embodiments, a β-globin promoter can include a total length equal to or greater than, e.g., 100 bp, 200 bp, 300 bp, 400 bp, 500 bp, 1 kb, 1.5 kb, 2 kb, 2.5 kb, 3 kb, 4 kb, or 5 kb of a nucleic acid sequence upstream of, e.g., immediately upstream of the first coding nucleotide of, a gene whose expression is regulated by the β-globin LCR, including without limitation any of epsilon (HBE1), G-gamma (HBG2), A-gamma (HBG1), delta (HBD), and beta (HBB) globin genes and/or one or more genes present in the hemoglobin β locus (11:5,225,463-5,227,070, complement). In various embodiments, a β-globin promoter can include a total length equal to or greater than, e.g., 100 bp, 200 bp, 300 bp, 400 bp, 500 bp, 1 kb, 1.5 kb, 2 kb, 2.5 kb, 3 kb, 4 kb, or 5 kb of a nucleic acid sequence upstream, e.g., immediately upstream, of Chromosome 11 NC_000011.10 position 5227021. In various embodiments, a β-globin promoter can have a total length equal to or greater than 70%, 75%, 80%, 85%, 90%, 91%, 92%, 93%, 94%, 95%, 96%, 97%, 98%, or 99% of the length of SEQ ID NO: 7. In any of the various embodiments provided herein, a β-globin promoter can be or include a nucleic acid having a sequence having at least 70%, 75%, 80%, 85%, 90%, 91%, 92%, 93%, 94%, 95%, 96%, 97%, 98%, or 99% identity with a corresponding contiguous portion of a β-globin promoter sequence present in a reference genome, optionally wherein the β-globin promoter includes the sequence of SEQ ID NO: 7.

In various embodiments, a β-globin LCR, such as a long β-globin LCR, causes expression of an operably linked coding sequence in erythrocytes. In various embodiments, the operably linked coding sequence is also operably linked with a β-globin promoter as set forth herein or otherwise known in the art.

The immunoglobulin heavy chain locus B cell LCR is an exemplary LCR that enhances expression (e.g., increases transcription, increases translation, and/or increases cell or tissue specificity) of operably linked coding sequences. Expression of a coding sequence can be enhanced when operably linked with an immunoglobulin heavy chain locus B cell LCR that includes the complete immunoglobulin heavy chain locus B cell LCR sequence and/or that includes an expression-regulatory fragment thereof. The immunoglobulin heavy chain locus B cell LCR includes DNAse hypersensitive sites (HS) understood by those of skill in the art to mediate at least some of the expression-enhancing effects of the immunoglobulin heavy chain locus B cell LCR. The immunoglobulin heavy chain locus B cell LCR includes four DNase l-hypersensitive sites (HS1, HS2, HS3, and HS4) in the 3′Cα region of the immunoglobulin heavy chain (IgH) locus functions as an enhancer-locus control region (LCR). Accordingly, an immunoglobulin heavy chain locus B cell LCR can be a complete immunoglobulin heavy chain locus B cell LCR including all of HS1-HS4, or can be an expression-regulatory fragment thereof that includes a subset of the hypersensitive sites HS1-HS4. These HS sites map to about 10-30 kb of the IgH C gene and can cause lymphoid cell-specific and developmentally regulated enhancer elements in transient transfection assays. It has been observed that this nucleic acid sequence can direct a similar pattern of expression when linked to c-myc genes in Burkitt Lymphoma and plasmacytoma cell lines. In Burkitt Lymphomas and plasmacytomas, control of c-myc by the B-cell LCR occurs because of characteristic chromosome translocations that cause c-myc genes to become juxtaposed with the IgH sequences, thereby resulting in aberrant c-myc transcription. Additional description of the B Cell LCR can be found, for example, in Madisen et al., Mol Cell Biol. 18(11):6281-92, 1998; Giannini et al., J. Immunol. 150:1772-1780, 1993; Madisen & Groudine, Genes Dev. 8:2212-2226, 1994; and Michaelson et al., Nucleic Acids Res. 23:975-981, 1995.

Particular embodiments can include immunoglobulin heavy chain locus B cell LCR positions Chromosome 14 - NC_000014.9 (105586437-106879844, complement) (1,293,408 bp) or an expression-regulatory fragment thereof. In various embodiments, an immunoglobulin heavy chain locus B cell LCR can have a total length equal to or greater than 70%, 75%, 80%, 85%, 90%, 91%, 92%, 93%, 94%, 95%, 96%, 97%, 98%, 99%, or 100% of immunoglobulin heavy chain locus B cell LCR positions 105586437-106879844. In various embodiments, an immunoglobulin heavy chain locus B cell LCR can include at least 10 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, or 30 kb of immunoglobulin heavy chain locus B cell LCR positions 105586437-106879844. In any of the various embodiments provided herein, a long LCR can be or include a nucleic acid having at least 70%, 75%, 80%, 85%, 90%, 91%, 92%, 93%, 94%, 95%, 96%, 97%, 98%, or 99% identity with a corresponding contiguous portion of immunoglobulin heavy chain locus B cell LCR positions 105586437-106879844.

In various embodiments, an Ad35 vector can include an immunoglobulin heavy chain locus B cell LCR as provided herein, e.g., in a payload that includes the immunoglobulin heavy chain locus B cell LCR and, optionally, a promoter of a gene that is typically operably linked with the immunoglobulin heavy chain locus B cell LCR in the human genome. In various embodiments, the gene operably linked with the immunoglobulin heavy chain locus B cell LCR is the immunoglobulin heavy chain gene. In various embodiments, an immunoglobulin heavy chain gene promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, an immunoglobulin heavy chain gene promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, the immunoglobulin heavy chain gene, e.g., in a reference genome. In some embodiments, the first coding nucleotide of a coding sequence of a gene that is typically operably linked with the immunoglobulin heavy chain locus B cell LCR in the human genome is the is the first coding nucleotide of immunoglobulin heavy chain gene.

In various embodiments, an immunoglobulin heavy chain locus B cell LCR, such as a long immunoglobulin heavy chain locus B cell LCR, causes expression of an operably linked coding sequence in B cells. In various embodiments, the operably linked coding sequence is also operably linked with an immunoglobulin heavy chain gene promoter as set forth herein or otherwise known in the art.

Another exemplary LCR is a T cell LCR of the T cell receptor alpha/delta locus that enhances expression of operably linked coding sequences. In the T cell receptor (TCR) alpha/delta locus, an LCR can regulate the differential tissue and developmental expression and the rearrangement of TCR alpha and delta genes. Expression of a coding sequence can be enhanced when operably linked with a T cell LCR of the T cell receptor alpha/delta locus LCR that includes the complete T cell LCR of the T cell receptor alpha/delta locus LCR sequence and/or that includes an expression-regulatory fragment thereof. The T cell LCR of the T cell receptor alpha/delta locus LCR includes DNAse hypersensitive sites (HS) understood by those of skill in the art to mediate at least some of the expression-enhancing effects of the T cell LCR of the T cell receptor alpha/delta locus LCR. The T cell LCR was identified as a region 3′ of the TCR alpha/delta locus that included eight T cell-specific nuclease hypersensitive domains (HS-1 to HS-8). Accordingly, a T cell LCR of the T cell receptor alpha/delta locus LCR can be a complete T cell LCR of the T cell receptor alpha/delta locus LCR including all of HS1-HS8, or can be an expression-regulatory fragment thereof that includes a subset of the hypersensitive sites HS1-HS8. It was observed in transgenic mice that a TCR alpha gene linked to this region is expressed at a high level, independent of the site of integration and correlated with gene copy number. This transgene was expressed in the alpha beta but not the gamma delta T cell subset and was activated at the right time during development. LCR function requires at least HS-2 to HS-6. Additional description of the B Cell LCR can be found, for example, in Diaz et al., Immunity 1(3):207-17, 1994.

In various embodiments, an Ad35 vector can include a T cell LCR of the T cell receptor alpha/delta locus LCR as provided herein, e.g., in a payload that includes the T cell LCR of the T cell receptor alpha/delta locus LCR and, optionally, a promoter of a gene that is typically operably linked with the T cell LCR of the T cell receptor alpha/delta locus LCR in the human genome. In various embodiments, the gene operably linked with the T cell LCR of the T cell receptor alpha/delta locus LCR is the TCR alpha on Chromosome 14, NC_000014.9 (21621904..22552132) or TCR delta locus on Chromosome 14, NC_000014.9 (22422546..22466577). In various embodiments, a TCR alpha or TCR delta promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a TCR alpha or TCR delta promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, TCR alpha or TCR delta, e.g., in a reference genome. In some embodiments, the first coding nucleotide of a coding sequence of a gene that is typically operably linked with the T cell LCR of the T cell receptor alpha/delta locus LCR in the human genome is the first coding nucleotide of TCR alpha or TCR delta.

In various embodiments, a T cell LCR of the T cell receptor alpha/delta locus LCR, such as a long T cell LCR of the T cell receptor alpha/delta locus LCR, causes expression of an operably linked coding sequence in T cells. In various embodiments, the operably linked coding sequence is also operably linked with a TCR alpha or TCR delta promoter as set forth herein or otherwise known in the art.

The adenosine deaminase LCR is an exemplary LCR that enhances expression of operably linked coding sequences. Expression of a coding sequence can be enhanced when operably linked with an adenosine deaminase LCR that includes the complete adenosine deaminase LCR sequence and/or that includes an expression-regulatory fragment thereof. The adenosine deaminase LCR includes DNAse hypersensitive sites (HS) understood by those of skill in the art to mediate at least some of the expression-enhancing effects of the adenosine deaminase LCR. The adenosine deaminase LCR includes hypersensitive sites 1-6. Accordingly, a adenosine deaminase LCR can be a complete adenosine deaminase LCR including all of HS1 -HS6, or can be an expression-regulatory fragment thereof that includes a subset of the hypersensitive sites HS1-HS6.

Particular embodiments can include adenosine deaminase LCR positions NC_000020.11 44629004-44651567 (22,564 bp) of human chromosome 20 or an expression-regulatory fragment thereof. In various embodiments, an adenosine deaminase LCR can have a total length equal to or greater than 70%, 75%, 80%, 85%, 90%, 91%, 92%, 93%, 94%, 95%, 96%, 97%, 98%, 99%, or 100% of adenosine deaminase LCR positions 44629004-44651567. In various embodiments, an adenosine deaminase LCR can include at least 10 kb, 15 kb, 16 kb, 17 kb, 18 kb, 19 kb, 20 kb, 21 kb, or 22 kb of adenosine deaminase LCR positions 44629004-44651567. In any of the various embodiments provided herein, a long LCR can be or include a nucleic acid having at least 70%, 75%, 80%, 85%, 90%, 91%, 92%, 93%, 94%, 95%, 96%, 97%, 98%, or 99% identity with a corresponding contiguous portion of adenosine deaminase LCR positions 44629004-44651567.

In various embodiments, an Ad35 vector can include an adenosine deaminase LCR as provided herein, e.g., in a payload that includes the adenosine deaminase LCR and, optionally, a promoter of a gene that is typically operably linked with the adenosine deaminase LCR in the human genome. In various embodiments, the gene operably linked with the adenosine deaminase LCR is adenosine deaminase (20:44,619,518-44,651,757, complement). In various embodiments, an adenosine deaminase promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, an adenosine deaminase promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, adenosine deaminase, e.g., in a reference genome. In some embodiments, the first coding nucleotide of a coding sequence of a gene that is typically operably linked with the adenosine deaminase LCR in the human genome is the first coding nucleotide of adenosine deaminase at chromosome 20 - NC_000020.11 44651607.

In various embodiments, an adenosine deaminase LCR, such as a long adenosine deaminase LCR, causes expression of an operably linked coding sequence in one or more of blood, intestine, and lymphoid tissue. In various embodiments, the operably linked coding sequence is also operably linked with an adenosine deaminase promoter as set forth herein or otherwise known in the art.

The apolipoprotein E/C LCR is an exemplary LCR that enhances expression of operably linked coding sequences. Expression of a coding sequence can be enhanced when operably linked with an apolipoprotein E/C LCR that includes the complete apolipoprotein E/C LCR sequence and/or that includes an expression-regulatory fragment thereof. The apolipoprotein E/C LCR includes DNAse hypersensitive sites (HS) understood by those of skill in the art to mediate at least some of the expression-enhancing effects of the apolipoprotein E/C LCR. The apolipoprotein E/C LCR includes hypersensitive sites 1-6. Accordingly, an apolipoprotein E/C LCR can be a complete apolipoprotein E/C LCR including all of HS1-HS6, or can be an expression-regulatory fragment thereof that includes a subset of the hypersensitive sites HS1-HS6.

In various embodiments, an Ad35 vector can include an apolipoprotein E/C LCR as provided herein, e.g., in a payload that includes the apolipoprotein E/C LCR and, optionally, a promoter of a gene that is typically operably linked with the apolipoprotein E/C LCR in the human genome. In various embodiments, the gene operably linked with the apolipoprotein E/C LCR is apolipoprotein E (19:44,905,795-44,909,394). In various embodiments, an apolipoprotein E promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a apolipoprotein E promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, apolipoprotein E, e.g., in a reference genome. In some embodiments, the first coding nucleotide of a coding sequence of a gene that is typically operably linked with the apolipoprotein E/C LCR in the human genome is the first coding nucleotide of apolipoprotein E at Chromosome 19 - NC_000019.10 (44906625).

In various embodiments, an apolipoprotein E/C LCR, such as a long apolipoprotein E/C LCR, causes expression of an operably linked coding sequence in erythrocytes. In various embodiments, the operably linked coding sequence is also operably linked with an apolipoprotein E/C promoter as set forth herein or otherwise known in the art.

The Th2 cytokine LCR is an exemplary LCR that enhances expression of operably linked coding sequences. Expression of a coding sequence can be enhanced when operably linked with a Th2 cytokine LCR that includes the complete Th2 cytokine LCR sequence and/or that includes an expression-regulatory fragment thereof. The Th2 cytokine LCR includes DNAse hypersensitive sites (HS) understood by those of skill in the art to mediate at least some of the expression-enhancing effects of the Th2 cytokine LCR. The Th2 cytokine LCR includes hypersensitive sites RHS5-RHS7. Accordingly, a Th2 cytokine LCR can be a complete Th2 cytokine LCR including all of RHS5-RHS7, or can be an expression-regulatory fragment thereof that includes a subset of the hypersensitive sites RHS5-RHS7.

Particular embodiments can include Th2 cytokine LCR positions NC_000005.10 (132629263-132642195) (12,933 bp) of human chromosome 5 or an expression-regulatory fragment thereof. In various embodiments, a Th2 cytokine LCR can have a total length equal to or greater than 70%, 75%, 80%, 85%, 90%, 91%, 92%, 93%, 94%, 95%, 96%, 97%, 98%, 99%, or 100% of Th2 cytokine LCR positions 132629263-132642195. In various embodiments, a Th2 cytokine LCR can include at least 1 kb, 2 kb, 3 kb, 4 kb, 5 kb, 6 kb, 7 kb, 8 kb, 9 kb, 10 kb, 11 kb, or 12 kb of Th2 cytokine LCR positions 132629263-132642195. In any of the various embodiments provided herein, a long LCR can be or include a nucleic acid having at least 70%, 75%, 80%, 85%, 90%, 91%, 92%, 93%, 94%, 95%, 96%, 97%, 98%, or 99% identity with a corresponding contiguous portion of Th2 cytokine LCR positions 132629263-132642195.

In various embodiments, an Ad35 vector can include a Th2 cytokine LCR as provided herein, e.g., in a payload that includes the Th2 cytokine LCR and, optionally, a promoter of a gene that is typically operably linked with the Th2 cytokine LCR in the human genome. In various embodiments, the gene operably linked with the Th2 cytokine LCR is a Th2 cytokine, e.g., IL-4, IL-13, or IL-5. In various embodiments, a Th2 cytokine promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a Th2 cytokine promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, Th2 cytokine, e.g., in a reference genome.

In various embodiments, a Th2 cytokine LCR, such as a long Th2 cytokine LCR, causes expression of an operably linked coding sequence in T cells. In various embodiments, the operably linked coding sequence is also operably linked with a Th2 cytokine promoter as set forth herein or otherwise known in the art.

The CD2 LCR is an exemplary LCR that enhances expression of operably linked coding sequences. Expression of a coding sequence can be enhanced when operably linked with a CD2 LCR that includes the complete CD2 LCR sequence and/or that includes an expression-regulatory fragment thereof. The CD2 LCR includes DNAse hypersensitive sites (HS) understood by those of skill in the art to mediate at least some of the expression-enhancing effects of the CD2 LCR. The CD2 LCR includes hypersensitive sites 1-3. Accordingly, a CD2 LCR can be a complete CD2 LCR including all of HS1-HS3, or can be an expression-regulatory fragment thereof that includes a subset of the hypersensitive sites HS1-HS3.

Particular embodiments can include CD2 LCR positions NC_000001.11 116769217-116774826 (5,610 bp) of human chromosome 1 or an expression-regulatory fragment thereof. In various embodiments, a CD2 LCR can have a total length equal to or greater than 70%, 75%, 80%, 85%, 90%, 91%, 92%, 93%, 94%, 95%, 96%, 97%, 98%, 99%, or 100% of CD2 LCR positions 116769217-116774826. In various embodiments, a CD2 LCR can include at least 1 kb, 2 kb, 3 kb, 4 kb, or 5 kb of CD2 LCR positions 116769217-116774826. In any of the various embodiments provided herein, a long LCR can be or include a nucleic acid having at least 70%, 75%, 80%, 85%, 90%, 91%, 92%, 93%, 94%, 95%, 96%, 97%, 98%, or 99% identity with a corresponding contiguous portion of CD2 LCR positions 116769217-116774826.

In various embodiments, an Ad35 vector can include a CD2 LCR as provided herein, e.g., in a payload that includes the CD2 LCR and, optionally, a promoter of a gene that is typically operably linked with the CD2 LCR in the human genome. In various embodiments, the gene operably linked with the CD2 LCR is CD2 (1:116,754,429-116,769,228). In various embodiments, a CD2 promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a CD2 promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, CD2, e.g., in a reference genome. In some embodiments, the first coding nucleotide of a coding sequence of a gene that is typically operably linked with the CD2 LCR in the human genome is the first coding nucleotide of CD2 at Chromosome 1 - NC_000001.11 (116754493).

In various embodiments, a CD2 LCR, such as a long CD2 LCR, causes expression of an operably linked coding sequence in T cells. In various embodiments, the operably linked coding sequence is also operably linked with a CD2 promoter as set forth herein or otherwise known in the art.

The S100β LCR is an exemplary LCR that enhances expression of operably linked coding sequences. Expression of a coding sequence can be enhanced when operably linked with a S100β LCR that includes the complete S100β LCR sequence and/or that includes an expression-regulatory fragment thereof. The S100β LCR includes DNAse hypersensitive sites (HS) understood by those of skill in the art to mediate at least some of the expression-enhancing effects of the S100β LCR.

In various embodiments, an Ad35 vector can include a S100β LCR as provided herein, e.g., in a payload that includes the S100β LCR and, optionally, a promoter of a gene that is typically operably linked with the S1 00β LCR in the human genome. In various embodiments, the gene operably linked with the S1 00β LCR is S1 00β (21 :46,598,603-46,605,242, complement). In various embodiments, a S1 00β promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a S100β promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, S100β, e.g., in a reference genome. In some embodiments, the first coding nucleotide of a coding sequence of a gene that is typically operably linked with the S100β LCR in the human genome is the first coding nucleotide of S1 00β (Chromosome 21 - NC_000021.9 (46602415)).

In various embodiments, a S100β LCR, such as a long S100β LCR, causes expression of an operably linked coding sequence in brain astrocytes. In various embodiments, the operably linked coding sequence is also operably linked with a S100β promoter as set forth herein or otherwise known in the art.

The growth hormone LCR is an exemplary LCR that enhances expression of operably linked coding sequences. Expression of a coding sequence can be enhanced when operably linked with a growth hormone LCR that includes the complete growth hormone LCR sequence and/or that includes an expression-regulatory fragment thereof. The growth hormone LCR includes DNAse hypersensitive sites (HS) understood by those of skill in the art to mediate at least some of the expression-enhancing effects of the growth hormone LCR. The growth hormone LCR includes hypersensitive sites 1-5. Accordingly, a growth hormone LCR can be a complete growth hormone LCR including all of HS1-HS5, or can be an expression-regulatory fragment thereof that includes a subset of the hypersensitive sites HS1-HS5.

Particular embodiments can include growth hormone LCR positions NC_000017.11 (63917193-63958852) (41,660 bp) of human chromosome 17, or an expression-regulatory fragment thereof. In various embodiments, a growth hormone LCR can have a total length equal to or greater than 70%, 75%, 80%, 85%, 90%, 91%, 92%, 93%, 94%, 95%, 96%, 97%, 98%, 99%, or 100% of growth hormone LCR positions 63917193-63958852. In various embodiments, a growth hormone LCR can include at least 10 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, or 30 kb of growth hormone LCR positions 63917193-63958852. In any of the various embodiments provided herein, a long LCR can be or include a nucleic acid having at least 70%, 75%, 80%, 85%, 90%, 91%, 92%, 93%, 94%, 95%, 96%, 97%, 98%, or 99% identity with a corresponding contiguous portion of growth hormone LCR positions 63917193-63958852.

In various embodiments, an Ad35 vector can include a growth hormone LCR as provided herein, e.g., in a payload that includes the growth hormone LCR and, optionally, a promoter of a gene that is typically operably linked with the growth hormone LCR in the human genome. In various embodiments, the gene operably linked with the growth hormone LCR is GH1 (growth hormone 1), CSHL1 (chorionic somatomammotropin hormone-like 1), CSH1 (chorionic somatomammotropin hormone 1 (placental lactogen)), GH2 (growth hormone 2), or CSH2 (chorionic somatomammotropin hormone 2). In various embodiments, a GH1, CSHL1, CSH1, GH2, or CSH2 promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a GH1, CSHL1, CSH1, GH2, or CSH2 promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, GH1, CSHL1, CSH1, GH2, or CSH2, e.g., in a reference genome. In some embodiments, the first coding nucleotide of a coding sequence of a gene that is typically operably linked with the growth hormone LCR in the human genome is the first coding nucleotide of growth hormone (17:63,917,202-63,918,838, complement) position NC_000017.11 (63918776).

In various embodiments, a growth hormone LCR, such as a long growth hormone LCR, causes expression of an operably linked coding sequence in the pituitary gland. In various embodiments, the operably linked coding sequence is also operably linked with a GH1, CSHL1, CSH1, GH2, or CSH2 promoter as set forth herein or otherwise known in the art.

The apolipoprotein B LCR is an exemplary LCR that enhances expression of operably linked coding sequences. Expression of a coding sequence can be enhanced when operably linked with an apolipoprotein B LCR that includes the complete apolipoprotein B LCR sequence and/or that includes an expression-regulatory fragment thereof. The apolipoprotein B LCR includes DNAse hypersensitive sites (HS) understood by those of skill in the art to mediate at least some of the expression-enhancing effects of the apolipoprotein B LCR.

In various embodiments, an Ad35 vector can include an apolipoprotein B LCR as provided herein, e.g., in a payload that includes the apolipoprotein B LCR and, optionally, a promoter of a gene that is typically operably linked with the apolipoprotein B LCR in the human genome. In various embodiments, the gene operably linked with the apolipoprotein B LCR is APOB (2:21,001,428-21,044,072, complement). In various embodiments, an APOB promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, an APOB promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, APOB, e.g., in a reference genome. In some embodiments, the first coding nucleotide of a coding sequence of a gene that is typically operably linked with the apolipoprotein B LCR in the human genome is the first coding nucleotide of an APOB at position Chromosome 2 - NC_000002.12 (21043945).

In various embodiments, an apolipoprotein B LCR, such as a long apolipoprotein B LCR, causes expression of an operably linked coding sequence in intestine and/or liver. In various embodiments, the operably linked coding sequence is also operably linked with an APOB promoter as set forth herein or otherwise known in the art.

The β myosin heavy chain LCR is an exemplary LCR that enhances expression of operably linked coding sequences. Expression of a coding sequence can be enhanced when operably linked with a β myosin heavy chain LCR that includes the complete β myosin heavy chain LCR sequence and/or that includes an expression-regulatory fragment thereof. The β myosin heavy chain LCR includes DNAse hypersensitive sites (HS) understood by those of skill in the art to mediate at least some of the expression-enhancing effects of the β myosin heavy chain LCR. The β myosin heavy chain LCR includes hypersensitive sites 1 and 2. Accordingly, a β myosin heavy chain LCR can be a complete β myosin heavy chain LCR including both HS1 and HS2, or can be an expression-regulatory fragment thereof that includes a subset of the hypersensitive sites (HS1 or HS2).

In various embodiments, an Ad35 vector can include a β myosin heavy chain LCR as provided herein, e.g., in a payload that includes the β myosin heavy chain LCR and, optionally, a promoter of a gene that is typically operably linked with the β myosin heavy chain LCR in the human genome. In various embodiments, the gene operably linked with the β myosin heavy chain LCR is β myosin heavy chain (14:23,412,739-23,435,676, complement). In various embodiments, a β myosin heavy chain promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a β myosin heavy chain promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, β myosin heavy chain, e.g., in a reference genome. In some embodiments, the first coding nucleotide of a coding sequence of a gene that is typically operably linked with the β myosin heavy chain LCR in the human genome is the first coding nucleotide of β myosin heavy chain at Chromosome 14 - NC_000014.9 (23433732).

In various embodiments, a β myosin heavy chain LCR, such as a long β myosin heavy chain LCR, causes expression of an operably linked coding sequence in heart muscle and/or skeletal muscle. In various embodiments, the operably linked coding sequence is also operably linked with a β myosin heavy chain promoter as set forth herein or otherwise known in the art.

The MHC Class I HLA-B7 LCR is an exemplary LCR that enhances expression of operably linked coding sequences. Expression of a coding sequence can be enhanced when operably linked with a MHC Class I HLA-B7 LCR that includes the complete MHC Class I HLA-B7 LCR sequence and/or that includes an expression-regulatory fragment thereof. The MHC Class I HLA-B7 LCR includes DNAse hypersensitive sites (HS) understood by those of skill in the art to mediate at least some of the expression-enhancing effects of the MHC Class I HLA-B7 LCR.

In various embodiments, an Ad35 vector can include a MHC Class I HLA-B7 LCR as provided herein, e.g., in a payload that includes the MHC Class I HLA-B7 LCR and, optionally, a promoter of a gene that is typically operably linked with the MHC Class I HLA-B7 LCR in the human genome. In various embodiments, the gene operably linked with the MHC Class I HLA-B7 LCR is MHC Class I HLA-B7. In various embodiments, a MHC Class I HLA-B7 promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a MHC Class I HLA-B7 promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, MHC Class I HLA-B7, e.g., in a reference genome.

In various embodiments, a MHC Class I HLA-B7 LCR, such as a long MHC Class I HLA-B7 LCR, causes expression of an operably linked coding sequence in many cell types, or ubiquitously. In various embodiments, the operably linked coding sequence is also operably linked with a MHC Class I HLA-B7 promoter as set forth herein or otherwise known in the art.

The MHC Class I HLA-G LCR is an exemplary LCR that enhances expression of operably linked coding sequences. Expression of a coding sequence can be enhanced when operably linked with a MHC Class I HLA-G LCR that includes the complete MHC Class I HLA-G LCR sequence and/or that includes an expression-regulatory fragment thereof. The MHC Class I HLA-G LCR includes DNAse hypersensitive sites (HS) understood by those of skill in the art to mediate at least some of the expression-enhancing effects of the MHC Class I HLA-G LCR.

In various embodiments, an Ad35 vector can include a MHC Class I HLA-G LCR as provided herein, e.g., in a payload that includes the MHC Class I HLA-G LCR and, optionally, a promoter of a gene that is typically operably linked with the MHC Class I HLA-G LCR in the human genome. In various embodiments, the gene operably linked with the MHC Class I HLA-G LCR is MHC Class I HLA-G. In various embodiments, a MHC Class I HLA-G promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a MHC Class I HLA-G promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, MHC Class I HLA-G, e.g., in a reference genome.

In various embodiments, a MHC Class I HLA-G LCR, such as a long MHC Class I HLA-G LCR, causes expression of an operably linked coding sequence in many cell types, or ubiquitously. In various embodiments, the operably linked coding sequence is also operably linked with a MHC Class I HLA-G promoter as set forth herein or otherwise known in the art.

The keratin 18 LCR is an exemplary LCR that enhances expression of operably linked coding sequences. Expression of a coding sequence can be enhanced when operably linked with a keratin 18 LCR that includes the complete keratin 18 LCR sequence and/or that includes an expression-regulatory fragment thereof. The keratin 18 LCR includes DNAse hypersensitive sites (HS) understood by those of skill in the art to mediate at least some of the expression-enhancing effects of the keratin 18 LCR. The keratin 18 LCR includes hypersensitive sites 1-4. Accordingly, a keratin 18 LCR can be a complete keratin 18 LCR including all of HS1-HS4, or can be an expression-regulatory fragment thereof that includes a subset of the hypersensitive sites HS1-HS4.

Particular embodiments can include keratin 18 LCR positions NC_000012.12 (52948039-52956706) (8,668 bp) of human chromosome 12 or an expression-regulatory fragment thereof. In various embodiments, a keratin 18 LCR can have a total length equal to or greater than 70%, 75%, 80%, 85%, 90%, 91%, 92%, 93%, 94%, 95%, 96%, 97%, 98%, 99%, or 100% of keratin 18 LCR positions 52948039-52956706. In various embodiments, a keratin 18 LCR can include at least 1 kb, 2 kb, 3 kb, 4 kb, 5 kb, 6 kb, 7 kb, or 8 kb of keratin 18 LCR positions 52948039-52956706. In any of the various embodiments provided herein, a long LCR can be or include a nucleic acid having at least 70%, 75%, 80%, 85%, 90%, 91%, 92%, 93%, 94%, 95%, 96%, 97%, 98%, or 99% identity with a corresponding contiguous portion of keratin 18 LCR positions 52948039-52956706.

In various embodiments, an Ad35 vector can include a keratin 18 LCR as provided herein, e.g., in a payload that includes the keratin 18 LCR and, optionally, a promoter of a gene that is typically operably linked with the keratin 18 LCR in the human genome. In various embodiments, the gene operably linked with the keratin 18 LCR is keratin 18 (12:52,948,870-52,952,905). In various embodiments, a keratin 18 promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a keratin 18 promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, keratin 18, e.g., in a reference genome. In some embodiments, the first coding nucleotide of a coding sequence of a gene that is typically operably linked with the keratin 18 LCR in the human genome is the first coding nucleotide of keratin 18 at Chromosome 12 -NC_000012.12 (52949174).

In various embodiments, a keratin 18 LCR, such as a long keratin 18 LCR, causes expression of an operably linked coding sequence in epithelial cells. In various embodiments, the operably linked coding sequence is also operably linked with a keratin 18 promoter as set forth herein or otherwise known in the art.

The Complement Component C4A/B LCR is an exemplary LCR that enhances expression of operably linked coding sequences. Expression of a coding sequence can be enhanced when operably linked with a Complement Component C4A/B LCR that includes the complete Complement Component C4A/B LCR sequence and/or that includes an expression-regulatory fragment thereof. The Complement Component C4A/B LCR includes DNAse hypersensitive sites (HS) understood by those of skill in the art to mediate at least some of the expression-enhancing effects of the Complement Component C4A/B LCR.

In various embodiments, an Ad35 vector can include a Complement Component C4A/B LCR as provided herein, e.g., in a payload that includes the Complement Component C4A/B LCR and, optionally, a promoter of a gene that is typically operably linked with the Complement Component C4A/B LCR in the human genome. In various embodiments, the gene operably linked with the Complement Component C4A/B LCR is C4A (6:31,982,056-32,002,680). In various embodiments, a C4A promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a C4A promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, C4A, e.g., in a reference genome. In some embodiments, the first coding nucleotide of a coding sequence of a gene that is typically operably linked with the Complement Component C4A/B LCR in the human genome is the first coding nucleotide of C4A at Chromosome 6 -NC_000006.12 (31982108).

In various embodiments, a Complement Component C4A/B LCR, such as a long Complement Component C4A/B LCR, causes expression of an operably linked coding sequence in liver. In various embodiments, the operably linked coding sequence is also operably linked with a C4A promoter as set forth herein or otherwise known in the art.

The red and green visual pigment (OPSIN) LCR is an exemplary LCR that enhances expression of operably linked coding sequences. Expression of a coding sequence can be enhanced when operably linked with a red and green visual pigment (OPSIN) LCR that includes the complete red and green visual pigment (OPSIN) LCR sequence and/or that includes an expression-regulatory fragment thereof. The red and green visual pigment (OPSIN) LCR includes DNAse hypersensitive sites (HS) understood by those of skill in the art to mediate at least some of the expression-enhancing effects of the red and green visual pigment (OPSIN) LCR. The red and green visual pigment (OPSIN) LCR includes hypersensitive sites 1-3. Accordingly, a red and green visual pigment (OPSIN) LCR can be a complete red and green visual pigment (OPSIN) LCR including all of HS1-HS3, or can be an expression-regulatory fragment thereof that includes a subset of the hypersensitive sites HS1-HS3.

Particular embodiments can include red and green visual pigment (OPSIN) LCR positions NC_000023.11 (154137727-154144286) (6,560 bp) of human chromosome X or an expression-regulatory fragment thereof. In various embodiments, a red and green visual pigment (OPSIN) LCR can have a total length equal to or greater than 70%, 75%, 80%, 85%, 90%, 91%, 92%, 93%, 94%, 95%, 96%, 97%, 98%, 99%, or 100% of red and green visual pigment (OPSIN) LCR positions 154137727-154144286. In various embodiments, a red and green visual pigment (OPSIN) LCR can include at least 1 kb, 2 kb, 3 kb, 4 kb, 5 kb, or 6 kb of red and green visual pigment (OPSIN) LCR positions 154137727-154144286. In any of the various embodiments provided herein, a long LCR can be or include a nucleic acid having at least 70%, 75%, 80%, 85%, 90%, 91%, 92%, 93%, 94%, 95%, 96%, 97%, 98%, or 99% identity with a corresponding contiguous portion of red and green visual pigment (OPSIN) LCR positions 154137727-154144286.

In various embodiments, an Ad35 vector can include a red and green visual pigment (OPSIN) LCR as provided herein, e.g., in a payload that includes the red and green visual pigment (OPSIN) LCR and, optionally, a promoter of a gene that is typically operably linked with the red and green visual pigment (OPSIN) LCR in the human genome. In various embodiments, the gene operably linked with the red and green visual pigment (OPSIN) LCR is opsin 1 (X:154,144,242-154,159,031), long-wave-sensitive (OPN1LW), opsin 1, medium-wave-sensitive (OPN1 MW), OPN1MW2, or OPN1MW3. In various embodiments, an OPN1LW, OPN1MW, OPN1 MW2, or OPN1 MW3 promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, an OPN1LW, OPN1MW, OPN1MW2, or OPN1MW3 promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, OPN1 LW, OPN1 MW, OPN1 MW2, or OPN1 MW3, e.g., in a reference genome. In some embodiments, the first coding nucleotide of a coding sequence of a gene that is typically operably linked with the red and green visual pigment (OPSIN) LCR in the human genome is the first coding nucleotide of OPN1LW at Chromosome X - NC_000023.11 (154144284) or OPN1MW at Chromosome X - NC_000023.11 (154182678).

In various embodiments, a red and green visual pigment (OPSIN) LCR, such as a long red and green visual pigment (OPSIN) LCR, causes expression of an operably linked coding sequence in cone photoreceptors. In various embodiments, the operably linked coding sequence is also operably linked with an OPN1LW, OPN1MW, OPN1 MW2, or OPN1 MW3 promoter as set forth herein or otherwise known in the art.

The α-globin LCR is an exemplary LCR that enhances expression of operably linked coding sequences. Expression of a coding sequence can be enhanced when operably linked with an α-globin LCR that includes the complete α-globin LCR sequence and/or that includes an expression-regulatory fragment thereof. The α-globin LCR includes DNAse hypersensitive sites (HS) understood by those of skill in the art to mediate at least some of the expression-enhancing effects of the α-globin LCR. The α-globin LCR includes hypersensitive sites MCS-R1 to MCS-R4. Accordingly, a α-globin LCR can be a complete α-globin LCR including all of MCS-R1 to MCS-R4, or can be an expression-regulatory fragment thereof that includes a subset of the hypersensitive sites MCS-R1 to MCS-R4.

Particular embodiments can include α-globin LCR positions NC_000016.10 (87808-152854) (65,047 bp) of human chromosome 16, or an expression-regulatory fragment thereof. In various embodiments, a α-globin LCR can have a total length equal to or greater than 70%, 75%, 80%, 85%, 90%, 91%, 92%, 93%, 94%, 95%, 96%, 97%, 98%, 99%, or 100% of α-globin LCR positions 87808-152854. In various embodiments, an α-globin LCR can include at least 10 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, or 30 kb of α-globin LCR positions 87808-152854. In any of the various embodiments provided herein, a long LCR can be or include a nucleic acid having at least 70%, 75%, 80%, 85%, 90%, 91%, 92%, 93%, 94%, 95%, 96%, 97%, 98%, or 99% identity with a corresponding contiguous portion of α-globin LCR positions 87808-152854.

In various embodiments, an Ad35 vector can include an α-globin LCR as provided herein, e.g., in a payload that includes the α-globin LCR and, optionally, a promoter of a gene that is typically operably linked with the α-globin LCR in the human genome. In various embodiments, the gene operably linked with the α-globin LCR is HBZ (hemoglobin, zeta), HBA2 (hemoglobin, alpha 2), HBA1 (hemoglobin, alpha 1), or HBQ1 (hemoglobin, theta 1) within the alpha-globin gene cluster (Major α-globin locus: 16:172,875-173,709). In various embodiments, a HBZ (hemoglobin, zeta), HBA2 (hemoglobin, alpha 2), HBA1 (hemoglobin, alpha 1), or HBQ1 (hemoglobin, theta 1) promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a HBZ (hemoglobin, zeta), HBA2 (hemoglobin, alpha 2), HBA1 (hemoglobin, alpha 1), or HBQ1 (hemoglobin, theta 1) promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, HBZ (hemoglobin, zeta), HBA2 (hemoglobin, alpha 2), HBA1 (hemoglobin, alpha 1), or HBQ1 (hemoglobin, theta 1), e.g., in a reference genome. In some embodiments, the first coding nucleotide of a coding sequence of a gene that is typically operably linked with the α-globin LCR in the human genome is the first coding nucleotide of HBA1 Chromosome 16 -NC_000016.10 (176717), HBA2 Chromosome 16 - NC_000016.10 (172913), HBZ Chromosome 16 - NC_000016.10 (152910), or HBQ1 Chromosome 16 - NC_000016.10 (180487).

In various embodiments, an α-globin LCR, such as a long α-globin LCR, causes expression of an operably linked coding sequence in erythrocytes. In various embodiments, the operably linked coding sequence is also operably linked with a promoter as set forth herein or otherwise known in the art.

The desmin LCR is an exemplary LCR that enhances expression of operably linked coding sequences. Expression of a coding sequence can be enhanced when operably linked with a desmin LCR that includes the complete desmin LCR sequence and/or that includes an expression-regulatory fragment thereof. The desmin LCR includes DNAse hypersensitive sites (HS) understood by those of skill in the art to mediate at least some of the expression-enhancing effects of the desmin LCR. The desmin LCR includes hypersensitive sites 1-5. Accordingly, a desmin LCR can be a complete desmin LCR including all of HS1-HS5, or can be an expression-regulatory fragment thereof that includes a subset of the hypersensitive sites HS1-HS5.

Particular embodiments can include desmin LCR positions NC_000002.12 (219399709-219418452) (18,743 bp) of human chromosome 2 or an expression-regulatory fragment thereof. In various embodiments, a desmin LCR can have a total length equal to or greater than 70%, 75%, 80%, 85%, 90%, 91%, 92%, 93%, 94%, 95%, 96%, 97%, 98%, 99%, or 100% of desmin LCR positions 219399709-219418452. In various embodiments, a desmin LCR can include at least 10 kb, 15 kb, 16 kb, 17 kb, or 18 kb of desmin LCR positions 219399709-219418452. In any of the various embodiments provided herein, a long LCR can be or include a nucleic acid having at least 70%, 75%, 80%, 85%, 90%, 91%, 92%, 93%, 94%, 95%, 96%, 97%, 98%, or 99% identity with a corresponding contiguous portion of desmin LCR positions 219399709-219418452.

In various embodiments, an Ad35 vector can include a desmin LCR as provided herein, e.g., in a payload that includes the desmin LCR and, optionally, a promoter of a gene that is typically operably linked with the desmin LCR in the human genome. In various embodiments, the gene operably linked with the desmin LCR is desmin (2:219,418,376-219,426,733). In various embodiments, a desmin promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a desmin promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, desmin, e.g., in a reference genome. In some embodiments, the first coding nucleotide of a coding sequence of a gene that is typically operably linked with the desmin LCR in the human genome is the first coding nucleotide of desmin at Chromosome 2 - NC_000002.12 (21941863).

In various embodiments, a desmin LCR, such as a long desmin LCR, causes expression of an operably linked coding sequence in heart muscle, skeletal muscle, and/or smooth muscle. In various embodiments, the operably linked coding sequence is also operably linked with a desmin promoter as set forth herein or otherwise known in the art.

The nuclear factor, erythroid 2 like 1 (NFE2L1 ) LCR is an exemplary LCR that enhances expression of operably linked coding sequences. Expression of a coding sequence can be enhanced when operably linked with a NFE2L1 LCR that includes the complete NFE2L1 LCR sequence and/or that includes an expression-regulatory fragment thereof. The NFE2L1 LCR includes DNAse hypersensitive sites (HS) understood by those of skill in the art to mediate at least some of the expression-enhancing effects of the NFE2L1 LCR.

Particular embodiments can include NFE2L1 LCR positions NC_000017.11 (48048359-48061545) (13, 186 bp) of human chromosome 17 or an expression-regulatory fragment thereof. In various embodiments, a NFE2L1 LCR can have a total length equal to or greater than 70%, 75%, 80%, 85%, 90%, 91%, 92%, 93%, 94%, 95%, 96%, 97%, 98%, 99%, or 100% of NFE2L1 LCR positions 48048359-48061545. In various embodiments, a NFE2L1 LCR can include at least 10 kb, 11 kb, 12 kb, or 13 kb of NFE2L1 LCR positions 48048359-48061545. In any of the various embodiments provided herein, a long LCR can be or include a nucleic acid having at least 70%, 75%, 80%, 85%, 90%, 91%, 92%, 93%, 94%, 95%, 96%, 97%, 98%, or 99% identity with a corresponding contiguous portion of NFE2L1 LCR positions 48048359-48061545.

In various embodiments, an Ad35 vector can include a NFE2L1 LCR as provided herein, e.g., in a payload that includes the NFE2L1 LCR and, optionally, a promoter of a gene that is typically operably linked with the NFE2L1 LCR in the human genome. In various embodiments, the gene operably linked with the NFE2L1 LCR is NFE2L1 (17:48,048,358-48,061,544). In various embodiments, a NFE2L1 promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a NFE2L1 promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, NFE2L1, e.g., in a reference genome. In some embodiments, the first coding nucleotide of a coding sequence of a gene that is typically operably linked with the NFE2L1 LCR in the human genome is the first coding nucleotide of NFE2L1 at Chromosome 17 - NC_000017.11 (48051119).

In various embodiments, a NFE2L1 LCR, such as a long NFE2L1 LCR, causes expression of an operably linked coding sequence in erythrocytes. In various embodiments, the operably linked coding sequence is also operably linked with a NFE2L1 promoter as set forth herein or otherwise known in the art.

The CD4 LCR is an exemplary LCR that enhances expression of operably linked coding sequences. Expression of a coding sequence can be enhanced when operably linked with a CD4 LCR that includes the complete CD4 LCR sequence and/or that includes an expression-regulatory fragment thereof. The CD4 LCR includes DNAse hypersensitive sites (HS) understood by those of skill in the art to mediate at least some of the expression-enhancing effects of the CD4 LCR. The CD4 LCR includes up to 17 hypersensitive sites DH1-DH17. Accordingly, a CD4 LCR can be a complete CD4 LCR including all of DH1-DH17, or can be an expression-regulatory fragment thereof that includes a subset of the hypersensitive sites DH1-DH17.

In various embodiments, an Ad35 vector can include a CD4 LCR as provided herein, e.g., in a payload that includes the CD4 LCR and, optionally, a promoter of a gene that is typically operably linked with the CD4 LCR in the human genome. In various embodiments, the gene operably linked with the CD4 LCR is CD4 (12:6,789,527-6,820,809). In various embodiments, a CD4 promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a CD4 promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, CD4, e.g., in a reference genome. In some embodiments, the first coding nucleotide of a coding sequence of a gene that is typically operably linked with the CD4 LCR in the human genome is the first coding nucleotide of CD4 at Chromosome 12 - NC_000012.12 (6800139).

In various embodiments, a CD4 LCR, such as a long CD4 LCR, causes expression of an operably linked coding sequence in CD4+ T Cells. In various embodiments, the operably linked coding sequence is also operably linked with a CD4 promoter as set forth herein or otherwise known in the art.

The α-lactalbumin LCR is an exemplary LCR that enhances expression of operably linked coding sequences. Expression of a coding sequence can be enhanced when operably linked with a α-lactalbumin LCR that includes the complete α-lactalbumin LCR sequence and/or that includes an expression-regulatory fragment thereof. The α-lactalbumin LCR includes DNAse hypersensitive sites (HS) understood by those of skill in the art to mediate at least some of the expression-enhancing effects of the α-lactalbumin LCR.

In various embodiments, an Ad35 vector can include a α-lactalbumin LCR as provided herein, e.g., in a payload that includes the α-lactalbumin LCR and, optionally, a promoter of a gene that is typically operably linked with the α-lactalbumin LCR in the human genome. In various embodiments, the gene operably linked with the α-lactalbumin LCR is α-lactalbumin (12:48,567,683-48,571,882). In various embodiments, an α-lactalbumin promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, an α-lactalbumin promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, α-lactalbumin, e.g., in a reference genome. In some embodiments, the first coding nucleotide of a coding sequence of a gene that is typically operably linked with the α-lactalbumin LCR in the human genome is the first coding nucleotide of α-lactalbumin at Chromosome 12 - NC_000012.12 (48570020).

In various embodiments, a α-lactalbumin LCR, such as a long α-lactalbumin LCR, causes expression of an operably linked coding sequence in mammary glands. In various embodiments, the operably linked coding sequence is also operably linked with an α-lactalbumin promoter as set forth herein or otherwise known in the art.

The CYP19/aromatase LCR is an exemplary LCR that enhances expression of operably linked coding sequences. Expression of a coding sequence can be enhanced when operably linked with a CYP19/aromatase LCR that includes the complete CYP19/aromatase LCR sequence and/or that includes an expression-regulatory fragment thereof. The CYP19/aromatase LCR includes DNAse hypersensitive sites (HS) understood by those of skill in the art to mediate at least some of the expression-enhancing effects of the CYP19/aromatase LCR.

In various embodiments, an Ad35 vector can include a CYP19/aromatase LCR as provided herein, e.g., in a payload that includes the CYP19/aromatase LCR and, optionally, a promoter of a gene that is typically operably linked with the CYP19/aromatase LCR in the human genome. In various embodiments, the gene operably linked with the CYP19/aromatase LCR is CYP19A1 (15:51,208,056-51,338,595). In various embodiments, a CYP19A1 promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a CYP19A1 promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, CYP19A1, e.g., in a reference genome. In some embodiments, the first coding nucleotide of a coding sequence of a gene that is typically operably linked with the CYP19/aromatase LCR in the human genome is the first coding nucleotide of CYP19A1 at Chromosome 15 - NC_000015.10 (51242912).

In various embodiments, a CYP19/aromatase LCR, such as a long CYP19/aromatase LCR, causes expression of an operably linked coding sequence in multiple various tissues. In various embodiments, the operably linked coding sequence is also operably linked with a CYP19A1 promoter as set forth herein or otherwise known in the art.

The C-fes proto-oncogene LCR is an exemplary LCR that enhances expression of operably linked coding sequences. Expression of a coding sequence can be enhanced when operably linked with a C-fes proto-oncogene LCR that includes the complete C-fes proto-oncogene LCR sequence and/or that includes an expression-regulatory fragment thereof. The C-fes proto-oncogene LCR includes DNAse hypersensitive sites (HS) understood by those of skill in the art to mediate at least some of the expression-enhancing effects of the C-fes proto-oncogene LCR.

In various embodiments, an Ad35 vector can include a C-fes proto-oncogene LCR as provided herein, e.g., in a payload that includes the C-fes proto-oncogene LCR and, optionally, a promoter of a gene that is typically operably linked with the C-fes proto-oncogene LCR in the human genome. In various embodiments, the gene operably linked with the C-fes proto-oncogene LCR is FES (15:90,884,420-90,895,775). In various embodiments, a FES promoter can have a total length equal to or greater than 100 bp, 200 bp, 300 bp, 400 bp, 500 bp, 1.0 kb, 1.5 kb, 2.0 kb, 2.5 kb, 3.0 kb, 4.0 kb, or 5.0 kb. In various embodiments, a FES promoter includes at least 100 bp, 200 bp, 300 bp, 400 bp, 500 bp, 1.0 kb, 1.5 kb, 2.0 kb, 2.5 kb, 3.0 kb, 4.0 kb, or 5.0 kb having at least 70%, 75%, 80%, 85%, 90%, 91%, 92%, 93%, 94%, 95%, 96%, 97%, 98%, or 99% identity with a corresponding nucleic acid sequence that is upstream of, e.g., immediately upstream of the first coding nucleotide of, FES, e.g., in a reference genome. In some embodiments, the first coding nucleotide of a coding sequence of a gene that is typically operably linked with the C-fes proto-oncogene LCR in the human genome is the first coding nucleotide of FES at Chromosome 15 - NC_000015.10 (90885046).

In various embodiments, a C-fes proto-oncogene LCR, such as a long C-fes proto-oncogene LCR, causes expression of an operably linked coding sequence in myeloid cells including macrophages and neutrophils. In various embodiments, the operably linked coding sequence is also operably linked with a FES promoter as set forth herein or otherwise known in the art.

### (IV) Coding Sequences Operably Linked With Long LCR

### (IV-b) Protein Therapy, E.g., Protein/enzyme Replacement Therapy

In particular embodiments, the coding sequence operably linked with long LCR includes a transgene encoding a therapeutic protein. The coding sequence refers to a nucleic acid sequence (used interchangeably with polynucleotide or nucleotide sequence) that encodes one or more therapeutic proteins as described herein. This definition includes various sequence polymorphisms, mutations, and/or sequence variants wherein such alterations do not substantially affect the function of the encoded one or more therapeutic proteins. The coding sequence or “gene” may include not only coding sequences but also regulatory regions such as promoters, enhancers, and termination regions. The term further can include all introns and other DNA sequences spliced from the mRNA transcript, along with variants resulting from alternative splice sites. Gene sequences encoding the molecule can be DNA or RNA that directs the expression of the one or more therapeutic proteins. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type.

A gene sequence encoding one or more therapeutic proteins can be readily prepared by synthetic or recombinant methods from the relevant amino acid sequence. In particular embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence. In particular embodiments, the gene sequence encoding the sequences can be codon optimized for expression in mammalian cells. A coding sequence for a therapeutic protein is herein referred to as a therapeutic gene.

A therapeutic gene can be selected to provide a therapeutically effective response against a condition that, in particular embodiments, is inherited. In particular embodiments, the condition can be Grave’s Disease, rheumatoid arthritis, pernicious anemia, Multiple Sclerosis (MS), inflammatory bowel disease, systemic lupus erythematosus (SLE), adenosine deaminase deficiency (ADA-SCID) or severe combined immunodeficiency disease (SCID), Wiskott-Aldrich syndrome (WAS), chronic granulomatous disease (CGD), Fanconi anemia (FA), Battens disease, adrenoleukodystrophy (ALD) or metachromatic leukodystrophy (MLD), muscular dystrophy, pulmonary alveolar proteinosis (PAP), pyruvate kinase deficiency, Schwachman-Diamond-Blackfan anemia, dyskeratosis congenita, cystic fibrosis, Parkinson’s disease, Alzheimer’s disease, or amyotrophic lateral sclerosis (Lou Gehrig’s disease). In particular embodiments, depending on the condition, the therapeutic gene may be a gene that encodes a protein and/or a gene whose function has been interrupted.

Exemplary therapeutic gene and gene products include: antibodies to CD4, CD5, CD7, CD52, etc.; antibodies; antibodies to IL1, IL2, IL6; an antibody to TCR specifically present on autoreactive T cells; IL4; IL10; IL12; IL13; IL1 Ra; sIL1RI; sIL1 RII; antibodies to TNF; ABCA3; ABCD1; ADA; AK2; APP; arginase; arylsulfatase A; A1AT; CD3D; CD3E; CD3G; CD3Z; CFTR; CHD7; chimeric antigen receptor (CAR); CIITA; CLN3; complement factor, CORO1A; CTLA; C1 inhibitor; C9ORF72; DCLRE1B; DCLRE1C; decoy receptors; DKC1; DRB1*1501/DQB1*0602; dystrophin; enzymes; Factor VIII, FANC family genes (FancA, FancB, FancC, FancD1 (BRCA2), FancD2, FancE, FancF, FancG, FancI, FancJ (BRIP1), FancL, FancM, FancN (PALB2), FancO (RAD51 C), FancP (SLX4), FancQ (ERCC4), FancR (RAD51), FancS (BRCA1 ), FancT (UBE2T), FancU (XRCC2), FancV (MAD2L2), and FancW (RFWD3)); Fas L; FUS; GATA1; globin family genes (ie. γ-globin); F8; glutaminase; HBA1; HBA2; HBB; IL7RA; JAK3; LCK; LIG4; LRRK2; NHEJ1; NLX2.1; neutralizing antibodies; ORAI1; PARK2; PARK7; phox; PINK1; PNP; PRKDC; PSEN1; PSEN2; PTPN22; PTPRC; P53; pyruvate kinase; RAG1; RAG2; RFXANK; RFXAP; RFX5; RMRP; ribosomal protein genes; SFTPB; SFTPC; SOD1; soluble CD40; STIM1; sTNFRI; sTNFRII; SLC46A1; SNCA; TDP43; TERT; TERC; TINF2; ubiquilin 2; WAS; WHN; ZAP70; γC; and other therapeutic genes described herein.

Therapeutically effective amounts may provide function to immune and other blood cells and/or microglial cells or may alternatively—depending on the treated condition—inhibit lymphocyte activation, induce apoptosis in lymphocytes, eliminate various subsets of lymphocytes, inhibit T cell activation, eliminate or inhibit autoreactive T cells, inhibit Th-2 or Th-1 lymphocyte activity, antagonize IL-1 or TNF, reduce inflammation, induce selective tolerance to an inciting agent, reduce or eliminate an immune-mediated condition; and/or reduce or eliminate a symptom of the immune-mediated condition. Therapeutically effective amounts may also provide functional DNA repair mechanisms; surfactant protein expression; telomere maintenance; lysosomal function; breakdown of lipids or other proteins such as amyloids; permit ribosomal function; and/or permit development of mature blood cell lineages which would otherwise not develop such as macrophages other white blood cell types.

As another example, a therapeutic gene can be selected to provide a therapeutically effective response against diseases related to red blood cells and clotting. In particular embodiments, the disease is a hemoglobinopathy like thalassemia, or a sickle cell disease/trait. The therapeutic gene may be, for example, a gene that induces or increases production of hemoglobin; induces or increases production of β-globin, γ-globin, or α-globin; or increases the availability of oxygen to cells in the body. The therapeutic gene may be, for example, HBB or CYB5R3. Exemplary effective treatments may, for example, increase blood cell counts, improve blood cell function, or increase oxygenation of cells in patients. In another particular embodiment, the disease is hemophilia. The therapeutic gene may be, for example, a gene that increases the production of coagulation/clotting factor VIII or coagulation/clotting factor IX, causes the production of normal versions of coagulation factor VIII or coagulation factor IX, a gene that reduces the production of antibodies to coagulation/clotting factor VIII or coagulation/clotting factor IX, or a gene that causes the proper formation of blood clots. Exemplary therapeutic genes include F8 and F9. Exemplary effective treatments may, for example, increase or induce the production of coagulation/clotting factors VIII and IX; improve the functioning of coagulation/clotting factors VIII and IX, or reduce clotting time in subjects.

The following references describe particular exemplary sequences of functional globin genes. References 1-4 relate to α-type globin sequences and references 4-12 relate to β-type globin sequences (including β and γ globin sequences): (1) GenBank Accession No. Z84721 (Mar. 19, 1997); (2) GenBank Accession No. NM_000517 (Oct. 31, 2000); (3) Hardison et al., J. Mol. Biol. 222(2):233-249, 1991; (4) A Syllabus of Human Hemoglobin Variants (1996), by Titus et al., published by The Sickle Cell Anemia Foundation in Augusta, GA (available online at globin.cse.psu.edu); (5) GenBank Accession No. J00179 (Aug. 26, 1993); (6) Tagle et al., Genomics 13(3):741-760, 1992; (7) Grovsfeld et al., Cell 51(6):975-985, 1987; (8) Li et al., Blood 93(7):2208-2216, 1999; (9) Gorman et al., J. Biol. Chem .275(46):35914-35919, 2000; (10) Slightom et al., Cell 21(3):627-638, 1980; (11) Fritsch et al., Cell 19(4): 959-972, 1980; (12) Marotta et al., J. Biol. Chem. 252(14):5040-5053, 1977. For additional coding and non-coding regions of genes encoding globins see, for example, by Marotta et al., Prog. Nucleic Acid Res. Mol. Biol. 19, 165-175, 1976, Lawn et al., Cell 21 (3), 647-651, 1980, and Sadelain et al., PNAS. 92:6728-6732, 1995.

An exemplary amino acid sequence of hemoglobin subunit β is provided, for example, at NCBI Accession No. P68871. An exemplary amino acid sequence for β-globin is provided, for example, at NCBI Accession No. NP_000509.

As another example, a therapeutic gene can be selected to provide a therapeutically effective response against a lysosomal storage disorder. In particular embodiments, the lysosomal storage disorder is mucopolysaccharidosis (MPS), type I; MPS II or Hunter Syndrome; MPS III or Sanfilippo syndrome; MPS IV or Morquio syndrome; MPS V; MPS VI or Maroteaux-Lamy syndrome; MPS VII or sly syndrome; α-mannosidosis; β-mannosidosis; glycogen storage disease type 1, also known as GSDI, von Gierke disease, or Tay Sachs; Pompe disease; Gaucher disease; Fabry disease. The therapeutic gene may be, for example a gene encoding or inducing production of an enzyme, or that otherwise causes the degradation of mucopolysaccharides in lysosomes. Exemplary therapeutic genes include IDUA or iduronidase, IDS, GNS, HGSNAT, SGSH, NAGLU, GUSB, GALNS, GLB1, ARSB, and HYAL1. Exemplary effective genetic therapies for lysosomal storage disorders may, for example, encode or induce the production of enzymes responsible for the degradation of various substances in lysosomes; reduce, eliminate, prevent, or delay the swelling in various organs, including the head (exp. Macrosephaly), the liver, spleen, tongue, or vocal cords; reduce fluid in the brain; reduce heart valve abnormalities; prevent or dilate narrowing airways and prevent related upper respiratory conditions like infections and sleep apnea; reduce, eliminate, prevent, or delay the destruction of neurons, and/or the associated symptoms.

As another example, a therapeutic gene can be selected to provide a therapeutically effective response against a hyperproliferative disease. In particular embodiments, the hyperproliferative disease is cancer. The therapeutic gene may be, for example, a tumor suppressor gene, a gene that induces apoptosis, a gene encoding an enzyme, a gene encoding an antibody, or a gene encoding a hormone. Exemplary therapeutic genes and gene products include (in addition to those listed elsewhere herein) 101 F6, 123F2 (RASSF1), 53BP2, abl, ABLI, ADP, aFGF, APC, ApoAl, ApoAIV, ApoE, ATM, BAI-1, BDNF, Beta*(BLU), bFGF, BLC1, BLC6, BRCA1, BRCA2, CBFA1, CBL, C-CAM, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-5, IL-6, IL-7, IL-8, IL-9, IL-11, ING1, interferon α, interferon β, interferon γ, IRF-1, JUN, KRAS, LUCA-1 (HYAL1), LUCA-2 (HYAL2), LYN, MADH4, MADR2, MCC, mda7, MDM2, MEN-1, MEN-11, MLL, MMAC1, MYB, MYC, MYCL1, MYCN, neu, NF-1, NF-2, NGF, NOEY1, NOEY2, NRAS, NT3, NT5, OVCA1, p16, p21, p27, p57, p73, p300, PGS, PIM1, PL6, PML, PTEN, raf, Rap1A, ras, Rb, RB1, RET, rks-3, ScFv, scFV ras, SEM A3, SRC, TALI, TCL3, TFPI, thrombospondin, thymidine kinase, TNF, TP53, trk, T-VEC, VEGF, VHL, WT1, WT-1, YES, and zac1. Exemplary effective genetic therapies may suppress or eliminate tumors, result in a decreased number of cancer cells, reduced tumor size, slow or eliminate tumor growth, or alleviate symptoms caused by tumors.

As another example, a therapeutic gene can be selected to provide a therapeutically effective response against an infectious disease. In particular embodiments, the infectious disease is human immunodeficiency virus (HIV). The therapeutic gene may be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and gene products include α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/a2MR/LRP; PVR; PRR1/HveC; and laminin receptor. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

### (IV-c) Antibodies, CARs, and TCRs

In addition to therapeutic genes and/or gene products, the coding sequence can also encode for therapeutic molecules, such as antibodies, chimeric antigen receptor molecules specific to one or more cancer antigen and/or T-cell receptor specific to one or more cancer antigen.

Significant progress has been made in genetically engineering T cells of the immune system to target and kill unwanted cell types, such as cancer cells. Many of these T cells have been genetically engineered to express chimeric antigen receptor (CAR) constructs. CARs are proteins including several distinct subcomponents that allow the genetically modified T cells to recognize and kill cancer cells. The subcomponents include at least an extracellular component and an intracellular component.

The extracellular component includes a binding domain that specifically binds a marker that is preferentially present on the surface of unwanted cells. When the binding domain binds such markers, the intracellular component directs the T cell to destroy the bound cancer cell. The binding domain is typically a single-chain variable fragment (scFv) derived from a monoclonal antibody (mAb), but it can be based on other formats which include an antibody-like antigen binding site.

The intracellular components provide activation signals based on the inclusion of an effector domain. First generation CARs utilized the cytoplasmic region of CD3ζ as an effector domain. Second generation CARs utilized CD3ζ in combination with cluster of differentiation 28 (CD28) or 4-1 BB (CD137), while third generation CARs have utilized CD3ζ in combination with CD28 and 4-1 BB within intracellular effector domains.

CAR generally also include one or more linker sequences that are used for a variety of purposes within the molecule. For example, a transmembrane domain can be used to link the extracellular component of the CAR to the intracellular component. A flexible linker sequence often referred to as a spacer region that is membrane-proximal to the binding domain can be used to create additional distance between a binding domain and the cellular membrane. This can be beneficial to reduce steric hindrance to binding based on proximity to the membrane. More compact spacers or longer spacers can be used, depending on the targeted cell marker. Other potential CAR subcomponents are described in more detail elsewhere herein. Components of CAR are now described in additional detail as follows: Binding Domains; Intracellular Signaling Components; Linkers; Transmembrane Domains; Junction Amino Acids; and Control Features Including Tag Cassettes. The description about binding domains is also relevant to antibodies as a therapeutic molecule.

Binding Domains. Binding domains include any substance that binds to a cellular marker to form a complex. The choice of binding domain can depend upon the type and number of cellular markers that define the surface of a target cell. Examples of binding domains include cellular marker ligands, receptor ligands, antibodies, peptides, peptide aptamers, receptors (e.g., T cell receptors), chimeric antigen receptors (CARs), or combinations and engineered fragments or formats thereof.

Antibodies are one example of binding domains and include whole antibodies or binding fragments of an antibody, e.g., Fv, Fab, Fab′, F(ab′)₂, and single chain (sc) forms and fragments thereof that bind specifically to a cellular marker. Antibodies or antigen binding fragments can include all or a portion of polyclonal antibodies, monoclonal antibodies, human antibodies, humanized antibodies, synthetic antibodies, non-human antibodies, recombinant antibodies, chimeric antibodies, bispecific antibodies, mini bodies, and linear antibodies.

Antibodies are produced from two genes, a heavy chain gene and a light chain gene. Generally, an antibody includes two identical copies of a heavy chain, and two identical copies of a light chain. Within a variable heavy chain and variable light chain, segments referred to as complementary determining regions (CDRs) dictate epitope binding. Each heavy chain has three CDRs (i.e., CDRH1, CDRH2, and CDRH3) and each light chain has three CDRs (i.e., CDRL1, CDRL2, and CDRL3). CDR regions are flanked by framework residues (FR).

In some instances, it is beneficial for the binding domain to be derived from the same species it will ultimately be used in. For example, for use in humans, it may be beneficial for the antigen binding domain to include a human antibody, humanized antibody, or a fragment or engineered form thereof. Antibodies from human origin or humanized antibodies have lowered or no immunogenicity in humans and have a lower number of non-immunogenic epitopes compared to non-human antibodies. Antibodies and their engineered fragments will generally be selected to have a reduced level or no antigenicity in human subjects.

In particular embodiments, the binding domain includes a humanized antibody or an engineered fragment thereof. In particular embodiments, a non-human antibody is humanized, where one or more amino acid residues of the antibody are modified to increase similarity to an antibody naturally produced in a human or fragment thereof. These nonhuman amino acid residues are often referred to as “import” residues, which are typically taken from an “import” variable domain. As provided herein, humanized antibodies or antibody fragments include one or more CDRs from nonhuman immunoglobulin molecules and framework regions wherein the amino acid residues including the framework are derived completely or mostly from human germline. In one aspect, the antigen binding domain is humanized. A humanized antibody can be produced using a variety of techniques known in the art, including CDR-grafting (see, e.g., European Patent No. EP 239,400; WO 91/09967; and US 5,225,539, US 5,530,101, and US 5,585,089), veneering or resurfacing (see, e.g., EP 592,106 and EP 519,596; Padlan, Molecular Immunology, 28(⅘):489-498, 1991; Studnicka et al., Protein Engineering, 7(6):805-81, 19944; and Roguska et al., PNAS, 91:969-973, 1994), chain shuffling (see, e.g., U.S. Pat. No. 5,565,332), and techniques disclosed in, e.g., U.S. Publication No. 2005/0042664, U.S. Publication No. 2005/0048617, U.S. Pat. No. 6,407,213, US Pat. No. 5,766,886, WO 9317105, Tan et al., J. Immunol., 169:1119-25, 2002, Caldas et al., Protein Eng., 13(5):353-60, 2000, Morea et al., Methods, 20(3):267-79, 2000, Baca et al., J. Biol. Chem., 272(16): 10678-84, 1997, Roguska et al., Protein Eng., 9(10):895-904, 1996, Couto et al., Cancer Res., 55 (23 Supp):5973s-5977s, 1995, Couto etal., Cancer Res., 55(8):1717-22, 1995, Sandhu, Gene, 150(2):409-10, 1994, and Pedersen et al., J. Mol. Biol., 235(3):959-73, 1994. Often, framework residues in the framework regions will be substituted with the corresponding residue from the CDR donor antibody to alter, for example improve, cellular marker binding. These framework substitutions are identified by methods well-known in the art, e.g., by modeling of the interactions of the CDR and framework residues to identify framework residues important for cellular marker binding and sequence comparison to identify unusual framework residues at particular positions. (See, e.g., U.S. Pat. No. 5,585,089; and Riechmann et al., Nature, 332:323, 1988).

Antibodies with binding domains that specifically bind a cellular marker can be prepared using methods of obtaining monoclonal antibodies, methods of phage display, methods to generate human or humanized antibodies, or methods using a transgenic animal or plant engineered to produce antibodies as is known to those of ordinary skill in the art (see, for example, US 6,291,161 and US 6,291,158). Phage display libraries of partially or fully synthetic antibodies are available and can be screened for an antibody or fragment thereof that can bind to a cellular marker. For example, binding domains may be identified by screening a Fab phage library for Fab fragments that specifically bind a cellular marker (see Hoet et al., Nat. Biotechnol. 23:344, 2005). Phage display libraries of human antibodies are also available. Additionally, traditional strategies for hybridoma development using a cellular marker as an immunogen in convenient systems (e.g., mice, HuMAb mouse® (GenPharm Int′l. Inc., Mountain View, CA), TC mouse® (Kirin Pharma Co. Ltd., Tokyo, JP), KM-mouse® (Medarex, Inc., Princeton, NJ), llamas, chicken, rats, hamsters, rabbits, etc.) can be used to develop binding domains. Once identified, the amino acid sequence of the antibody and gene sequence encoding the antibody can be isolated and/or determined.

In some instances, scFvs can be prepared according to methods known in the art (see, for example, Bird et al., Science 242:423-426 1988; and Huston et al., Proc. Natl. Acad. Sci. USA 85:5879-5883, 1988). ScFv molecules can be produced by linking VH and VL regions of an antibody together using flexible polypeptide linkers. If a short polypeptide linker is employed (e.g., between 5-10 amino acids) intrachain folding is prevented. Interchain folding is also required to bring the two variable regions together to form a functional epitope binding site. For examples of linker orientations and sizes see, e.g., Hollinger et al., Proc Natl Acad. Sci. U.S.A. 90:6444-6448, 1993, U.S. Publication No. 2005/0100543, U.S. Publication No. 2005/0175606, U.S. Publication No. 2007/0014794, and WO2006/020258 and WO2007/024715. More particularly, linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. scFv are commonly used as the binding domains of CAR.

Additional examples of antibody-based binding domain formats include scFv-based grababodies and soluble VH domain antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

In particular embodiments, a VL region in a binding domain of the present disclosure is derived from or based on a VL of a known monoclonal antibody and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the known monoclonal antibody. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

In particular embodiments, a binding domain VH region of the present disclosure can be derived from or based on a VH of a known monoclonal antibody and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of a known monoclonal antibody. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

In particular embodiments, a binding domain includes or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (VL) or to a heavy chain variable region (VH), or both, wherein each CDR includes zero changes or at most one, two, or three changes, from a monoclonal antibody or fragment or derivative thereof that specifically binds to a cellular marker of interest.

An alternative source of binding domains includes sequences that encode random peptide libraries or sequences that encode an engineered diversity of amino acids in loop regions of alternative non-antibody scaffolds, such as single chain (sc) T-cell receptor (scTCR) (see, e.g., Lake et al., Int. Immunol. 11:745, 1999; Maynard et al., J. Immunol. Methods 306:51, 2005; US 8,361,794), fibrinogen domains (see, e.g., Weisel et al., Science 230:1388, 1985), Kunitz domains (see, e.g., US 6,423,498), designed ankyrin repeat proteins (DARPins; Binz et al., J. Mol. Biol. 332:489, 2003 and Binz et al., Nat. Biotechnol. 22:575, 2004), fibronectin binding domains (adnectins or monobodies; Richards et al., J. Mol. Biol. 326:1475, 2003; Parker et al., Protein Eng. Des. Selec. 18:435, 2005 and Hackel et al., J. Mol. Biol. 381:1238-1252, 2008), cysteine-knot miniproteins (Vita et al., Proc. Nat′l. Acad. Sci. (USA) 92:6404-6408, 1995; Martin et al., Nat. Biotechnol. 21:71, 2002 and Huang et al., Structure 13:755, 2005), tetratricopeptide repeat domains (Main et al., Structure 11:497, 2003 and Cortajarena et al., ACS Chem. Biol. 3:161, 2008), leucine-rich repeat domains (Stumpp et al., J. Mol. Biol. 332:471, 2003), lipocalin domains (see, e.g., WO 2006/095164, Beste et al., Proc. Nat′l. Acad. Sci. (USA) 96:1898, 1999 and Schönfeld et al., Proc. Nat′l. Acad. Sci. (USA) 106:8198, 2009), V-like domains (see, e.g., US 2007/0065431), C-type lectin domains (Zelensky & Gready, FEBS J. 272:6179, 2005; Beavil et al., Proc. Nat′l. Acad. Sci. (USA) 89:753, 1992 and Sato et al., Proc. Nat′l. Acad. Sci. (USA) 100:7779, 2003), mAb2 or Fc-region with antigen binding domain (Fcab™ (F-Star Biotechnology, Cambridge UK; see, e.g., WO 2007/098934 and WO 2006/072620), armadillo repeat proteins (see, e.g., Madhurantakam et al., Protein Sci. 21: 1015, 2012; WO 2009/040338), affilin (Ebersbach et al., J. Mol. Biol. 372: 172, 2007), affibody, avimers, knottins, fynomers, atrimers, cytotoxic T-lymphocyte associated protein-4 (Weidle et al., Cancer Gen. Proteo. 10:155, 2013), or the like (Nord et al., Protein Eng. 8:601, 1995; Nord et al., Nat. Biotechnol. 15:772, 1997; Nord et al., Euro. J. Biochem. 268:4269, 2001; Binz et al., Nat. Biotechnol. 23:1257, 2005; Boersma & Plückthun, Curr. Opin. Biotechnol. 22:849, 2011).

Peptide aptamers include a peptide loop (which is specific for a cellular marker) attached at both ends to a protein scaffold. This double structural constraint increases the binding affinity of peptide aptamers to levels comparable to antibodies. The variable loop length is typically 8 to 20 amino acids and the scaffold can be any protein that is stable, soluble, small, and non-toxic. Peptide aptamer selection can be made using different systems, such as the yeast two-hybrid system (e.g., Gal4 yeast-two-hybrid system), or the LexA interaction trap system.

In particular embodiments, a binding domain is a sc T cell receptor (scTCR) including Vα/β and Cα/β chains (e.g., Vα-Cα, Vβ-Cβ, Vα-Vβ) or including a Vα-Cα, Vβ-Cβ, Vα-Vβ pair specific for a cellular marker peptide-MHC complex.

In particular embodiments, engineered binding domains include Vα, Vβ, Cα, or Cβ regions derived from or based on a Vα, Vβ, Cα, or Cβ and includes one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the referenced Vα, Vβ, Cα, or Cβ. An insertion, deletion or substitution may be anywhere in a V_{L}, V_{H}, Vα, Vβ, Cα, or Cβ region, including at the amino- or carboxy-terminus or both ends of these regions, provided that each CDR includes zero changes or at most one, two, or three changes and provides a target binding domain containing a modified Vα, Vβ, Cα, or Cβ region can still specifically bind its target with an affinity and action similar to wild type.

In particular embodiments, engineered binding domains include a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a known or identified binding domain, wherein each CDR includes zero changes or at most one, two, or three changes, from a known or identified binding domain or fragment or derivative thereof that specifically binds to the targeted cellular marker.

The precise amino acid sequence boundaries of a given CDR or FR can be readily determined using any of a number of well-known schemes, including those described by: Kabat et al. (1991) “Sequences of Proteins of Immunological Interest,” 5th Ed. Public Health Service, National Institutes of Health, Bethesda, Md. (Kabat numbering scheme); Al-Lazikani et al., J Mol Biol 273: 927-948,1997 (Chothia numbering scheme); Maccallum et al., J Mol Biol 262: 732-745, 1996 (Contact numbering scheme); Martin et al., Proc. Natl. Acad. Sci., 86: 9268-9272, 1989 (AbM numbering scheme); Lefranc et al., Dev Comp Immunol 27(1): 55-77, 2003 (IMGT numbering scheme); and Honegger & Pluckthun, J Mol Biol 309(3): 657-670, 2001 (“Aho” numbering scheme). The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a”, and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In particular embodiments, the antibody CDR sequences disclosed herein are according to Kabat numbering.

A CAR is an engineered receptor designed to bind to certain targets and elicit a response. CARs include several distinct subcomponents that, when expressed on a cell, allow the genetically modified cell to recognize and kill unwanted cells, such as cancer cells or virally-infected cells. The subcomponents include at least an extracellular component and an intracellular component. The extracellular component includes a binding domain that specifically binds a marker that is preferentially present on the surface of unwanted cells. When the binding domain binds such markers, the intracellular component activates the genetically modified cell to destroy the bound cell. CAR additionally include a transmembrane domain that links the extracellular component to the intracellular component, and other subcomponents that can increase the CAR’s function. For example, the inclusion of one or more linker sequences, such as a spacer region, can allow the CAR to have additional conformational flexibility, often increasing the binding domain’s ability to bind the targeted cell marker.

The extracellular domain of a CAR includes a binding domain. Binding domains were discussed previously and can include antibodies, scFvs, ligands, peptides, peptide aptamers, or receptors.

In particular embodiments, engineered CAR include a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a known or identified TCR Vα, Vβ, Cα, or Cβ, wherein each CDR includes zero changes or at most one, two, or three changes, from a TCR or fragment or derivative thereof that specifically binds to the targeted cellular marker.

In particular embodiments, engineered CAR include Vα, Vβ, Cα, or Cβ regions derived from or based on a Vα, Vβ, Cα, or Cβ of a known or identified TCR (e.g., a high-affinity TCR) and includes one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the Vα, Vβ, Cα, or Cβ of a known or identified TCR. An insertion, deletion or substitution may be anywhere in a Vα, Vβ, Cα, or Cβ region, including at the amino- or carboxy-terminus or both ends of these regions, provided that each CDR includes zero changes or at most one, two, or three changes and provides a target binding domain containing a modified Vα, Vβ, Cα, or Cβ region can still specifically bind its target with an affinity and action similar to wild type.

In particular embodiments, a binding domain of a CAR includes or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (VL) or to a heavy chain variable region (VH), or both, wherein each CDR includes zero changes or at most one, two, or three changes, from a monoclonal antibody or fragment or derivative thereof that specifically binds to a cellular marker of interest.

In particular embodiments, a VL region in a CAR of the present disclosure is derived from or based on a VL of a known monoclonal antibody and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the known monoclonal antibody. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

In particular embodiments, a binding domain VH region in a CAR of the present disclosure can be derived from or based on a VH of a known monoclonal antibody and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of a known monoclonal antibody. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

Particular cellular markers associated with prostate cancer include PSMA, WT1, ProstateStem Cell antigen (PSCA), and SV40 T. Particular cellular markers associated with breast cancer include HER2 and ERBB2. Particular cellular markers associated with ovarian cancer include L1-CAM, extracellular domain of MUC16 (MUC-CD), folate binding protein (folate receptor), Lewis Y, mesothelin, and WT-1. Particular cellular markers associated with pancreatic cancer include mesothelin, CEA and CD24. Particular cellular markers associated with multiple myeloma include BCMA, GPRC5D, CD38, and CS-1. Particular markers associated with leukemia and/or lymphoma include CLL-1, CD123, CD33, and PD-L1.

In particular embodiments, the binding domain of a CAR binds the cellular marker Her2. In particular embodiments, the binding domain that binds HER2 is derived from trastuzumab (Herceptin). In particular embodiments, the binding domain includes a variable light chain including a CDRL1 sequence including SEQ ID NO: 8, a CDRL2 sequence including SEQ ID NO: 9, and a CDRL3 sequence including SEQ ID NO: 10, and a variable heavy chain including a CDRH1 sequence including SEQ ID NO: 11, a CDRH2 sequence including SEQ ID NO: 12, and a CDRH3 sequence including SEQ ID NO: 13.

In particular embodiments, the binding domain of a CAR binds the cellular marker PD-L1. In particular embodiments, the binding domain that binds PD-L1 is derived from at least one of pembrolizumab or FAZ053 (Novartis). In particular embodiments, the binding domain includes a variable light chain including a CDRL1 sequence including SEQ ID NO: 14, a CDRL2 sequence including SEQ ID NO: 15, and a CDRL3 sequence including SEQ ID NO: 16, and a variable heavy chain including a CDRH1 sequence including SEQ ID NO: 17, a CDRH2 sequence including SEQ ID NO: 18, and a CDRH3 sequence including SEQ ID NO: 19.

An exemplary binding domain for PD-L1 can include or be derived from Avelumab or Atezolizumab. In particular embodiments, the variable heavy chain of Avelumab includes SEQ ID NO: 20.

In particular embodiments, the variable light chain of Avelumab includes SEQ ID NO: 21.

In particular embodiments, the CDR regions of Avelumab include: CDRH1 (SEQ ID NO: 22); CDRH2 (SEQ ID NO: 23); CDRH3 (SEQ ID NO: 24); CDRL1 (SEQ ID NO: 25); CDRL2 (SEQ ID NO: 26); and CDRL3 (SEQ ID NO: 27).

In particular embodiments, the variable heavy chain of Atezolizumab includes SEQ ID NO: 28. In particular embodiments, the variable light chain of Atezolizumab includes SEQ ID NO: 29.

In particular embodiments, the CDR regions of Atezolizumab include: CDRH1 (SEQ ID NO: 30); CDRH2 (SEQ ID NO: 31); CDRH3 (SEQ ID NO: 32); CDRL1 (SEQ ID NO: 33); CDRL2 (SEQ ID NO: 34); and CDRL3 (SEQ ID NO: 35).

In particular embodiments, the binding domain of a CAR binds the cellular marker PSMA. In particular embodiments, the binding domain includes a variable light chain including a CDRL1 sequence including SEQ ID NO: 36, a CDRL2 sequence including SEQ ID NO: 37, a CDRL3 sequence including SEQ ID NO: 38. In particular embodiments, the binding domain includes a variable heavy chain including a CDRH1 sequence including SEQ ID NO: 39, a CDRH2 sequence including SEQ ID NO: 40, and a CDRH3 sequence including SEQ ID NO: 41.

In particular embodiments, the binding domain of a CAR binds the cellular marker MUC16. In particular embodiments, the binding domain is human or humanized and includes a variable light chain including a CDRL1 sequence including SEQ ID NO: 42, a CDRL2 sequence including GAS, a CDRL3 sequence including SEQ ID NO: 43. In particular embodiments, the binding domain is human or humanized and includes a variable heavy chain including a CDRH1 sequence including SEQ ID NO: 44, a CDRH2 sequence including SEQ ID NO: 45, and a CDRH3 sequence including SEQ ID NO: 46.

In particular embodiments, the binding domain of a CAR binds the cellular marker FOLR. In particular embodiments, the binding domain that binds FOLR is derived from farletuzumab. In particular embodiments, the binding domain includes a variable light chain including a CDRL1 sequence including SEQ ID NO: 47, a CDRL2 sequence including SEQ ID NO: 48, and a CDRL3 sequence including SEQ ID NO: 49, and a variable heavy chain including a CDRH1 sequence including SEQ ID NO: 50, a CDRH2 sequence including SEQ ID NO: 51, and a CDRH3 sequence including SEQ ID NO: 52.

An exemplary binding domain for mesothelin can include or be derived from Amatuximab.

In particular embodiments, t.he variable heavy chain of Amatuximab includes SEQ ID NO: 53. In particular embodiments, the variable light chain of Amatuximab includes SEQ ID NO: 54.

In particular embodiments, the CDR regions of Amatuximab include: CDRH1 (SEQ ID NO: 55); CDRH2 (SEQ ID NO: 56); CDRH3 (SEQ ID NO: 57); CDRL1 (SEQ ID NO: 58); CDRL2 (SEQ ID NO: 59); and CDRL3 (SEQ ID NO: 60).

Also contemplated are binding domains specific for infectious disease agents, for instance by binding to an infectious agent antigen. These include for instance viral antigens or other viral markers, for instance which are expressed by virally infected cells. Exemplary viruses include adenoviruses, arenaviruses, bunyaviruses, coronaviruses, flaviviruses, hantaviruses, hepadnaviruses, herpesviruses, papillomaviruses, paramyxoviruses, parvoviruses, picornaviruses, poxviruses, orthomyxoviruses, retroviruses, reoviruses, rhabdoviruses, rotaviruses, spongiform viruses or togaviruses. In additional embodiments, viral antigen markers include peptides expressed by CMV, cold viruses, Epstein-Barr, flu viruses, hepatitis A, B, and C viruses, herpes simplex, HIV, influenza, Japanese encephalitis, measles, polio, rabies, respiratory syncytial, rubella, smallpox, varicella zoster or West Nile virus.

As further particular examples, cytomegaloviral antigens include envelope glycoprotein B and CMV pp65; Epstein-Barr antigens include EBV EBNAI, EBV P18, and EBV P23; hepatitis antigens include the S, M, and L proteins of HBV, the pre-S antigen of HBV, HBCAG DELTA, HBV HBE, hepatitis C viral RNA, HCV NS3 and HCV NS4; herpes simplex viral antigens include immediate early proteins and glycoprotein D; HIV antigens include gene products of the gag, pol, and env genes such as HIV gp32, HIV gp41, HIV gp120, HIV gp160, HIV P17/24, HIV P24, HIV P55 GAG, HIV P66 POL, HIV TAT, HIV GP36, the Nef protein and reverse transcriptase; influenza antigens include hemagglutinin and neuraminidase; Japanese encephalitis viral antigens include proteins E, M-E, M-E-NS1, NS1, NS1-NS2A and 80% E; measles antigens include the measles virus fusion protein; rabies antigens include rabies glycoprotein and rabies nucleoprotein; respiratory syncytial viral antigens include the RSV fusion protein and the M2 protein; rotaviral antigens include VP7sc; rubella antigens include proteins E1 and E2; and varicella zoster viral antigens include gpl and gpll. Additional particular exemplary viral antigen sequences include: Nef (66-97) (SEQ ID NO: 61); Nef (116-145) (SEQ ID NO: 62); Gag p17 (17-35) (SEQ ID NO: 63); Gag p17-p24 (253-284) (SEQ ID NO: 64); and Pol 325-355 (RT 158-188) (SEQ ID NO: 65). See Fundamental Virology, Second Edition, eds. Fields, B. N. and Knipe, D. M. (Raven Press, New York, 1991) for additional examples of viral antigens.

Intracellular Signaling Components. The intracellular or otherwise the cytoplasmic signaling components of a CAR are responsible for activation of the cell in which the CAR is expressed. The term “intracellular signaling components” or “intracellular components” is thus meant to include any portion of the intracellular domain sufficient to transduce an activation signal. Intracellular components of expressed CAR can include effector domains. An effector domain is an intracellular portion of a fusion protein or receptor that can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from co-receptor or co-stimulatory molecule.

An effector domain can include one, two, three or more receptor signaling domains, intracellular signaling components (e.g., cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRα, FcRβ (FcεR1b), FcRy, Fyn, HVEM (LIGHTR), ICOS, LAG3, LAT, Lck, LRP, NKG2D, NOTCH1, pTα, PTCH2, OX40, ROR2, Ryk, SLAMF1, Slp76, TCRα, TCRβ, TRIM, Wnt, Zap70, or any combination thereof. In particular embodiments, exemplary effector domains include signaling and co-stimulatory domains selected from: CD86, FcyRlla, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8α, CD8β, IL2Rβ, IL2Rγ, IL7Ra, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRy (FCER1G), FcyRlla, FcRβ (Fcε Rib), DAP10, and DAP12. In particular embodiments, variants of CD3ζ retain at least one, two, three, or all ITAM regions.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling components include the cytoplasmic sequences of the CD3ζ chain, and/or co- receptors that act in concert to initiate signal transduction following binding domain engagement.

A co-stimulatory domain is domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance expansion, effector function, and survival of human CART cells in vitro and augments human T cell persistence and anti-cancer activity in vivo (Song et al. Blood. 2012; 119(3):696-706). Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Ry, IL7Rα, ITGA4, VLA1, CD49a, ITGA4, IA4, CD49D, ITGA6, VLA-6, CD49f, ITGAD, CD11d, ITGAE, CD103, ITGAL, CD11a, ITGAM, CDI Ib, ITGAX, CD11c, ITGBI, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, LyI08), SLAM (SLAMF1, CD150, IPO-3), BLAME (SLAMF8), SELPLG (CD162), LTBR, LAT, GADS, SLP-76, PAG/Cbp, and CD19a.

In particular embodiments, the amino acid sequence of the intracellular signaling component includes a variant of CD3ζ and a portion of the 4-1BB intracellular signaling component.

In particular embodiments, the intracellular signaling component includes (i) all or a portion of the signaling domain of CD3ζ, (ii) all or a portion of the signaling domain of 4-1BB, or (iii) all or a portion of the signaling domain of CD3ζ and 4-1BB.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (IL1R, IL2R, IL7R, or IL15R).

Linkers. As used herein, a linker can be any portion of a CAR molecule that serves to connect two other subcomponents of the molecule. Some linkers serve no purpose other than to link other components while many linkers serve an additional purpose. Linkers in the context of linking VL and VH of antibody derived binding domains of scFv are described above. Linkers can also include spacer regions, and junction amino acids.

Spacer regions are a type of linker region that are used to create appropriate distances and/or flexibility from other linked components. In particular embodiments, the length of a spacer region can be customized for individual cellular markers on unwanted cells to optimize unwanted cell recognition and destruction. The spacer can be of a length that provides for increased responsiveness of the cell following antigen binding, as compared to in the absence of the spacer. In particular embodiments, a spacer region length can be selected based upon the location of a cellular marker epitope, affinity of a binding domain for the epitope, and/or the ability of the modified cells expressing the molecule to proliferate in vitro and/or in vivo in response to cellular marker recognition. Spacer regions can also allow for high expression levels in modified cells.

In particular embodiments, a spacer region includes a hinge region that a type 11 C-lectin interdomain (stalk) region or a cluster of differentiation (CD) molecule stalk region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

A “stalk region” of a type 11 C-lectin or CD molecule refers to the portion of the extracellular domain of the type 11 C-lectin or CD molecule that is located between the C-type lectin-like domain (CTLD; e.g., similar to CTLD of natural killer cell receptors) and the hydrophobic portion (transmembrane domain). For example, the extracellular domain of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat′l. Acad. Sci. USA 89:153, 1992; and Figdor et al., Nat. Rev. Immunol. 2:11, 2002). These type 11 C-lectin or CD molecules may also have junction amino acids (described below) between the stalk region and the transmembrane region or the CTLD. In another example, the 233 amino acid human NKG2A protein (GenBank Accession No. P26715.1) has a hydrophobic portion (transmembrane domain) ranging from amino acids 71-93 and an extracellular domain ranging from amino acids 94-233. The CTLD includes amino acids 119-231 and the stalk region includes amino acids 99-116, which may be flanked by additional junction amino acids. Other type 11 C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

As further description regarding spacer regions, an extracellular component of a fusion protein optionally includes an extracellular, non-signaling spacer or linker region, which, for example, can position the binding domain away from the host cell (e.g., T cell) surface to enable proper cell/cell contact, antigen binding and activation (Patel et al., Gene Therapy 6: 412-419, 1999). As indicated, an extracellular spacer region of a fusion binding protein is generally located between a hydrophobic portion or transmembrane domain and the extracellular binding domain, and the spacer region length may be varied to maximize antigen recognition (e.g., tumor recognition) based on the selected target molecule, selected binding epitope, or antigen-binding domain size and affinity (see, e.g., Guest etal., J. Immunother. 28:203-11, 2005; PCT Publication No. WO 2014/031687). In certain embodiments, a spacer region includes an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. An immunoglobulin hinge region may be an IgG, IgA, IgD, IgE, or IgM hinge region. An IgG hinge region may be an IgG1, IgG2, IgG3, or IgG4 hinge region. Other examples of hinge regions used in the fusion binding proteins described herein include the hinge region present in the extracellular regions of type 1 membrane proteins, such as CD8α, CD4, CD28, and CD7, which may be wild-type or variants thereof.

In certain embodiments, an extracellular spacer region includes all or a portion of an Fc domain selected from: a CH1 domain, a CH2 domain, a CH3 domain, a CH4 domain, or any combination thereof. The Fc domain or portion thereof may be wildtype of altered (e.g., to reduce antibody effector function). In certain embodiments, the extracellular component includes an immunoglobulin hinge region, a CH2 domain, a CH3 domain, or any combination thereof disposed between the binding domain and the hydrophobic portion.

Junction amino acids can be a linker which can be used to connect the sequences of CAR domains when the distance provided by a spacer is not needed and/or wanted. Junction amino acids are short amino acid sequences that can be used to connect co-stimulatory intracellular signaling components. In particular embodiments, junction amino acids are 9 amino acids or less.

Junction amino acids can be a short oligo- or protein linker, preferably between 2 and 9 amino acids (e.g., 2, 3, 4, 5, 6, 7, 8, or 9 amino acids) in length to form the linker. In particular embodiments, a glycine-serine doublet can be used as a suitable junction amino acid linker. In particular embodiments, a single amino acid, e.g., an alanine, a glycine, can be used as a suitable junction amino acid.

Transmembrane Domains. As indicated, transmembrane domains within a CAR molecule, often serving to connect the extracellular component and intracellular component through the cell membrane. The transmembrane domain can anchor the expressed molecule in the modified cell’s membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the α, β or ζ chain of a T-cell receptor, CD28, CD27, CD3 epsilon, CD45, CD4, CD5, CD8, CD9, CD16, CD22; CD33, CD37, CD64, CD80, CD86, CD134, CD137 and CD154. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Ry, IL7R a, ITGA1, VLA1, CD49a, ITGA4, IA4, CD49D, ITGA6, VLA-6, CD49f, ITGAD, CDI Id, ITGAE, CD103, ITGAL, CDI la, ITGAM, CDI Ib, ITGAX, CDIIc, ITGB1, CD29, ITGB2, CD18, ITGB7, TNFR2, DNAM1(CD226), SLAMF4 (CD244, 2B4), CD84, CD96 (Tactile), CEACAM1, CRT AM, Ly9(CD229), PSGL1, CD100 (SEMA4D), SLAMF6 (NTB-A, LyI08), SLAM (SLAMF1, CD150, IPO-3), BLAME (SLAMF8), SELPLG (CD162), LTBR, PAG/Cbp, NKG2D, or NKG2C.

In particular embodiments, a transmembrane domain has a three-dimensional structure that is thermodynamically stable in a cell membrane, and generally ranges in length from 15 to 30 amino acids. The structure of a transmembrane domain can include an α helix, a β barrel, a β sheet, a β helix, or any combination thereof.

A transmembrane domain can include one or more additional amino acids adjacent to the transmembrane region, e.g., one or more amino acid within the extracellular region of the CAR (e.g., up to 15 amino acids of the extracellular region) and/or one or more additional amino acids within the intracellular region of the CAR (e.g., up to 15 amino acids of the intracellular components). In one aspect, the transmembrane domain is from the same protein that the signaling domain, co-stimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins to minimize interactions with other unintended members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell. In particular embodiments, the transmembrane domain includes the amino acid sequence of the CD28 transmembrane domain.

Transduction markers may be selected from at least one of a truncated CD19 (tCD19; see Budde et al., Blood 122: 1660, 2013); a truncated human EGFR (tEGFR; see Wang et al., Blood 118: 1255, 2011); an extracellular domain of human CD34; and/or RQR8 which combines target epitopes from CD34 (see Fehse et al., Mol. Therapy 1 (5 Pt 1 ):448-456, 2000) and CD20 antigens (see Philip et al., Blood 124: 1277-1278, 2014).

In particular embodiments, a polynucleotide encoding an iCaspase9 construct (iCasp9) may be inserted into a CAR nucleotide construct as a suicide switch.

Control features may be present in multiple copies in a CAR or can be expressed as distinct molecules with the use of a skipping element. In particular embodiments, a transduction marker includes tEGFR. Exemplary transduction markers and cognate pairs are described in U.S. Pat. No. 8,802,374.

One advantage of including at least one control feature in a CAR is that CAR expressing cells administered to a subject can be depleted using the cognate binding molecule for the control feature, or by using a second modified cell expressing a CAR and having specificity for the control feature. Elimination of modified cells may be accomplished using depletion agents specific for a control feature.

In certain embodiments, modified cells expressing a chimeric molecule may be detected or tracked in vivo by using antibodies that bind with specificity to a control feature, or by other cognate binding molecules that specifically bind the control feature, which binding partners for the control feature are conjugated to a fluorescent dye, radio-tracer, iron-oxide nanoparticle or other imaging agent known in the art for detection by X-ray, CT-scan, MRI-scan, PET-scan, ultrasound, flow-cytometry, near infrared imaging systems, or other imaging modalities (see, e.g., Yu et al., Theranostics 2:3, 2012).

Thus, modified cells expressing at least one control feature with a CAR can be, e.g., more readily identified, isolated, sorted, induced to proliferate, tracked, and/or eliminated as compared to a modified cell without a tag cassette.

A T-cell receptor (TCR) is a molecule found on the surface of T cells which is responsible for a T-cells recognition of peptides bound to major histocompatibility complex (MHC).

TCR refer to naturally occurring T cell receptors. HSC can be modified in vivo to express a selected TCR. CAR/TCR hybrids refer to proteins having an element of a TCR and an element of a CAR. For example, a CAR/TCR hybrid could have a naturally occurring TCR binding domain with an effector domain that the TCR binding domain is not naturally associated with. A CAR/TCR hybrid could have a mutated TCR binding domain and an ITAM signaling domain. A CAR/TCR hybrid could have a naturally occurring TCR with an inserted non-naturally occurring spacer region or transmembrane domain.

Particular CAR/TCR hybrids include TRuC® (T Cell Receptor Fusion Construct) hybrids; TCR2 Therapeutics, Cambridge, MA. By way of example, the production of TCR fusion proteins is described in International Patent Publications WO 2018/026953 and WO 2018/067993, and in Application Publication US 2017/0166622.

In particular embodiments, CAR/TCR hybrids include a “T-cell receptor (TCR) fusion protein” or “TFP”. A TFP includes a recombinant polypeptide derived from the various polypeptides including the TCR that is generally capable of i) binding to a surface antigen on target cells and ii) interacting with other polypeptide components of the intact TCR complex, typically when co-located in or on the surface of a T-cell.

### (IV-d) CRISPR

The CRISPR (Clustered Regularly Interspaced Short Palindromic Repeats)/Cas (CRISPR-associated protein) nuclease system is an engineered nuclease system used for genetic engineering that is based on a bacterial system. It is based in part on the adaptive immune response of many bacteria and archaea. When a virus or plasmid invades a bacterium, segments of the invader’s DNA are converted into CRISPR RNAs (crRNA) by the bacteria’s “immune” response. The crRNA then associates, through a region of partial complementarity, with another type of RNA called tracrRNA to guide a Cas nuclease to a region homologous to the crRNA in the target DNA called a “protospacer.” The Cas nuclease cleaves the DNA to generate blunt ends at the double-strand break at sites specified by a 20-nucleotide complementary strand sequence contained within the crRNA transcript. In some instances, the Cas nuclease requires both the crRNA and the tracrRNA for site-specific DNA recognition and cleavage.

Guide RNA (gRNA) is one example of a targeting element. In its simplest form, gRNA provides a sequence that targets a site within a genome based on complementarity (e.g., crRNA). As explained below, however, gRNA can also include additional components. For example, in particular embodiments, gRNA can include a targeting sequence (e.g., crRNA) and a component to link the targeting sequence to a cutting element. This linking component can be tracrRNA. In particular embodiments, as described below, gRNA including crRNA and tracrRNA can be expressed as a single molecule referred to as single gRNA (sgRNA). gRNA can also be linked to a cutting element through other mechanisms such as through a nanoparticle or through expression or construction of a dual or multi-purpose molecule.

In particular embodiments, targeting elements (e.g., gRNA) can include one or more modifications (e.g., a base modification, a backbone modification), to provide the nucleic acid with a new or enhanced feature (e.g., improved stability). Modified backbones may include those that retain a phosphorus atom in the backbone and those that do not have a phosphorus atom in the backbone. Suitable modified backbones containing a phosphorus atom may include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable targeting elements having inverted polarity can include a single 3′ to 3′ linkage at the 3′-most internucleotide linkage (i.e. a single inverted nucleoside residue in which the nucleobase is missing or has a hydroxyl group in place thereof). Various salts (e.g., potassium chloride or sodium chloride), mixed salts, and free acid forms can also be included.

Targeting elements can include one or more phosphorothioate and/or heteroatom internucleoside linkages, in particular —CH₂—NH—O—CH₂—, —CH₂—N(CH₃)—O—CH₂— (i.e. a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—).

In particular embodiments, targeting elements can include a morpholino backbone structure. For example, the targeting elements can include a 6-membered morpholino ring in place of a ribose ring. In some of these embodiments, a phosphorodiamidate or other non-phosphodiester internucleoside linkage replaces a phosphodiester linkage.

In particular embodiments, targeting elements can include one or more substituted sugar moieties. Suitable polynucleotides can include a sugar substituent group selected from: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly suitable are O((CH₂)ₙO) mCH₃, O(CH2)ₙOCH₃, O(CH₂)ₙNH₂, O(CH₂)ₙCH₃, O(CH₂)ₙONH₂, and O(CH₂)ₙON((CH₂)ₙCH₃)₂, where n and m are independently from 1 to 10.

Examples of cutting elements include nucleases. CRISPR-Cas loci have more than 50 gene families and there are no strictly universal genes, indicating fast evolution and extreme diversity of loci architecture. Exemplary Cas nucleases include CasI, CasIB, Cas2, Cas3, Cas4, Cas5, Cas6, Cas7, Cas8, Cas9 (also known as Csnl and Csxl2), CaslO,, Cpfl, C2c3, C2c2 and C2clCsyl, Csy2, Csy3, Csel, Cse2, Cscl, Csc2, Csa5, Csn2, Csm2, Csm3, Csm4, Csm5, Csm6, CmrI, Cmr3, Cmr4, Cmr5, Cmr6, Cpfl, CsbI, Csb2, Csb3, Csxl7, Csxl4, CsxIO, Csxl6, CsaX, Csx3, Csxl, Csxl5, CsfI, Csf2, Csf3, and Csf4.

There are three main types of Cas nucleases (type 1, type 11, and type 111), and 10 subtypes including 5 type 1, 3 type 11, and 2 type III proteins (see, e.g., Hochstrasser and Doudna, Trends Biochem Sci, 40(l):58-66, 2015). Type 11 Cas nucleases include CasI, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref. Seq. No. NP 269215, and the amino acid sequence of Streptococcus thermophilus wild-type Cas9 polypeptide is set forth, e.g., in NBCI Ref. Seq. No. WP_01 1681470.

In particular embodiments, Cas9 refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme, in some embodiments, includes one or more catalytic domains of a Cas9 protein derived from bacteria such as Corynebacter, Sutterella, Legionella, Treponema, Filif actor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g. the two catalytic domains are derived from different bacterial species.

As indicated previously, the CRISPR/Cas system has been engineered such that, in certain cases, crRNA and tracrRNA can be combined into one molecule called a single gRNA (sgRNA). In this engineered approach, the sgRNA guides Cas to target any desired sequence (see, e.g., Jinek et al., Science 337:816-821, 2012; Jinek et al., eLife 2:e00471, 2013; Segal, eLife 2:e00563, 2013). Thus, the CRISPR/Cas system can be engineered to create a double-strand break at a desired target in a genome of a cell, and harness the cell’s endogenous mechanisms to repair the induced break by HDR, or NHEJ. Particular embodiments described herein utilize homology arms to promote HDR at defined integration sites.

Useful variants of the Cas9 nuclease include a single inactive catalytic domain, such as a RuvC″ or HNH″ enzyme or a nickase. A Cas9 nickase has only one active functional domain and, in some embodiments, cuts only one strand of the target DNA, thereby creating a single strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include N854A and N863A. A double-strand break is introduced using a Cas9 nickase if at least two DNA-targeting RNAs that target opposite DNA strands are used. A double-nicked induced double-strand break is repaired by HDR or NHEJ. This gene editing strategy generally favors HDR and decreases the frequency of indel mutations at off-target DNA sites. The Cas9 nuclease or nickase, in some embodiments, is codon-optimized for the target cell or target organism.

Particular embodiments can utilize Staphylococcus aureus Cas9 (SaCas9). Particular embodiments can utilize SaCas9 with mutations at one or more of the following positions: E782, N968, and/or R1015. Particular embodiments can utilize SaCas9 with mutations at one or more of the following positions: E735, E782, K929, N968, A1021, K1044 and/or R1015. In some embodiments, the variant SaCas9 protein includes one or more of the following mutations: R1015Q, R1015H, E782K, N968K, E735K, K929R, A1021T, and/or K1044N. In some embodiments, the variant SaCas9 protein includes mutations at D10A, D556A, H557A, N580A, e.g., D10A/H557A and/or D10A/D556A/H557A/N580A. In some embodiments, the variant SaCas9 protein includes one or more mutations selected from E735, E782, K929, N968, R1015, A1021, and/or K1044. In some embodiments, the SaCas9 variants can include one of the following sets of mutations: E782K/N968K/R1015H (KKH variant); E782K/K929R/R1015H (KRH variant); or E782K/K929R/N968K/R1015H (KRKH variant).

A Class 11, Type V CRISPR-Cas class exemplified by Cpf1 has been identified Zetsche et al., Cell 163(3): 759-771, 2015. The Cpf1 nuclease particularly can provide added flexibility in target site selection by means of a short, three base pair recognition sequence (TTN), known as the protospacer-adjacent motif or PAM. Cpf1′s cut site is at least 18bp away from the PAM sequence. Moreover, staggered DSBs with sticky ends permit orientation-specific donor template insertion, which is advantageous in non-dividing cells.

Particular embodiments can utilize engineered Cpf1s. For example, US 2018/0030425 describes engineered Cpf1 nucleases from Lachnospiraceae bacterium ND2006 and Acidaminococcus sp. BV3L6 with altered and improved target specificity. Particular variants include Lachnospiraceae bacterium ND2006, e.g., at least including amino acids 19-1246 with mutations (i.e., replacement of the native amino acid with a different amino acid, e.g., alanine, glycine, or serine), at one or more of the following positions: S202, N274, N278, K290, K367, K532, K609, K915, Q962, K963, K966, K1002, and/or S1003. Particular Cpf1 variants can also include Acidaminococcus sp. BV3L6 Cpf1 (AsCpf1) with mutations (i.e., replacement of the native amino acid with a different amino acid, e.g., alanine, glycine, or serine (except where the native amino acid is serine)), at one or more of the following positions: N178, S186, N278, N282, R301, T315, S376, N515, K523, K524, K603, K965, Q1013, Q1014, and/or K1054.

Other Cpf1 variants include Cpf1 homologs and orthologs of the Cpf1 polypeptides disclosed in Zetsche et al. (Cell 163: 759-771, 2015) as well as the Cpf1 polypeptides disclosed in U.S. Pat. Publication No. 2016/0208243. Other engineered Cpf1 variants are known to those of ordinary skill in the art and included within the scope of the current disclosure (see, e.g., WO/2017/184768).

As indicated previously, embodiments utilize homology arms to facilitate targeted insertion of genetic constructs utilizing homology directed repair. Homology arms can be any length with sufficient homology to a genomic sequence at a cleavage site, e.g. 70%, 80%, 85%, 90%, 95%, or 100% homology with the nucleotide sequences flanking the cleavage site, e.g., within 50 bases or less of the cleavage site, e.g., within 30 bases, within 15 bases, within 10 bases, within 5 bases, or immediately flanking the cleavage site, to support HDR between it and the genomic sequence to which it bears homology. Homology arms are generally identical to the genomic sequence, for example, to the genomic region in which the double stranded break (DSB) occurs. However, as indicated, absolute identity is not required.

Particular embodiment can utilize homology arms with 25, 50, 100, or 200 nucleotides, or more than 200 nucleotides of sequence homology between a homology-directed repair template and a targeted genomic sequence (or any integral value between 10 and 200 nucleotides, or more). In particular embodiments, homology arms are 40 nucleotides (nt) - 1000 nt in length. In particular embodiments, homology arms 500-2500 base pairs, 700 – 2000 base pairs, or 800 -1800 base pairs. In particular embodiments, homology arms include at least 800 base pairs or at least 850 base pairs. The length of homology arms can also be symmetric or asymmetric. For additional information regarding homology arms, see Richardson et al., Nat Biotechnol., 34(3):339-44, 2016.

Additional information regarding CRISPR-Cas systems and components thereof are described in, US8697359, US8771945, US8795965, US8865406, US8871445, US8889356, US8889418, US8895308, US8906616, US8932814, US8945839, US8993233, and US8999641; and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016/205711, WO2017/106657, WO2017/127807; and applications related thereto.

### (IV-e) Base Editing System

Base editing refers to the selective modification of a nucleic acid sequence by converting a base or base pair within genomic DNA or cellular RNA to a different base or base pair (Rees & Liu, Nature Reviews Genetics, 19:770-788, 2018). There are two general classes of DNA base editors: (i) cytosine base editors (CBEs) that convert guanine-cytosine base pairs into thymine-adenine base pairs, and (ii) adenine base editors (ABEs) that convert adenine-thymine base pairs to guanine cytosine base pairs.

DNA base editors can insert such point mutations in non-dividing cells without generating double-strand breaks. Due to the lack of double-strand breaks, base editors do not result in excess undesired editing by-products, such as insertions and deletions (indels). For example, base editors can generate fewer than 10%, 9%, 8%, 7%, 6%, 5.5%, 5%, 4.5%, 4%, 3.5%, 3%, 2.5%, 2%, 1.5%, 1%, 0.5%, or 0.1% indels as compared to technologies that do rely on double-strand breaks.

Components of most base-editing systems include (1) a targeted DNA binding protein, (2) a nucleobase deaminase enzyme, and (3) a DNA glycosylase inhibitor.

Any nuclease of the CRISPR system can be disabled and used within a base editing system. Exemplary Cas nucleases include Cas1, Cas1B, Cas2, Cas3, Cas4, Cas5, Cas6, Cas7, Cas8, Cas9 (also known as Csnl and Csx12), CaslO,, Cpfl, C2c3, C2c2 and C2clCsyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Cpfl, Csbl, Csb2, Csb3, Csxl7, Csxl4, CsxlO, Csxl6, CsaX, Csx3, Csxl, Csxl5, Csf1, Csf2, Csf3, Csf4 and mutations thereof.

Nucleases from other gene-editing systems may also be used. For example, base-editing systems can utilize zinc finger nucleases (ZFNs) (Urnov et al., Nat Rev Genet., 11 (9):636-46, 2010) and transcription activator like effector nucleases (TALENs) (Joung etal., Nat Rev Mol Cell Biol. 14(1 ):49-55, 2013). For additional information regarding DNA-binding nucleases, see US2018/0312825A1.

In particular embodiments, the nucleobase deaminase enzyme includes a cytidine deaminase domain or an adenine deaminase domain.

In particular embodiments, CBE utilizing a cytidine deaminase domain convert guanine-cytosine base pairs into thymine-adenine base pairs by deaminating the exocyclic amine of the cytosine to generate uracil. Examples of cytosine deaminase enzymes include APOBEC1, APOBEC3A, APOBEC3G, CDA1, and AID. APOBEC1 particularly accepts single stranded (ss)DNA as a substrate but is incapable of acting on double stranded (ds)DNA.

Most base-editing systems also include a DNA glycosylase inhibitor that serves to override natural DNA repair mechanisms that might otherwise repair the intended base editing. In particular embodiments, the DNA glycosylase inhibitor includes an uracil glycosylase inhibitor, such as the uracil DNA glycosylase inhibitor protein (UGI) described in Wang et al. (Gene 99, 31-37, 1991).

Components of base editors can be fused directly (e.g., by direct covalent bond) or via linkers. For example, the catalytically disabled nuclease can be fused via a linker to the deaminase enzyme and/or a glycosylase inhibitor. Multiple glycosylase inhibitors can also be fused via linkers. As will be understood by one of ordinary skill in the art, linkers can be used to link any peptides or portions thereof.

Exemplary linkers include polymeric linkers (e.g., polyethylene, polyethylene glycol, polyamide, polyester); amino acid linkers; carbon-nitrogen bond amide linkers; cyclic or acyclic, substituted or unsubstituted, branched or unbranched aliphatic or heteroaliphatic linkers; monomeric, dimeric, or polymeric aminoalkanoic acid linkers; aminoalkanoic acid (e.g., glycine, ethanoic acid, alanine, β-alanine, 3-aminopropanoic acid, 4-aminobutanoic acid, 5-pentanoic acid) linkers; monomeric, dimeric, or polymeric aminohexanoic acid (Ahx) linkers;. carbocyclic moiety (e.g., cyclopentane, cyclohexane) linkers; aryl or heteroaryl moiety linkers; and phenyl ring linkers.

Linkers can also include functionalized moieties to facilitate attachment of a nucleophile (e.g., thiol, amino) from a peptide to the linker. Any electrophile may be used as part of the linker. Exemplary electrophiles include activated esters, activated amides, Michael acceptors, alkyl halides, aryl halides, acyl halides, and isothiocyanates.

In particular embodiments, linkers range from 4 -100 amino acids in length. In particular embodiments, linkers are 4 amino acids, 9 amino acids, 14 amino acids, 16 amino acids, 32 amino acids, or 100 amino acids.

Numerous base-editing (BE) systems formed by linking targeted DNA binding proteins with cytidine deaminase enzymes and DNA glycosylase inhibitors (e.g., UGI) have been described. These complexes include for example, BE1 ([APOBEC1-16 amino acid (aa) linker-Sp dCas9 (D10A, H840A)] Komer et al., Nature, 533, 420-424, 2016), BE2 ([APOBEC1-16aalinker-Sp dCas9 (D10A, H840A)-4aa linker-UGI] Komer et al., 2016 supra), BE3 ([APOBEC1-16aa linker-Sp nCas9 (D10A)-4aa linker-UGI] Komer et al., supra), HF-BE3 ([APOBEC1-16aa linker-HF nCas9 (D10A)-4aa linker-UGI] Rees et al., Nat. Commun. 8, 15790, 2017), BE4, BE4max ([APOBEC1-32aa linker-Sp nCas9 (D10A)-9aa linker-UGI-9aa linker-UGI] Koblan et al., Nat. Biotechnol 10.1038/nbt.4172, 2018; Komer et al., Sci. Adv., 3, eaao4774, 2017), BE4-GAM ([Gam-16aa linker-APOBEC1-32aa linker-Sp nCas9 (D10A)-9aa linker-UGI-9aa linker-UGI] Komer et al., 2017 supra), YE1-BE3 ([APOBEC1 (W90Y, R126E)-16aalinker-Sp nCas9 (D10A)-4aa linker-UGI] Kim et al., Nat. Biotechnol. 35, 475-480, 2017), EE-BE3 ([APOBEC1 (R126E, R132E)-16aa linker-Sp nCas9 (D10A)-4aa linker-UGI] Kim et al., 2017 supra), YE2-BE3 ([APOBEC1 (W90Y, R132E)-16aa linker-Sp nCas9 (D10A)-4aa linker-UGI]Kim et al., 2017 supra), YEE-BE3 ([APOBEC1 (W90Y, R126E, R132E)-16aalinker-Sp nCas9 (D10A)-4aa linker-UGI] Kim et al., 2017 supra), VQR-BE3 ([APOBEC1-16aa linker-Sp VQR nCas9 (D10A)-4aa linker-UGI] Kim etal., 2017 supra), VRER-BE3 ([APOBEC1-16aa linker-Sp VRER nCas9 (D10A)-4aa linker-UGI] Kim etal., Nat. Biotechnol. 35, 475-480, 2017), Sa-BE3 ([APOBEC1-16aalinker-Sa nCas9 (D10A)-4aa linker-UGI] Kim et al., 2017 supra), SA-BE4 ([APOBEC1-32aa linker-Sa nCas9 (D10A)-9aa linker-UGI-9aa linker-UGI] Komer et al., 2017 supra), SaBE4-Gam ([Gam-16aa linker-APOBEC1-32aa linker-Sa nCas9 (D10A)-9aa linker-UGI-9aa linker-UGI] Komer et al., 2017 supra), SaKKH-BE3 ([APOBEC1-16aa linker-Sa KKH nCas9 (D10A)-4aa linker-UGI] Kim etal., 2017 supra), Cas12a-BE ([APOBEC1-16aalinker-dCas12a-14aalinker-UGI], Li etal., Nat. Biotechnol. 36, 324-327, 2018), Target-AID ([Sp nCas9 (D10A)-100aa linker-CDA1-9aa linker-UGI] Nishida et al., Science, 353, 10.1126/science.aaf8729, 2016), Target-AID-NG ([Sp nCas9 (D10A)-NG-100aa linker-CDA1-9aa linker-UGI] Nishimasu et al., Science, 361 (6408): 1259-1262, 2018), xBE3 ([APOBEC1-16aa linker-xCas9(D10A)-4aa linker-UGI] Hu et al., Nature, 556, 57-63, 2018), eA3A-BE3 ([APOBEC3A (N37G)-16aa linker-Sp nCas9(D10A)-4aa linker-UGI] Gerkhe et al., Nat. Biotechnol., 10.1038/nbt.4199, 2018), A3A-BE3 ([hAPOBEC3A-16aa linker-Sp nCas9(D10A)-4aa linker-UGI] Wang et al., Nat. Biotechnol. 10.1038/nbt.4198, 2018), and BE-PLUS ([10X GCN4-Sp nCas9(D10A) / ScFv-rAPOBEC1-UGI] Jiang et al., Cell. Res, 10.1038/s41422-018-0052-4, 2018). For additional examples of BE complexes, including adenine deaminase base editors, see Rees & Liu Nat. Rev Genet. 2018 Dec; 19(12): 770-788.

For additional information regarding base editors, see US2018/0312825A1, WO2018/165629A, Urnov et al, Nat Rev Genet. 2010; 11(9):636-46; Joung et al., Nat Rev Mol Cell Biol. 2013; 14(1):49-55; Charpentier et al., Nature.; 495(7439):50-1, 2013; and Rees & Liu, Nature Reviews Genetics, 19:770-788, 2018.

### (IV-f) Small RNAs

Small RNAs are short, non-coding RNA molecules that play a role in regulating gene expression. In particular embodiments, small RNAs are less than 200 nucleotides in length. In particular embodiments, small RNAs are less than 100 nucleotides in length. In particular embodiments, small RNAs are less than 50 nucleotides in length. In particular embodiments, small RNAs are less than 20 nucleotides in length. Small RNAs include but microRNA (miRNA, Piwi-interacting RNA (piRNA), small interfering RNA (siRNA), small nucleolar RNA (snoRNA), tRNA-derived small RNA (tsRNA) small rDNA-derived RNA (srRNA), and small nuclear RNA. Additional classes of small RNAs continue to be discovered.

In particular embodiments, interfering RNA molecules that are homologous to target mRNA can lead to its degradation, a process referred to as RNA interference (RNAi) (Carthew, Curr. Opin. Cell. Biol. 13: 244-248, 2001). RNAi occurs in cells naturally to remove foreign RNAs (e.g., viral RNAs). Natural RNAi proceeds via fragments cleaved from free double-strand RNA (dsRNA) which direct the degradative mechanism to other similar RNA sequences. Alternatively, RNAi can be manufactured, for example, to silence the expression of target genes. Exemplary RNAi molecules include small hairpin RNA (shRNA, also referred to as short hairpin RNA) and small interfering RNA (siRNA).

Without limiting the disclosure, and without being bound by theory, RNA interference is typically a two-step process. In the first step, the initiation step, input dsRNA is digested into 21-23 nucleotide (nt) siRNA, probably by the action of Dicer, a member of the ribonuclease (RNase) III family of dsRNA-specific ribonucleases, which processes (cleaves) dsRNA (introduced directly or via a transgene or a virus) in an ATP-dependent manner. Successive cleavage events degrade the RNA to 19-21 base pair (bp) duplexes (siRNA), each with 2-nucleotide 3′ overhangs (Hutvagner & Zamore, Curr. Opin. Genet. Dev. 12: 225-232, 2002; Bernstein, Nature 409:363-366, 2001).

In an effector step, the siRNA duplexes bind to a nuclease complex to form the RNA-induced silencing complex (RISC). An ATP-dependent unwinding of the siRNA duplex is required for activation of the RISC. The active RISC then targets the homologous transcript by base pairing interactions and typically cleaves the mRNA into 12 nucleotide fragments from the 3′ terminus of the siRNA (Hutvagner & Zamore, Curr. Opin. Genet. Dev. 12: 225-232, 2002; Hammond et al., Nat. Rev. Gen. 2:110-119, 2001; Sharp, Genes. Dev. 15:485-490, 2001). Research indicates that each RISC contains a single siRNA and an RNase (Hutvagner & Zamore, Curr. Opin. Genet. Dev. 12: 225-232, 2002).

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC (Hutvagner & Zamore, Curr. Opin. Genet. Dev. 12: 225-232, 2002; Hammond et al., Nat. Rev. Gen. 2:110-119, 2001; Sharp, Genes. Dev. 15:485-490, 2001). RNAi is also described in Tuschl (Chem. Biochem. 2: 239-245, 2001); Cullen (Nat. Immunol. 3:597-599, 2002); and Brantl (Biochem. Biophys. Act. 1575:15-25, 2002).

Synthesis of RNAi molecules suitable for use with the present disclosure can be performed as follows. First, an mRNA sequence can be scanned downstream of the start codon of targeted transgene. Occurrence of each AA and the 3′ adjacent 19 nucleotides is recorded as potential siRNA target sites. In particular embodiments, the siRNA target sites can be selected from the open reading frame, as untranslated regions (UTRs) are richer in regulatory protein binding sites. UTR-binding proteins and/or translation initiation complexes may interfere with binding of the siRNA endonuclease complex (Tuschl, Chem. Biochem. 2: 239-245, 2001). It will be appreciated though, that siRNAs directed at untranslated regions may also be effective, as demonstrated for Glyceraldehyde 3-phosphate dehydrogenase (GAPDH) wherein siRNA directed at the 5′ UTR mediated a 90% decrease in cellular GAPDH mRNA and completely abolished protein level. Second, potential target sites can be compared to an appropriate genomic database using any sequence alignment software, such as the Basic Local Alignment Search Tool (BLAST) software available from the National Center for Biotechnology Information (NCBI) server. Putative target sites which exhibit significant homology to other coding sequences can be filtered out.

Qualifying target sequences can be selected as templates for siRNA synthesis. Selected sequences can include those with low G/C content as these have been shown to be more effective in mediating gene silencing as compared to those with G/C content higher than 55%. Several target sites can be selected along the length of the target gene for evaluation. For better evaluation of the selected siRNAs, a negative control can be used. Negative control siRNA can include the same nucleotide composition as the siRNAs but lack significant homology to the genome. Thus, a scrambled nucleotide sequence of the siRNA may be used, provided it does not display any significant homology to other genes.

A sense strand is designed based on the sequence of the selected portion. The antisense strand is routinely the same length as the sense strand and includes complementary nucleotides. In particular embodiments, the strands are fully complementary and blunt-ended when aligned or annealed. In other embodiments, the strands align or anneal such that 1-, 2- or 3-nucleotide overhangs are generated, i.e., the 3′ end of the sense strand extends 1, 2 or 3 nucleotides further than the 5′ end of the antisense strand and/or the 3′ end of the antisense strand extends 1, 2 or 3 nucleotides further than the 5′ end of the sense strand. Overhangs can include nucleotides corresponding to the target gene sequence (or complement thereof). Alternatively, overhangs can include deoxyribonucleotides, for example deoxythymines (dTs), or nucleotide analogs, or other suitable non-nucleotide material.

To facilitate entry of the antisense strand into RISC (and thus increase or improve the efficiency of target cleavage and silencing), the base pair strength between the 5′ end of the sense strand and 3′ end of the antisense strand can be altered, e.g., lessened or reduced. In particular embodiments, the base-pair strength is less due to fewer G:C base pairs between the 5′ end of the first or antisense strand and the 3′ end of the second or sense strand than between the 3′ end of the first or antisense strand and the 5′ end of the second or sense strand. In particular embodiments, the base pair strength is less due to at least one mismatched base pair between the 5′ end of the first or antisense strand and the 3′ end of the second or sense strand. Preferably, the mismatched base pair is selected from the group including G:A, C:A, C:U, G:G, A:A, C:C and U:U. In another embodiment, the base pair strength is less due to at least one wobble base pair, e.g., G:U, between the 5′ end of the first or antisense strand and the 3′ end of the second or sense strand. In another embodiment, the base pair strength is less due to at least one base pair including a rare nucleotide, e.g., inosine (I). In particular embodiments, the base pair is selected from the group including an I:A, I:U and I:C. In yet another embodiment, the base pair strength is less due to at least one base pair including a modified nucleotide. In particular embodiments, the modified nucleotide is selected from, for example, 2-amino-G, 2-amino-A, 2,6-diamino-G, and 2,6-diamino-A.

ShRNAs are single-stranded polynucleotides with a hairpin loop structure. The single-stranded polynucleotide has a loop segment linking the 3′ end of one strand in the double-stranded region and the 5′ end of the other strand in the double-stranded region. The double-stranded region is formed from a first sequence that is hybridizable to a target sequence, such as a polynucleotide encoding transgene, and a second sequence that is complementary to the first sequence, thus the first and second sequence form a double stranded region to which the linking sequence connects the ends of to form the hairpin loop structure. The first sequence can be hybridizable to any portion of a polynucleotide encoding transgene. The double-stranded stem domain of the shRNA can include a restriction endonuclease site.

Transcription of shRNAs is initiated at a polymerase III (Pol 111) promoter and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of 21-23 nucleotides (Brummelkamp et al., Science. 296(5567):550-553, 2002; Lee et al., Nature Biotechnol. 20(5):500-505, 2002; Miyagishi & Taira, Nature Biotechnol. 20(5):497-500, 2002; Paddison et al., Genes & Dev. 16(8): 948-958, 2002; Paul et al., Nature Biotechnol. 20(5):505-508, 2002; Sui, Proc. Natl. Acad. Sci. USA. 99(6):5515-5520, 2002; Yu et al., Proc. Natl. Acad. Sci. USA. 99(9):6047-6052, 2002).

The stem-loop structure of shRNAs can have optional nucleotide overhangs, such as 2-bp overhangs, for example, 3′ UU overhangs. While there may be variation, stems typically range from 15 to 49, 15 to 35, 19 to 35, 21 to 31 bp, or 21 to 29 bp, and the loops can range from 4 to 30 bp, for example, 4 to 23 bp. In particular embodiments, shRNA sequences include 45-65 bp; 50-60 bp; or 51, 52, 53, 54, 55, 56, 57, 58, or 59 bp. In particular embodiments, shRNA sequences include 52 or 55 bp. In particular embodiments siRNAs have 15-25 bp. In particular embodiments siRNAs have 16, 17, 18, 19, 20, 21, 22, 23, or 24 bp. In particular embodiments siRNAs have 19 bp. The skilled artisan will appreciate, however, that siRNAs having a length of less than 16 nucleotides or greater than 24 nucleotides can also function to mediate RNAi. Longer RNAi agents have been demonstrated to elicit an interferon or Protein kinase R (PKR) response in certain mammalian cells which may be undesirable. Preferably the RNAi agents do not elicit a PKR response (i.e., are of a sufficiently short length). However, longer RNAi agents may be useful, for example, in situations where the PKR response has been downregulated or dampened by alternative means.

Small RNAs may also be used to activate gene expression.

### (IV-g) Pairing of Particular Coding Sequences and Particular LCRs

The present disclosure includes the recognition that an LCR, such as a long LCR can control expression (e.g., the level or cell type specificity of expression) of an operably linked coding nucleic acid sequence. Exemplary expression patterns (e.g., cell type and/or tissue type) associated with particular LCRs of the present disclosure are provided in Table 1. Accordingly, in various embodiments, a transposon payload can include an LCR, such as a long LCR, operably linked with a coding nucleic acid sequence encoding a product for expression in one or more cell or tissue types in which the LCR is known to drive expression. To provide but a few examples, a transposon payload of the present expression can include (i) a β-Globin LCR operably linked with a coding sequence encoding a protein for expression in erythrocytes, e.g., hematopoietic stem cells; (2) an immunoglobulin heavy chain LCR operably linked with a coding sequence encoding a protein for expression in B cells; or (3) a T Cell Receptor α/δ LCR or CD2 LCR operably linked with a coding sequence encoding a protein for expression in T cells. For example, a protein for expression in a hematopoietic stem cell can be a protein for treatment of a disorder selected from thalassemia, sickle cell anemia, or hemophilia; a protein for expression in B cells can be an antibody such as a therapeutic antibody; and a protein for expression in T cells can be a T Cell Receptor (TCR) such as an engineered TCR or a chimeric antigen receptor (CAR). Thus the present disclosure includes among other things (i) a β-Globin LCR operably linked with a coding sequence encoding a protein capable of partially or completely functionally replacing γ-globin, β-globin, or Factor VIII, or a gene editing CRISPR-Cas for correction of a mutation that causes sickle cell anemia; (2) an immunoglobulin heavy chain LCR operably linked with a coding sequence encoding an antibody; or (3) a T Cell Receptor α/δ LCR or CD2 LCR operably linked with a coding sequence encoding TCR or CAR.

### (V) Transposases

A transposase refers to an enzyme that is a component of a functional nucleic acid-protein complex capable of transposition and which is mediating transposition. Transposase also refers to integrases from retrotransposons or of retroviral origin. A transposition reaction includes a transposase and a transposase or an integrase enzyme. In particular embodiments, the efficiency of integration, the size of the DNA sequence that can be integrated, and the number of copies of a DNA sequence that can be integrated into a genome can be improved by using such transposable elements. Transposons include a short nucleic acid sequence with terminal repeat sequences upstream and downstream of a larger segment of DNA. Transposases bind the terminal repeat sequences and catalyze the movement of the transposon to another portion of the genome.

### (V-a) Use of Sleeping Beauty Transposase SB100x

Sleeping Beauty (SB) is a transposase derived from the genome of salmonid fish. SB is described in Ivics et al., Cell 91, 501-510, 1997; Izsvak etal., J. Mol. Biol., 93-102, 302(1), 2000; Geurts etal., Molecular Therapy, 8(1):108-117, 2003; Mates et al., Nature Genetics 41, 753-761, 2009; and U.S. Pat. Nos. 6,489,458; 7,148,203; and 7,160,682; U.S. Publication Nos. 2011/117072; 2004/077572; and 2006/252140.

Systematic mutagenesis studies have been undertaken to increase the activity of the SB transposase. For example, Yant et al., undertook the systematic exchange of the N-terminal 95 AA of the SB transposase for alanine (Mol. Cell Biol. 24: 9239-9247, 2004). Ten of these substitutions caused hyperactivity between 200-400% as compared to SB10 as a reference. SB16, described in Baus et al., Mol. Therapy 12: 1148-1156, 2005) was reported to have a 16-fold activity increase as compared to SB10. Additional hyperactive SB variants are described in Zayed et al. (Mol Therapy, 9(2):292-304, 2004) and U.S. Pat. No. 9,840,696. After screening several variants of SB transposase, the SB100X was found to be 100-fold more efficient than the first-generation transposase.

SB transposons need to circularize in order to transpose (Yant, et al., Nature Biotechnology, 20: 999-1005, 2002). Furthermore, there is an inverse linear relationship, for transposons between 1.9 and 7.2 kb, between the length of the transposon and transposition frequency. In other words, SB transposase mediate the delivery of larger transposons less efficiently compared to smaller transposons (Geurts, et al., Mol Ther., 8(1):108-17, 2003).

### (V-a-i) Inverted Repeat Sequences and Positions

In particular embodiments, the sequence encoding the IR(inverted repeat)/DR(direct repeat) and chromosomal sequence of Sleeping Beauty includes SEQ ID NO: 66. In particular embodiments, the sequence encoding the IR/DR and chromosomal sequence of Sleeping Beauty includes SEQ ID NO: 67. In particular embodiments, the IR/DR encoding sequence of Sleeping Beauty includes SEQ ID NO: 68. In particular embodiments, the sequence encoding the IR/DR and chromosomal sequence of Sleeping Beauty includes SEQ ID NO: 69. In particular embodiments, the sequence encoding the IR/DR and chromosomal sequence of Sleeping Beauty includes SEQ ID NO: 70. In particular embodiments, the sequence encoding the IR/DR of Sleeping Beauty includes SEQ ID NO: 71. In particular embodiments, the sequence encoding the IR/DR and chromosomal sequence of Sleeping Beauty includes SEQ ID NO: 72. In particular embodiments, the sequence encoding the IR/DR of Sleeping Beauty includes SEQ ID NO: 73.

### (V-a-ii) Transposase Sequences

In certain embodiments, the Sleeping Beauty transposase enzyme has the sequence SEQ ID NO: 74.

In certain embodiments, the hyperactive Sleeping Beauty is SB100X. In particular embodiments, SB100X has the sequence SEQ ID NO: 75.

### (V-b) Other Transposases

In addition to SB, a number of transposases have been described in the art that facilitate insertion of nucleic acids into the genome of vertebrates, including humans. Examples of such transposases include piggyBac™ (e.g., derived from lepidopteran cells and/or the Myotis lucifugus); mariner (e.g., derived from Drosophila); frog prince (e.g., derived from Rana pipiens); Tol1; Tol2 (e.g., derived from medaka fish); TcBuster™ (e.g., derived from the red flour beetle Tribolium castaneum), Helraiser, Himar1, Passport, Minos, Ac/Ds, PIF, Harbinger, Harbinger3-DR, HSmar1, and spinON.

### (V-b-i) Components and Sequences

The piggyBac™ (PB) transposase is a compact functional transposase protein that is described in, for example, Fraser et al., Insect Mol. Biol., 5:141-51, 1996; Mitra et al., EMBO J. 27:1097-1109, 2008; Ding et al., Cell, 122:473-83, 2005; and U.S. Pats. No. 6,218,185; 6,551,825; 6,962,810; 7,105,343; and 7,932,088. Hyperactive piggyBac™ transposases are described in U.S. Pat. No. 10,131,885.

In particular embodiments, PB transposase has the sequence as set forth in SEQ ID NO; 76 (GenBank ABS12111.1).

In particular embodiments, a Frog Prince transposase has the sequence as set forth in SEQ ID NO; 77 (GenBank: AAP49009.1). See also US2005/0241007.

In particular embodiments, a TcBuster transposase has the sequence as set forth in SEQ ID NO: 78 (GenBank: ABF20545.1).

In particular embodiments, a Tol2 transposase has the sequence set forth in SEQ ID NO: 79 (GenBank: BAA87039.1).

Additional information on DNA transposons can be found, for instance, in Muñoz-López & García Pérez, Curr Genomics, 11(2):115-128, 2010.

### (VI) Regulatory Components

The term “regulatory components” includes promoters, enhancers, transcription termination signals, polyadenylation sequences, and other expression control sequences. Regulatory components referred to in the invention include those which control expression of nucleic acid sequence host cells.

### (VI-a) Promoters

A promoter is a non-coding genomic DNA sequence, usually upstream (5′) to the relevant coding sequence, to which RNA polymerase binds before initiating transcription. This binding aligns the RNA polymerase so that transcription will initiate at a specific transcription initiation site. The nucleotide sequence of the promoter determines the nature of the enzyme and other related protein factors that attach to it and the rate of RNA synthesis. The RNA is processed to produce messenger RNA (mRNA) which serves as a template for translation of the RNA sequence into the amino acid sequence of the encoded polypeptide. The 5′ non-translated leader sequence is a region of the mRNA upstream of the coding region that may play a role in initiation and translation of the mRNA. The 3′ transcription termination/polyadenylation signal is a non-translated region downstream of the coding region that functions in the plant cell to cause termination of the RNA synthesis and the addition of polyadenylate nucleotides to the 3′ end.

Promoters can include general promoters, tissue-specific promoters, cell-specific promoters, and/or promoters specific for the cytoplasm. Promoters may include strong promoters, weak promoters, constitutive expression promoters, and/or inducible (conditional) promoters. Inducible promoters control expression in response to certain conditions, signals or cellular events. For example, the promoter may be an inducible promoter that requires a particular ligand, small molecule, transcription factor or hormone protein in order to effect transcription from the promoter. Particular examples of promoters include the AFP (α-fetoprotein) promoter, amylase 1C promoter, aquaporin-5 (AP5) promoter, αl -antitrypsin promoter, β-act promoter, β-globin promoter, [β-Kin promoter, B29 promoter, CCKAR promoter, CD14 promoter, CD43 promoter, CD45 promoter, CD68 promoter, CEA promoter, c-erbB2 promoter, CMV (cytomegalovirus viral) promoter, minCMV promoter, COX-2 promoter, CXCR4 promoter, desmin promoter, E2F-1 promoter, EF1α (elongation factor lα) promoter, EGR1 promoter, elF4A1 promoter, elastase-1 promoter, endoglin promoter, FerH promoter, FerL promoter, fibronectin promoter, Flt-1 promoter, GAPDH promoter, GFAP promoter, GP11b promoter, GRP78 promoter, GRP94 promoter, HE4 promoter, hGR1/1 promoter, hNIS promoter, Hsp68 promoter, Hsp68 minimal promoter, HSP70 promoter, HSV-1 virus TK gene promoter, hTERT promoter, ICAM-2 promoter, kallikrein promoter, LP promoter, major late promoter (MLP), Mb promoter, Rho promoter, MT (metallothionein) promoter, MUC1 promoter, Nphsl promoter, OG-2 promoter, PGK (Phospho Glycerate kinase) promoters, PGK-1 promoter, polymerase III (Pol 111) promoter, PSA promoter, ROSA promoter, Rous Sarcoma Virus (RSV) long-terminal repeat (LTR) promoter, SP-B promoter, Survivn promoter, SV40 (simian virus 40) promoter, SYN1 promoter, SYT8 gene promoter, TRP1 promoter, Tyr promoter, ubiquitin B promoter, and WASP promoter.

### (VI-a-i) Sources of Promoters

Promoters may be obtained as native promoters or composite promoters. Native promoters, or minimal promoters, refer to promoters that include a nucleotide sequence from the 5′ region of a given gene. A native promoter includes a core promoter and its natural 5′UTR. In particular embodiments, the 5 UTR includes an intron. Composite promoters refer to promoters that are derived by combining promoter elements of different origins or by combining a distal enhancer with a minimal promoter of the same or different origin.

### (VI-a-ii) Sequences of Exemplary Promoters and Variations on Sequences

In particular embodiments, the SV40 promoter includes the sequence set forth in SEQ ID NO: 80. In particular embodiments, the dESV40 promoter (SV40 promoter with deletion of the enhancer region) includes the sequence set forth in SEQ ID NO: 81. In particular embodiments, the human telomerase catalytic subunit (hTERT) promoter includes the sequence set forth in SEQ ID NO: 82. In particular embodiments, the RSV promoter derived from the Schmidt-Ruppin A strain includes the sequence set forth in SEQ ID NO: 83. In particular embodiments, the hNIS promoter includes the sequence set forth in SEQ ID NO: 84. In particular embodiments, the human glucocorticoid receptor 1A (hGR 1/Ap/e) promoter includes the sequence set forth in SEQ ID NO: 85.

In particular embodiments, promoters include wild type promoter sequences and sequences with optional changes (including insertions, point mutations or deletions) at certain positions relative to the wild-type promoter. In particular embodiments, promoters vary from naturally occurring promoters by having 1 change per 20 nucleotide stretch, 2 changes per 20 nucleotide stretch, 3 changes per 20 nucleotide stretch, 4 changes per 20 nucleotide stretch, or 5 changes per 20 nucleotide stretch. In particular embodiments, the natural sequence will be altered in 1, 2, 3, 4, 5, 6, 7, 8, 9, or 10 bases. The promoter may vary in length, including from about 50 nucleotides of LTR sequence to 100, 200, 250 or 350 nucleotides of LTR sequence, with or without other viral sequence.

### (VI-a-iii) Expression Patterns of Promoters

Some promoters are specific to a tissue or cell and some promoters are non-specific to a tissue or cell. Each gene in mammalian cells has its own promoter and some promoters can only be activated in certain cell types. A non-specific promoter, or ubiquitous promoter, aids in initiation of transcription of a gene or nucleotide sequence that is operably linked with the promoter sequence in a wide range of cells, tissues and cell cycles. In particular embodiments, the promoter is a non-specific promoter. In particular embodiments, a non-specific promoter includes CMV promoter, RSV promoter, SV40 promoter, mammalian elongation factor 1 α (EF1α) promoter, β-act promoter, EGR1 promoter, elF4A1 promoter, FerH promoter, FerL promoter, GAPDH promoter, GRP78 promoter, GRP94 promoter, HSP70 promoter, β-Kin promoter, PGK-1 promoter, ROSA promoter, and/or ubiquitin B promoter.

A specific promoter aids in cell specific expression of a nucleotide sequence that is operably linked with the promoter sequence. In particular embodiments, a specific promoter is active in a B cells, monocytic cells, leukocytes, macrophages, pancreatic acinar cells, endothelial cells, astrocytes, and/or any other cell type or cell cycle. In particular embodiments, the promoter is a specific promoter. In particular embodiments, an SYT8 gene promoter regulates gene expression in human islets (Xu, et al., Nat Struct Mol Biol., 2011, 18: 372-378). In particular embodiments, kallikrein promoter regulates gene expression in ductal cell specific salivary glands. In particular embodiments, the amylase 1C promoter regulates gene expression in acinar cells. In particular embodiments, the aquaporin-5 (AP5) promoter regulates gene expression in acinar cells (Zheng and Baum, Methods MolBiol., 434: 205-219, 2008). In particular embodiments, the B29 promoter regulates gene expression in B cells. In particular embodiments, the CD14 promoter regulates gene expression in monocytic cells. In particular embodiments, the CD43 promoter regulates gene expression in leukocytes and platelets. In particular embodiments, the CD45 promoter regulates gene expression in hematopoietic cells. In particular embodiments, the CD68 promoter regulates gene expression in macrophages. In particular embodiments, the desmin promoter regulates gene expression in muscle cells. In particular embodiments, the elastase-1 promoter regulates gene expression in pancreatic acinar cells. In particular embodiments, the endoglin promoter regulates gene expression in endothelial cells. In particular embodiments, the fibronectin promoter regulates gene expression in differentiating cells or healing tissue. In particular embodiments, the Flt-1 promoter regulates gene expression in endothelial cells. In particular embodiments, the GFAP promoter regulates gene expression in astrocytes. In particular embodiments, the GPllb promoter regulates gene expression in megakaryocytes. In particular embodiments, the ICAM-2 promoter regulates gene expression in endothelial cells. In particular embodiments, the Mb promoter regulates gene expression in muscle. In particular embodiments, the Nphsl promoter regulates gene expression in podocytes. In particular embodiments, the OG-2 promoter regulates gene expression in osteoblasts, odontoblasts. In particular embodiments, the SP-B promoter regulates gene expression in lung cells. In particular embodiments, the SYN1 promoter regulates gene expression in neurons. In particular embodiments, the WASP promoter regulates gene expression in hematopoietic cells.

In particular embodiments, the promoter is a tumor-specific promoter. In particular embodiments, the AFP promoter regulates gene expression in hepatocellular carcinoma. In particular embodiments, the CCKAR promoter regulates gene expression in pancreatic cancer. In particular embodiments, the CEA promoter regulates gene expression in epithelial cancers. In particular embodiments, the c-erbB2 promoter regulates gene expression in breast and pancreas cancer. In particular embodiments, the COX-2 promoter regulates gene expression in tumors. In particular embodiments, the CXCR4 promoter regulates gene expression in tumors. In particular embodiments, the E2F-1 promoter regulates gene expression in tumors. In particular embodiments, the HE4 promoter regulates gene expression in tumors. In particular embodiments, the LP promoter regulates gene expression in tumors. In particular embodiments, the MUC1 promoter regulates gene expression in carcinoma cells. In particular embodiments, the PSA promoter regulates gene expression in prostate and prostate cancers. In particular embodiments, the Survivn promoter regulates gene expression in tumors. In particular embodiments, the TRP1 promoter regulates gene expression in melanocytes and melanoma. In particular embodiments, the Tyr promoter regulates gene expression in melanocytes and melanoma.

### (VI-b) Micro RNA Sites

In various embodiments, a microRNA control system can refer to a method or composition in which expression of a gene is regulated by the presence of microRNA sites (e.g., nucleic acid sequences with which a microRNA can interact). In particular embodiments, a microRNA control system regulated expression of a gene such that the gene is expressed exclusively in target cells, such as HSPCs e.g., tumor infiltrating HSPCs. In some embodiments, a nucleic acid (e.g., a therapeutic gene) encoding a protein or nucleic acid of interest (e.g., an anti-cancer agent such as a CAR, TCR, antibody, and/or checkpoint inhibitor, e.g., an αPD-L1 antibody (e.g., an αPD-L1γ1 antibody) that is a checkpoint inhibitor) includes, is associated with, or is operatively linked with a microRNA site, a plurality of same microRNA sites, or a plurality of distinct microRNA sites. While those of skill in the art will be familiar with means and techniques of associating a microRNA site with a nucleic acid or portion thereof having a sequence that encodes a gene of interest, certain non-limiting examples are provided herein. For example, a gene of interest (e.g., a sequence encoding an αPD-L1γ1 antibody) can be present in a nucleic acid such that expression of the gene of interest is regulated by the presence of one or more microRNA sites that suppress expression in cells that are not tumor-infiltrating leukocyte cells, but do not suppress expression in tumor-infiltrating leukocytes. In certain particular examples, a gene of interest (e.g., a sequence encoding an αPD-L1y1 antibody) can be present in a nucleic acid such that expression of the gene of interest is regulated by the presence of one or more miR423-5p microRNA sites that suppress expression in cells that are not tumor-infiltrating leukocyte cells, but do not suppressed expression in tumor-infiltrating leukocytes. In various embodiments, a microRNA control system can include a nucleic acid that includes, or in which expression of a protein or nucleic acid of interest is regulated by, one or more microRNA sites, e.g., 1, 2, 3, 4, 5, 6, 7, 8, 9, 10, or more microRNA sites. In various embodiments, a microRNA control system can include a nucleic acid that includes, or in which expression of a protein or nucleic acid of interest is regulated by, one or more miR423-5p microRNA sites, e.g., 1, 2, 3, 4, 5, 6, 7, 8, 9, 10, or more miR423-5p microRNA sites. In some particular embodiments, a microRNA control system can include a nucleic acid that encodes αPD-L1y1 antibody and includes, or in which expression of αPD-L1y1 antibody is regulated by, one or more miR423-5p microRNA sites, e.g., 1, 2, 3, 4, 5, 6, 7, 8, 9, 10, or more miR423-5p microRNA sites, e.g., miR423-5p microRNA sites.

### (Vl-c) Pairings of Particular Regulatory Components, Particular Coding Sequences, and/or Particular Long LCRs

A transposon payload of the present disclosure can include an LCR, such as a long LCR, operably linked with a coding nucleic acid sequence (e.g., a nucleic acid sequence encoding a protein), where the coding nucleic acid sequence is also operably linked with a promoter. In various embodiments, a transposon payload includes coding nucleic acid sequence operably linked with both (i) an LCR and (ii) a promoter that is typically operably linked with the LCR in a human genome. In other words, a transposon payload can include an LCR together with a promoter with which it is naturally paired, where both together drive expression of a coding nucleic acid sequence. In various embodiments, a promoter naturally paired with an LCR is a promoter as shown in Table 2 In various embodiments, a promoter is a nucleic acid sequence immediately upstream of a start codon of a coding sequence that is naturally paired with the LCR in a human genome, e.g., a nucleic acid sequence including 100 bp, 200 bp, 300 bp, 400 bp, 500 bp, 1,000 bp, 1,500 bp, 2,000 bp, 3,000 bp, 4,000 bp, 5,000 bp, or more nucleotides immediately upstream of the start codon, e.g., in a reference genome. In various embodiments, a promoter is a nucleic acid sequence that includes a nucleic acid sequence that is includes, e.g., 100 bp-5,000 bp, 100 bp-4,000 bp, 100 bp-3,000 bp, 100 bp-2,000 bp, 100 bp-1,000 bp, 1,000 bp-5,000 bp, 1,000 bp-4,000 bp, 1,000 bp-3,000 bp, or 1,000 bp-2,000 bp immediately upstream of a start codon of a coding sequence that is naturally paired with the LCR in a human genome. In various embodiments, a coding sequence naturally paired with the LCR in a human genome is a coding sequence shown in Table 1 or Table 2.

In various embodiments, a transposon payload includes a coding nucleic acid sequence operably linked with both (i) an LCR and (ii) a promoter that is not typically operably linked with the LCR in a human genome. The present disclosure encompasses the recognition that an LCR may have evolved in a particular context but can be applied to control expression of coding nucleic acid sequences with which it is not typically operably linked in the human genome and/or to drive expression of a coding nucleic acid sequence expression of which is also driven by a promoter with which the LCR is not typically associated in the human genome. Accordingly, an LCR may be paired with a promoter and/or gene with which it is naturally operably linked (e.g., in a transposon payload including a β-Globin LCR operably linked with a coding nucleic acid sequence encoding β-globin or γ-globin together with a β-globin promoter), or may be paired with a promoter and/or gene with which it is not naturally operably linked (e.g., a β-Globin LCR operably linked with a coding nucleic acid sequence encoding a replacement for Factor VIII, such as ET3).

TABLE 2.

| | | | |
|---|---|---|---|
| LCRs | Exemplary Tissue | Exemplary Promoter | Exemplary Coding Sequence (transgene/therapeutic gene) |
| β-Globin LCR | Erythrocytes | β-promoter | downstream beta-globin genes (epsilon, G-gamma, A-gamma, delta and beta, or HBE1, HBG2, HBG1, HBD and HBB) |
| Adenosine Deaminase LCR | Enriched in blood, intestine, and lymphoid tissue | ADA promoter | Adenosine Deaminase |
| Apolipo-protein E/C-1 LCR | Adrenal gland, Liver | APOE promoter, APOC-I promoter, APOC-II promoter | APOE, APOC-I, APOC-II |
| T Cell Receptor α/δ LCR | T Cells | | TCR gene and Dad1 anti-apoptosis gene |
| CD2 LCR | T Cells | | CD2 |
| S100β LCR | Brain Astrocytes | S100β promoter | S100β |
| Growth Hormone LCR | Pituitary Gland | Human growth hormone (hGH) promoter | GH1 (growth hormone 1), CSHL1 (chorionic somatomammotropin hormone-like 1), CSH1 (chorionic somatomammotropin hormone 1 (placental lactogen)), GH2 (growth hormone 2) and CSH2 (chorionic somatomammotropin hormone 2) |
| Apolipo-protein B LCR | Intestine, Liver | | APOB |
| β Myosin Heavy Chain LCR | Heart Muscle, Skeletal Muscle | | β Myosin Heavy Chain |
| MHC Class I HLA-B7 LCR | All Cells | | |
| Immunoglobulin Heavy Chain LCR | B Cells | | |
| Immunoglobulin Cα ½ LCR | B cells | | |
| Keratin 18 LCR | Epithelial Cells | KRT18 promoter | Keratin 18 (KRT18) |
| MHC Class I HLA G LCR | All Cells | HLA-G promoter | HLA-G |
| Complement Component C4A/B LCR | Liver | | C4A |
| Red and Green Visual Pigment LCR (OPSIN LCR) | Cone Photoreceptors | | opsin 1, long-wave-sensitive; OPN1LW; opsin 1, medium-wave-sensitive; OPN1MW, OPN1 MW2, and OPN1 MW3 |
| CD4 LCR | CD4+ T Cells | | CD4 |
| α-Lactalbumin LCR | Mammary Glands | | α-Lactalbumin |
| Desmin LCR | Heart Muscle, Skeletal Muscle, Smooth Muscle | | Desmin |
| CYP19/aroma tase LCR | Multiple tissues | | CYP19A1 |
| C-fes Proto-Oncogene LCR | Myeloid cells including macrophages and neutrophils | | FES |
| α-globin locus control region | Erythrocytes | | HBZ (hemoglobin, zeta), HBA2 (hemoglobin, alpha 2), HBA1 (hemoglobin, alpha 1) and HBQ1 (hemoglobin, theta 1) genes within the alpha-globin gene cluster |
| nuclear factor, erythroid 2 like 1 (NFE2L1) LCR | Erythrocytes | | NFE2L1 |

### (VII) Vectors

### (VII-a) Vector Features That Can Be Optimized to Improve Large Payload Integration

Adenoviral genomes are linear, non-segmented double-stranded DNA ranging from 26 kb to 45 kb in length, depending on the serotype. The adenoviral DNA is flanked on both ends by inverted terminal repeats (ITRs), which act as a self-primer to promote primase-independent DNA synthesis and to facilitate integration into the host genome. Adenoviral genomes also contain a packaging signal, which facilities proper viral transcript packaging and is located on the left arm of the genome. Viral transcripts encode several proteins including early transcriptional units, E1, E2, E3, and E4 and late transcriptional units which encode structural components of the Ad virion (Lee et al., Genes Dis., 4(2):43-63, 2017).

The adenovirus is a large, icosahedral-shaped, non-enveloped virus. The viral capsid includes three types of proteins including fiber, penton, and hexon based proteins. The hexon makes up the majority of the viral capsid, forming the 20 triangular faces. The penton base is located at the 12 vertices of the capsid and the fiber (also referred to as knobbed fiber) protrudes from each penton base. These proteins, the penton and fiber, are of particular importance in receptor binding and internalization as the facilitate the attachment of the capsid to a host cell (Lee et al., Genes Dis., 4(2):43-63, 2017).

Adenoviruses are particularly suited for gene therapy because of their stable and safe genome. The double stranded characteristic of Ad vectors increases the vectors stability and reduces genetic shift or drift compared to single-stranded DNA or RNA viruses. Reducing errors during DNA replication, Ad vectors use a proof-reading DNA polymerase. Furthermore, Ad vectors do not integrate their DNA with the host’s genome, rather they transfer episomal DNA to the nucleus of the host cell.

Ad vectors are also susceptible to genetic modification and research have made modification to further improve their use in gene therapy.

### (VII-b) Serotypes and Pseudotypes

Human adenoviruses (Ads) are classified into six subgroups containing over 50 serotypes. The groups are labeled A to F. Group B Ads include Ad3, Ad7, Ad11, Ad14, Ad16, Ad21, Ad34, Ad 35, and Ad50. Ad5 is classified into Group C. Because there are more than 50 human Ad serotypes, Ad vectors can be modified to target different host cells of interest. Different Ad serotypes bind to different cellular receptors and use different entry mechanisms.

The infectivity of different Ad serotypes is limited to a number of human cell lines. Infectivity studies revealed that Ad5 and Ad3 are particularly suitable for infecting and targeting endothelial or lymphoid cells, whereas Ad9, Ad11 and Ad35 efficiently infected human bone marrow cells. Therefore, the knob domain of the fiber protein of Ad9, Ad11 and Ad35 are excellent candidates for retargeting the Ad5 vector to human bone marrow cells. Other possible serotypes include Ad7.

In particular embodiments, the Ad vector is a recombinant vector. In particular embodiments, Ad5/35 is a recombinant Ad5 vector expressing a modified fiber protein including a fiber tail domain of Ad5 and the fiber shaft and knob domains of Ad35. In particular embodiments, the Ad vector is selected from Ad5, Ad35, Ad5/35. Ad5/35++, or Ad35++.

In particular embodiments, an Ad vector includes a nucleic acid that encodes a CD46 binding adenoviral fiber polypeptide. A fiber polypeptide refers to a polypeptide including: (a) an N-terminal tail domain or equivalent thereof, which interacts with the penton base protein of the capsid and contains the signals necessary for transport of the protein to the cell nucleus; (b) one or more shaft domains or equivalents thereof; and (c) a C-terminal knob domain or equivalent thereof that contains the determinants for receptor binding. The C-terminal domain of the fiber polypeptide that is able to form into a homotrimer that binds to CD46 is referred to as a fiber knob. The C-terminal portion of the fiber protein can trimerize and form a fiber structure that binds to CD46. Only the fiber knob is required for CD46-targeting. Thus, the second nucleic acid module encodes an adenoviral fiber including one or more human adenoviral knob domain, or equivalent thereof, that bind to CD46. When multiple knob domains are encoded, the knob domains may be the same or different, so long as they each bind to CD46. As used herein, a knob domain “functional equivalent” is knob domain with one or more amino acid deletions, substitutions, or additions that retains binding to CD46 on the surface of CD34+ cells.

An adenoviral fiber polypeptide also includes a shaft domain. The shaft domain is not critical for CD46 binding. In particular embodiments, the shaft domain can include one or more shaft domains from the different human Ad serotypes. In particular embodiments, the shaft domain can include any portion of a shaft domain, or mutant thereof, that permits fiber knob trimerization. In particular embodiments, the shaft domain is selected from Ad5 shaft domains, Ad35 shaft domains, and functional equivalents thereof. As used herein, a functional equivalent of a shaft domain is any portion of a shaft domain, or mutant thereof, that permits fiber knob trimerization. Where more than 1 shaft domain or equivalent is present, each shaft domain or equivalent can be identical, or one or more copies of the shaft domain or equivalent may differ in a single recombinant polypeptide.

An adenoviral fiber polypeptide also includes a tail domain. The adenoviral tail domain or a mutant thereof interacts with the penton base protein of the capsid (on a helper Ad virus) and contains the signals necessary for transport of the protein to the cell nucleus. The tail domain used is one that will interact with the penton based protein of the helper Ad virus capsid being used for HD-Ad production. Thus, if an Ad5 helper virus is used, the tail domain will be derived from Ad5; if an Ad35 helper virus is used, the tail domain will be from Ad 35, etc.

In particular embodiments, an Ad vector includes an Ad5/35 vector. In particular embodiments, an Ad5/35 vector is a chimeric Ad vector with an Ad35 fiber knob and Ad5 shaft.

In particular embodiments, an Ad vector includes an Ad5/35++ vector. In particular embodiments, an Ad5/35++ vector is a chimeric Ad5/35 vector with a mutant Ad35 fiber knob. The vector is mutated to increase the affinity to CD46 by 25-fold and increases cell transduction efficiency at lower multiplicity of infection (MOI) (Li and Lieber, FEBS Letters, 593(24): 3623-3648, 2019).

In particular embodiments, an Ad vector includes an Ad35 vector. In particular embodiments, an Ad35 vector is a class B Ad vector with an Ad35 fiber knob and shaft.

In particular embodiments, an Ad vector includes an Ad35++ vector. In particular embodiments, an Ad35++ vector is an Ad35 vector with an enhanced Ad35 fiber knob and an Ad35 shaft.

In particular embodiments, an Ad vector includes Ad3, Ad7, Ad11, Ad14, Ad16, Ad21, Ad34, or Ad50.

### (VII-c) Components

In particular embodiments, the vector includes components including a payload, regulatory components, integration elements, selection cassette, and a stuffer sequence.

### (VII-c-i) Payload

In particular embodiments, a vector includes a payload (e.g., a transposon payload). In particular embodiments, the payload encodes a gene of interest. In particular embodiments, the payload can include additional elements for the expression such as an intron sequence, a signal sequence, a nuclear localization sequence, a transcription termination sequence, or a site for initiation of translation of the IRES type. Additional description of payloads can be found herein.

### (VII-c-ii) Regulatory Components

In particular embodiments, the vector includes regulatory components. Regulatory components are described in more detail in section VI. Regulatory components can include enhancers, promoters, and other sequences that that regulate gene expression.

In particular embodiments, regulatory components facilitate transcription of the sequence encoding the payload into RNA and/or the translation of an mRNA into a protein. Suitable promoters include, for example, those of eukaryotic or viral origin. Suitable promoters can be constitutive or regulatable (e.g., inducible). Examples of suitable promoters include, for example, the AFP (α-fetoprotein) promoter, amylase 1C promoter, aquaporin-5 (AP5) promoter, αl -antitrypsin promoter, β-act promoter, β-globin promoter, β-Kin promoter, B29 promoter, CCKAR promoter, CD14 promoter, CD43 promoter, CD45 promoter, CD68 promoter, CEA promoter, c-erbB2 promoter, CMV (cytomegalovirus viral) promoter, COX-2 promoter, CXCR4 promoter, desmin promoter, E2F-1 promoter, EF1 α (elongation factor lα) promoter, EGR1 promoter, elF4A1 promoter, elastase-1 promoter, endoglin promoter, FerH promoter, FerL promoter, fibronectin promoter, Flt-1 promoter, GAPDH promoter, GFAP promoter, GPllb promoter, GRP78 promoter, GRP94 promoter, HE4 promoter, hGR1/1 promoter, hNIS promoter, Hsp68 promoter, HSP70 promoter, HSV-1 virus TK gene promoter, hTERT promoter, ICAM-2 promoter, kallikrein promoter, LP promoter, major late promoter (MLP), Mb promoter, Rho promoter, MT (metallothionein) promoter, MUC1 promoter, Nphsl promoter, OG-2 promoter, PGK (Phospho Glycerate kinase) promoters, PGK-1 promoter, polymerase III (Pol III) promoter, PSA promoter, ROSA promoter, Rous Sarcoma Virus (RSV) long-terminal repeat (LTR) promoter, SP-B promoter, Survivn promoter, SV40 (simian virus 40) promoter, SYN1 promoter, SYT8 gene promoter, TRP1 promoter, Tyr promoter, ubiquitin B promoter, and WASP promoter.

### (VII-c-iii) Integration Elements

Various SB transposases are known in the art. Examples of SB transposases known in the art include, without limitation, SB, SB11, SB12, HSB1, HSB2, HSB3, HSB4, HSB5, HSB13, HSB14, HSB15, HSB16, HSB17, SB100x, and SB150x. In particular embodiments, the present disclosure utilizes an SB100x transposase. In some embodiments, an SB100x or an SB150x transposase can be used. In some embodiments, any SB transposase can be used.

SB transposases transpose nucleic acid transposon payloads that are positioned between SB inverted terminal repeats (ITRs). Various SB ITRs are known in the art. In some embodiments, an SB ITR is a 230 bp sequence including imperfect direct repeats of 32 bp in length that serve as recognition signals for the transposase. Engineered SB ITRs are known in the art, including SB ITRs known as pT, pT2, pT3, pT2B, and pT4. In some embodiments, pT4 ITRs are used, e.g., to flank a transposon payload of the present disclosure, e.g., for transposition by an SB100x transposase.

### (VII-c-iv) Selection Elements

In particular embodiments vectors include a selection element including a selection cassette. In particular embodiments, a selection cassette includes a promoter, a cDNA that adds resistance to a selection agent, and a poly A sequence enabling stopping the transcription of this independent transcriptional element.

A selection cassette can encode proteins that (a) confer resistance to antibiotics or other toxins, (b) complement auxotrophic deficiencies, or (c) supply critical nutrients not available from complex media, e.g., the gene encoding D-alanine racemase for Bacilli. Any number of selection systems may be used to recover transformed cell lines. In particular embodiments, a positive selection cassette includes resistance genes to neomycin, hygromycin, ampicillin, puromycin, phleomycin, zeomycin, blasticidin, viomycin. In particular embodiments, a positive selection cassette includes the DHFR (dihydrofolate reductase) gene providing resistance to methotrexate, the MGMT P140K gene responsible for the resistance to O⁶BG/BCNU, the HPRT (Hypoxanthine phosphoribosyl transferase) gene responsible for the transformation of specific bases present in the HAT selection medium (aminopterin, hypoxanthine, thymidine) and other genes for detoxification with respect to some drugs. In particular embodiments, the selection agent includes neomycin, hygromycin, puromycin, phleomycin, zeomycin, blasticidin, viomycin, ampicillin, O⁶BG/BCNU, methotrexate, tetracycline, aminopterin, hypoxanthine, thymidine kinase, DHFR, Gln synthetase, or ADA.

In particular embodiments, negative selection cassettes include a gene for transformation of a substrate present in the culture medium into a toxic substance for the cell that expresses the gene. These molecules include detoxification genes of diptheria toxin (DTA) (Yagi et al., Anal Biochem. 214(1):77-86, 1993; Yanagawa et al., Transgenic Res. 8(3):215-221, 1999), the kinase thymidine gene of the Herpes virus (HSV TK) sensitive to the presence of ganciclovir or FIAU. The HPRT gene may also be used as a negative selection by addition of 6-thioguanine (6TG) into the medium. and for all positive and negative selections, a poly A transcription termination sequence from different origins, the most classical being derived from SV40 poly A, or a eukaryotic gene poly A (bovine growth hormone, rabbit β-globin, etc.).

In particular embodiments, the selection cassette includes MGMT P140K as described in Olszko et al. (Gene Therapy 22: 591-595, 2015). In particular elements, the selection agent includes O⁶BG/BCNU.

The drug resistant gene MGMT encoding human alkyl guanine transferase (hAGT) is a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG but retain their ability to repair DNA damage (Maze et al., J. Pharmacol. Exp. Ther. 290: 1467-1474, 1999). P140K^{MGMT}-based drug resistant gene therapy has been shown to confer chemoprotection to mouse, canine, rhesus macaques, and human cells, specifically hematopoietic cells (Zielske et al., J. Clin. Invest. 112:1561-1570, 2003; Pollok et al., Hum. Gene Ther. 14: 1703-1714, 2003; Gerull et al., Hum. Gene Ther. 18: 451-456, 2007; Neff et al., Blood 105: 997-1002, 2005; Larochelle et al., J. Clin. Invest. 119: 1952-1963, 2009; Sawai et al., Mol. Ther. 3: 78-87, 2001).

In particular embodiments, combination with an in vivo selection cassette will be a critical component for diseases without a selective advantage of gene-corrected cells. For example, in SCID and some other immunodeficiencies and FA, corrected cells have an advantage and only transducing the therapeutic gene into a “few” HSPCs is sufficient for therapeutic efficacy. For other diseases like hemoglobinopathies (i.e., sickle cell disease and thalassemia) in which cells do not demonstrate a competitive advantage, in vivo selection of the gene corrected cells, such as in combination with an in vivo selection cassette such as MGMT P140K, will select for the few transduced HSPCs, allowing an increase in the gene corrected cells and in order to achieve therapeutic efficacy. This approach can also be applied to HIV by making HSPCs resistant to HIV in vivo rather than ex vivo genetic modification.

### (VII-c-v) Stuffer Sequence

In particular embodiments, the vector includes a stuffer sequence. In particular embodiments, the stuffer sequence may be added to render the vector genome at a size near that of wild-type length. Stuffer is a term generally recognized in the art intended to define functionally inert sequence intended to extend the length

The stuffer sequence is used to achieve efficient packaging and stability of the vector. In particular embodiments, the stuffer sequence is used to render the vector genome size between 70% and 110% of that of the wild type virus.

The stuffer sequences can be any DNA, preferably of mammalian origin. In a preferred embodiment of the invention, stuffer sequences are non-coding sequences of mammalian origin, for example intronic fragments.

The stuffer sequence, when used to keep the size of the vector a predetermined size, can be any non-coding coding sequence or sequence that allows the vector genome to remain stable in dividing or nondividing cells. These sequences can be derived from other viral genomes (e.g. Epstein bar virus) or organism (e.g. yeast). For example, these sequences could be a functional part of centromeres and/or telomeres.

### (VII-d) Helper-dependent Adenoviral Vectors

Helper-dependent adenoviral vectors (HDAd) are engineered to lack all viral coding sequences, efficiently transduce a wide variety of cell types, and can mediate long-term transgene expression with negligible chronic toxicity. Deletion of the viral coding sequences and leaving only the cis-acting elements necessary for vector genome replication (ITRs) and encapsidation (ψ), cellular immune response against the Ad vector is reduced. HDAd vectors have a large cloning capacity of up to 37 kb, allowing for the delivery of large payloads. These payloads can include large therapeutic genes or even multiple transgenes and large regulatory components to enhance, prolong, and regulate transgene expression. Like other adenoviral vectors, the HDAd genome remains episomal and does not integrate with the host genome (Rosewell et al., J Genet Syndr Gene Ther. Suppl 5:001, 2011).

In some HDAd vector systems, one viral genome (a helper) encodes all of the proteins required for replication but has a conditional defect in the packaging sequence, making it less likely to be packaged into a virion. A second viral genome includes only viral inverted terminal repeats (ITRs), a therapeutic payload, and a normal packaging sequence, which allows this second viral genome to be selectively packaged into HDAd viral vectors and isolated from the producer cells. HDAd viral vectors can be further purified from helper vectors by physical means. In general, some contamination of helper vectors and/or helper genomes in HDAd viral vectors and HDAd viral vector formulations can occur and can be tolerated.

In some HDAd vector systems, a helper genome utilizes a Cre/loxP system. In certain such HDAd vector systems, the HDAd donor vector genome includes 500 bp of noncoding adenoviral DNA that includes the adenoviral ITRs which are required for vector genome replication, and ψ which is the packaging sequence required for encapsidation of the vector genome into the capsid. It has also been observed that the HDAd donor vector genome can be most efficiently packaged when it has a total length of about 27.7 kb to about 37 kb, which length can be composed, e.g., of a therapeutic payload and or a “stuffer” sequence. The HDAd donor vector genome can be delivered to cells, such as 293 cells that expresses Cre recombinase, optionally where the HDAd donor vector genome is delivered to the cells in a non-viral vector form, such as a bacterial plasmid form (e.g., where the HDAd donor vector genome is constructed as a bacterial plasmid (pHDAd) and is liberated by restriction enzyme digestion). The same cells can be transduced with the helper genome, which can include an E1-deleted, Ad vector bearing a packaging sequence flanked by IoxP sites so that following infection of 293 cells expressing Cre recombinase, the packaging sequence is excised from the helper genome by Cre-mediated site-specific recombination between the IoxP sites. Thus, the HDAd donor vector genome can be transfected into 293 cells that express Cre and are transduced with a helper genome bearing a packaging signal (ψ) flanked by IoxP sites such that Cre-mediated excision of ψ renders the helper virus genome unpackageable, but still able to provide all of the necessary trans-acting factors for propagation of the HDAd. After excision of the packaging sequence, a helper genome is unpackageable but still able to undergo DNA replication and thus trans-complement the replication and encapsidation of the HDAd donor vector genome. In some embodiments, to prevent generation of replication competent Ad (RCA; E1⁺) as a consequence of homologous recombination between the helper and HDAd donor vector genomes present in 293 cells a “stuffer” sequence can be inserted into the E3 region to render any E1⁺ recombinants too large to be packaged. Similar HDAd production systems have been developed using FLP (e.g., FLPe)/frt site-specific recombination, where FLP-mediated recombination between frt sites flanking the packaging signal of the helper genome selects against encapsidation of helper genomes in 293 cells that express FLP. Alternative strategies to select against the helper vectors have been developed.

An HDAd5/35 vector is a helper-dependent chimeric Ad5/35 vector with a Ad35 fiber knob and an Ad5 shaft. An HDAd5/35++ vector is a helper-dependent chimeric Ad5/35 vector with a mutant Ad35 fiber knob. The vector is mutated to increase the affinity to CD46 by 25-fold and increases cell transduction efficiency at lower multiplicity of infection (MOI) (Li & Lieber, FEBS Letters, 593(24): 3623-3648, 2019). An HDAd35 vector is a helper-dependent Ad35 vector. An HDAd35++ vector is a helper-dependent Ad35 vector with a mutant Ad35 fiber knob which enhances its affinity to CD46 and increases cell transduction efficiency.

### (VII-e) Vector-targeted Cell Types (and Vector Molecular Targets)

### (VII-e-i) HSCs

In particular embodiments, vector-targeted cell types include hematopoietic stem cells (HSCs). HSCs are targeted for in vivo genetic modification by binding CD46. Vectors can include mutations to increase the specificity and/or strength of CD46 binding. HSC can also be identified by the following marker profiles: CD34+, Lin-CD34+CD38-CD45RA-CD90+CD49f+ (HSC1) and CD34+CD38-CD45RA-CD90- CD49f+ (HSC2). Human HSC1 can be identified by the following profiles: CD34+/CD38-/CD45RA-/CD90+ or CD34+/CD45RA-/CD90+ and mouse LT-HSC can be identified by Lin-Sca1+ckit+CD150+CD48-Flt3-CD34- (where Lin represents the absence of expression of any marker of mature cells including CD3, Cd4, CD8, CD11b, CD11c, NK1.1, Gr1, and TER119). In particular embodiments, HSC are identified by a CD164+ profile. In particular embodiments, HSC are identified by a CD34+/CD164+ profile. For additional information regarding HSC marker profiles, see WO2017/218948.

### (VII-e-ii) T Cells

Several different subsets of T-cells have been discovered, each with a distinct function. For example, a majority of T-cells have a T-cell receptor (TCR) existing as a complex of several proteins. The actual T-cell receptor is composed of two separate peptide chains, which are produced from the independent T-cell receptor alpha and beta (TCRα and TCRβ) genes and are called α- and β-TCR chains.

y8 T-cells represent a small subset of T-cells that possess a distinct T-cell receptor (TCR) on their surface. In γδ T-cells, the TCR is made up of one γ-chain and one δ-chain. This group of T-cells is much less common (2% of total T-cells) than the αβ T-cells.

CD3 is expressed on all mature T cells. Activated T-cells express 4-1 BB (CD137), CD69, and CD25. CD5 and transferrin receptor are also expressed on T-cells.

T-cells can further be classified into helper cells (CD4+ T-cells) and cytotoxic T-cells (CTLs, CD8+ T-cells), which include cytolytic T-cells. T helper cells assist other white blood cells in immunologic processes, including maturation of B cells into plasma cells and activation of cytotoxic T-cells and macrophages, among other functions. These cells are also known as CD4+ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T-cells destroy virally infected cells and tumor cells, and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body.

In particular embodiments, CARs are genetically modified to be expressed in cytotoxic T-cells.

“Central memory” T-cells (or “TCM”) as used herein refers to an antigen experienced CTL that expresses CD62L or CCR7 and CD45RO on the surface thereof, and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells.

“Effector memory” T-cell (or “TEM”) as used herein refers to an antigen experienced T-cell that does not express or has decreased expression of CD62L on the surface thereof as compared to central memory cells and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T-cells are positive for granzyme B and perforin as compared to memory or naive T-cells.

“Naive” T-cells as used herein refers to a non-antigen experienced T cell that expresses CD62L and CD45RA and does not express CD45RO as compared to central or effector memory cells. In particular embodiments, naive CD8+ T lymphocytes are characterized by the expression of phenotypic markers of naive T-cells including CD62L, CCR7, CD28, CD127, and CD45RA.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

### (VII-e-iii) B Cells

B cells are mediators of the humoral response and are responsible for production and release of antibodies specific to an antigen. Several types of B cells exist which can be characterized by key markers. In general, immature B cells express CD19, CD20, CD34, CD38, and CD45R, and as they mature the key expressed markers are CD19 and IgM.

### (VII-e-iv) Tumors

In particular embodiments, vectors can target tumors. In particular embodiments, tumors are targeted by targeting receptors present on tumor cells and not on healthy cells. Tumors can be targeted for in vivo genetic modification by binding αv integrins. The αv integrins play an important role in angiogenesis. The αvβ3 and αvβ5 integrins are absent or expressed at low levels in normal endothelial cells but are induced in angiogenic vasculature of tumors (Brooks et al., Cell, 79: 1157-1164, 1994; Hammes et al., Nature Med, 2: 529-533, 1996). Aminopeptidase N/CD13 has recently been identified as an angiogenic receptor for the NGR motif (Burg et al., Cancer Res, 59:2869-74, 1999). Aminopeptidase N/CD13 is strongly expressed in the angiogenic blood vessels of cancer and in other angiogenic tissues.

In particular embodiments, vectors can target tumors by targeting cancer cell antigen epitopes. Cancer cell antigens are expressed by cancer cells or tumors.

In particular embodiments, cancer cell antigen epitopes are preferentially expressed by cancer cells. “Preferentially expressed” means that a cancer cell antigen is found at higher levels on cancer cells as compared to other cell types. In some instances, a cancer antigen epitope is only expressed by the targeted cancer cell type. In other instances, the cancer antigen is expressed on the targeted cancer cell type at least 25%, 35%, 45%, 55%, 65%, 75%, 85%, 95%, 96%, 97%, 98%, 99%, or 100% more than on non-targeted cells.

In particular embodiments, cancer cell antigens are significantly expressed on cancerous and healthy tissue. In particular embodiments, significantly expressed means that the use of a bi-specific antibody was stopped during development based on on-target/off-cancer toxicities. In particular embodiments, significantly expressed means the use of a bi-specific antibody requires warnings regarding potential negative side effects based on on-target/off-cancer toxicities. As one example, cetuximab is anti-EGFR antibody associated with a severe skin rash thought to be due to EGFR expression in the skin. Another example is Herceptin (trastuzumab), which is an anti-HER2 (ERBB2) antibody. Herceptin is associated with cardiotoxicity due to target expression in the heart. Moreover, targeting Her2 with a CAR-T cell was lethal in a patient due to on-target, off-cancer expression in the lung.

Table 3 provides examples of cancer antigens that are more likely to be co-expressed in particular cancer types.

TABLE 3:

| | |
|---|---|
| Cancer Antigens Likely to be Co-Expressed | Cancer Type |
| CD19, CD20, CD22, ROR1, CD33, CD56, CLL-1, WT-1, CD123, PD-L1, EFGR | Leukemia/Lymphoma |
| B-cell maturation antigen (BCMA), PD-L1, EFGR | Multiple Myeloma |
| PSMA, WT1, Prostate Stem Cell antigen (PSCA), SV40 T, PD-L1, EFGR | Prostate Cancer |
| HER2, ERBB2, ROR1, PD-L1, EFGR, MUC16, folate receptor (FOLR), CEA | Breast Cancer |
| CD133, PD-L1, EFGR | Stem Cell Cancer |
| L1-CAM, MUC16, FOLR, Lewis Y, ROR1, mesothelin, WT-1, PD-L1, EFGR, CD56 | Ovarian Cancer |
| mesothelin, PD-L1, EFGR | Mesothelioma |
| carboxy-anhydrase-IX (CAlX); PD-L1, EFGR | Renal Cell Carcinoma |
| GD2, PD-L1, EFGR | Melanoma |
| mesothelin, CEA, CD24, ROR1, PD-L1, EFGR, MUC16 | Pancreatic Cancer |
| ROR1, PD-L1, EFGR, mesothelin, MUC16, FOLR, CEA, CD56 | Lung Cancer |
| mesothelin, PD-L1, EFGR | Cholangiocarcinoma |
| MUC16, PD-L1, EFGR, | Bladder Cancer |
| ROR1, glypican-2, CD56, disialoganglioside, PD-L1, EFGR, | Neuroblastoma |
| CEA, PD-L1, EFGR, | Colorectal Cancer |
| CD56, PD-L1, EFGR, | Merkel Cell Carcinoma |

In more particular examples, cancer cell antigens include: Mesothelin, MUC16, FOLR, PD-L1, ROR1, glypican-2 (GPC2), disialoganglioside (GD2), HER2, EGFR, EGFRvIII, CEA, CD56, CLL-1, CD19, CD20, CD123, CD30, CD33 (full length), CD33 (DeltaE2 variant), CD33 (with C-terminal truncation), BCMA, IGFR, MUC1, VEGFR, PSMA, PSCA, IL13Ra2, FAP, EpCAM, CD44, CD133, Tro-2, CD200, FLT3, GCC, and WT1. As will be understood by one of ordinary skill in the art, targeted antigens can lack signal peptides.

CD56, also known as neural cell adhesion molecule 1 (NCAM1), is a type I membrane glycoprotein involved in cell-cell and cell-matrix adhesion. Its extracellular domain has five IgG-like domains at the N-terminus and two fibronectin type III domains in the membrane-proximal region.

Disialoganglioside GalAcbeta1-4(NeuAcalpha2-8NeuAcalpha2-3)Galbeta1-4Glcbeta1-1Cer (GD2) is expressed on various tumors, including neuroblastoma. The disialoganglioside antigen GD2 includes a backbone of oligosaccharides flanked by sialic acid and lipid residues. See, e.g., Cheresh (Surv. Synth. Pathol. Res. 4:97, 1987) and U.S. Pat. No. 5,653,977.

EGFR variant III (EGFRvlll), a tumor specific mutant of EGFR, is a product of genomic rearrangement which is often associated with wild-type EGFR gene amplification. EGFRvIII is formed by an in-frame deletion of exons 2-7, leading to deletion of 267 amino acids with a glycine substitution at the junction. The truncated receptor loses its ability to bind ligands but acquires constitutive kinase activity. Interestingly, EGFRvIII frequently co-expresses with full length wild-type EGFR in the same tumor cells. Moreover, EGFRvIII expressing cells exhibit increased proliferation, invasion, angiogenesis and resistance to apoptosis.

EGFRvIII is most often found in glioblastoma multiforme (GBM). It is estimated that 25-35% of GBM carries this truncated receptor. Moreover, its expression often reflects a more aggressive phenotype and poor prognosis. Besides GBM, expression of EGFRvIII has also been reported in other solid tumors such as non-small cell lung cancer, head and neck cancer, breast cancer, ovarian cancer and prostate cancer. In contrast, EGFRvIII is not expressed in healthy tissues.

In particular embodiments, a targeted cancer antigen epitope can have high expression by a targeted cancer cell or tumor or low expression by a targeted cancer cell or tumor. In particular embodiments, high and low expression can be determined using flow cytometry or fluorescence-activated cell-sorting (FACs). As is understood by one of ordinary skill in the art of flow cytometry, “hi”, “lo”, “+” and “-” refer to the intensity of a signal relative to negative or other populations. In particular embodiments, positive expression (+) means that the marker is detectable on a cell using flow cytometry. In particular embodiments, negative expression (-) means that the marker is not detectable using flow cytometry. In particular embodiments, “hi” means that the positive expression of a marker of interest is brighter as measured by fluorescence (using for example FACS) than other cells also positive for expression. In these embodiments, those of ordinary skill in the art recognize that brightness is based on a threshold of detection. Generally, one of skill in the art will analyze a negative control tube first, and set a gate (bitmap) around the population of interest by FSC and SSC and adjust the photomultiplier tube voltages and gains for fluorescence in the desired emission wavelengths, such that 97% of the cells appear unstained for the fluorescence marker with the negative control. Once these parameters are established, stained cells are analyzed, and fluorescence recorded as relative to the unstained fluorescent cell population. In particular embodiments, and representative of a typical FACS plot, hi implies to the farthest right (x line) or highest top line (upper right or left) while lo implies within the left lower quadrant or in the middle between the right and left quadrant (but shifted relative to the negative population). In particular embodiments, “hi” refers to greater than 20-fold of +, greater than 30-fold of +, greater than 40-fold of +, greater than 50-fold of +, greater than 60-fold of +, greater than 70-fold of +, greater than 80-fold of +, greater than 90-fold of +, greater than 100-fold of +, or more of an increase in detectable fluorescence relative to + cells. Conversely, “lo” can refer to a reciprocal population of those defined as “hi”.

### (VII-e-v) Other Targets

In addition to HSCs, T Cells, B Cells, and tumors (or cancer cells), vectors can target other antigens for bacteria and fungi.

Antigens targeting bacteria can be derived from, for example, anthrax, gram-negative bacilli, chlamydia, diphtheria, Helicobacter pylori, Mycobacterium tuberculosis, pertussis toxin, pneumococcus, rickettsiae, staphylococcus, streptococcus and tetanus.

As particular examples of bacterial antigen markers, anthrax antigens include anthrax protective antigen; gram-negative bacilli antigens include lipopolysaccharides; diphtheria antigens include diphtheria toxin; Mycobacterium tuberculosis antigens include mycolic acid, heat shock protein 65 (HSP65), the 30 kDa major secreted protein and antigen 85A; pertussis toxin antigens include hemagglutinin, pertactin, FIM2, FIM3 and adenylate cyclase; pneumococcal antigens include pneumolysin and pneumococcal capsular polysaccharides; rickettsiae antigens include rompA; streptococcal antigens include M proteins; and tetanus antigens include tetanus toxin.

Antigens targeting fungi can be derived from, for example, candida, coccidiodes, cryptococcus, histoplasma, leishmania, plasmodium, protozoa, parasites, schistosomae, tinea, toxoplasma, and Trypanosoma cruzi.

As particular examples of fungal antigens, coccidiodes antigens include spherule antigens; cryptococcal antigens include capsular polysaccharides; histoplasma antigens include heat shock protein 60 (HSP60); leishmania antigens include gp63 and lipophosphoglycan; plasmodium falciparum antigens include merozoite surface antigens, sporozoite surface antigens, circumsporozoite antigens, gametocyte/gamete surface antigens, protozoal and other parasitic antigens including the blood-stage antigen pf 155/RESA; schistosomae antigens include glutathione-S-transferase and paramyosin; tinea fungal antigens include trichophytin; toxoplasma antigens include SAG-1 and p30; and Trypanosoma cruzi antigens include the 75-77 kDa antigen and the 56 kDa antigen.

### (VII-f) Example Vectors

In particular embodiments, a vector includes a HDAd5/35++ vector with a payload, LCR, regulatory components, integration elements, selection cassette, and stuffer sequence. In particular embodiments, the payload includes a human γ-globin gene. In particular embodiments, the LCR includes the β-globin LCR. In particular embodiments, the regulatory components include a β-globin promoter. In particular embodiments, the integration elements include the Sleeping Beauty 100X transposase. In particular embodiments, the selection cassette includes MGMT(P140K). In particular embodiments, the vector further includes an EF1α promoter.

In various embodiments, a vector including an LCR of the present disclosure, such as a long LCR, provides increased expression of an operably linked coding nucleic acid sequence, e.g., in a target cell type or tissue such as a cell type or tissue in which the LCR controls express as shown in Table 1. In various embodiments, a vector including an LCR of the present disclosure provides increased expression of an operably linked coding nucleic acid sequence, e.g., in a target cell type or tissue, as compared to a reference vector that does not include an LCR. In various embodiments, a vector including a long LCR of the present disclosure provides increased expression of an operably linked coding nucleic acid sequence, e.g., in a target cell type or tissue, as compared to a reference vector that does not include a long LCR, e.g., a reference vector that includes a shorter LCR such as a mini-LCR. In various embodiments, the increase can be an increase of at least 10%, 15%, 20%, 30%, 40%, 50%, 60%, 70%, 80%, 90%, or 100% of the reference level of expression. In some embodiments, a vector including an LCR of the present disclosure, such as a long LCR, causes expression of an operably linked coding nucleic acid sequence that is at least 10%, 15%, 20%, 30%, 40%, 50%, 60%, 70%, 80%, 90%, or 100% of a reference level of expression of a reference endogenous coding nucleic acid sequence in healthy subjects, e.g., in a target cell type or tissue.

In various embodiments, a vector including an LCR of the present disclosure, such as a long LCR, provides decreased expression of an operably linked coding nucleic acid sequence in one or more non-target cell types or tissues such as a cell type or tissue that is not a cell type or tissue shown in Table 1 as a cell type or tissue in which the LCR controls expression. In various embodiments, a vector including an LCR of the present disclosure, such as a long LCR, provides decreased expression of an operably linked coding nucleic acid sequence in one or more non-target cell types or tissues as compared to a reference vector that does not include an LCR. In various embodiments, a vector including an LCR of the present disclosure, such as a long LCR, provides decreased expression of an operably linked coding nucleic acid sequence in one or more non-target cell types or tissues as compared to a reference vector that does not include a long LCR, e.g., a reference vector that includes a shorter LCR such as a mini-LCR. In various embodiments, the decrease can be a decrease of at least 10%, 15%, 20%, 30%, 40%, 50%, 60%, 70%, 80%, 90%, or 100% of the reference level of expression. For example, in particular embodiments, use of a β-globin long LCR decreases expression of an operably linked coding sequence, such as a coding sequence encoding γ-globin or β-globin, in cells that are not erythroid cells, as compared to a reference vector that does not include a β-globin long LCR, e.g., a reference vector that includes a shorter LCR such as a β-globin mini-LCR.

As those of skill in the art will appreciate, increased expression in target cells and/or tissues (e.g., resulting from use of a long LCR of the present disclosure, such as a long LCR) decreases the minimum therapeutically effective dosage of a vector in a gene therapy and therefore decreases immunotoxicity of the minimum therapeutically effective dosage and/or the risk of immunotoxicity. Those of skill in the art will further reappreciate, decreased expression in non-target cells and/or tissues (e.g., resulting from use of a long LCR of the present disclosure, such as a long LCR) decreases immunotoxicity and/or the risk of immunotoxicity, In certain particular examples, use of a β-globin long LCR increases expression of an operably linked coding nucleic acid sequence in hematopoietic stem cells and/or decreases expression of an operably linked coding nucleic acid sequence in non-erythroid cells, thereby decreasing gene therapy immunotoxicity and/or the risk thereof. In various embodiments, increased expression from viral vector transposon payloads in target cells and/or the ability to deliver a larger dosage of viral vector due to decreases immunotoxicity improves the total expression of an agent encoded by a transposon payload that can be achieved in target cells or tissues of a subject receiving gene therapy. Accordingly, vectors including an LCR of the present disclosure, such as a long LCR, can provide increased therapeutic efficacy as compared to reference vectors, such as reference vectors that do not include an LCR or do not include a long LCR.

### (VIII) Formulations

The adenoviral donor vector, large payload adenoviral vectors, adenoviral genomes, and adenoviral systems described herein can be formulated for administration to a subject. Formulations include a recombinant large payload adenoviral vector, adenoviral genome, and/or adenoviral system associated with a therapeutic gene (“active ingredient”) and one or more pharmaceutically acceptable carriers.

In particular embodiments, the formulations include active ingredients of at least 0.1% w/v or w/w of the formulation; at least 1% w/v or w/w of formulation; at least 10% w/v or w/w of formulation; at least 20% w/v or w/w of formulation; at least 30% w/v or w/w of formulation; at least 40% w/v or w/w of formulation; at least 50% w/v or w/w of formulation; at least 60% w/v or w/w of formulation; at least 70% w/v or w/w of formulation; at least 80% w/v or w/w of formulation; at least 90% w/v or w/w of formulations; at least 95% w/v or w/w of formulation; or at least 99% w/v or w/w of formulation.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the active ingredients or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The formulations disclosed herein can be formulated for administration by, for example, injection. For injection, formulation can be formulated as aqueous solutions, such as in buffers including Hanks’ solution, Ringer’s solution, or physiological saline, or in culture media, such as Iscove’s Modified Dulbecco’s Medium (IMDM). The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

Any formulation disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington’s Pharmaceutical Sciences, 18th Ed. Mack Printing Company, 1990. Moreover, formulations can be prepared to meet sterility, pyrogenicity, general safety, and purity standards as required by US FDA Office of Biological Standards and/or other relevant foreign regulatory agencies.

### (IX) Applications

### (IX-a) In Vivo Therapy

The formulations disclosed herein can be used for treating subjects (humans, veterinary animals (dogs, cats, reptiles, birds, etc.), livestock (horses, cattle, goats, pigs, chickens, etc.), and research animals (monkeys, rats, mice, fish, etc.). Treating subjects includes delivering therapeutically effective amounts. Therapeutically effective amounts include those that provide effective amounts, prophylactic treatments, and/or therapeutic treatments.

Formulations described herein can be administered in concert with HSPC mobilization. In particular embodiments, administration of adenoviral donor vector occurs concurrently with administration of one or more mobilization factors. In particular embodiments, administration of adenoviral donor vector follows administration of one or more mobilization factors. In particular embodiments, administration of adenoviral donor vector follows administration of a first one or more mobilization factors and occurs concurrently with administration of a second one or more mobilization factors.

The actual dose and amount of adenoviral donor vector and, in particular embodiments, of an adenoviral donor vector and mobilization factors, administered to a particular subject and concordant mobilization procedure and schedule can be determined by a physician, veterinarian, or researcher taking into account parameters such as physical and physiological factors including target; body weight; type of condition; severity of condition; upcoming relevant events, when known; previous or concurrent therapeutic interventions; idiopathy of the subject; and route of administration, for example. In addition, in vitro and in vivo assays can optionally be employed to help identify optimal dosage ranges.

Therapeutically effective amounts of adenoviral donor vector associated with a therapeutic gene can include doses ranging from, for example, 1 x 10⁷ to 50 x 10⁸ infection units (IU) or from 5 x 10⁷ to 20 x 10⁸ IU. In other examples, a dose can include 5 x 10⁷ IU, 6 x 10⁷ IU, 7x 10⁷ IU, 8x 10⁷ IU, 9x 10⁷ IU, 1 x 10⁸ IU, 2 x 10⁸ IU, 3 x 10⁸ IU, 4x 10⁸ IU, 5x 10⁸ IU, 6x 10⁸ IU, 7 x 10⁸ IU, 8 x 10⁸ IU, 9 x 10⁸ IU, 10 x 10⁸ IU, or more. In particular embodiments, a therapeutically effective amount of adenoviral donor vector associated with a therapeutic gene includes 4 x 10⁸ IU. In particular embodiments, a therapeutically effective amount of adenoviral donor vector associated with a therapeutic gene can be administered subcutaneously or intravenously. In particular embodiments, a therapeutically effective amount of an adenoviral donor vector associated with a therapeutic gene can be administered following administration with one or more mobilization factors.

In particular embodiments, a therapeutically effective amount of G-CSF includes 0.1 µg/kg to 100 µg/kg. In particular embodiments, a therapeutically effective amount of G-CSF includes 0.5 µg/kg to 50 µg/kg. In particular embodiments, a therapeutically effective amount of G-CSF includes 0.5 µg/kg, 1 µg/kg, 2 µg/kg, 3 µg/kg, 4 µg/kg, 5 µg/kg, 6 µg/kg, 7 µg/kg, 8 µg/kg, 9 µg/kg, 10 µg/kg, 11 µg/kg, 12 µg/kg, 13 µg/kg, 14 µg/kg, 15 µg/kg, 16 µg/kg, 17 µg/kg, 18 µg/kg, 19 µg/kg, 20 µg/kg, or more. In particular embodiments, a therapeutically effective amount of G-CSF includes 5 µg/kg. In particular embodiments, G-CSF can be administered subcutaneously or intravenously. In particular embodiments, G-CSF can be administered for 1 day, 2 consecutive days, 3 consecutive days, 4 consecutive days, 5 consecutive days, or more. In particular embodiments, G-CSF can be administered for 4 consecutive days. In particular embodiments, G-CSF can be administered for 5 consecutive days. In particular embodiments, as a single agent, G-CSF can be used at a dose of 10 µg/kg subcutaneously daily, initiated 3, 4, 5, 6, 7, or 8 days before adenoviral donor vector delivery. In particular embodiments, G-CSF can be administered as a single agent followed by concurrent administration with another mobilization factor. In particular embodiments, G-CSF can be administered as a single agent followed by concurrent administration with AMD3100. In particular embodiments, a treatment protocol includes a 5 day treatment where G-CSF can be administered on day 1, day 2, day 3, and day 4 and on day 5, G-CSF and AMD3100 are administered 6 to 8 hours prior to adenoviral donor vector administration.

Therapeutically effective amounts of GM-CSF to administer can include doses ranging from, for example, 0.1 to 50 µg/kg or from 0.5 to 30 µg/kg. In particular embodiments, a dose at which GM-CSF can be administered includes 0.5 µg/kg, 1 µg/kg, 2 µg/kg, 3 µg/kg, 4 µg/kg, 5 µg/kg, 6 µg/kg, 7 µg/kg, 8 µg/kg, 9 µg/kg, 10 µg/kg, 11 µg/kg, 12 µg/kg, 13 µg/kg, 14 µg/kg, 15 µg/kg, 16 µg/kg, 17 µg/kg, 18 µg/kg, 19 µg/kg, 20 µg/kg, or more. In particular embodiments, GM-CSF can be administered subcutaneously for 1 day, 2 consecutive days, 3 consecutive days, 4 consecutive days, 5 consecutive days, or more. In particular embodiments, GM-CSF can be administered subcutaneously or intravenously. In particular embodiments, GM-CSF can be administered at a dose of 10 µg/kg subcutaneously daily initiated 3, 4, 5, 6, 7, or 8 days before adenoviral donor vector delivery. In particular embodiments, GM-CSF can be administered as a single agent followed by concurrent administration with another mobilization factor. In particular embodiments, GM-CSF can be administered as a single agent followed by concurrent administration with AMD3100. In particular embodiments, a treatment protocol includes a 5 day treatment where GM-CSF can be administered on day 1, day 2, day 3, and day 4 and on day 5, GM-CSF and AMD3100 are administered 6 to 8 hours prior to adenoviral donor vector administration. A dosing regimen for Sargramostim (GM-CSF) can include 200 µg/m², 210 µg/m², 220 µg/m², 230 µg/m², 240 µg/m², 250 µg/m², 260 µg/m², 270 µg/m², 280 µg/m², 290 µg/m², 300 µg/m², or more. In particular embodiments, Sargramostim can be administered for one day, two consecutive days, three consecutive days, four consecutive days, five consecutive days, or more. In particular embodiments, Sargramostim can be administered subcutaneously or intravenously. In particular embodiments, a dosing regimen for Sargramostim can include 250 µg/m²/day intravenous or subcutaneous and can be continued until a targeted cell amount is reached in the peripheral blood or can be continued for 5 days. In particular embodiments, Sargramostim can be administered as a single agent followed by concurrent administration with another mobilization factor. In particular embodiments, Sargramostim can be administered as a single agent followed by concurrent administration with AMD3100. In particular embodiments, a treatment protocol includes a 5 day treatment where Sargramostim can be administered on day 1, day 2, day 3, and day 4 and on day 5, Sargramostim and AMD3100 are administered 6 to 8 hours prior to adenoviral donor vector administration.

In particular embodiments, a therapeutically effective amount of AMD3100 includes 0.1 mg/kg to 100 mg/kg. In particular embodiments, a therapeutically effective amount of AMD3100 includes 0.5 mg/kg to 50 mg/kg. In particular embodiments, a therapeutically effective amount of AMD3100 includes 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, or more. In particular embodiments, a therapeutically effective amount of AMD3100 includes 4 mg/kg. In particular embodiments, a therapeutically effective amount of AMD3100 includes 5 mg/kg. In particular embodiments, a therapeutically effective amount of AMD3100 includes 10 µg/kg to 500 µg/kg or from 50 µg/kg to 400 µg/kg. In particular embodiments, a therapeutically effective amount of AMD3100 includes 100 µg/kg, 150 µg/kg, 200 µg/kg, 250 µg/kg, 300 µg/kg, 350 µg/kg, or more. In particular embodiments, AMD3100 can be administered subcutaneously or intravenously. In particular embodiments, AMD3100 can be administered subcutaneously at 160-240 µg/kg 6 to 11 hours prior to adenoviral donor vector delivery. In particular embodiments, a therapeutically effective amount of AMD3100 can be administered concurrently with administration of another mobilization factor. In particular embodiments, a therapeutically effective amount of AMD3100 can be administered following administration of another mobilization factor. In particular embodiments, a therapeutically effective amount of AMD3100 can be administered following administration of G-CSF. In particular embodiments, a treatment protocol includes a 5-day treatment where G-CSF is administered on day 1, day 2, day 3, and day 4 and on day 5, G-CSF and AMD3100 are administered 6 to 8 hours prior to adenoviral donor vector injection.

Therapeutically effective amounts of SCF to administer can include doses ranging from, for example, 0.1 to 100 µg/kg/day or from 0.5 to 50 µg/kg/day. In particular embodiments, a dose at which SCF can be administered includes 0.5 µg/kg/day, 1 µg/kg/day, 2 µg/kg/day, 3 µg/kg/day, 4 µg/kg/day, 5 µg/kg/day, 6 µg/kg/day, 7 µg/kg/day, 8 µg/kg/day, 9 µg/kg/day, 10 µg/kg/day, 11 µg/kg/day, 12 µg/kg/day, 13 µg/kg/day, 14 µg/kg/day, 15 µg/kg/day, 16 µg/kg/day, 17 µg/kg/day, 18 µg/kg/day, 19 µg/kg/day, 20 µg/kg/day, 21 µg/kg/day, 22 µg/kg/day, 23 µg/kg/day, 24 µg/kg/day, 25 µg/kg/day, 26 µg/kg/day, 27 µg/kg/day, 28 µg/kg/day, 29 µg/kg/day, 30 µg/kg/day, or more. In particular embodiments, SCF can be administered for 1 day, 2 consecutive days, 3 consecutive days, 4 consecutive days, 5 consecutive days, or more. In particular embodiments, SCF can be administered subcutaneously or intravenously. In particular embodiments, SCF can be injected subcutaneously at 20 µg/kg/day. In particular embodiments, SCF can be administered as a single agent followed by concurrent administration with another mobilization factor. In particular embodiments, SCF can be administered as a single agent followed by concurrent administration with AMD3100. In particular embodiments, a treatment protocol includes a 5-day treatment where SCF can be administered on day 1, day 2, day 3, and day 4 and on day 5, SCF and AMD3100 are administered 6 to 8 hours prior to adenoviral donor vector administration.

In particular embodiments, growth factors GM-CSF and G-CSF can be administered to mobilize HSPC in the bone marrow niches to the peripheral circulating blood to increase the fraction of HSPCs circulating in the blood. In particular embodiments, mobilization can be achieved with administration of G-CSF/Filgrastim (Amgen) and/or AMD3100 (Sigma). In particular embodiments, mobilization can be achieved with administration of GM-CSF/Sargramostim (Amgen) and/or AMD3100 (Sigma). In particular embodiments, mobilization can be achieved with administration of SCF/Ancestim (Amgen) and/or AMD3100 (Sigma). In particular embodiments, administration of G-CSF/Filgrastim precedes administration of AMD3100. In particular embodiments, administration of G-CSF/Filgrastim occurs concurrently with administration of AMD3100. In particular embodiments, administration of G-CSF/Filgrastim precedes administration of AMD3100, followed by concurrent administration of G-CSF/Filgrastim and AMD3100. US 20140193376 describes mobilization protocols utilizing a CXCR4 antagonist with a S1P receptor 1 (S1PR1) modulator agent. US 20110044997 describes mobilization protocols utilizing a CXCR4 antagonist with a vascular endothelial growth factor receptor (VEGFR) agonist.

Therapeutic large-payload adenoviral vector(s) can be administered concurrently with or following administration of steroids, IL-1 receptor antagonist, and/or an IL-6 receptor antagonist administration. These protocols can alleviate potential side effects of treatments.

IL-1 receptor antagonists are known and include ADC-1001 (Alligator Bioscience, Lund, Sweden), FX-201 (Flexion Therapeutics, Burlington, MA), fusion proteins available from Bioasis Technologies (Richmond, Canada), GQ-303 (Genequine Biotherapeutics GmbH, Hamburg, Germany), HL-2351 (Handok, Inc., Seoul, South Korea), MBIL-1 RA (ProteoThera, Inc., Newton, MA), Anakinra (Pivor Pharmaceuticals, Vancouver, Canada), human immunoglobin G or Globulin S (GC Pharma, Gyeonggi-do, South Korea). IL-6 receptor antagonists are also known in the art and include tocilizumab, BCD-089 (Biocad, Russia), HS-628 (Zhejiang Hisun Pharm, Taizhou City, China), and APX-007 (Apexigen, San Carlos, CA).

In particular embodiments, an HSC enriching agent, such as a CD19 immunotoxin or 5-FU can be administered to enrich for HSPCs. CD19 immunotoxin can be used to deplete all CD19 lineage cells, which accounts for 30% of bone marrow cells. Depletion encourages exit from the bone marrow. By forcing HSPCs to proliferate (whether via CD19 immunotoxin of 5-FU, this stimulates their differentiation and exit from the bone marrow and increases transgene marking in peripheral blood cells.

Therapeutically effective amounts can be administered through any appropriate administration route such as by, injection, infusion, perfusion, and more particularly by administration by one or more of bone marrow, intravenous, intradermal, intraarterial, intranodal, intralymphatic, intraperitoneal injection, infusion, or perfusion).

### (IX-b) Ex Vivo Therapy and in Vitro Uses

he methods and compositions provided herein are disclosed at least in part for use in in vivo gene therapy. However, for the avoidance of doubt, the present disclosure expressly includes the use of compositions and methods provided herein for ex-vivo engineering of cells and/or tissues, as well as in vitro uses including the engineering of cells and/or tissues for research purposes.

### (IX-c) Treating a Particular Blood Disorder (e.g., Hemophilia, Thalassemia)

In particular embodiments, methods and formulations disclosed herein can be used to treat blood disorders. In particular embodiments, formulations are administered to subjects to treat hemophilia, β-thalassemia major, Diamond Blackfan anemia (DBA), paroxysmal nocturnal hemoglobinuria (PNH), pure red cell aplasia (PRCA), refractory anemia, severe aplastic anemia, and/or blood cancers such as leukemia, lymphoma, and myeloma.

In particular embodiments, a therapeutically effective treatment induces or increases expression of HbF, induces or increases production of hemoglobin and/or induces or increases production of β-globin. In particular embodiments, a therapeutically effective treatment improves blood cell function, and/or increases oxygenation of cells.

In particular embodiments, methods of the present disclosure can restore bone marrow function in a subject in need thereof. In particular embodiments, restoring bone marrow function can include improving bone marrow repopulation with gene corrected cells as compared to a subject in need thereof not administered a therapy described herein. Improving bone marrow repopulation with gene corrected cells can include increasing the percentage of cells that are gene corrected. In particular embodiments, the cells are selected from white blood cells and bone marrow derived cells. In particular embodiments, the percentage of cells that are gene corrected can be measured using an assay selected from quantitative real time PCR and flow cytometry.

In particular embodiments, methods of the present disclosure can be used to treat FA. In particular embodiments, therapeutic efficacy can be observed through lymphocyte reconstitution, improved clonal diversity and thymopoiesis, reduced infections, and/or improved patient outcome. Therapeutic efficacy can also be observed through one or more of weight gain and growth, improved gastrointestinal function (e.g., reduced diarrhea), reduced upper respiratory symptoms, reduced fungal infections of the mouth (thrush), reduced incidences and severity of pneumonia, reduced meningitis and blood stream infections, and reduced ear infections. In particular embodiments, treating FA with methods of the present disclosure include increasing resistance of bone marrow derived cells to mitomycin C (MMC). In particular embodiments, the resistance of bone marrow derived cells to MMC can be measured by a cell survival assay in methylcellulose and MMC.

### (IX-c-i) LCRs, Promoters, Coding Sequences, and Vectors for Treating Blood Disorder

In various embodiments, the present disclosure includes treatment of a blood disorder using an adenoviral donor vector of the present disclosure that includes a β-globin long LCR, a β-globin promoter, and a coding nucleic acid sequence that encodes a protein or agent for treatment of the blood disorder. In various embodiments, the blood disorder is thalassemia and the protein is a β-globin or γ-globin protein, or a protein that otherwise partially or completely functionally replaces β-globin or γ-globin. In various embodiments, the blood disorder is hemophilia and the protein is ET3 or a protein that otherwise partially or completely functionally replaces Factor VIII. In various embodiments, the blood disorder is a point mutation disease such as sickle cell anemia, and the agent is a gene editing protein.

ET3 can have the following amino acid sequence: SEQ ID NO 99. In various embodiments, a Factor VIII replacement protein can have an amino acid sequence having at least 70%, 75%, 80%, 85%, 90%, 91%, 92%, 93%, 94%, 95%, 96%, 97%, 98%, 99%, or 100% identity to the SEQ ID NO: 99.

β-globin can have the following amino acid sequence: SEQ ID NO 100. In various embodiments, a β-globin replacement protein can have an amino acid sequence having at least 70%, 75%, 80%, 85%, 90%, 91%, 92%, 93%, 94%, 95%, 96%, 97%, 98%, 99%, or 100% identity to SEQ ID NO: 100.

γ-globin can have the following amino acid sequence: SEQ ID NO 101. In various embodiments, a γ-globin replacement protein can have an amino acid sequence having at least 70%, 75%, 80%, 85%, 90%, 91%, 92%, 93%, 94%, 95%, 96%, 97%, 98%, 99%, or 100% identity to SEQ ID NO: 101.

### (IX-c-ii) Dosages and Formulations

A vector can be formulated such that it is pharmaceutically acceptable for administration to cells or animals, e.g., to humans. A vector may be administered in vitro, ex vivo, or in vivo. In various instances, a vector can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

In various embodiments, a composition including a vector as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

As disclosed herein, a vector can be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, a vector can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intracisternal injection and infusion. A parenteral route of administration can be, for example, administration by injection, transnasal administration, transpulmonary administration, or transcutaneous administration. Administration can be systemic or local by intravenous injection, intramuscular injection, intraperitoneal injection, subcutaneous injection.

In various embodiments, a vector of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A vector can be administered parenterally in the form of an injectable formulation including a sterile solution or suspension in water or another pharmaceutically acceptable liquid. For example, the vector can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of vector included in the pharmaceutical preparations is such that a suitable dose within the designated range is provided. Nonlimiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for subcutaneous injection.

In some embodiments, a vector described herein can be therapeutically delivered to a subject by way of local administration. As used herein, “local administration” or “local delivery,” can refer to delivery that does not rely upon transport of the vector or vector to its intended target tissue or site via the vascular system. For example, the vector may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, the compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described in U.S. Pat. No. 6,302,855, may also be used, for example, with an injection system as described herein.

Pharmaceutical forms of vector formulations suitable for injection can include sterile aqueous solutions or dispersions. A formulation can be sterile and must be fluid to allow proper flow in and out of a syringe. A formulation can also be stable under the conditions of manufacture and storage. A carrier can be a solvent or dispersion medium containing, for example, water and saline or buffered aqueous solutions. Preferably, isotonic agents, for example, sugars or sodium chloride can be used in the formulations.

In addition, one skilled in the art may also contemplate additional delivery method may be via electroporation, sonophoresis, intraosseous injections methods or by using gene gun. Vectors may also be implanted into microchips, nano-chips or nanoparticles.

A suitable dose of a vector described herein can depend on a variety of factors including, e.g., the age, sex, and weight of a subject to be treated, the condition or disease to be treated, and the particular vector used. Other factors affecting the dose administered to the subject include, e.g., the type or severity of the condition or disease. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. A suitable means of administration of a vector can be selected based on the condition or disease to be treated and upon the age and condition of a subject. Dose and method of administration can vary depending on the weight, age, condition, and the like of a patient, and can be suitably selected as needed by those skilled in the art. A specific dosage and treatment regimen for any particular subject can be adjusted based on the judgment of a medical practitioner.

A vector solution can include a therapeutically effective amount of a composition described herein. Such effective amounts can be readily determined by one of ordinary skill in the art based, in part, on the effect of the administered composition, or the combinatorial effect of the composition and one or more additional active agents, if more than one agent is used. A therapeutically effective amount can be an amount at which any toxic or detrimental effects of the composition are outweighed by therapeutically beneficial effects.

### (IX-d) Treating a Type of Cancer

In particular embodiments, methods and formulations disclosed herein can be used to treat cancer. In particular embodiments, formulations are administered to subjects to treat acute lymphoblastic leukemia (ALL), acute myelogenous leukemia (AML), chronic lymphocytic leukemia (CLL), chronic myelogenous leukemia (CML), chronic myelomonocytic leukemia, diffuse large B-cell lymphoma, follicular lymphoma, Hodgkin’s lymphoma, juvenile myelomonocytic leukemia, multiple myeloma, myelodysplasia, and/or non-Hodgkin’s lymphoma.

Additional exemplary cancers that may be treated include astrocytoma, atypical teratoid rhabdoid tumor, brain and central nervous system (CNS) cancer, breast cancer, carcinosarcoma, chondrosarcoma, chordoma, choroid plexus carcinoma, choroid plexus papilloma, clear cell sarcoma of soft tissue, diffuse large B-cell lymphoma, ependymoma, epithelioid sarcoma, extragonadal germ cell tumor, extrarenal rhabdoid tumor, Ewing sarcoma, gastrointestinal stromal tumor, glioblastoma, HBV-induced hepatocellular carcinoma, head and neck cancer, kidney cancer, lung cancer, malignant rhabdoid tumor, medulloblastoma, melanoma, meningioma, mesothelioma, multiple myeloma, neuroglial tumor, not otherwise specified (NOS) sarcoma, oligoastrocytoma, oligodendroglioma, osteosarcoma, ovarian cancer, ovarian clear cell adenocarcinoma, ovarian endometrioid adenocarcinoma, ovarian serous adenocarcinoma, pancreatic cancer, pancreatic ductal adenocarcinoma, pancreatic endocrine tumor, pineoblastoma, prostate cancer, renal cell carcinoma, renal medullo carcinoma, rhabdomyosarcoma, sarcoma, schwannoma, skin squamous cell carcinoma, and stem cell cancer. In various particular embodiments, the cancer is ovarian cancer. In various particular embodiments the cancer is breast cancer.

### (IX-d-i) LCRs, Promoters, Coding Sequences, and Vectors for Treating the Type of Cancer

The adenoviral donor vectors described herein are useful for the treatment of cancers. In embodiments of such adenoviral donor vectors, as well as adenoviral donor genomes, transposition systems, and adenoviral production systems, the provided long LCRs can be used to mediate transfer of gene(s) to target cells useful to treat cancers. One of ordinary skill in the art will recognize appropriate promoters, coding sequences, and vector structures that will be useful for treating specific types of cancer. In addition, examples of such elements are described herein.

In particular embodiments, the adenoviral donor vectors can include a sequence that expresses a cancer-specific or cancer-targeted therapeutic gene. Examples of such cancer-targeted therapeutic genes include an antibody fragment that binds a cancer antigen (e.g., CD19, ROR1, or others - include those described herein), wherein the sequence of the antibody fragment is contiguous with and in the same reading frame as a nucleic acid sequence encoding a TCR subunit or portion thereof. Such TFPs are able to associate with one or more endogenous (or alternatively, one or more exogenous, or a combination of endogenous and exogenous) TCR subunits in order to form a functional TCR complex.

In particular embodiments, a therapeutic gene can encode an antibody or a binding fragment of an antibody, such as a Fab or an scFv. Exemplary antibodies (including scFvs) that can be expressed include those provided described in WO2014164553A1, US20170283504, US7083785B2, US10189906B2, US10174095B2, WO2005102387A2, US20110206701A1, WO2014179759A1, US20180037651A1, US20180118822A1, WO2008047242A2, WO1996016990A1, WO2005103083A2, and WO1999062526A2. Antibodies described herein in relation to binding domains can also be used, as well as atezolizumab, blinatumomab, brentuximab, cetuximab, cirmtuzumab, farletuzumab, gemtuzumab, OKT3, oregovomab, promiximab, pembrolizumab, and trastuzumab.

Immune checkpoint inhibitors can also be used. Immune checkpoint inhibitors refer to compounds that inhibit the function of an immune inhibitory checkpoint protein. Inhibition includes reduction of function and full blockade. Preferred immune checkpoint inhibitors are antibodies that specifically recognize immune checkpoint proteins. In particular embodiments, immune checkpoint inhibitors enhance the proliferation, migration, persistence and/or cytoxicity activity of CD8+ T cells in a subject and in particular the tumor-infiltrating of CD8+ T cells of the subject. Accordingly, exemplary immune checkpoint inhibitors of the present disclosure include αPD-L1γ1 antibody (alternatively referred to as αPD-L1γ₁). αPD-L1γ1 is further described in Engeland et al. 2014 Mol Ther 22(11):1949-1959.

Examples of PD-1 and PD-L1 antibodies are described in US 7,488,802; US 7,943,743; US 8,008,449; US 8,168,757; US 8,217,149, WO03042402, WO2008156712, WO2010089411, WO2010036959, WO2011066342, WO2011159877, WO2011082400, and WO2011161699. In some embodiments, the PD-1 blockers include anti-PD-L1 antibodies. In other embodiments the PD-1 blockers include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224 is a fusion protein of B7-DC; an antibody Fc portion; BMS-936559 (MDX-1105-01) for PD-L1 (B7-H1) blockade.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9 (Ga19). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011/155607, WO2013/006490 and WO2010/117057.

Additional particular immune checkpoint inhibitors include atezolizumab, BMS-936559, ipilimumab, MEDl0680, MEDl4736, MSB0010718C, pembrolizumab, pidilizumab, and tremelimumab. See also WO 1998/42752; WO 2000/37504; WO 2001/014424; WO 2004/035607; US 2005/0201994; US 2002/0039581; US 2002/086014; US 5,811,097; US 5,855,887; US 5,977,318; US 6,051,227; US 6,984,720; US 6,682,736; US 6,207,156; US 6,682,736; US 7,109,003; US 7,132,281; EP1212422B1; Hurwitz et al., Proc. Natl. Acad. Sci. USA, 95(17):10067-10071 (1998); Camacho et al., J. Clin. Oncology, 22(145): Abstract No. 2505 (2004) (antibody CP-675206); and Mokyr et al., Cancer Res, 58:5301-5304 (1998).

### (IX-d-ii) Dosages and Formulations

In the context of cancers, therapeutically effective amounts can decrease the number of tumor cells, decrease the number of metastases, decrease tumor volume, increase life expectancy, induce apoptosis of cancer cells, induce cancer cell death, induce chemo- or radiosensitivity in cancer cells, inhibit angiogenesis near cancer cells, inhibit cancer cell proliferation, inhibit tumor growth, prevent metastasis, prolong a subject’s life, reduce cancer-associated pain, reduce the number of metastases, and/or reduce relapse or re-occurrence of the cancer following treatment.

Particular embodiments, formulations are administered to subjects to prevent or delay cancer reoccurrence or prevent or delay cancer onset in carriers of high-risk germ line mutations. In particular embodiments, formulations are administered to subjects to receive higher therapeutic doses of temozolomide (TMZ) and benzylguanine or BCNU. Due to strong myelosupressvive off-target effects, it remains a challenge to deliver an effective dose of TMZ and benzylguanine to tumors. Patients may currently receive TMZ and benzylguanine for treatments associated with acute myeloid leukemia (AML), esophageal cancer, Head & Neck Cancer, High-Grade Glioma, myelodysplastic syndrome, non-small cell lung cancer, NSCLC; Refractory AML, small cell lung cancer, anaplastic astrocytoma, brain tumors, breast cancer (e.g., metastatic), colorectal cancer (e.g., metastatic), diffuse intrinsic brainstem glioma, Ewing sarcoma, glioblastoma multiforme (GBM), malignant glioma, melanoma, metastatic malignant melanoma, recurrent malignant melanoma, nasopharyngeal cancer, metastatic breast cancer, and pediatric cancers.

Patients with MGMT-expressing tumors would benefit from administration of a therapeutic large-payload adenoviral vector with an active ingredient (such as a CAR, TCR, or antibody) combined with the MGMT P140k in vivo selection cassette. Ex vivo approaches have shown the applicability of this approach. In particular embodiments, therapeutic amounts of TMZ and benzylguanine or BCNU are administered to reduce the tumor burden or volume.

### (IX-e) Treating a Point Mutation Condition (e.g., Sickle Cell)

In particular embodiments, methods and formulations disclosed herein can be used to treat point mutation conditions. In particular embodiments, formulations are administered to subjects to treat sickle cell disease, cystic fibrosis, Tay-Sachs disease, and/or phenylketonuria. In various embodiments, a transposon payload of the present disclosure encodes a CRISPR-Cas for corrective editing of a nucleic acid lesion. In various embodiments, a transposon payload of the present disclosure encodes a base editor for corrective editing of a nucleic acid lesion.

### (IX-f) Treating a Particular Enzyme Deficiency

In particular embodiments, methods and formulations disclosed herein can be used to treat particular enzyme deficiency. In particular embodiments, formulations are administered to subjects to treat Hurler’s syndrome, selective IgA deficiency, hyper IgM, IgG subclass deficiency, Niemann-Pick disease, Tay-Sachs disease, Gaucher disease, Fabry disease, Krabbe disease, glucosemia, maple syrup urine disease, phenylketonuria, glycogen storage disease, Friedreich ataxia, Zellweger syndrome, adrenoleukodystrophy, complement disorders, and/or mucopolysaccharidoses.

In particular embodiments, methods of the present disclosure can normalize primary and secondary antibody responses to immunization in a subject in need thereof. Normalizing primary and secondary antibody responses to immunization can include restoring B-cell and/or T-cell cytokine signaling programs functioning in class switching and memory response to an antigen. Normalizing primary and secondary antibody responses to immunization can be measured by a bacteriophage immunization assay. In particular embodiments, restoration of B-cell and/or T-cell cytokine signaling programs can be assayed after immunization with the T-cell dependent neoantigen bacteriophage ψX174. In particular embodiments, normalizing primary and secondary antibody responses to immunization can include increasing the level of IgA, IgM, and/or IgG in a subject in need thereof to a level comparable to a reference level derived from a control population. In particular embodiments, normalizing primary and secondary antibody responses to immunization can include increasing the level of IgA, IgM, and/or IgG in a subject in need thereof to a level greater than that of a subject in need thereof not administered a gene therapy described herein. The level of IgA, IgM, and/or IgG can be measured by, for example, an immunoglobulin test. In particular embodiments, the immunoglobulin test includes antibodies binding IgG, IgA, IgM, kappa light chain, lambda light chain, and/or heavy chain. In particular embodiments, the immunoglobulin test includes serum protein electrophoresis, immunoelectrophoresis, radial immunodiffusion, nephelometry and turbidimetry. Commercially available immunoglobulin test kits include MININEPH™ (Binding site, Birmingham, UK), and immunoglobulin test systems from Dako (Denmark) and Dade Behring (Marburg, Germany). In particular embodiments, a sample that can be used to measure immunoglobulin levels includes a blood sample, a plasma sample, a cerebrospinal fluid sample, and a urine sample.

In particular embodiments, methods of the present disclosure can be used to treat SCID-X1. In particular embodiments, methods of the present disclosure can be used to treat SCID (e.g., JAK 3 kinase deficiency SCID, purine nucleoside phosphorylase (PNP) deficiency SCID, adenosine deaminase (ADA) deficiency SCID, MHC class II deficiency or recombinase activating gene (RAG) deficiency SCID). In particular embodiments, therapeutic efficacy can be observed through lymphocyte reconstitution, improved clonal diversity and thymopoiesis, reduced infections, and/or improved patient outcome. Therapeutic efficacy can also be observed through one or more of weight gain and growth, improved gastrointestinal function (e.g., reduced diarrhea), reduced upper respiratory symptoms, reduced fungal infections of the mouth (thrush), reduced incidences and severity of pneumonia, reduced meningitis and blood stream infections, and reduced ear infections. In particular embodiments, treating SCIDX-1 with methods of the present disclosure include restoring functionality to the yC-dependent signaling pathway. The functionality of the yC-dependent signaling pathway can be assayed by measuring tyrosine phosphorylation of effector molecules STAT3 and/or STAT5 following in vitro stimulation with IL-21 and/or IL-2, respectively. Tyrosine phosphorylation of STAT3 and/or STAT5 can be measured by intracellular antibody staining.

### (IX-i) Other Uses

### (IX-i-i) HIV (representative Infectious Agent)

Particular embodiments include treatment of secondary, or acquired, immune deficiencies such as immune deficiencies caused by trauma, viruses, chemotherapy, toxins, and pollution. As previously indicated, acquired immunodeficiency syndrome (AIDS) is an example of a secondary immune deficiency disorder caused by a virus, the human immunodeficiency virus (HIV), in which a depletion of T lymphocytes renders the body unable to fight infection. Thus, as another example, a gene can be selected to provide a therapeutically effective response against an infectious disease. In particular embodiments, the infectious disease is human immunodeficiency virus (HIV). The therapeutic gene may be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and gene products include α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC; and laminin receptor. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

The Exemplary Embodiments and Example(s) below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (X) Exemplary Embodiments

1. An adenoviral donor vector including: (a) an adenoviral capsid; and (b) a linear, double-stranded DNA genome including: (i) a transposon payload of at least 10 kb; (ii) transposon inverted repeats (IRs) that flank the transposon payload; and (iii) recombinase direct repeats (DRs) that flank the transposon inverted repeats.

2. An adenoviral donor genome including: (a) a transposon payload of at least 10 kb; (b) transposon inverted repeats (IRs) that flank the transposon payload; and (c) recombinase direct repeats (DRs) that flank the transposon inverted repeats.

3. An adenoviral transposition system including: (a) the adenoviral donor vector of embodiment 1; and (b) an adenoviral support vector including (i) the adenoviral capsid; and (ii) an adenoviral support genome including a nucleic acid sequence encoding a transposase.

4. An adenoviral transposition system including: (a) the adenoviral donor genome of embodiment 2; and (b) an adenoviral support genome including a nucleic acid sequence encoding a transposase.

5. An adenoviral production system including: (a) a nucleic acid including the adenoviral donor genome of embodiment 2; and (b) a nucleic acid including an adenoviral helper genome including a conditional packaging element.

6. The vector, genome, or system of any one of embodiments 1-5, wherein the transposon payload includes a Long LCR, optionally wherein the Long LCR is a β-globin Long LCR including β-globin LCR HS1 to HS5.

7. The vector, genome, or system of embodiment 6, wherein the Long LCR has a length of at least 27 kb.

8. The vector, genome, or system of any one of embodiments 1-6, wherein the transposon payload includes an LCR set forth in Table 1.

9. The vector, genome, or system of any one of embodiments 1-6, wherein the transposon payload has a length of at least 15 kb, at least 16 kb, at least 17 kb, at least 18 kb, at least 19 kb, at least 20 kb, at least 21 kb, at least 22 kb, at least 23 kb, at least 24 kb, at least 25 kb, at least 30 kb, at least 35 kb, at least 38 kb, or at least 40 kb.

10. The vector, genome, or system of any one of embodiments 1-6, wherein the transposon payload has a length of 10 kb-35 kb, 10 kb-30 kb, 15 kb-35 kb, 15 kb-30 kb, 20 kb-35 kb, or 20 kb-30 kb.

11. The vector, genome, or system of any one of embodiments 1-6, wherein the transposon payload has a length of 10 kb-32.4 kb, 15 kb-32.4 kb, or 20 kb-32.4 kb.

12. The vector, genome, or system of any one of embodiments 1-11, wherein the transposon payload includes a nucleic acid sequence that encodes a protein, optionally wherein the protein is a therapeutic protein.

13. The vector, genome, or system of embodiment 12, wherein the protein is selected from the group including a β globin replacement protein and a γ-globin replacement protein.

14. The vector, genome, or system of embodiment 12, wherein the protein is a Factor VIII replacement protein.

15. The vector, genome, or system of embodiment 12 or 13, wherein the nucleic acid sequence that encodes the protein is operably linked with a promoter, optionally wherein the promoter is a β globin promoter.

16. The vector, genome, or system of any one of embodiments 1-15, wherein the transposon inverted repeats are Sleeping Beauty (SB) inverted repeats, optionally wherein the SB inverted repeats are pT4 inverted repeats.

17. The vector, genome, or system of any one of embodiments 3-15, wherein the transposase is a Sleeping Beauty (SB) transposase, optionally wherein the transposase is Sleeping Beauty 100x (SB100x).

18. The vector, genome, or system of any one of embodiments 1-17, wherein the recombinase direct repeats are FRT sites.

19. The vector, genome, or system of any one of embodiments 3-18, wherein the adenoviral support genome includes a nucleic acid encoding a recombinase.

20. The vector, genome, or system of embodiment 19, wherein the recombinase is a FLP recombinase.

21. The vector, genome, or system of any one of embodiments 1-20, wherein the transposon payload includes a β-globin long LCR, the transposon payload includes a nucleic acid sequence that encodes β-globin operably linked with a β-globin promoter, the inverted repeats are SB inverted repeats, and the recombinase direct repeats are FRT sites.

22. The vector, genome, or system of any one of embodiments 1-21, wherein in the transposon payload includes a selection cassette, optionally wherein the selection cassette includes a nucleic acid sequence that encodes mgmt^{P140K}.

23. The vector, genome, or system of any one of embodiments 1-22, wherein the adenoviral capsid is modified for increased affinity to CD46, optionally wherein the adenoviral capsid is an Ad35++ capsid.

24. The adenoviral production system of any one of embodiments 5-23, wherein the adenoviral helper genome conditional packaging element includes a packaging sequence flanked by recombinase direct repeats.

25. The adenoviral production system of embodiment 24, wherein the recombinase direct repeats that flank the packaging sequence of the conditional packaging element are LoxP sites.

26. A cell including a vector, genome, or system according to any one of embodiments 1-25.

27. A cell including in its genome the transposon payload of any one of embodiments 1-25, wherein the transposon payload present in the genome of the cell is flanked by the transposon inverted repeats.

28. The cell of embodiment 26 or 27, wherein the cell is a hematopoietic stem cell.

29. An adenovirus-producing cell including an adenoviral production system according to any one of embodiments 5-25, optionally wherein the cell is a HEK293 cell.

30. A method of modifying a cell, the method including contacting the cell with a vector, genome, or system according to any one of embodiments 1-25.

31. A method of modifying a cell of a subject, the method including administering to the subject a vector, genome, or system according to any one of embodiments 1-25.

32. A method of modifying a cell of a subject without isolation of the cell from the subject, the method including administering to the subject a vector, genome, or system according to any one of embodiments 1-25.

33. A method of treating a disease or condition in a subject in need thereof, the method including administering to the subject a vector, genome, or system according to any one of embodiments 1-25.

34. The method of any one of embodiments 31-33, wherein the adenoviral donor vector is administered to the subject intravenously.

35. The method of any one of embodiments 31-34, wherein the method includes administering to the subject a mobilization agent, optionally wherein the mobilization agent includes one or more of granulocyte-colony stimulating factor (G-CSF), a CXCR4 antagonist, and a CXCR2 agonist.

36. The method of embodiment 35, wherein the CXCR4 antagonist is AMD3100.

37. The method of embodiment 35 or 36, wherein the CXCR2 agonist is GRO-β.

38. The method of any one of embodiments 31-37, wherein the transposon payload includes a selection cassette and the method includes administering a selection agent to the subject.

39. The method of embodiment 38, wherein the selection cassette encodes mgmt^{P140K} and the selection agent is O⁶BG/BCNU.

40. The method of any one of embodiments 31-39, wherein the method causes integration and/or expression of at least one copy of the transposon payload in at least 20%, 30%, 40%, 50%, 60%, 70%, 80%, 90%, or 95% of cells expressing CD46.

41. The method of any one of embodiments 31-39, wherein the method causes integration and/or expression of at least one copy of the transposon payload in at least 20%, 30%, 40%, 50%, 60%, 70%, 80%, 90%, or 95% of hematopoietic stem cells and/or erythroid Ter119⁺ cells.

42. The method of any one of embodiments 31-41, wherein the method causes integration of an average of at least 2 copies of the transposon payload in the genomes of cells including at least 1 copy of the transposon payload.

43. The method of any one of embodiments 31-42, wherein the method causes integration of an average of at least 2.5 copies of the transposon payload in the genomes of cells including at least 1 copy of the transposon payload.

44. The method of any one of embodiments 31-43, wherein the method causes expression of a protein encoded by the transposon payload at a level that is at least about 20% of the level of reference, optionally wherein the reference is expression of an endogenous reference protein in the subject or in a reference population.

45. The method of any one of embodiments 31-43, wherein the method causes expression of a protein encoded by the transposon payload at a level that is at least about 25% of the level of reference, optionally wherein the reference is expression of an endogenous reference protein in the subject or in a reference population.

46. The method of any one of embodiments 31-45, wherein the subject is a subject suffering from thalassemia intermedia, wherein the transposase payload includes a β-globin Long LCR including β-globin LCR HS1 to HS5 and a nucleic acid sequence encoding a β globin replacement protein and/or γ-globin replacement protein operably linked with a β globin promoter.

47. The method of any one of embodiments 31-45, wherein the subject is a subject suffering from hemophilia, wherein the transposase payload includes a β-globin Long LCR including β-globin LCR HS1 to HS5 and a nucleic acid sequence encoding a Factor VIII replacement protein operably linked with a β globin promoter.

48. The method of embodiment 47, wherein expression of the protein in the subject reduces at least one symptom of thalassemia intermedia and/or treats thalassemia intermedia.

### XI Experimental Examples

### Example 1. Large Payload Adenoviral Vector Gene Therapy

Introduction. For gene therapy of hemoglobinopathies such as thalassemia major and Sickle Cell Anemia to be successful, the transferred gene is preferably expressed in erythroid cells at high levels, without position effects of integration and transcriptional silencing. The β-globin locus control region (LCR) is thought to be beneficial in such use. For gene therapy applications, a β-globin LCR containing HS1 to HS5 has been shown to confer high-level expression upon cis-linked genes in transgenic mice (Grosveld et al., Cell 51:975-985, 1987). However, this version of the LCR is too large to be used in lentivirus vectors (insert capacity 8 kb) and, therefore truncated “mini” or “micro” LCR versions have been developed. For example, in ongoing clinical trials in thalassemia patients a lentivirus containing a 2.7 kb mini-LCR (covering HS2-HS4) and a 266 bp β-globin promoter is being used (Negre et al., Curr Gene Ther 15: 64-81, 2015). A 5.9 kb β-globin LCR version was previously employed that contained HS1 to HS4 and the β-globin promoter for expression of γ-globin in CD46 transgenic mice or CD46/Hbb^{th3} thalassemic mice (Wang et al., J Clin Invest 129:598-615, 2019). With the in vivo HSPC transduction/selection approach, γ-globin marking was achieved in nearly 100% of peripheral blood erythrocytes, while the level of γ-globin expression was 10-15% of that of adult mouse α-globin with an average integrated vector copy number (VCN) of 2-3 copies per cell.

For a complete cure of β₀/β₀ thalassemia or Sickle Cell Anemia, it is generally thought that a therapeutic globin (either γ- or β-globin) expression level of 20% in erythroid cells is required (Fitzhugh et al., Blood 130:1946-1948, 2017). One way to reach this level is by increasing the VCN by improving HSPC transduction or increasing the vector dose. Such approaches, however, have historically been observed in other contexts to increase the risk of toxicity, at least in part due to random integration pattern of utilized vector systems. In this Example, stronger transcriptional elements, namely a longer LCR version, were utilized to increase γ-globin expression per RBCs after in vivo HSPC transduction of CD46-transgenic mice.

We developed a novel in vivo HSPC transduction approach that does not require leukapheresis, myeloablation, and HSPC transplantation (Richter et al., Blood, 128: 2206-2217, 2016). The approach involves a new vector platform suitable for in vivo HSPC transduction, i.e. helper-dependent, capsid-modified adenovirus vectors (HDAd5/35++). Features of these vectors include CD46-affinity enhanced fibers that allow for efficient transduction of primitive HSCs while avoiding infection of non-hematopoietic tissues after i.v. injection and an insert capacity of up to 30 b. Due to limited accessibility, HSPCs localized in the bone marrow cannot be transduced by intravenously injected vectors, including HDAd5/35++ vectors, even when the vector targets receptors that are present on bone marrow cells (Ni et al., Hum Gene Ther, 16: 664-677, 2005 and Ni et al., Cancer Gene Ther, 13: 1072-1081, 2006). A combination of granulocyte-colony-stimulating factor (G-CSF) and the CXCR4 antagonists AMD3100 (Mozobil™, Plerixa™) has been shown to efficiently mobilize primitive progenitor cells in animal models and in humans (Fruehauf et al., Cytotherapy, 11: 992-1001, 2009 and Yannaki et al., Hum Gene Ther, 24: 852-860, 2013). G-CSF/AMD3100 was used to mobilize HSPCs from the bone marrow into the peripheral blood stream followed by an intravenous injection of HDAd5/35++ vectors. This was shown previously in human CD46 transgenic mice (Richter et al., Blood, 128: 2206-2217, 2016; Li et al., Mol Ther Methods Clin Dev, 9: 390-401, 2018; Li et al., Blood, 131: 2915-2928. 2018; Wang et al., J Clin Invest, 129: 598-615. 2019; Wang et al., Blood Adv, 3: 2883-2894, 2019; and Wang et al., Mol Ther Methods Clin Dev, 8: 52-64, 2018), humanized mice (Richter et al., Blood, 128: 2206-2217, 2016) and rhesus macaques (Harworth et al., ASCGT 21th Annual meeting, 2018, DOl: 10.1016/j.ymthe.2018.05.001). HSPCs transduced in the periphery home back to the bone marrow where they persist long-term. Without a proliferative advantage, in vivo transduced HSPCs do not efficiently exit the bone marrow and contribute to downstream differentiation. Short-term treatment of animals with O⁶BG/BCNU provides a proliferation stimulus to mgmt^{P140K} gene-modified HSPCs and subsequent stable transgene expression in >80% of peripheral blood cells (Wang et al., Mol Ther Methods Clin Dev, 8: 52-64, 2018).

HD-Ad5/35++ genomes do not integrate into the host cell genome and are lost upon cell division. For gene therapy purposes and to trace in vivo transduced HSPCs long-term, HD-Ad5/35++ vectors were modified to allow for transgene integration. This was done by incorporating a hyperactive Sleeping Beauty transposase system (SB100) (Zhang et al., PLoS One, 8: e75344, 2013; Hausl et al., Mol Ther, 18: 1896-1906, 2010; and Yant et al., Nat Biotechnol, 20: 999-1005, 2002). The transposase, co-expressed in trans from a second vector, recognizes specific DNA sequences (inverted repeats, “IRs”) flanking the transgene cassette and triggers the integration into TA dinucleotides of the chromosomal DNA. Unlike retrovirus integration, SB100x-mediated integration does not depend on the transcriptional status of the targeted genes (Yant et al., Mol Cell Biol, 25: 2085-2094, 2005). Several studies have demonstrated SB100x-mediated transgene integration is random and has not been associated with the activation of proto-oncogenes (Richter et al., Blood, 128: 2206-2217, 2016; Wang et al., Mol Ther Methods Clin Dev, 8: 52-64, 2018; Zhang et al., PLoS One, 8: e75344, 2013; Hausl et al., Mol Ther, 18: 1896-1906, 2010; and Yant et al., Nat Biotechnol, 20: 999-1005, 2002). An advantage of the SB100x-based integration system is that it does not depend on an efficient homologous DNA repair machinery of the cell. The latter is critical in HSPCs, which show low activity of DNA repair and recombination enzymes (Beerman et al., Cell Stem Cell, 15: 37-50, 2014). It was demonstrated that in vivo HSC co-infection with a HDAd35++-transposon vector and a SB100x/Flpe expressing vector in CD46-transgenic mice (Richter et al., Blood, 128: 2206-2217, 2016; Wang et al., J Clin Invest, 129: 598-615. 2019; Li et al., Mol Ther, 27: 2195-2212, 2019; Li et al., Mol Ther Methods Clin Dev, 9: 142-152, 2018; and Wang et al., J Virol, 79: 10999-11013, 2005) and human CD34+ cells (Li et al., Mol Ther, 27: 2195-2212, 2019) resulted in random transgene integration of 2 transgene copies/cell without a preference for genes.

The human genome is organized in a 3-D structure with long-range interactions between regulatory regions (i.e. transcription factor binding sites) usually through loop forming. Most of these interactions occur in the context of topologically associating domains (TADs). TADs are considered functional units of chromosome organization in which enhancers interact with other regulatory regions to control transcription. TAD/LCR border insulation is thought to restrict the search space of enhancers and promoters and to prevent unwanted regulatory contacts to be formed. Boundaries at both side of these domains are conserved between different mammalian cell types and even across species.

Currently used lentivirus and rAAV gene transfer vectors can accommodate only small enhancers/promoters, often resulting in suboptimal level and tissue specificity of transgene expression, transgene silencing, and unintentional interactions with regulatory regions surrounding the vector integration site. In the worst-case scenario, the latter can lead to the activation of proto-oncogenes.

To increase the safety and efficacy of gene therapy, TADs should be used for gene addition strategies. The median size of TAD is 880 kb. With further advancement of high-throughput chromosome conformation capture (3C) assay and its subsequent 4C, 5C and Hi-C protocols as well as fiber-Seq assays, the interrogation of regulatory genome will progress at a rapid speed and, for gene therapy purposes, could deliver TADs that contain only critical core elements.

The b-globin Locus Control Region (LCR) falls under the definition of a TAD. The human β-globin gene cluster lies in chromosome 11 and spans 100 kb. It has been proposed that the β-globin locus forms an erythroid-specific spatial structure composed of cis-regulatory elements and active β-globin genes, termed the active chromatin hub (ACH) (Tolhuis et al., Mol Cell, 10: 1453-1465, 2002). A core ACH is developmentally conserved, and includes the upstream 5′ DNAse hypersensitivity regions 1 to 5, called the globin LCR, and the downstream 3′HS1 as well as erythroid-specific transacting factors (Kim et al., Mol Cell Biol, 27: 4551-65, 2007). For gene therapy of hemoglobinopathies such as thalassemia major and Sickle Cell Anemia to be successful, it is essential that the transferred gene be expressed in erythroid cells at high levels, without position effects of integration and transcriptional silencing. To achieve this, the β-globin locus control region (LCR) is thought to be needed (Ellis et al., Clin Genet, 59: 17-24, 2001). For gene therapy applications, it is notable that a 23 kb β-globin LCR containing HS1 to HS5 conferred high-level, erythroid-specific, position independent expression upon cis-linked genes in transgenic mice (Grosveld et al., Cell, 51: 975-985, 1987). However, this version of the LCR is too large to be used in lentivirus vectors (insert capacity 8 kb) and, therefore truncated “mini” or “micro” LCR versions have been developed. For example, in ongoing clinical trials in thalassemia patients a lentivirus containing a 2.7 kb mini-LCR (covering HS2-HS4) and a 266 bp β-globin promoter is being used (Negre et al., Curr Gene Ther, 15: 64-81, 2015). In previous in vivo HSPC transduction studies, a 5.9kb β-globin LCR version that contained HS1 to HS4 and the β-globin promoter for expression of γ-globin in CD46 transgenic mice or CD46/Hbb^{th3} thalassemic mice was employed (Wang et al., J Clin Invest, 129: 598-615. 2019). With this in vivo HSPC transduction/selection approach, γ-globin marking was achieved in nearly 100% of peripheral blood erythrocytes, however the level of γ-globin expression was only 10-15% of that of adult mouse α-globin with an average integrated vector copy number (VCN) of 2-3 copies per cell. For a cure of β₀/β₀ thalassemia or Sickle Cell Anemia, it is generally thought that a level therapeutic globin (either γ- or β-globin) of 20% in erythroid cells is required (Fitzhugh et al., Blood, 130: 1946-1948, 2017). One way to reach this bar is by increasing the VCN by improving HSPC transduction or increasing the vector dose, which, however, bears the risk of increased genotoxicity considering the random integration pattern of this vector system. Therefore, focus was placed on utilizing a 29 kb LCR version to increase γ-globin expression per RBCs after in vivo HSPC transduction of CD46-transgenic wildtype and thalassemic mice.

Results. As a model for the in vivo transduction studies with intravenously injected HDAd5/35++ vectors, transgenic mice were used that contain the complete human CD46 locus and therefore express hCD46 in a pattern and at a level similar to humans (hCD46tg mice) (Kemper et al., (2001) Clin Exp Immunol 124: 180-189).

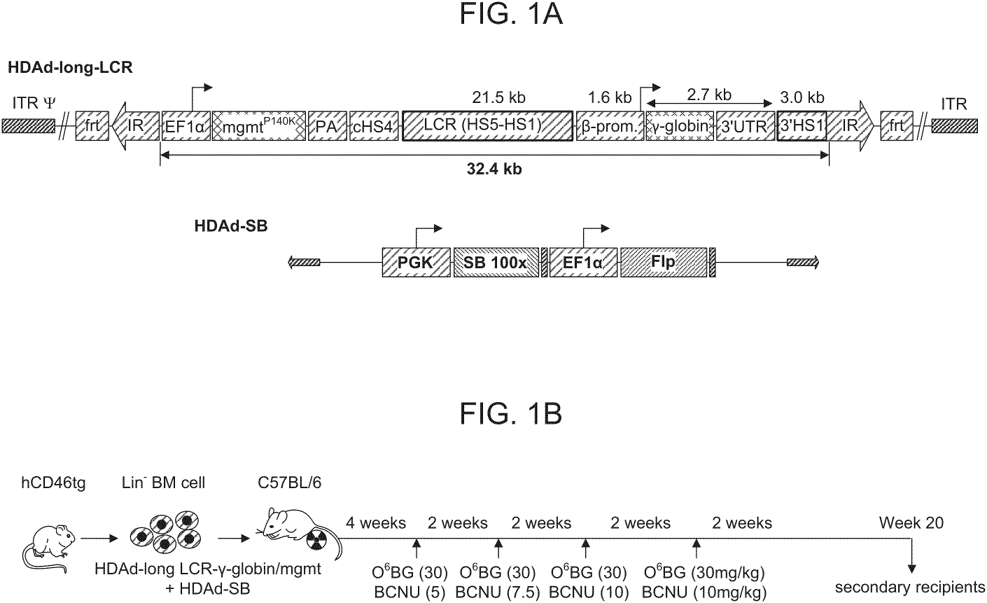

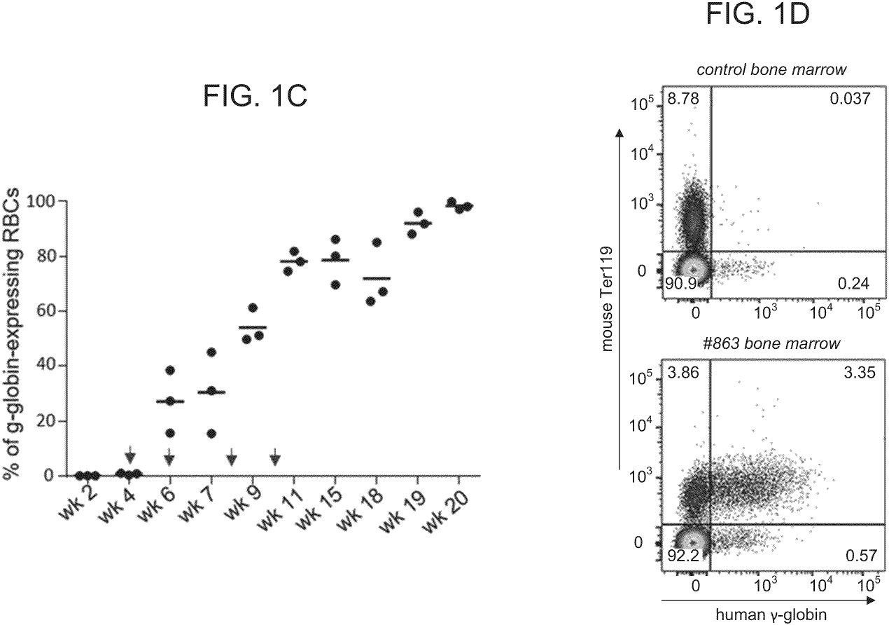

HDAd5/35++ vector containing a long β-globin LCR. In the studies described in Wang et al. (J. Clin Invest. 129(2):598-615, 2019), a HDAd5/35++ vector was used expressing γ-globin under the control of a 4.3 kb mini LCR (encompassing the core elements of HS1 to HS4; Lisowski et al., Blood 110:4175-4178, 2007) linked to a 1.6 kb β-globin promoter (Wang et al., J Clin Invest 129:598-615, 2019; Li et al., Mol Ther Methods Clin Dev 9: 142-152, 2018). In the present Example, an HDAd5/35++ vector was constructed that contained the following elements to maximize γ-globin gene expression: i) a 21.5 kb LCR including the full-length HS5 to HS1 regions, ii) a 1.6 kb β-globin promoter, iii) a β-globin 3′UTR to stabilize γ-globin mRNA, and iv) a 3′ HS1 region. The vector was named HDAd-long-LCR (FIG. 1A). To mediated integration the LCR-vectors are used in combination with a SB100x/Flpe expressing HDAd vectors (FIG. 1A).

In various embodiments, a 3′ HS1 has the following nucleic acid sequence of chr11 positions 5206867-5203839. In various embodiments, a 3′ HS1 has the following nucleic acid sequence as shown in SEQ ID NO: 102, or a sequence having at least 80% sequence identity to SEQ ID NO: 102, e.g., a sequence having at least 80%, 85%, 90%, 91%, 92%, 93%, 94%, 95%, 96%, 97%, 98%, or 99% identity to SEQ ID NO: 102.

Ex vivo HSPC transduction/transplantation study. HDAd-long-LCR contained a 32.4 kb transposon. While the SB system has been shown to be capable of delivering large cargos (Rostovskaya et al., Nucleic Acids Res 40: e150, 2012), it was unknown whether it could mediate the chromosomal integration of a 32.4 kb transposon. An ex vivo HSPC transduction was, therefore, performed in a setting where the transduction efficacy could be controlled. CD46tg mouse bone marrow lineage-negative (Lin⁻) cells, a cell fraction enriched for HSPCs, were transduced ex vivo with HDAd-long-LCR + HDAd-SB (FIGS. 1A, 1B). Ex vivo transduced cells were then transplanted into lethally irradiated C57BI/6 mice. Engraftment rates at week 4 were >95% based on CD46-positive PBMCs. The presence of the mgtm^{P140K} mutant gene in the vector allows for in vivo selection of transduced cells with O⁶BG/BCNU (Wang et al., Mol Ther Methods Clin Dev 8: 52-64, 2018). One month after transplantation, mice were subjected to four rounds of O⁶BG/BCNU treatment to selectively expand progenitors with integrated γ-globin/mgmt transgenes (FIG. 1A). With each round of in vivo selection, the percentage of γ-globin-positive peripheral red blood cells (RBCs) increased, reaching >95% at week 20, the end of the study (FIG. 1C). At week 20, animals were sacrificed and bone marrow mononuclear cells (MNCs) were analyzed. The average VCN measured by qPCR was 2.8 copies per cell. γ-globin expression was detected by flow cytometry in 85.46(+/-5.9)% of erythroid Ter119⁺cells and in 14.54(+/-2.3)% non-erythroid (Ter119⁻) bone marrow MNCs (FIG. 1D).

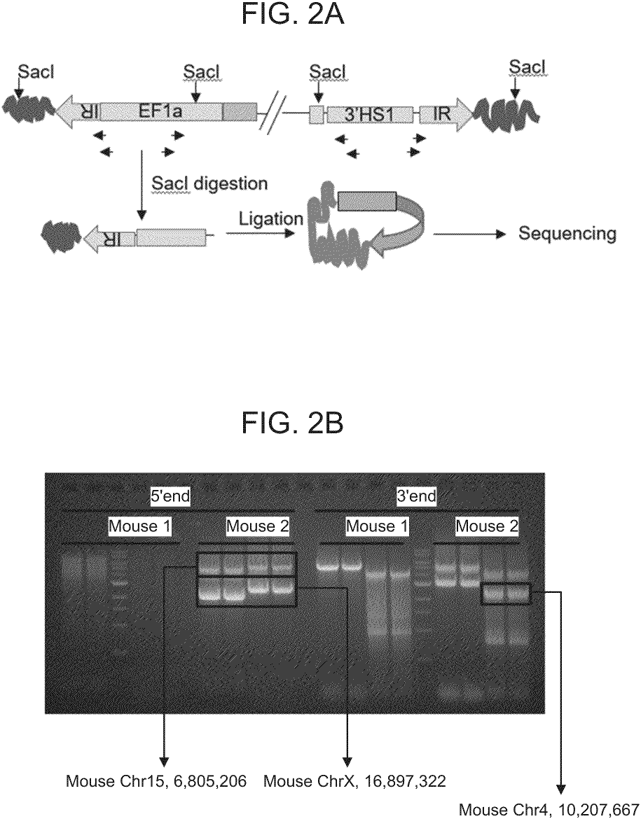

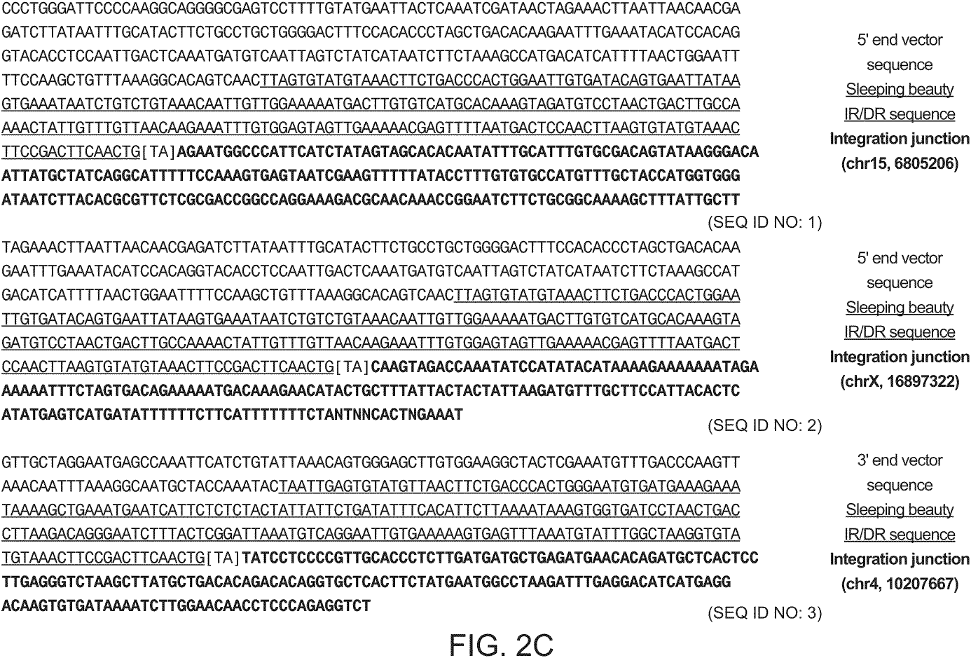

To demonstrate that γ-globin expression originated from SB100x integrated transgenes, an inverse PCR (iPCR) analysis was performed on genomic DNA from bone marrow mononuclear cells (MNCs) harvested at week 20 after transplantation. The iPCR protocol involves the digestion of genomic DNA with Sacl, a re-ligation/circularization step, nested PCR and sequencing of vector/chromosome junctions (FIG. 2A). (FIG. 2B) shows three representative PCR products and the localization of the integration sites on chromosomes 4, 15, and X. Sequencing of the products demonstrated vector/chromosome junctions typical for SB100x mediated integration including the TA di-nucleotides at the vector IR/DR-chromosome junctions (FIG. 2C). In summary, in the ex vivo HSPC transduction study, the long globin LCR conferred high-level γ-globin expression originating from SB100x integrated transposons.

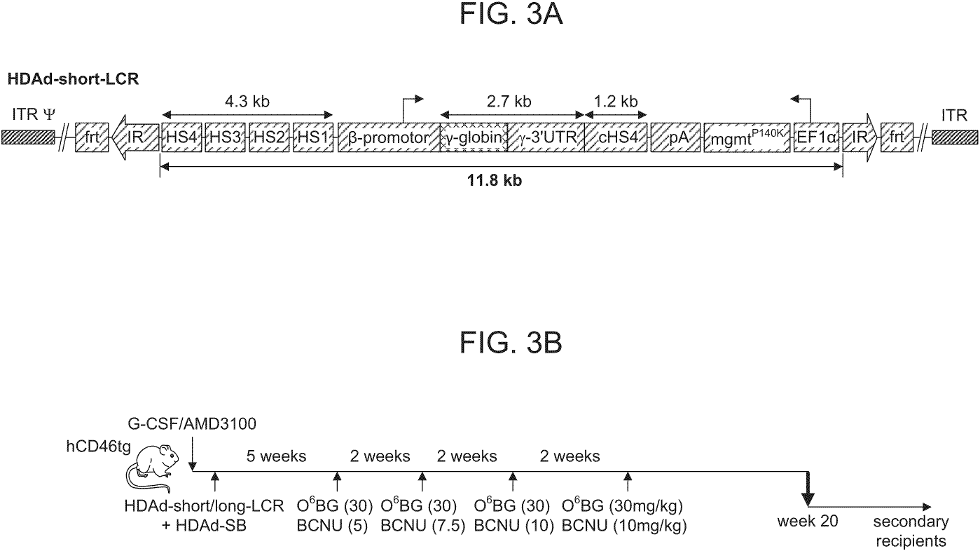

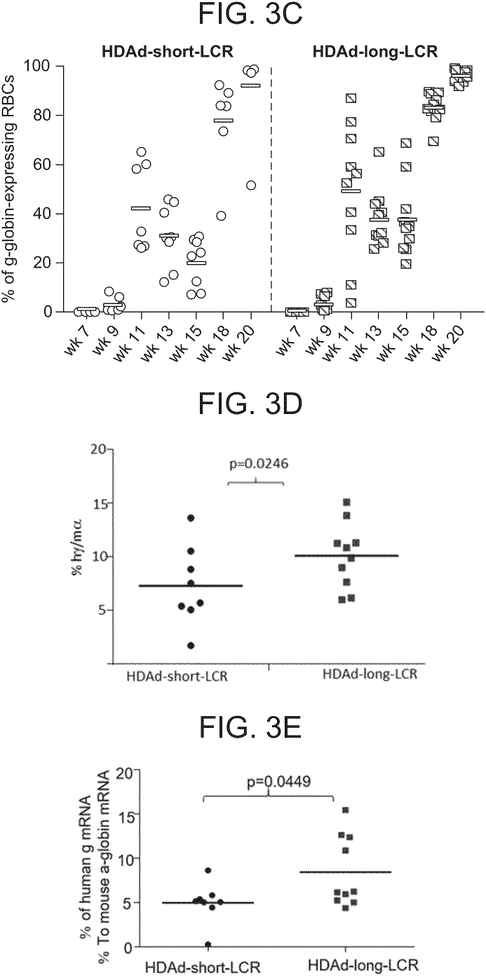

In vivo HSPC transduction in CD46b transgenic mice with HDAd5/35++ vectors containing the short vs long LCRs. A side-by-side comparison of HDAd-long-LCR and the previously used vector (Wang et al., J Clin Invest 129: 598-615, 2019; Li et al., Mol Ther Methods Clin Dev 9: 142-152, 2018) containing the miniLCR (herein referred to as “HDAd-short-LCR”) was performed (FIG. 3A). CD46-transgenic mice were mobilized with G-CSF/AMD3100 and intravenously injected with the vectors. Four rounds of O⁶BG/BCNU selection were initiated at week 5 after in vivo transduction, and mice were followed for 20 weeks (FIG. 3B). Week 20 bone marrow Lin⁻ cells were then transplanted into lethally irradiated C57BI/6 mice and secondary recipients were monitored for another 16 weeks. As in the ex vivo HSPC transduction study, the percentage of γ-globin-positive RBCs increased with each round of in vivo selection reaching >95% for both vectors at week 20 (FIG. 3C). HPLC performed on RBC lysates from week 20 samples showed a significantly higher γ-globin/adult mouse α-globin percentage for the HDAd-long-LCR vector (FIG. 3D). This difference was also reflected at the mRNA level (FIG. 3E).

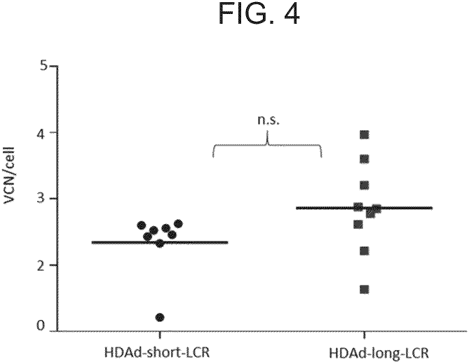

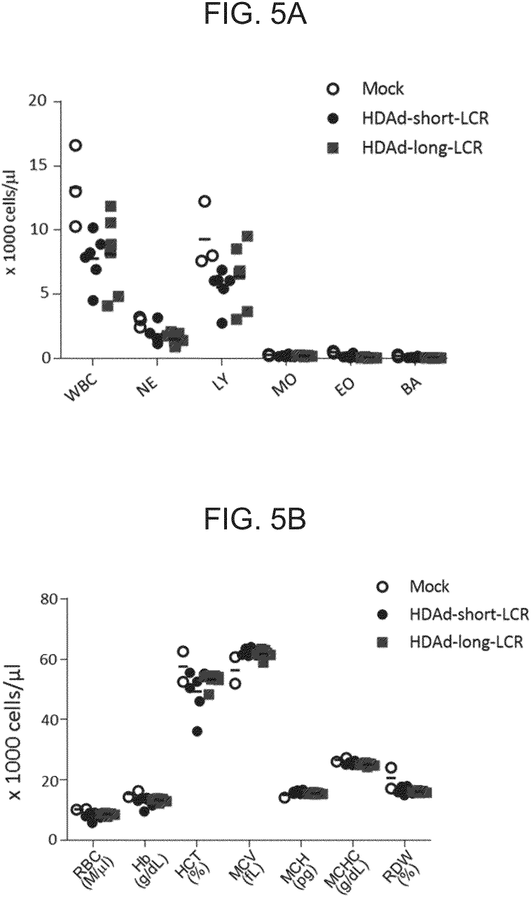

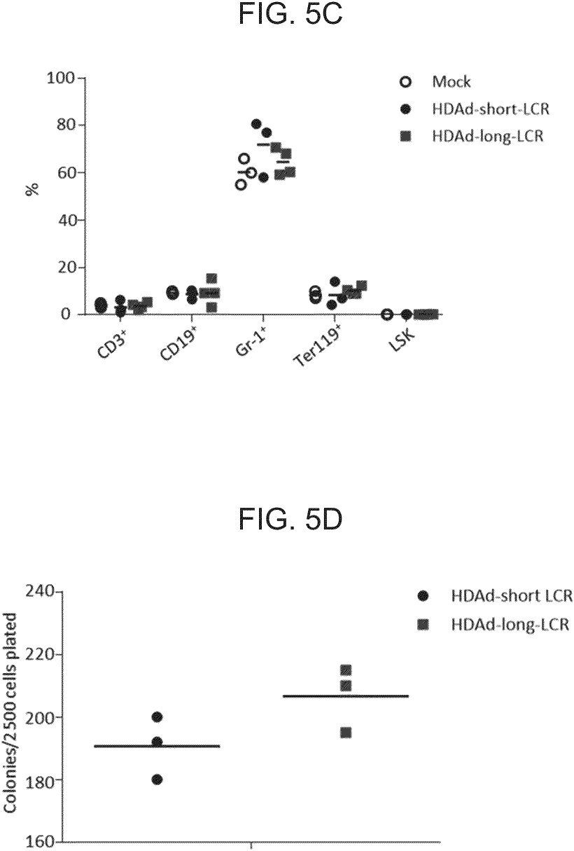

The vector copy number in bone marrow MNCs measured at week 20 by qPCR was 2.5-3 copies per cell (FIG. 4) and not significantly different between the vectors. This indicated that the integration of the “short” 11.8 kb transposon was as efficient as the integration of the “long” 32.4 kb transposon. In vivo HSPC transduction with the vectors did not cause hematological abnormalities (week 20) in spite of γ-globin expression in the vast majority of erythroid cells (FIGS. 5A-5B). The composition of cellular bone marrow (FIG. 5C) and the colony forming-potential of bone marrow Lin⁻ cells (FIG. 5D) were not significant between groups.

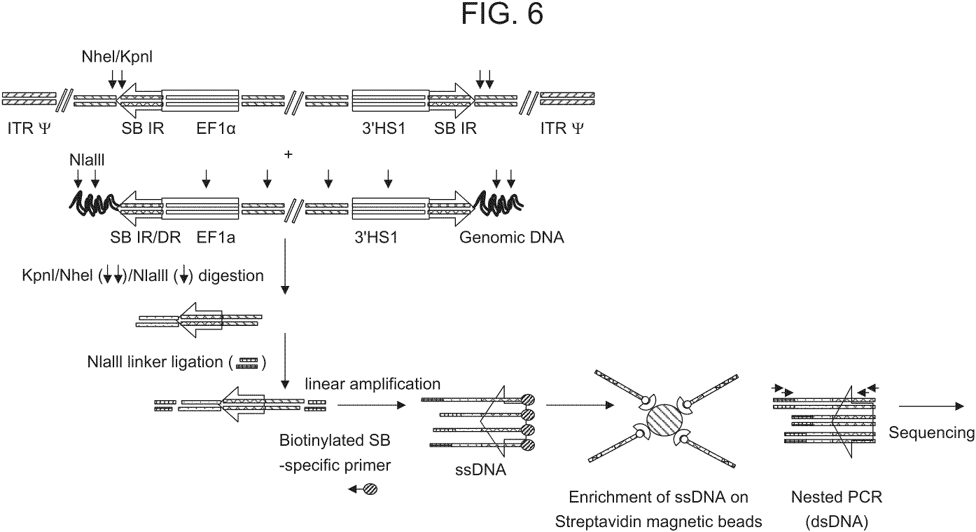

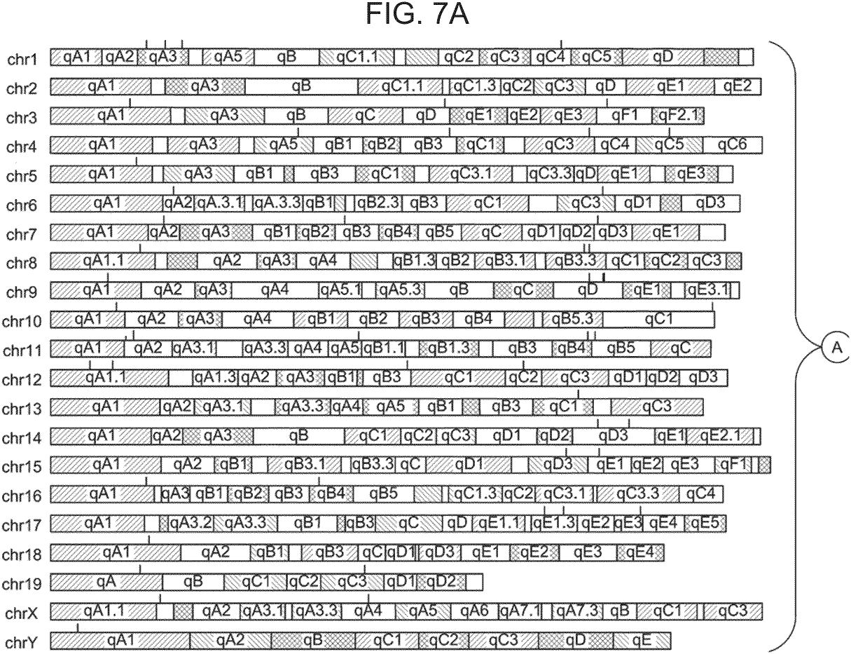

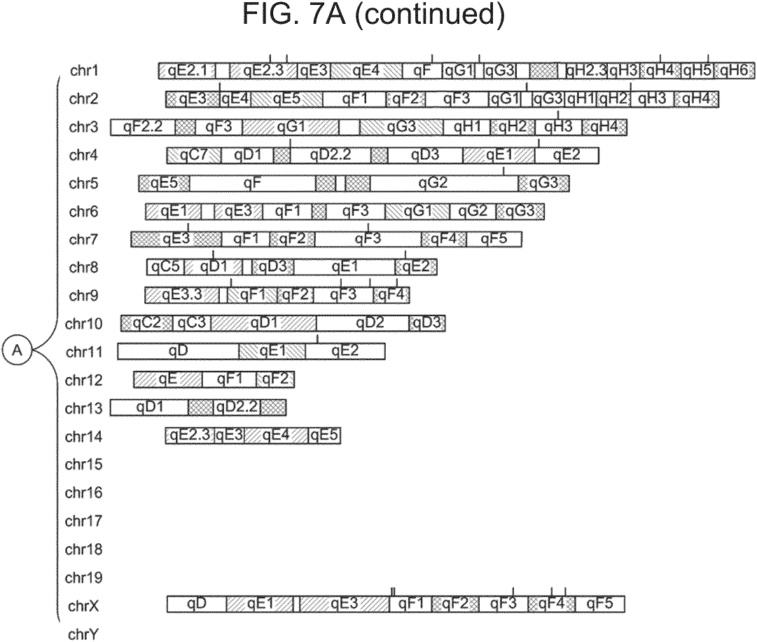

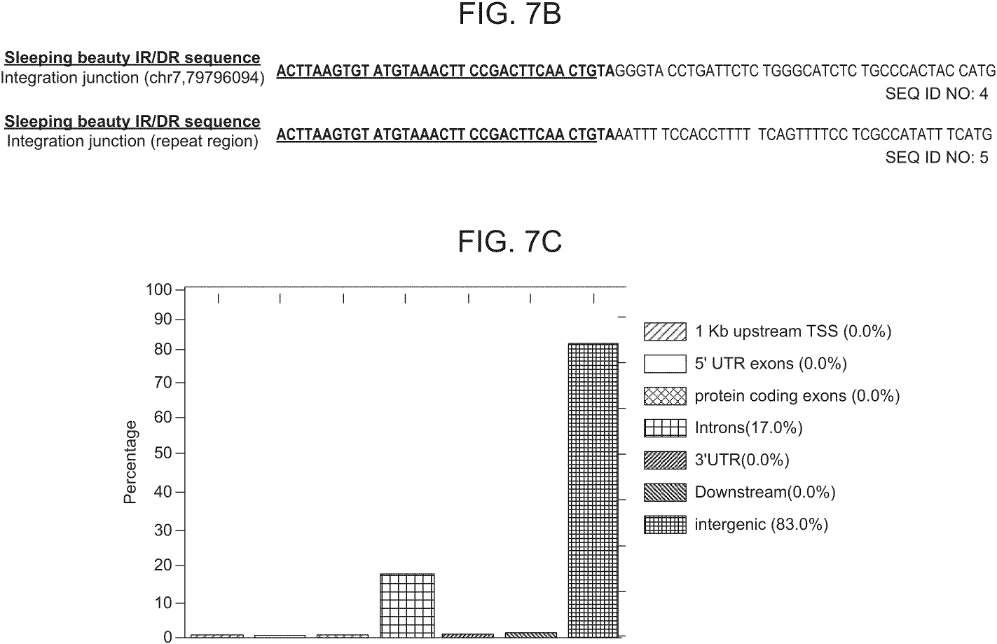

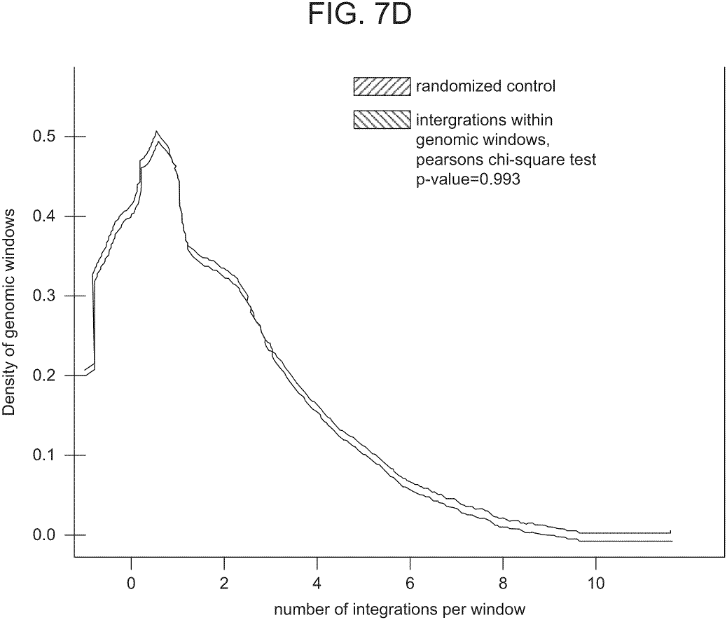

Bone marrow Lin⁻ cells harvested at week 20 were also used to perform a genome-wide integration analysis using linear amplification-mediated PCR (LAM-PCR), followed by sequencing of integration junctions (FIG. 6). In genomic DNA samples pooled from five mice, a total of 76 distinct SB100x-mediated integration sites were identified (FIG. 7A, on two pages). IR/DR/chromosome junction contained TA dinucleotides (FIG. 7B). The vast majority of integrations were within intergenic and intronic regions at a frequency of 82% and 19%, respectively (FIG. 7C). No integration within or near a proto-oncogene was found. The integration was random without preferential integration in any given window of the whole mouse genome (FIG. 7D).

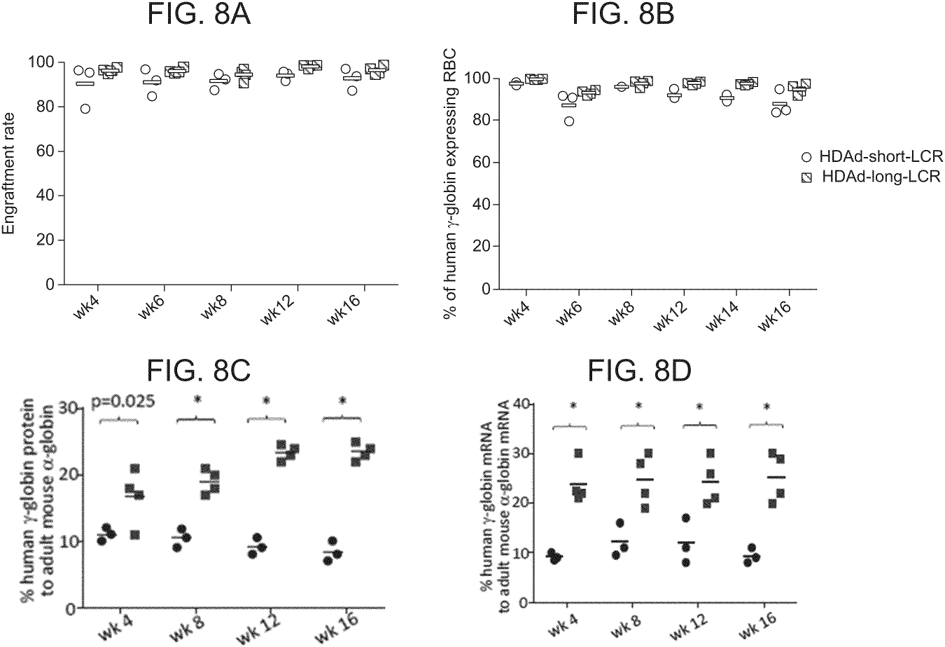

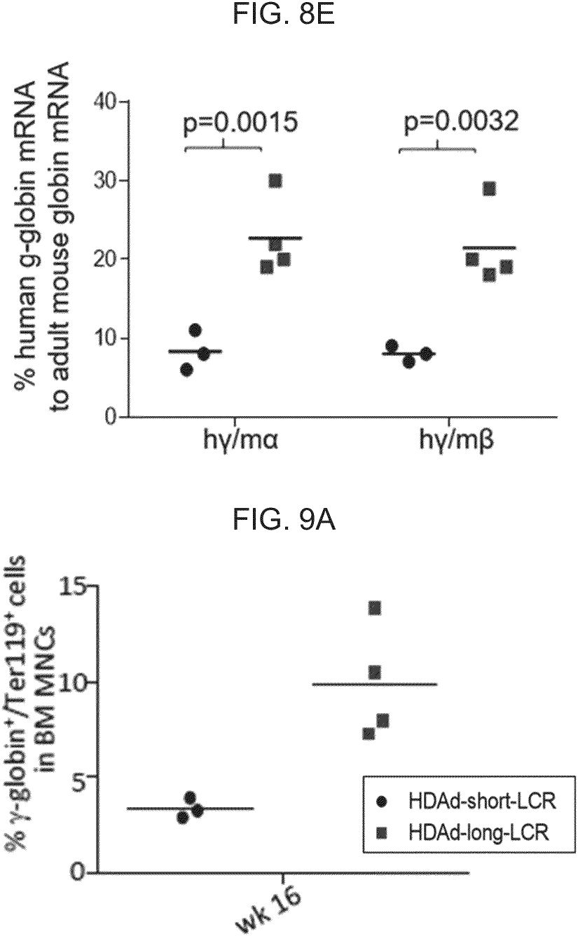

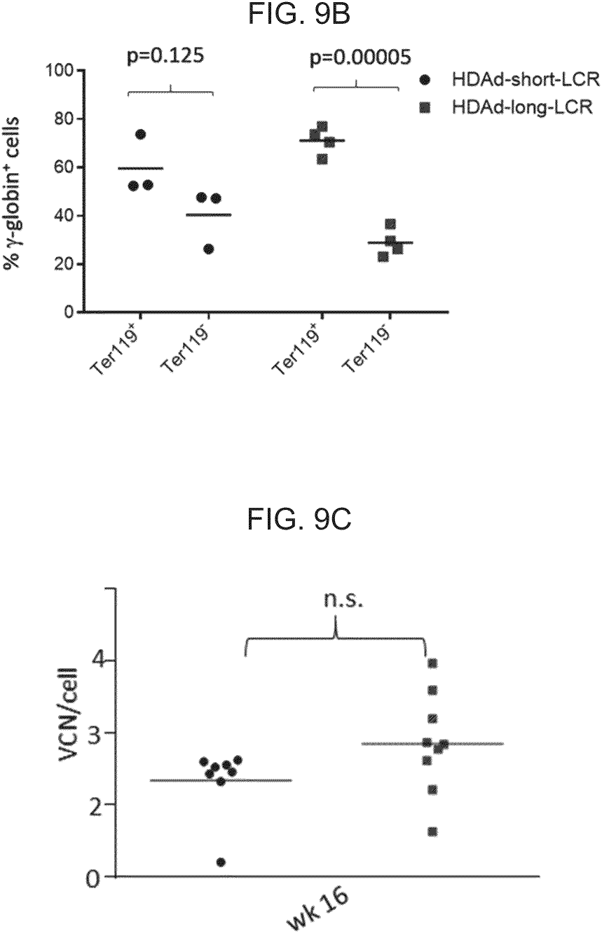

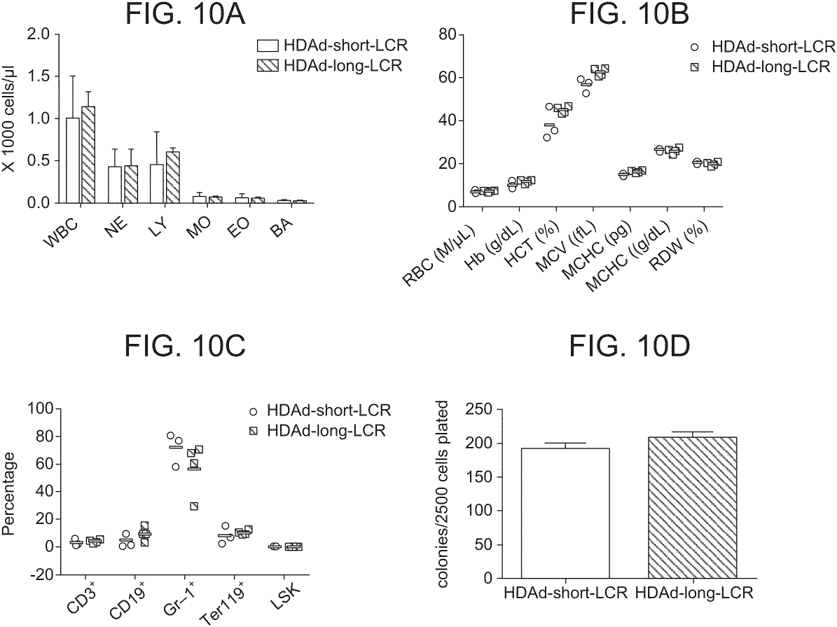

Analysis of secondary recipients. To demonstrate that in vivo transduction and SB100x-mediated integration occurred in long-term repopulating HSPCs, bone marrow Lin⁻ cells harvested at week 20 after in vivo HSPC transduction were transplanted into lethally irradiated C57BI/6 mice (without the hCD46 transgene). The ability of transplanted cells to drive the multi-lineage reconstitution in secondary recipients was assessed over a period of 16 weeks. Engraftment rates based on hCD46 expression in PBMCs were 95% and remained stable (FIG. 8A). γ-globin marking of RBCs measured by flow cytometry was in the range of 90 to 95% and stable (FIG. 8B). There was no significant difference between the two vectors in the percentage of γ-globin⁺ RBCs. The average integrated vector copy number also did not differ significantly between the two vectors. To measure γ-globin expression levels HPLC (FIG. 8C) and qRT-PCR (FIGS. 8D, 8E) were used. In both analyses, the percentage of γ-globin to mouse adult globin chains was greater for the HDAd-long-LCR vector. γ-globin levels for this vector were in the range of 20-25% of mouse α-globin implying that they would be curative for hemoglobinopathies. In addition to conferring higher γ-globin expression levels, the long LCR also provided more stringent erythroid-specific expression as shown by a significantly higher percentage of γ-globin expressing bone marrow cells in the erythroid (Ter119⁺) fraction vs the non-erythroid fraction (Ter119⁻) (FIGS. 9A, 9B). The vector number copy per cell in bone marrow MNCs were not statistically significant between HDAd-short-LCR and HADad-long-LCR when harvested at week 16 after in vivo HSPC transduction (FIG. 9C). As in the “primary” in vivo HSPC transduced mice, no effect of high-level globin expression on the cellular composition of the bone marrow or hematological parameters in the peripheral blood were observed in secondary recipients (FIGS. 10A-10D).

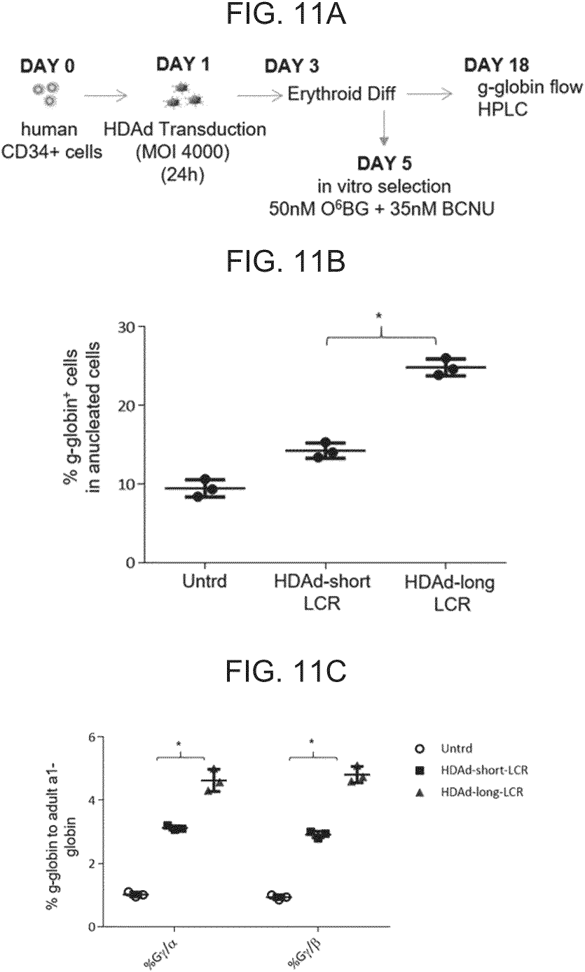

Comparison of the two vectors after human CD34+ transduction, in vitro selection, and erythroid differentiation. The function of the human β-globin LCR in a heterologous system like mouse erythroid cells could be suboptimal due to lack of conservation of transcription factors that bind within the LCR. An in vitro study in human cells was, therefore, performed (FIG. 11A). Human CD34+ cells obtained from GCSF-mobilized healthy donors were transduced with HDAd-long-LCR + HDAd-SB or HDAd-short-LCR + HDAd-SB at a total MOI of 4000 vp/cells, i.e. a MOI that confers the transduction of the majority of CD34+ cells (Li et al., Mol Ther Methods Clin Dev 9: 390-401, 2018). Transduced cells were then subjected to erythroid differentiation (ED) and O⁶BG/BCNU selection for cells with integrated transgenes. During expansion of transduced cells over 18 days, most of episomal vectors are lost. At the end of ED, significantly higher percentages of γ-globin+ anucleated cells (i.e. reticulocytes that lost the nucleus) were found for the HDAd-long-LCR + HDAd-SB setting by flow cytometry (FIG. 11B). HPLC analysis also demonstrated significantly higher γ-globin chain levels in HDAd-long-LCR + HDAd-SB-transduced cells (FIG. 11C).

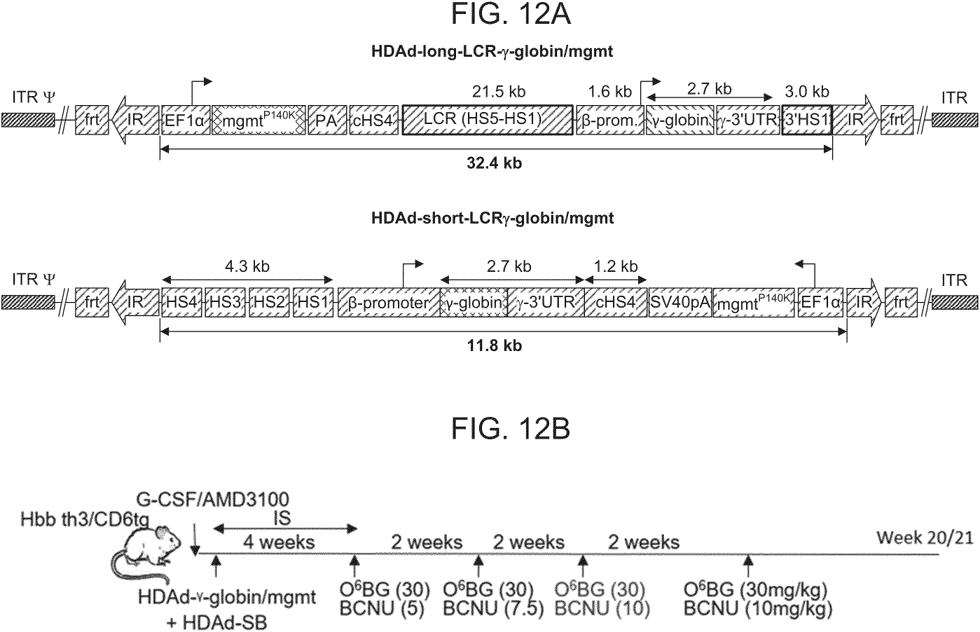

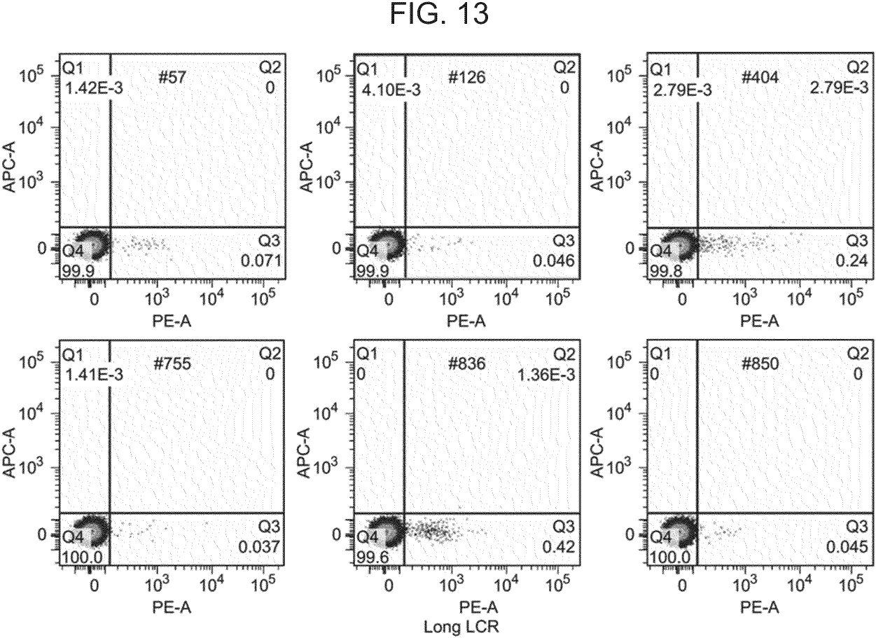

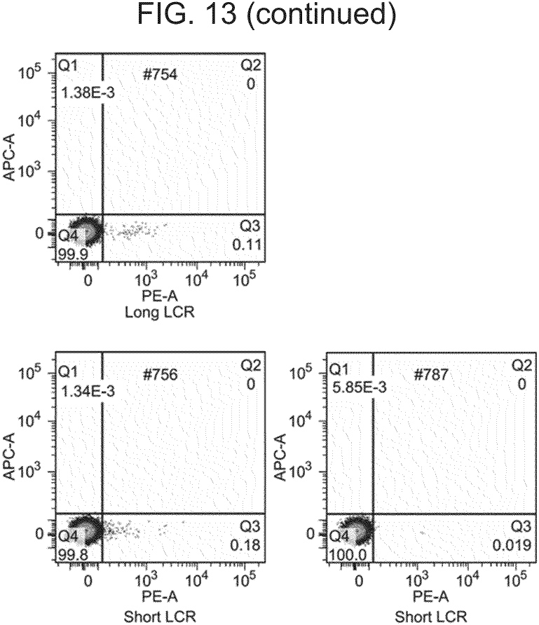

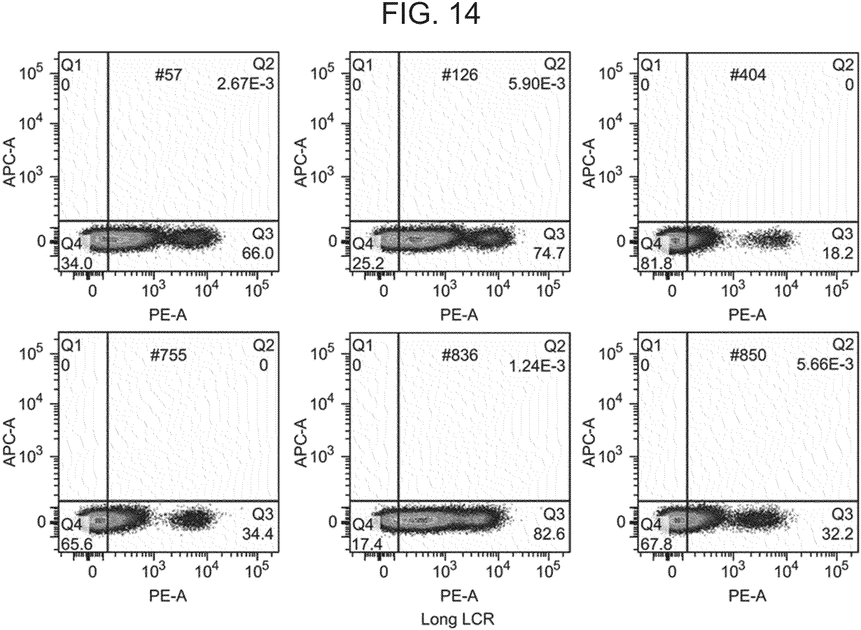

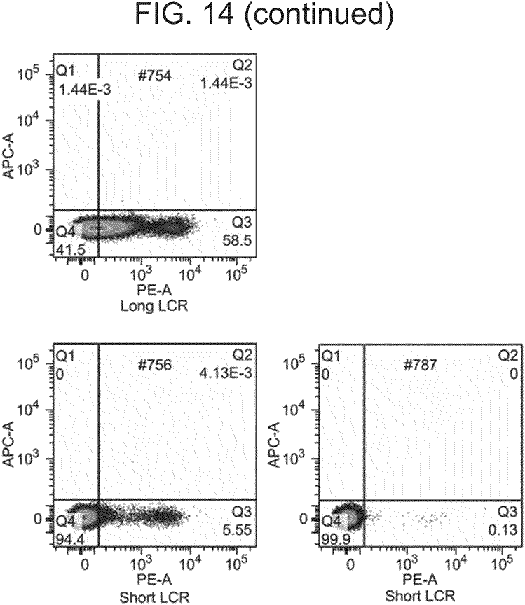

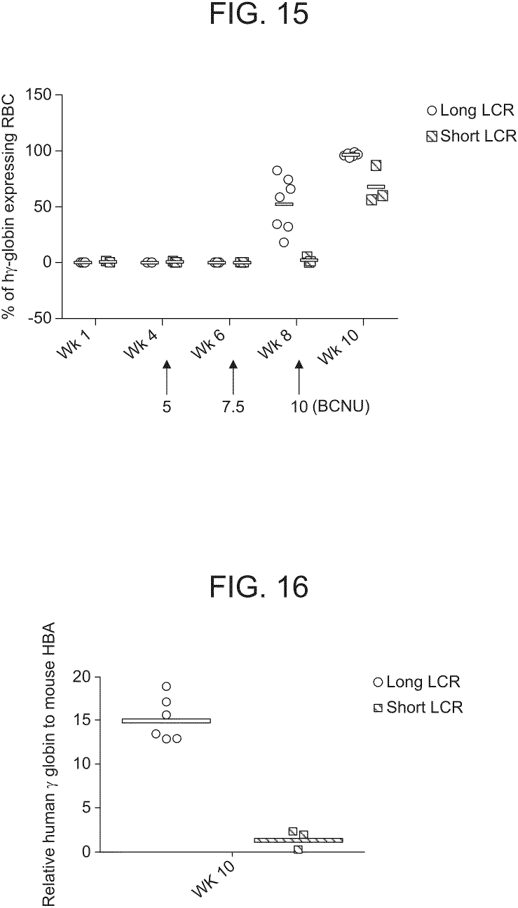

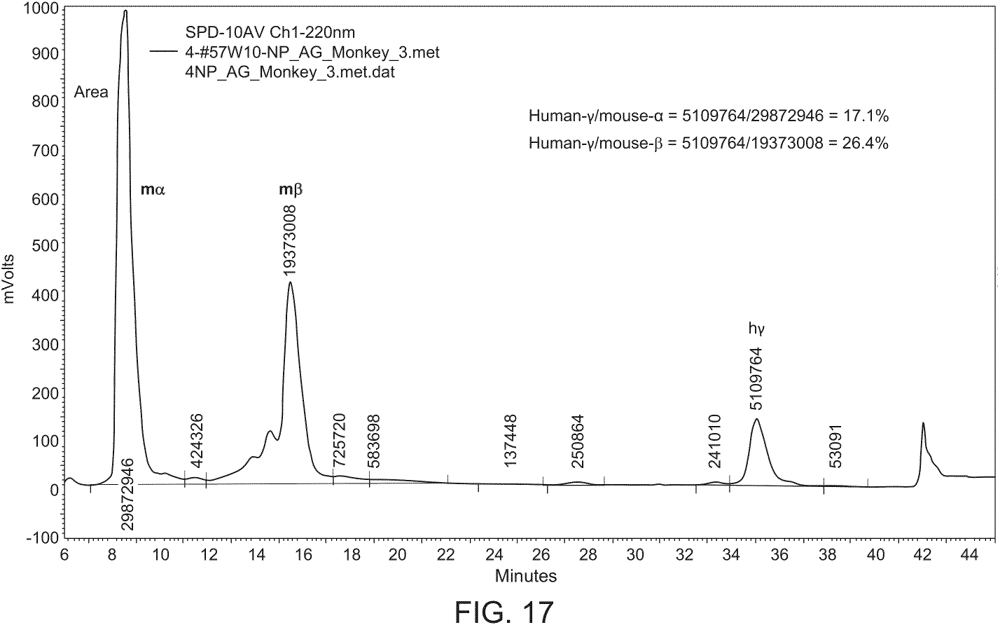

Structure of exemplary an HDAd-long-LCR vector and an HDAd-short-LCR vector. In the HDAd-long-LCR, the γ-globin gene under the control of a 21.5 kb β-globin LCR (chr11:5292319-5270789), a 1.6 kb β-globin promoter (chr11:5228631-5227023) and a 3′ HS1 region (chr11:5206867-5203839) also derived from the β-globin locus. For RNA stabilization in erythroid cells a β-globin gene UTR was linked to the 3′ end of the g-globin gene. The vector also contains an expression cassette for mgmt^{p140k} allowing for in vivo selection of transduced HSPCs and HSPC progeny. The γ-globin and mgmt. expression cassettes are separated by a chicken globin HS4 insulator. The 32.4 kb LCR-γ-globin/mgtm transposon is flanked by inverted repeats (IRs) that are recognized by SB100x and by frt sites that allow for circularization of the transposon by FIpe recombinase. In the HDAd-short-LCR, instead of the 21.5 kb HS-HS5 LCR and 3′ HS1 present in HDAd-long-LCR, this vector contains a 4.3 kb mini-LCR including the core regions of DNase hypersensitivity sites (HS) 1 to 4. The length of the transposon is 11.8 kb. (FIG. 12A) hCD46tg mice were mobilized and IV injected with either HDAd-short-LCR + HDAd-SB or HDAd-long-LCR + HDAd-SB (4 x 10¹⁰vp of a 1:1 mixture of both viruses). Five weeks later, O⁶BG/BCNU treatment was started. With each cycle, the BCNU concentration was increased from 2.5 mg/kg, to 7.5 mg/kg, and 10 mg/kg. The O6BG concentration was 30 mg/kg in all three treatments. Mice were followed until week 20 when animals were sacrificed for analysis. (FIG. 12B)

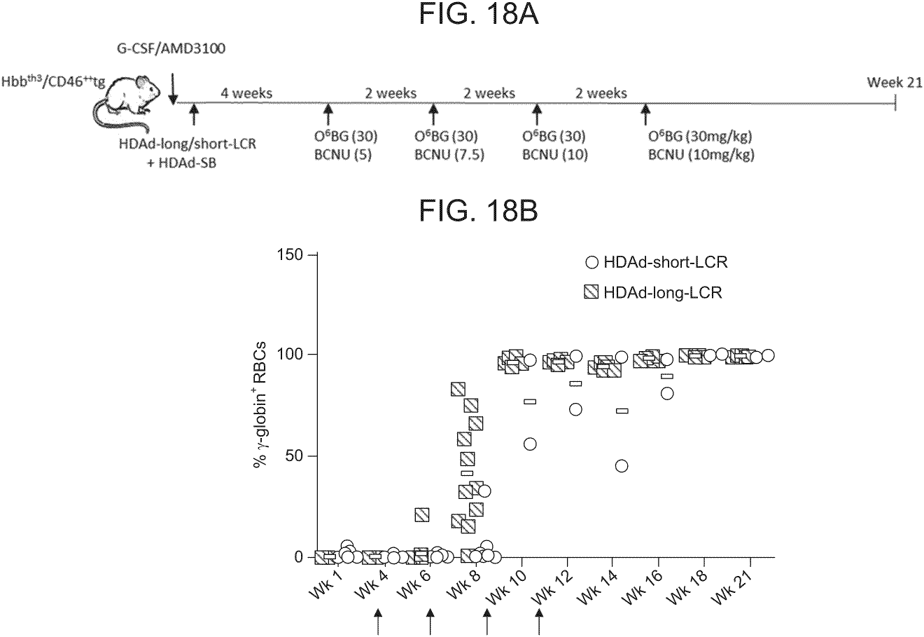

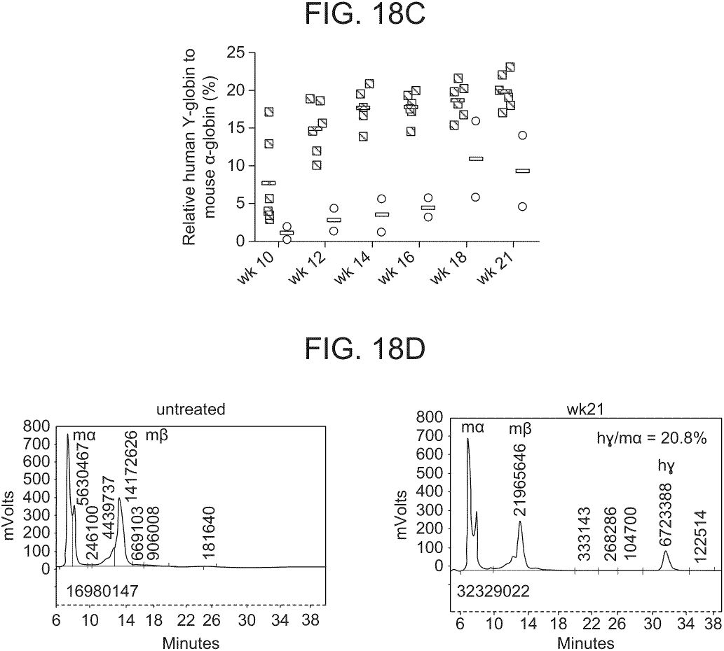

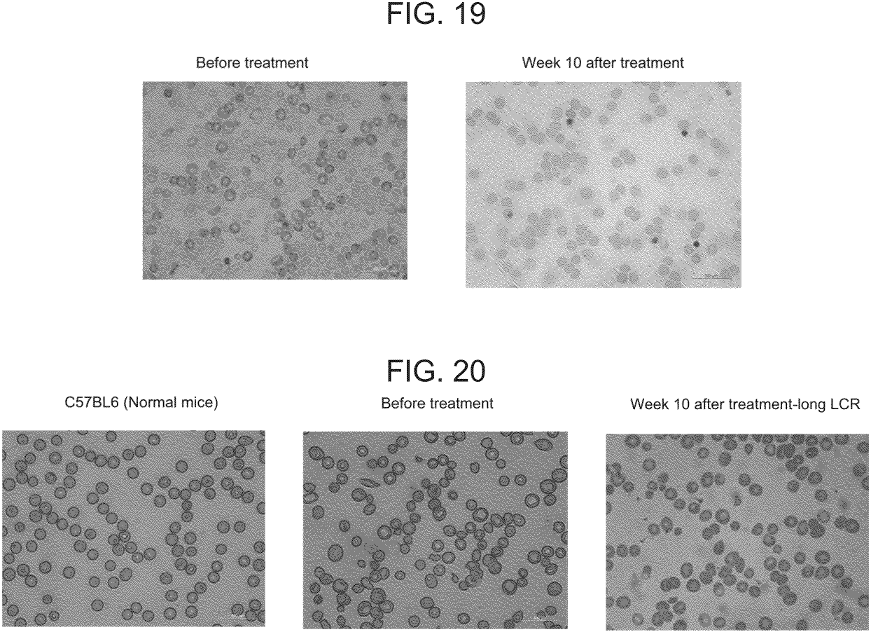

Studies in a mouse model for thalassemia intermedia: γ-globin levels. For these studies (CD46+/+) mice were bred with Hbb^{th3} mice heterozygous for the mouse Hbb-beta1 and -beta2 gene deletion (Yang et al., Proc Natl Acad Sci U S A, 92: 11608-11612, 1995). Resulting Hbb^{th3}/CD46^{+/+} mice has the typical phenotype of thalassemia intermedia (Wang et al., J Clin Invest, 129: 598-615. 2019). Hbb^{th3}/CD46^{+/+} mice were mobilized and IV injected with HDAd-long-LCR and HDAd-short LCR (FIG. 18A). Four weeks later, 4 rounds of in vivo selection with increasing doses of O⁶BG/BCNU were initiated. γ-globin marking in peripheral red blood cells was on average 40% already the second cycle of in vivo selection and reached 100% in all mice after the third cycle of in vivo selection for mice transduced with HDAd-long-LCR (FIG. 18B). For mice transduced with HDAd-short-LCR, it required four in vivo selection cycles to reach 100% γ-globin marking in RBCs. At 100% marking rate, the percentage of human γ-globin chains vs adult mouse α-globin (measured by HPLC) increased over time (most likely due to the disease background) reaching an average of 20% by week 21 after treatment (FIGS. 18C and 18D). These data demonstrate the superiority of HDAd-long-LCR by i) requiring less intense in vivo selection and ii) achieving γ-globin expression levels, that, in theory, should be curative in patients with SCA and thalassemia major.

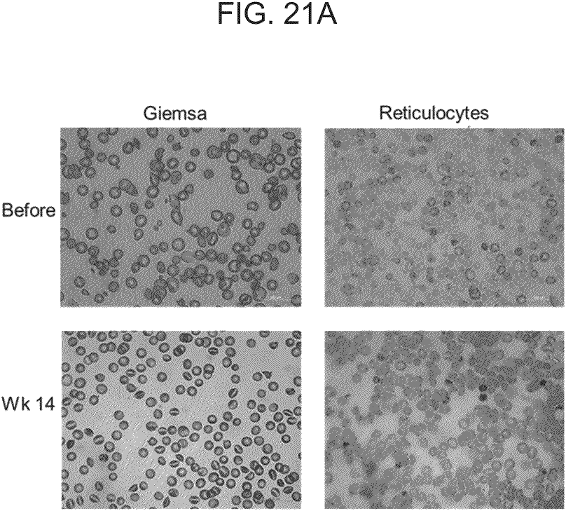

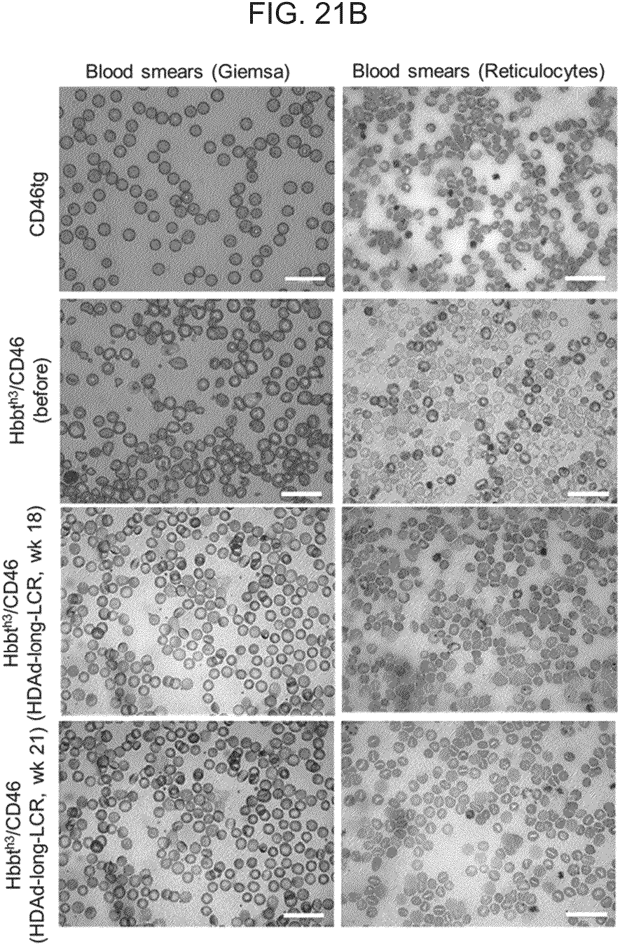

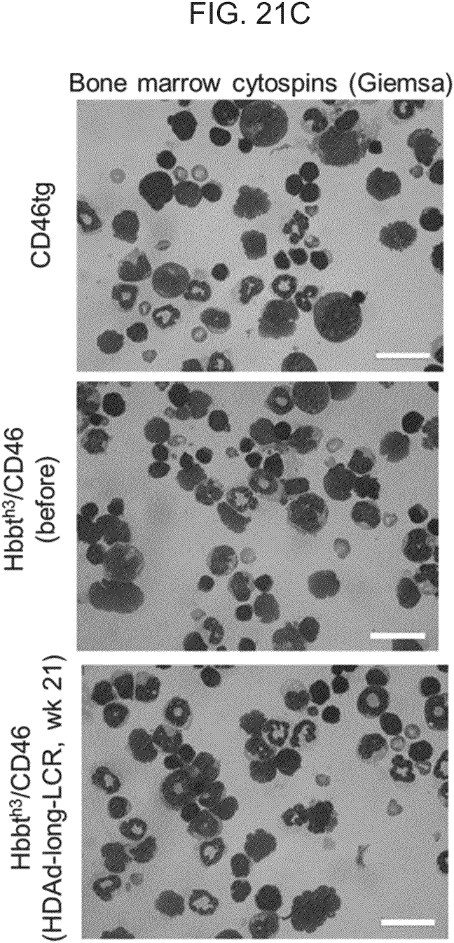

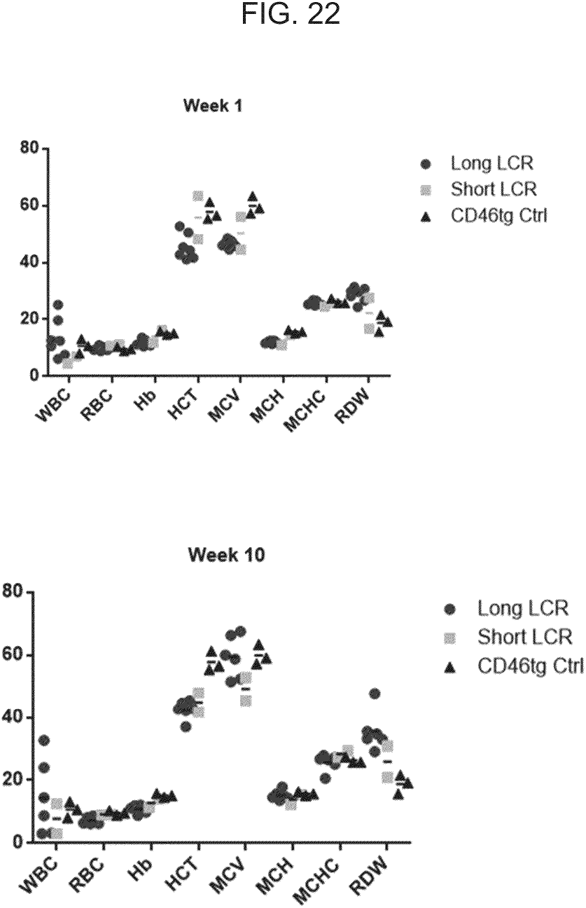

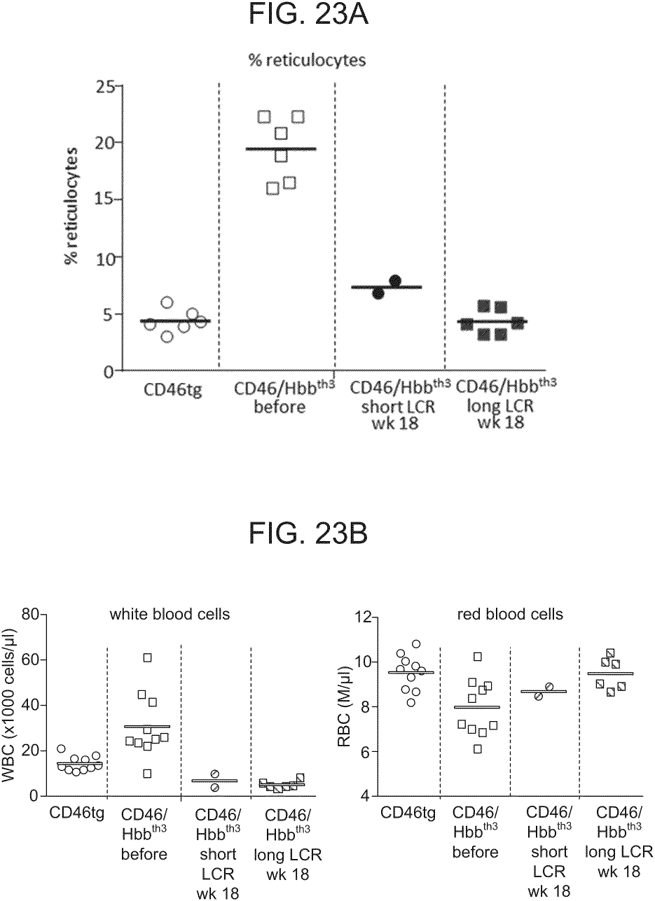

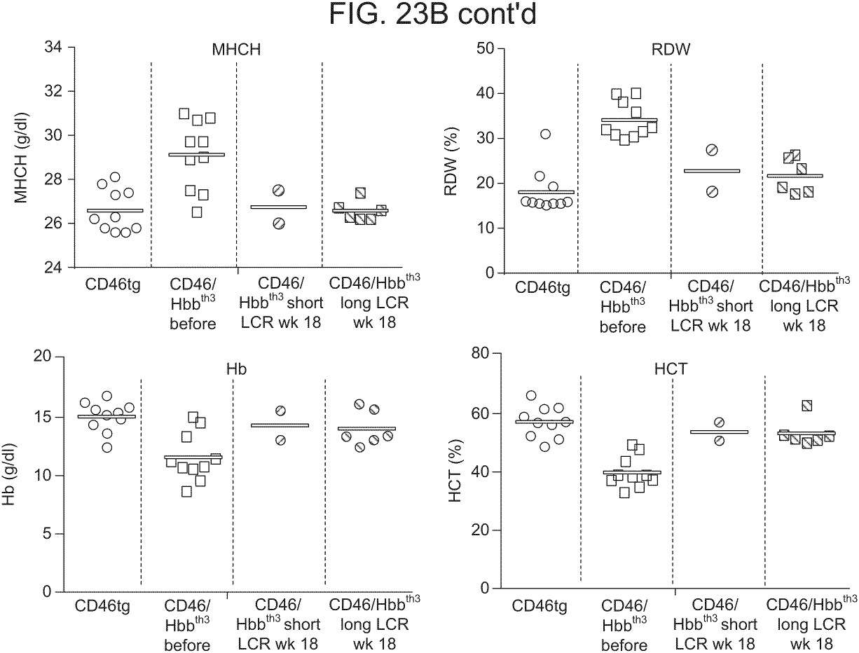

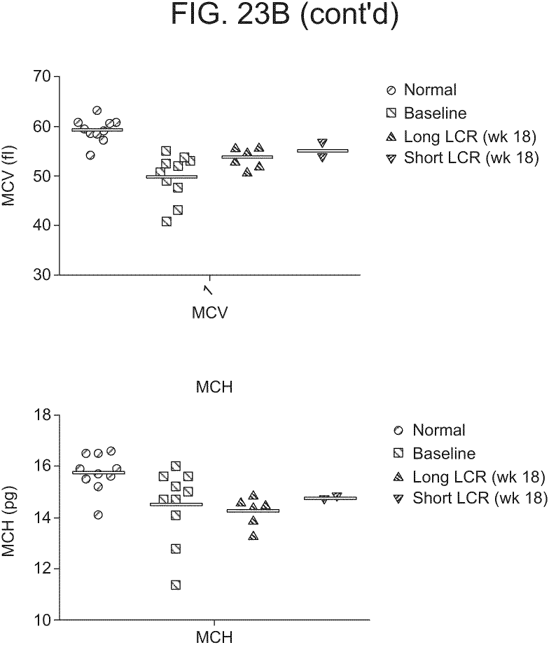

Studies in a mouse model for thalassemia intermedia: correction of hematological parameters. Phenotypic correction is shown at different time points. At week 14, blood cell morphology stained with Giemsa stain and May-Grünwald stain are shown (FIG. 21A). At week 21 after treatment, mice were sacrificed. Indicative of the reversal of the thalassemic phenotype in peripheral blood smears of the treated CD46^{+/+}/Hbb^{th3} mice, the hypochromic, highly fragmented and anisopoikilocytic baseline RBCs were replaced by near normochromic, well-shaped RBCs (FIG. 21B, left panels). Reticulocytes were counted on blood smears from thalassemic and mice treated with HDAd-long-LCR at week 21 (FIG. 21B, right panel) In bone marrow cytospins, in contrast to the blockade of erythroid lineage maturation in bone marrow of CD46^{+/+}/Hbb^{th3} mice, represented by the prevalence of pro-erythroblasts and basophilic erythroblasts, in cytospins from control and treated CD46^{+/+}/Hbb^{th3} mice, maturing erythroblasts predominated and were represented by polychromatic and orthochromatic erythroblasts (FIG. 21C). The normalized erythrocyte parameters of mice transduced with long LCR, short LCR, and control CD46tg vectors are shown (FIG. 22). The percentage of reticulocytes counted on blood smears returned from an average of 20% in thalassemic mice to normal values (5%) mice treated with HDAd-long-LCR at week 18 (FIG. 23A). Hematological parameters at week 18 post in vivo transduction were indistinguishable from their control CD46tg counterparts, suggesting complete phenotypic correction. This included a normalization in white and red blood cell counts as well as erythroid cell features (Hb, HCT, MHCH, and RDW) (FIG. 23B). Furthermore, differences were not significant between normal, baseline, long LCR, and short LCR vectors in MCV and MCH cells at week 18 (FIG. 23B).

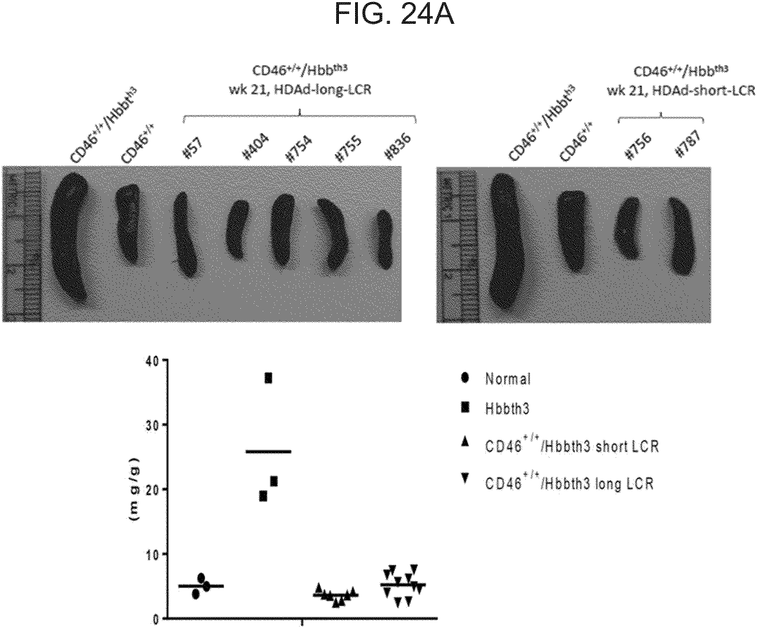

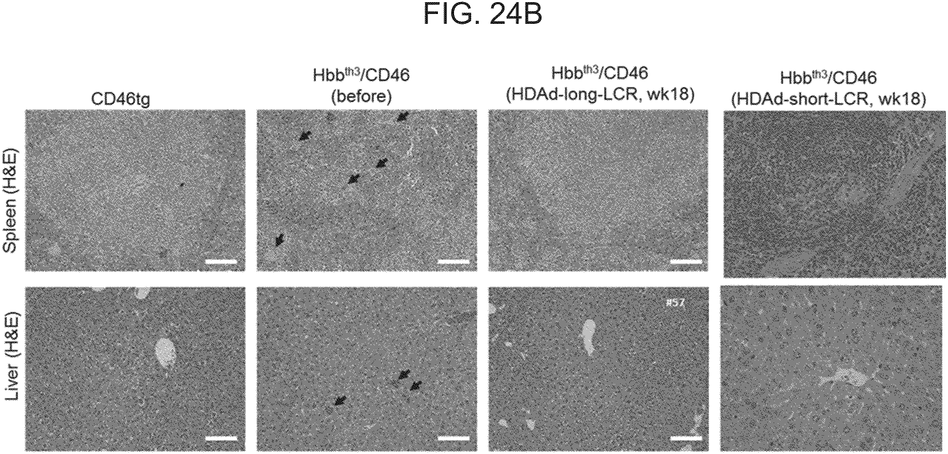

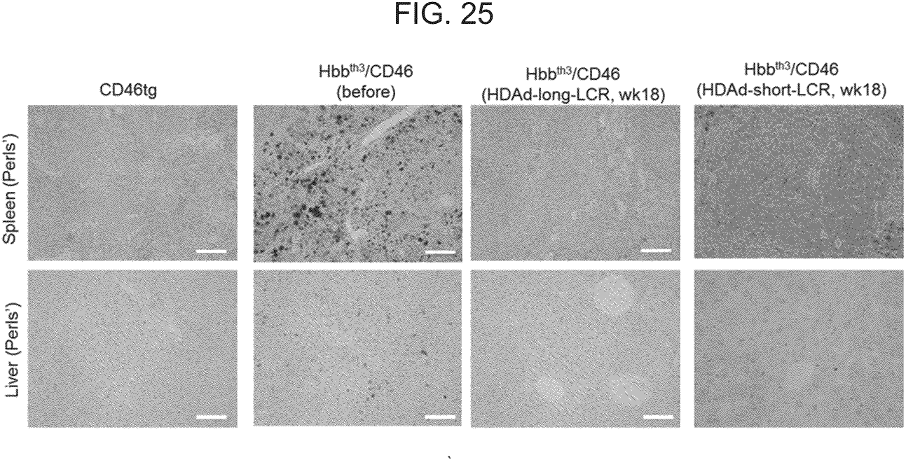

Studies in a mouse model for thalassemia intermedia: correction of extramedullary hematopoiesis and hemosiderosis. Spleen size, a measurable characteristic of compensatory hemopoiesis was reduced to normal in animals treated with HDAd-long-LCR (FIG. 24A). In contrast to Hbb^{th3}/CD46 mice, no foci of extramedullary erythropoiesis were observed on spleen and liver sections (FIG. 24B). Intense parenchymal hemosiderosis was prominent in the untreated CD46^{+/+}/Hbb^{th3} mice whereas only background iron accumulation in the CD46tg and the treated CD46^{+/+}/Hbb^{th3} mice could be detected (FIG. 25).

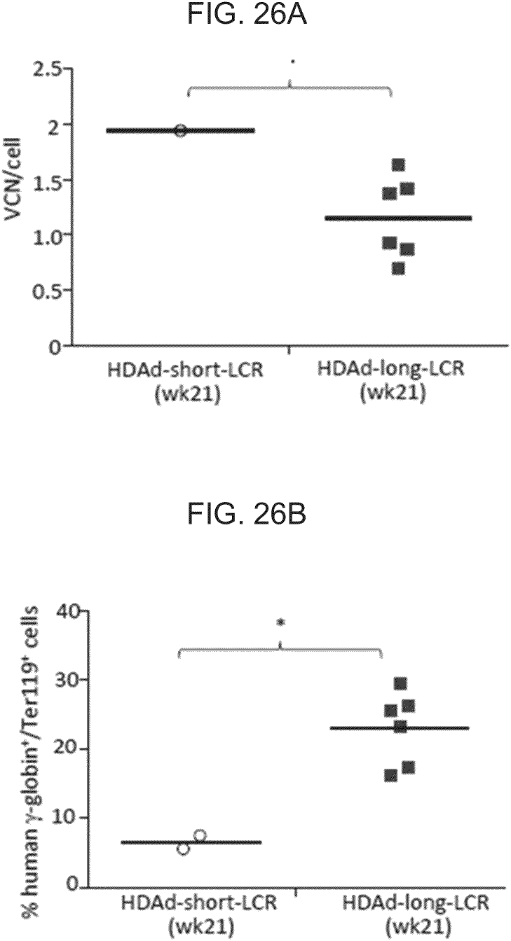

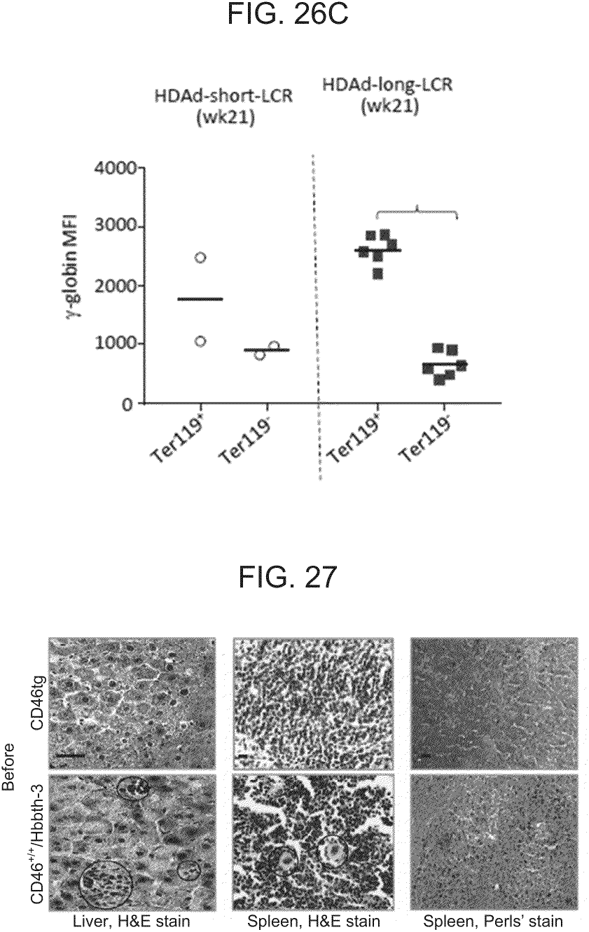

Bone marrow was harvested at week 21 after in vivo HSC transduction of Hbb^{th3}/CD46tg mice. (FIG. 26A) Vector copy number per cell in bone marrow MNCs. The difference between the two groups is not significant but could become significant if analyzed with greater sample size. (FIGS. 26B, 26C) Erythroid specificity of γ-globin expression. (FIG. 26B) Percentage of γ-globin expressing erythroid (Ter119⁺) and non-erythroid (Ter119⁻) cells. *p<0.05. Statistical analyses were performed using two-way ANOVA.

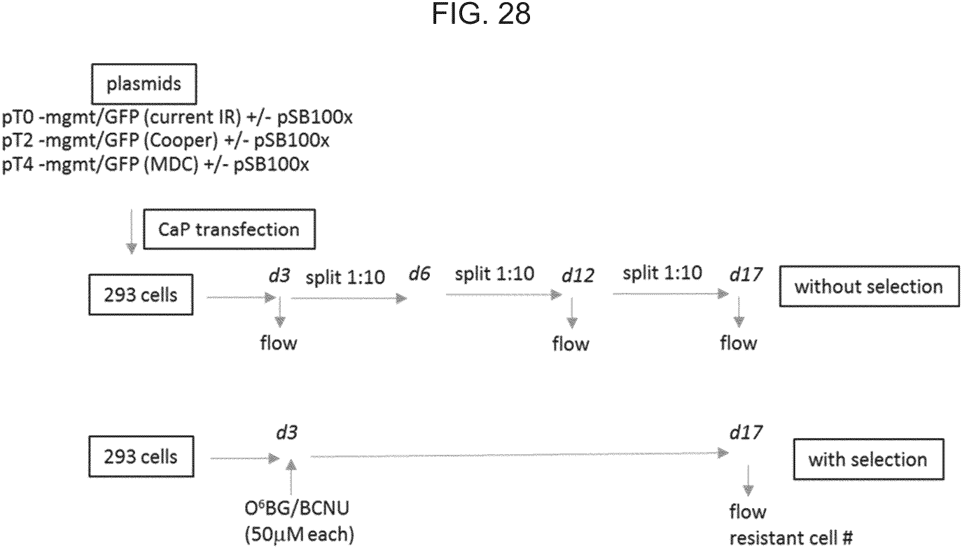

Extramedullary hemopoiesis by hematoxylin/eosin staining in liver and spleen sections from CD46tg and CD46^{+/+}/Hbb^{th-3} mice prior to administration of an adenoviral donor vector (FIG. 27). Iron deposition is shown by Perl’s staining as cytoplasmic blue pigments of hemosiderin in spleen.

In summary, the ex vivo and in vivo HSPC transduction studies with CD46-transgenic mice as well as the in vitro studies with human HSPCs demonstrated a superiority of the vector containing the long LCR. The SB100x-mediated integration frequency was not compromised by the long transposon. In addition to conferring higher γ-globin expression levels, the long LCR also provided more stringent erythroid-specific expression. Importantly, after treatment with HDAd-long-LCR, less intense O⁶BG/BCNU selection was required to achieve a complete cure in a mouse model for thalassemia intermedia.

### Materials and Methods.

Component Positions: HS5➔HS1 (21.5kb): Chr11, 5292319➔5270789 (SEQ ID NO: 6); β-promoter: chr11, 5228631➔5227018 (SEQ ID NO: 7); and 3′HS1: Chr11, 5206867➔5203839 (SEQ ID NO: 102).

HDAd vectors: The generation of HDAd-SB and HDAd-short-LCR vector has been described previously (Richter et al., Blood 128: 2206-2217, 2016; Li et al., Mol Ther Methods Clin Dev 9: 142-152, 2018). For the generation of the HDAd-long-LCR vector, corresponding shuttle plasmids were based on the cosmid vector pWE15 (Stratagene, La Jolla, CA). pWE.Ad5-SB-mgmt contains the Ad5 5′ITR (nucleotides 1 through 436) and 3′ITR (nucleotides 35741 through 35938), the human EF1α promoter-mgmt(p140k)-SV40pA-cHS4 cassette derived from pBS-µLCR-γ-globin-mgmt (Wang et al., (2019) J Clin Invest 129: 598-615), SB100x-specific IR/DR sites and FRT sites. The GFP-BGHpA fragment in the pAd.LCR-β-GFP (containing a 21.5-kb human β-globin LCR (Wang etal., (2005) J Virol 79: 10999-11013) was replaced by the human γ-globin gene and its 3′UTR region (Chr 11:5,247,139 → 5,249,804) (pAd-long-LCR-β-γ-globin). The plasmid pAd-long-LCR-β-γ-globin contains a 21.5-kb human β-globin LCR and 3.0-kb human β-globin 3′HS1. The 28.9-kb fragment containing LCR-β-γ-globin-3′HS1 was inserted downstream of the cassette of EF1α-mgmt-SV40pA-cHS4 into pWE.Ad5-SB-mgmt (pWE.Ad5-SB-long-LCR-γ-globin/mgmt). The complete long-LCR-γ-globin/mgmt cassette was flanked by SB100x-specific IR/DR sites and FRT sites. The resulting plasmids were packaged into phages using Gigapack III Plus Packaging Extract (Stratagene, La Jolla, CA) and propagated. To generate the HD-Ad-long-LCR-γ-globin/mgmt virus, the viral genomes were released by I-Ceul digestion from the plasmid for rescue in 116 cells. There are two known variants of the HBG1 gene in the human population with a single amino acid variation (76-lsoleucine or 76-Threonine). The 76-Ile HBG1 variant was used which has a range in frequency from 13% in Europeans to 73% in East Asians.

To generate HDAd viruses, the viral genomes were released by Fsel digestion from the plasmid for rescue in 116 cells (Palmer et al. Mol Ther 8: 846-852, 2003) with Ad5/35++-Acr helper virus. This helper virus is a derivative of AdNG163-5/35++, an Ad5/35++ helper vector containing chimeric fibers composed of the Ad5 fiber tail, the Ad35 fiber shaft, and the affinity-enhanced Ad35++ fiber knob (Richter, et al., (2016) Blood 128: 2206-2217). A human codon-optimized AcrIIA4-T2A-AcrIIA2 sequence that was recently shown to inhibit SpCas9 activity was synthesized (Li et al., Mol Ther Methods Clin Dev 9: 390-401, 2018) and cloned into a shuttle plasmid pBS-CMV-pA (pBS-CMV-Acr-pA). Subsequently, the 2.0-kb CMV-Acr-pA cassette was amplified from pBS-CMV-Acr-pA and inserted into the Swal sites of pNG163-2-5/35++ (Richter et al., Blood 128: 2206-2217 2016) by In-Fusion HD cloning kit (Takara). The viral genome was then released by Pacl digestion and the Ad5/35++-Acr helper virus was rescued and propagated in 293 cells. The Ad5/35++-Acr helper virus contains chimeric fibers composed of the Ad5 fiber tail, the Ad35 fiber shaft, and the affinity-enhanced Ad35++ fiber knob (Wang et al., J Virol 82: 10567-10579, 2008). The generation of HDAd-SB has been described previously (Richter et al., Blood 128: 2206-2217, 2016). Helper virus contamination levels were below 0.05%. All preparations were free of bacterial endotoxin.

CD34⁺ cell culture: CD34⁺ cells from G-CSF-mobilized adult donors were recovered from frozen stocks and incubated overnight in Iscove’s modified Dulbecco’s medium (IMDM) supplemented with 10% heat-inactivated FCS, 1% BSA 0.1 mmol/l 2-mercaptoethanol, 4 mmol/l glutamine and penicillin/streptomycin, Flt3 ligand (Flt3L, 25 ng/ml), interleukin 3 (10 ng/ml), thrombopoietin (TPO) (2 ng/ml), and stem cell factor (SCF) (25 ng/ml). Flow cytometry demonstrated that >98% of cells were CD34-positive. Cytokines and growth factors were from Peprotech (Rocky Hill, NJ). CD34⁺ cells were transduced with virus in low attachment 12 well plates.

Erythroid in vitro differentiation: Differentiation of human HSPCs into erythroid cells were carried out based on the protocol described in Douay et al., Methods Mol Biol 482: 127-140, 2009. In brief, in step 1, cells at a density of 10⁴ cells/ml were incubated for 7 days in IMDM supplemented with 5% human plasma, 2 IU/ml heparin, 10 µg/ml insulin, 330 µg/ml transferrin, 1 µM hydrocortisone, 100 ng/ml SCF, 5 ng/ml IL-3, 3 U/ml erythropoietin (Epo), glutamine, and Pen-Strep. In step 2, cells at a density of 1x10⁵ cells/ml were incubated for 3 days in IMDM supplemented with 5% human plasma, 2 IU/ml heparin, 10 µg/ml insulin, 330 µg/ml transferrin, 100 ng/ml SCF, 3 U/ml Epo, glutamine, and Pen/Strep. In step 3, cells at a density of 1x10⁶ cells/ml cells were incubated for 12 days in IMDM supplemented with 5% human plasma, 2 IU/ml heparin, 10 µg/ml insulin, 330 µg/ml transferrin, 3 U/ml Epo, glutamine, and Pen/Strep.

In vitro selection of transduced CD34+ cells: Transduced CD34+ cells were selected with O⁶BG/BCNU on day 3 in step 1 of the in vitro differentiation protocol. Briefly, CD34+ cells were incubated with 50 µM O⁶BG for one hour and then incubated with 35 µM BCNU for another two hours. Cells were then washed twice and resuspended in fresh step 1 medium.

Lin⁻ cell culture: Lineage negative cells were isolated form total mouse bone marrow cells by MACS using the Lineage Cell Depletion kit from Miltenyi Biotech (Bergisch Gladbach, Germany). Lin⁻ cells were cultured in IMDM supplemented with 10% FCS, 10% BSA, Pen-Strep, glutamine, 10 ng/ml human TPO, 20 ng/ml mouse SCF and 20 ng/ml human Flt-3L.

Globin HPLC: Individual globin chain levels were quantified on a Shimadzu Prominence instrument with an SPD-10AV diode array detector and an LC-10AT binary pump (Shimadzu, Kyoto, Japan). A 40%-60% gradient mixture of 0.1% trifluoroacetic acid in water/acetonitrile was applied at a rate of 1 mL/min using a Vydac C4 reversed-phase column (Hichrom, UK).

Flow cytometry: Cells were resuspended at 1x10⁶ cells/100 µL in PBS supplemented with 1 % FCS and incubated with FcR blocking reagent (Miltenyi Biotech, Auburn CA) for ten minutes on ice. Next the staining antibody solution was added in 100 µL per 10⁶ cells and incubated on ice for 30 minutes in the dark. After incubation, cells were washed once in FACS buffer (PBS, 1% FBS). For secondary staining the staining step was repeated with a secondary staining solution. After the wash, cells were resuspended in FACS buffer and analyzed using a LSRII flow cytometer (BD Biosciences, San Jose, CA). Debris was excluded using a forward scatter-area and sideward scatter-area gate. Single cells were then gated using a forward scatter-height and forward scatter-width gate. Flow cytometry data were then analyzed using FlowJo (version 10.0.8, FlowJo, LLC). For flow analysis of LSK cells, cells were stained with biotin-conjugated lineage detection cocktail (cat #: 130-092-613; Miltenyi Biotec, San Diego, CA) and antibodies against c-Kit (cat #:12-1171-83) and Sca-1 (cat #: 25-5981-82) as well as APC-conjugated streptavidin. Other antibodies from eBioscience (San Diego, CA) included anti-mouse LY-6A/E (Sca-1 )-PE-Cyanine7 (clone D7), anti-mouse CD117 (c-Kit)-PE (Clone 2B8), anti-mouse CD3-APC (clone 17A2; cat #:17-0032-82), anti-mouse CD19-PE-Cyanine7 (clone eBio1D3; cat #: 25-0193-82), and anti-mouse Ly-66 (Gr-1)-PE, (clone RB6-8C5; cat #: 12-5931-82). Anti-mouse Ter-119-APC (clone: Ter-119; cat #: 116211) was from Biolegend (San Diego, CA).

For intracellular flow cytometry detecting human γ-globin expression and real-time reverse transcription PCR methods, see Wang et al. (J. Clin Invest. 129(2):598-615, 2019).

Measurement of vector copy number: Total DNA from bone marrow cells was extracted using the Quick-DNA miniprep kit (Zymo Research). Viral DNA extracted from HDAd-short LCR-γ-globin/mgmt virus was serially diluted and used for a standard curve. qPCR was conducted in triplicate using the power SYBR Green PCR master mix on a StepOnePlus real-time PCR system (Applied Biosystems). 9.6 ng DNA (9600 pg/6 pg/cell = 1600 cells) was used for a 10 µL reaction. The following primer pairs were used: human γ-globin forward (SEQ ID NO: 86), and reverse (SEQ ID NO: 87).

Integration site analysis (LAM-PCR). For a depiction of the procedure, see FIG. 6. The randomized data for FIG. 7D was created using a Poisson Regression Insertion Model (PRIM) to calculate the expected insertion rate for non-overlapping 20 kilobase windows along the length of each chromosome in the mouse reference genome (mm9). The PRIM algorithm generated a statistical model based on the number of TA dinucleotides within each window, the chromosome in which the window resides, and the total number of unique insertions. For each window, the expected number of insertions was calculated and compared to the observed number of insertions to produce a p-value. Bonferroni-correction was then applied to identify windows that showed enrichment for detection of inserted transposons. Random sequences from the reference genome containing TA were then generated, mapped using Bowtie2 and plotted against the real integration data. Calculations and plots were made using ggplot2 in R. Figures were drawn using HOMER and ChIPseeker.

Integration site analysis (inverse PCR). Junctions in total bone marrow cells were analyzed by inverse PCR as described elsewhere with modifications (Wang et al., J Virol 79: 10999-11013, 2005). Briefly, genomic DNA from bone marrow cells was isolated by Quick-DNA™ miniprep kit (Zymo Research) following the manufacturer’s instructions. 5-10 µg of DNA was digested with Sacl and re-ligated under conditions that promote intramolecular reaction. The ligation mixture was purified with phenol/chloroform extraction and ethanol precipitation and then used for nested PCR (30 cycles each) using KOD Hot Start DNA polymerase. The following primers were used: EF1α p1 forward (SEQ ID NO: 88) and reverse (SEQ ID NO: 89); EF1α p2 forward (SEQ ID NO: 90) and reverse (SEQ ID NO: 91); 3′HS1 p1 forward (SEQ ID NO: 92) and reverse (SEQ ID NO: 93); and 3′HS1 p2 forward (SEQ ID NO: 94) and reverse (SEQ ID NO: 95).

In the above table, the underlined bases are used for downstream cloning. PCR amplicons were gel purified, cloned, sequenced and aligned to identify the integration sites.

Animals: All experiments involving animals were conducted in accordance with controlling institutional guidelines and in accordance with the Office of Laboratory Animal Welfare (OLAW) Public Health Assurance (PHS) policy, USDA Animal Welfare Act and Regulations, the Guide for the Care and Use of Laboratory Animals and the controlling Institutional Animal Care and Use Committee (IACUC) policies.

Ex vivo and in vivo HSPC transduction studies were performed with a C57BI/6-based transgenic mouse model (hCD46tg) that contained the complete human CD46 locus. These mice express hCD46 in a pattern and at a level similar to humans (Kemper et al., Clin Exp Immunol 124: 180-189, 2001).

Breeding and screening of CD46+/+/Hbb^{th3} mice: After three rounds of backcrossing, Hbb^{th3} mice homozygosity for CD46 was confirmed by PCR on gDNA [using CD46F (SEQ ID NO: 96) and CD46Rprimers (SEQ ID NO: 97) as well as by flow cytometry that allowed measuring CD46 MFI.

Bone marrow Lin⁻ cell transplantation: Recipients were female C57BL/6 mice, 6 - 8 weeks old. On the day of transplantation, recipient mice were irradiated with 1000 Rad. Four hours after irradiation 1x10⁶ Lin⁻ cells were injected intravenously through the tail vein. This protocol was used for transplantation of ex vivo transduction Lin⁻ cells and for transplantation into secondary recipients.

HSPC mobilization and in vivo transduction: This procedure was described previously in Richter et al., Blood 128: 2206-2217, 2016. HSPCs were mobilized in mice by s.c. injections of human recombinant G-CSF (5 µg/mouse/day, 4 days) (Amgen Thousand Oaks, CA) followed by an s.c. injection of AMD3100 (5 mg/kg) (Sigma-Aldrich) on day 5. In addition, animals received Dexamethasone (10 mg/kg) i.p. 16 h and 2 h before virus injection. Thirty and 60 minutes after AMD3100, animals were intravenously injected with HDAd vectors through the retro-orbital plexus with a dose of 4x10¹⁰ vp for each virus per injection. Four weeks later, in vivo selection of O⁶BG/BCNU was initiated.

Secondary bone marrow transplantation: Recipients were female C57BL/6 mice, 6-8 weeks old from the Jackson Laboratory. On the day of transplantation, recipient mice were irradiated with 1000 Rad. Bone marrow cells from in vivo transduced CD46tg mice were isolated aseptically and lineage-depleted cells were isolated using MACS. Four hours after irradiation cells were injected intravenously at 1x10⁶ cells per mouse. At week 20, secondary recipients were either sacrificed and CD46+ cells from blood, bone marrow and spleen were isolated by MACS or subjected to mobilization and in vivo transduction, as described above. All secondary recipients received immunosuppression starting at week 4.

Hematological analyses: Blood samples were collected into EDTA-coated tubes, and analysis was performed on a HemaVet 950FS (Drew Scientific).

Tissue analysis: Spleen and liver tissue sections of 2.5 µm thickness were fixed in 4% formaldehyde for at least 24 hours, dehydrated and embedded in paraffin. Staining with hematoxylin-eosin was used for histological evaluation of extramedullary hemopoiesis. Hemosiderin was detected in tissue sections by Perl’s Prussian blue staining. Briefly, the tissue sections were treated with a mixture of equal volumes (2%) of potassium ferrocyanide and hydrochloric acid in distilled water and then counterstained with neutral red. The spleen size was assessed as the ratio of spleen weight (mg)/body weight (g).

Blood analysis and bone marrow cytospins: Blood samples were collected into EDTA-coated tubes and analysis was performed on a HemaVet 950FS (Drew Scientific, Waterbury, CT) or ProCyteDx™ (IDEXX, Westbrook, Maine) machine. Peripheral blood smears were prepared and stained with May-Grünwald/Giemsa for 5 and 15 minutes respectively (Merck, Darmstadt, Germany). Suspensions of bone marrow cells were centrifuged onto slides using a cytospin device and stained with May-Grünwald/Giemsa. The investigators who counted the reticulocytes on blood smears have been blinded to the sample group allocation. Only animal numbers appeared on the slides (5 slides per animal, 5 random 1 cm² sections).

Statistical analyses: Data are presented as means ± standard error of the mean (SEM). For comparisons of multiple groups, one-way and two-way analysis of variance (ANOVA) with Bonferroni post-testing for multiple comparisons was employed. Differences between groups for one grouping variable were determined by the unpaired, two-tailed Student’s t-test. For non-parametric analyses the Kruskal-Wallis test was used. Statistical analysis was performed using GraphPad Prism version 6.01 (GraphPad Software Inc., La Jolla, CA). *p≤0.05, ** p≤0.0002, ***p ≤0.00003. A P value less than 0.05 was considered significant.

Discussion. One of these, the human ß-globin gene cluster lies in chromosome 11 and spans ~100 kb. It has been proposed that the β-globin locus forms an erythroid-specific spatial structure composed of cis-regulatory elements and active β-globin genes, termed the active chromatin hub (ACH) (Tolhius et al., Mol Cell, 10:1453-1465, 2002). A core ACH is developmentally conserved and includes the upstream 5′ DNAse hypersensitivity regions 1 to 5, called the globin LCR, and the downstream 3′HS1 as well as erythroid-specific transacting factors (Kim et al., Mol Cell Biol., 27:4551-65, 2007). For gene therapy applications, it is notable that a 23 kb β-globin LCR containing HS1 to HS5 plus a 3 kb 3′HS1 region conferred high-level, erythroid-specific, position independent expression upon cis-linked genes in transgenic mice (Grosveld, Cell, 51 :975-985, 1987). A tool to deliver a transgene under the control of this LCR is available with 30+ kb HDAd vectors.

The correction of many genetic diseases requires high level and tissue-restricted expression of the therapeutic gene, which can be accomplished by employing LCRs (Li et al., Blood 100: 3077-3086, 2002). For a cure of β-thalassemia major and Sickle Cell Anemia, it is thought that around 20% gene marking in HSPCs and 20% therapeutic-globin chain (β- or γ-globin) production in erythroid cells are required (Fitzhugh et al., Blood 130: 1946-1948, 2017). Due to size limitations, only truncated forms of the β-globin LCR can be used in lentivirus vectors which makes it difficult to meet the requirements for corrective gene expression levels (Uchida et al., Nat Commun 10: 4479, 2019). A strategy to increase expression levels after lentivirus-mediated HSPC transduction is to increase the vector dose and thus the number of integrated transgene copies. This approach however enhances the risk of genotoxicity and tumorigenicity. Other attempts are focused on further optimizing globin expression cassettes (Uchida et al., Nat Commun 10: 4479, 2019). HDAd vectors, having an insert capacity of 30 kb, are an ideal tool to develop the latter concept. In this Example, a HDAd5/35++ vector carrying a 29 kb γ-globin expression cassette was generated and tested after in vitro and in vivo HSPC transduction in CD46-transgenic mice.

In the HDAd vector system, the integration of the γ-globin cassette is mediated by the SB100x transposase. Non-viral gene transfer using the SB/transposon system is being used clinically for CD19 CAR T-cell therapy (Kebriaei et al., J Clin Invest 126: 3363-3376, 2016), age-related macular degeneration (Hudecek et al., Crit Rev Biochem Mol Biol 52: 355-380, 2017; Thumann etal., Mol Ther Nucleic Acids 6: 302-314, 2017), and Alzheimer’s disease (Eyjolfsdottir et al., Alzheimers Res Ther 8: 30, 2016). HD-Ad mediated SB gene transfer was pioneered by the Kay and Ehrhardt groups. In their studies, transposons were relatively small; 4 kb-6 kb (Hausl et al., Mol Ther 18: 1896-1906, 2010; Yant et al., Nat Biotechnol20: 999-1005, 2002). The current Example demonstrates for the first time that SB100x is capable of integrating a 32.4kb transposon at an efficacy comparable to that of a 11.8kb transposon, based on comparable VCNs (2-3 copies per cell). Per se this finding contradicts the observation that the efficacy of SB-mediated integration inversely correlates with the size of the SB transposon (Karsi et al., Mar Biotechnol (NY) 3: 241-245, 2001). The system appears to be lifted from the size limitation. First, in order to form a catalytically primed transposon/transposase complex, the two ends of the transposon must be held together in close physical proximity by transposase molecules (Hudecek etal., Crit Rev Biochem Mol Biol 52: 355-380, 2017). This limitation has been addressed by incorporating frt sides into the HDAd vector, which are recognized by the co-expressed Flpe recombinase, leading to a circularization of the transposon (Yant et al., Nat Biotechnol.. 20: 999-1005, 2002). The second mechanism limiting transposition of large constructs is a suicidal transpositional mechanism called auto-integration, i.e. the integration into TA dinucleotide inside the transposon (Wang et al., PLoS Genet 10: e1004103, 2014). The unseen differences in the VCN between HDAd-short-LCR and HDAd-long-LCR could be related to in vivo selection, which enriches for HSPCs and progenitors with a certain level of mgtm^{P140K} expression, i.e. for cells that have reached a threshold VCN.

Because of the powerful O⁶BG/BCNU in vivo selection system, nearly 100% of peripheral blood erythrocytes contained γ-globin. While this in vivo selection approach does not affect the cellular composition in the bone marrow, it results in leukopenia. Efforts are therefore focused on alternative approaches that do not involve the cytotoxic drug BCNU. Notably, as supported by the studies in the murine thalassemia model (Wang et al., J Clin Invest 129: 598-615, 2019), pharmaceutical in vivo selection might not be necessary in patients with hemoglobinopathies because gene-corrected HSPCs will have a proliferative advantage over non-corrected cells (Perumbeti etal., Blood 114: 1174-1185, 2009).

Given comparable VCNs for HDAd-short-LCR and HDAd-long-LCRs in primary animals and secondary recipients, γ-globin levels (measured by HPLC and qRT-PCR) in RBCs and bone marrow erythroid progenitors were significantly higher for the vector containing the long LCR. Interestingly, the differences between the two vectors were more pronounced in secondary recipients. This implies that RBCs that originated from transduced long-term repopulating HSPCs have higher γ-globin levels. Furthermore, HDAd-long-LCR displayed stronger erythroid specificity. These effects can be attributed to the additional LCR elements in HDAd-long-LCR that result in better access for transcription factors due to the LCR’s chromatin opening ability (Li et al., Blood 100: 3077-3086, 2002), and/or the binding of additional transcription factors that result in increased transcription of the γ-globin gene. Another feature of the LCR is noteworthy, namely its ability to act as an autonomous regulatory unit, implying less transactivation of neighboring genes after random integration. In this context using a more complete LCR version decreases potential genotoxicity of the approach.

In summary, the current Example describes, among other things, a vector that after in vivo HSPC transduction in mice confers γ-globin levels that meet gene expression thresholds thought to be curative for thalassemia major and Sickle Cell Anemia.

### Example 2: SB Transposase ITRs

The present Example compares marking of target cells by a transposon payload encoding GFP and an MGMT^{P140K} selectable marker, where the transposon payload was flanked by three different SB ITRs. The present Example includes three plasmids in which the mgmt/GFP transposon payload is flanked by (i) pT0 ITRs; (ii) pT2 ITRs; or (iii) pT4 ITRs, which plasmids were otherwise identical. In this Example, 293 cells were transfected with the three plasmids including the mgmt./GFP transposon payload, with or without a support plasmid encoding pSB100x. T2 is an IR developed by Cooper lab and currently used clinically for CAR T-cell therapy (Srour et al., Blood 235(11):862-865, 2020; PMID 31961918). T4 is another version of an IR developed by the Izcvak lab (Kebriaei et al., Trends Genet. 33(11)852-870, 2017; PMID: 28964527). The inventors are not aware of any prior side-by-side comparison of T0, T2, and T4.

Cells were cultured for 17 days with or without selection. Culture samples were drawn on days 3, 12, and 17 for cells not under selection, and on day 17 for cells under selection by a single addition of 50 µM O⁶BG/BCNU on day 3 (see FIG. 28). In one series, the cells were passaged 1:10 at days 3, 6, and 12 to eliminate episomal plasmids. GFP expression (analyzed at day 17) represents expression from integrated transposons. In another series, an O⁶BG/BCNU selection step was included to enrich for cells with integrated mgmt.

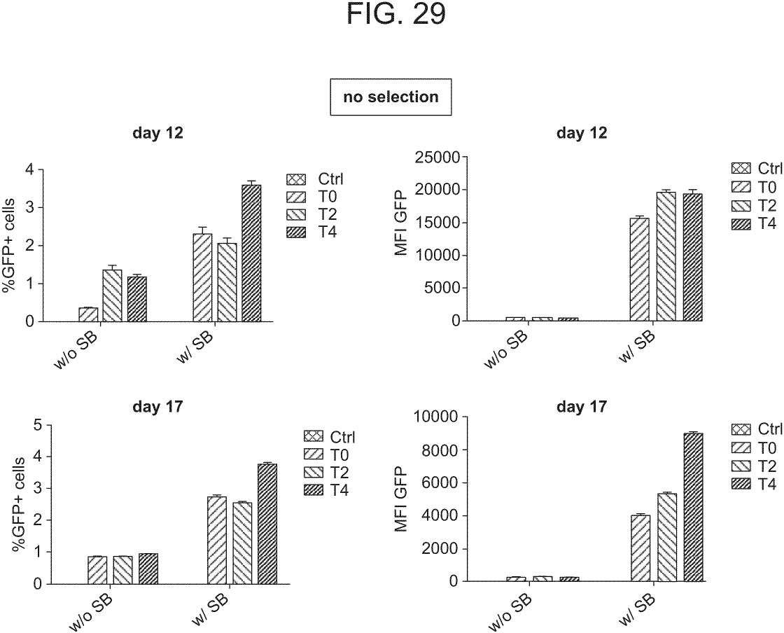

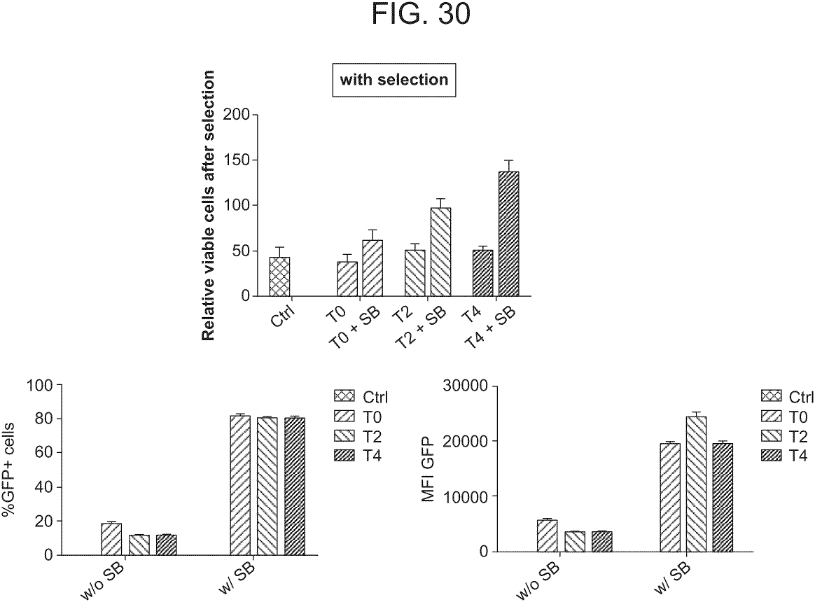

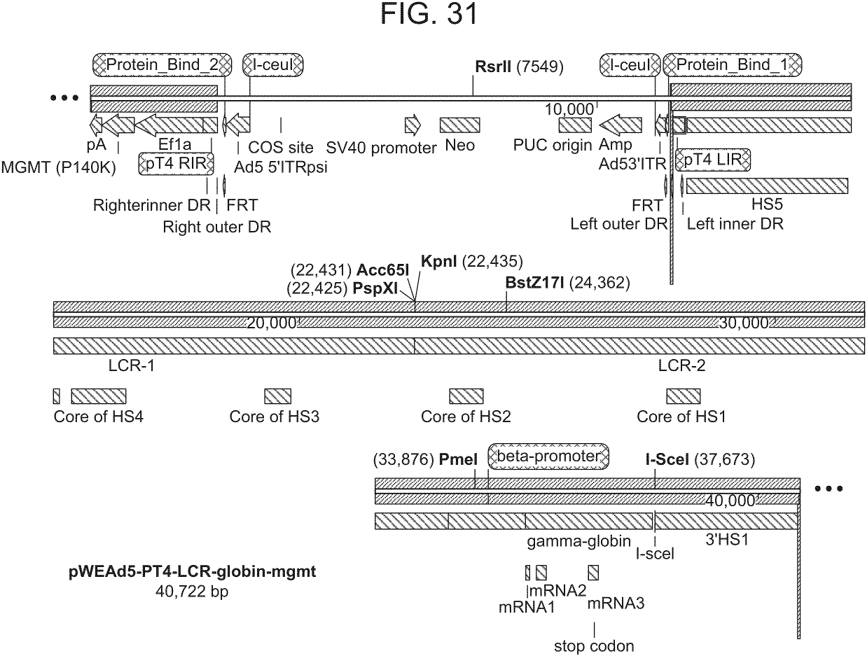

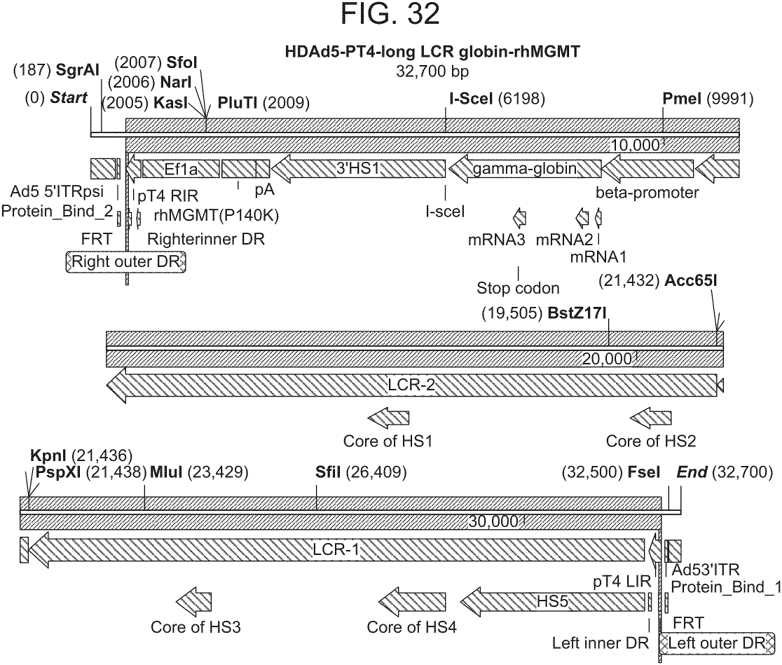

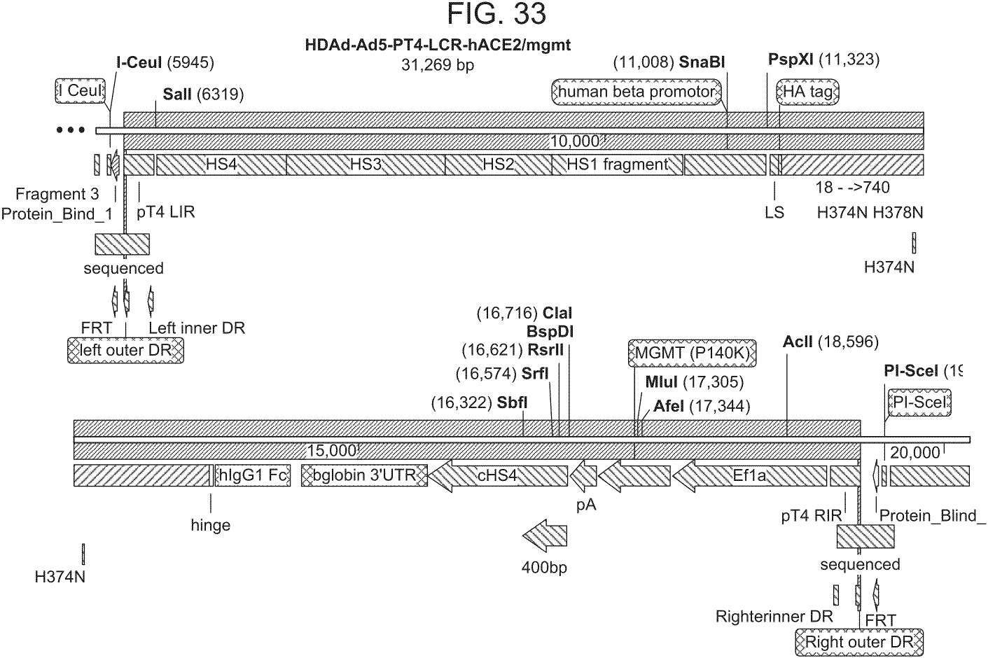

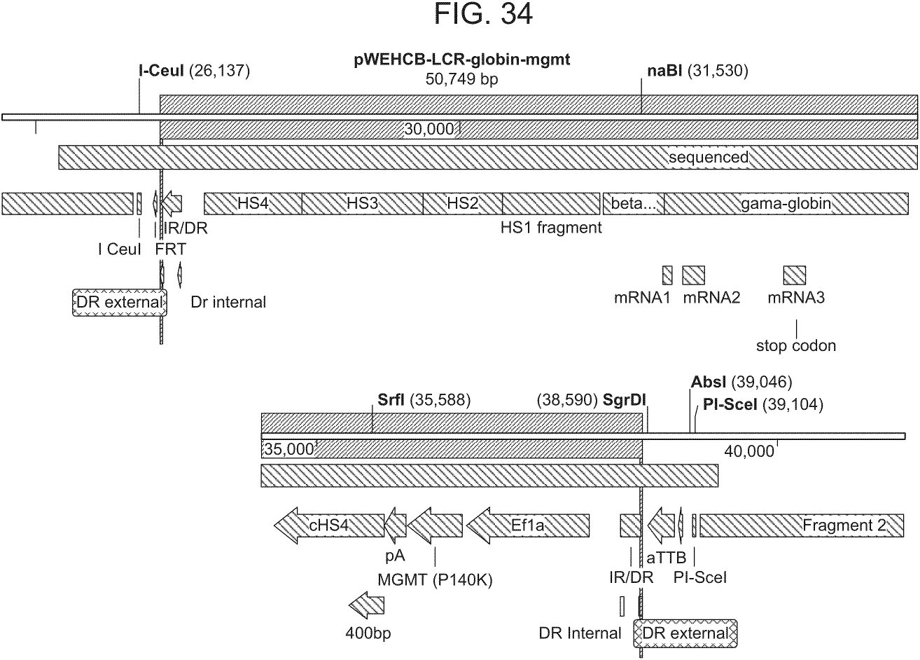

Cells were analyzed for GFP by flow cytometry. In the absence of SB100x, GFP expression originates from residual episomal plasmids and, as expect, no difference was observed. FIG. 29 shows the percentage of GFP-expressing 293 cells on days 12 and 17 of culture for cells cultured with or without SB100x plasmid for each of the T0, T2, and T4 plasmids. In the presence of SB100x, integration occurred. The percentage of GFP+ cells was comparable for T0 and T2, but significantly higher for T4 (p<0.01). The GFP MFI reflects the GFP expression level, i.e. the number of integrated transposon copies per cell. Again, the MFI for T4 was significantly higher. There was also a significant difference between T0 and T2. In conclusion, while all IRs may be suitable of use in methods and compositions of the present disclosure, including gene therapy, the T4 IR is superior in mediating SB100x integration. FIG. 30 shows the percentage of GFP-expressing 293 cells on day 17 of culture for cells under selection with O⁶BG/BCNU for cells cultured with or without SB100x plasmid for each of T0, T2, and T4 plasmids. Relative number of resistant cells. O6^{B}G/BCNU selection should kill cells where transposon (GFP/mgtm) integration did not occur. The background of surviving cells without SB is probably due to episomal vector. In the presence of SB, the difference between T0 vs T2 and T2 vs T4 was significant, again underscoring the superiority of T4. As expected, GFP expression should be comparable in all cells that survived O⁶BG/BCNU selection.

### Example 3: Transposons Engineered for Efficient Integration

The present Example provides exemplary transposon payloads that can be efficiently integrated into target cell genomes. Exemplified transposons have lengths ranging from 2.8 to 31.8 kb and efficient integration will be observed across the provided range of transposon lengths in accordance with the present invention. Transposons of the present Example are flanked by Sleeping Beauty IRs that can be targeted by Sleeping Beauty transposases including without limitation SB100x. Comparison of a transposon provided in the present Example to a shorter transposon of the present Example (or other reference transposon) will not demonstrate length dependence, and/or will demonstrate a degree of length dependence that that is lower than would be expected by a person of skill in the art, based on the frequency and/or efficiency of integration. In various embodiments, for example, the frequency and/or efficiency of integration can be measured by the number of transposon integration events per target genome and/or by the number of target genomes that include at least one (or at least two, or at least three) transposon integration events.

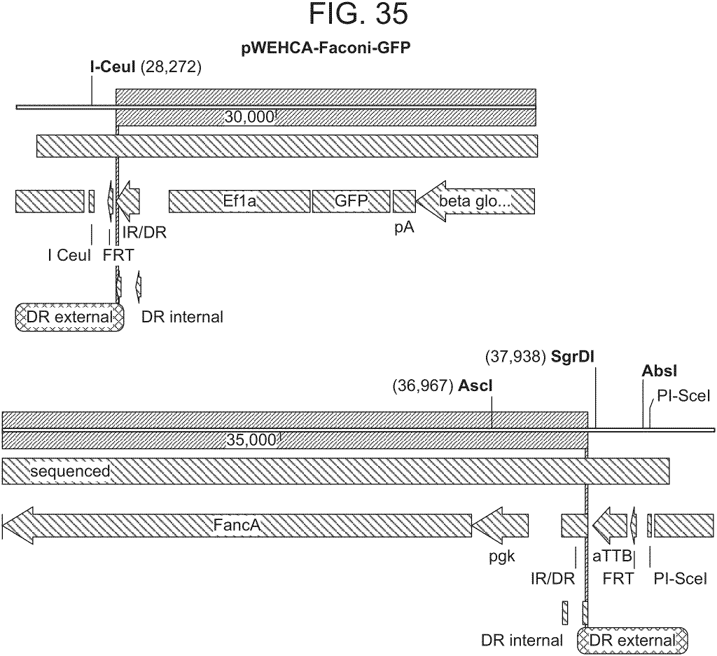

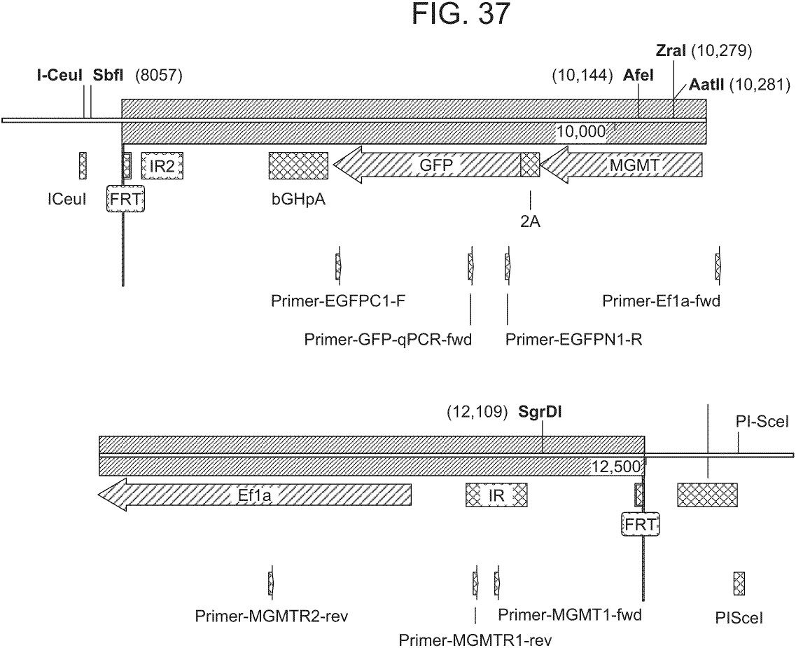

Various exemplary transposon payloads are provided in FIGS. 31-43. Certain of the representations provided in the figures include a transposon payload in a circularized plasmid format. Those of skill in the art will appreciate that the transposon payload can be readily utilized, using techniques of molecular biology, in other contexts, e.g., in a viral vector genome.

The present Example includes a nucleic acid referred to herein as PWEAd5-PT4LCR-globin/mgmt or pWEAd5-PT4-LCR-globin-mgmt, which includes a transposon having a length of 31.776 kb (FIG. 31). The transposon payload is flanked by transposon inverted repeats (IRs, in particular Sleeping Beauty IRs), which are in turn flanked by recombinase direct repeats (DRs, in particular FRT DRs). The transposon includes: (i) a gamma-globin coding sequence operably linked with a beta promoter, a long LCR including HS1-HS5, and a 3′HS1 and (ii) an MGMT^{P140K} selection cassette in which an MGMT^{P140K} coding sequence is operably linked with an Ef1a promoter.

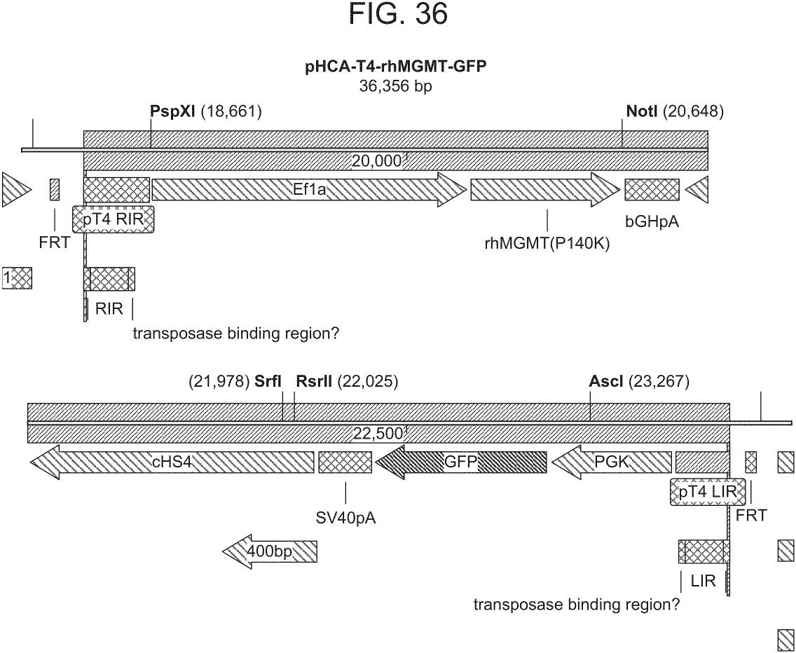

The present Example includes a nucleic acid referred to herein as HDAd5-PT4-long LCR globin-rhMGMT which includes a transposon having a length of 31.772 kb (FIG. 32). The transposon payload is flanked by transposon inverted repeats (IRs, in particular Sleeping Beauty IRs), which are in turn flanked by recombinase direct repeats (DRs, in particular FRT DRs). The transposon includes: (i) a gamma-globin coding sequence operably linked with a beta promoter, a long LCR including HS1-HS5, and a 3′HS1 and (ii) an MGMT^{P140K} selection cassette in which an MGMT^{P140K} coding sequence is operably linked with an Ef1a promoter.

The present Example includes a nucleic acid referred to herein as HDAd-Ad5-PT4-LCR-hACE2/mgmt which includes a transposon having a length of 13.173 kb (FIG. 33). The transposon payload is flanked by transposon inverted repeats (IRs, in particular pT4 Sleeping Beauty IRs), which are in turn flanked by recombinase direct repeats (DRs, in particular FRT DRs). The transposon includes: (i) a recombinant human ACE2 coding sequence operably linked with a beta promoter, and an LCR including HS1-HS4 and (ii) an MGMT^{P140K} selection cassette in which an MGMT^{P140K} coding sequence is operably linked with an Ef1a promoter.

The present Example includes a nucleic acid referred to herein as pWEHCB-microLCR-globin/mgmt which includes a transposon having a length of 12.169 kb (FIG. 34). The transposon payload is flanked by transposon inverted repeats (IRs, in particular Sleeping Beauty IRs), which are in turn flanked by recombinase direct repeats (DRs, in particular FRT DRs). The transposon includes: (i) a gamma globin coding sequence operably linked with a beta promoter, and a micro LCR including HS1-HS4 and (ii) an MGMT^{P140K} selection cassette in which an MGMT^{P140K} coding sequence is operably linked with an Ef1a promoter.

The present Example includes a nucleic acid referred to herein as pWEHCA-Faconi-GFP which includes a transposon having a length of 9.382 kb (FIG. 35). The transposon payload is flanked by transposon inverted repeats (IRs, in particular Sleeping Beauty IRs), which are in turn flanked by recombinase direct repeats (DRs, in particular FRT DRs). The transposon includes: (i) a FancA coding sequence operably linked with a pgk promoter and (ii) a GFP coding sequence operably linked with an Ef1a promoter.

The present Example includes a nucleic acid referred to herein as pHCA-T4-rhMGMT-GFP which includes a transposon having a length of 5.49 kb (FIG. 36). The transposon payload is flanked by transposon inverted repeats (IRs, in particular pT4 Sleeping Beauty IRs), which are in turn flanked by recombinase direct repeats (DRs, in particular FRT DRs). The transposon includes: (i) a GFP coding sequence operably linked with a PGK promoter and (ii) an MGMT^{P140K} selection cassette in which an MGMT^{P140K} coding sequence is operably linked with an EF1a promoter.

The present Example includes a nucleic acid which includes a transposon having a length of 3.797 kb (FIT. 37). The transposon payload is flanked by transposon inverted repeats (IRs, in particular Sleeping Beauty IRs), which are in turn flanked by recombinase direct repeats (DRs, in particular FRT DRs). The transposon includes: (i) a GFP coding sequence and (ii) an MGMT^{P140K} coding sequence, operably linked with an EF1a promoter.

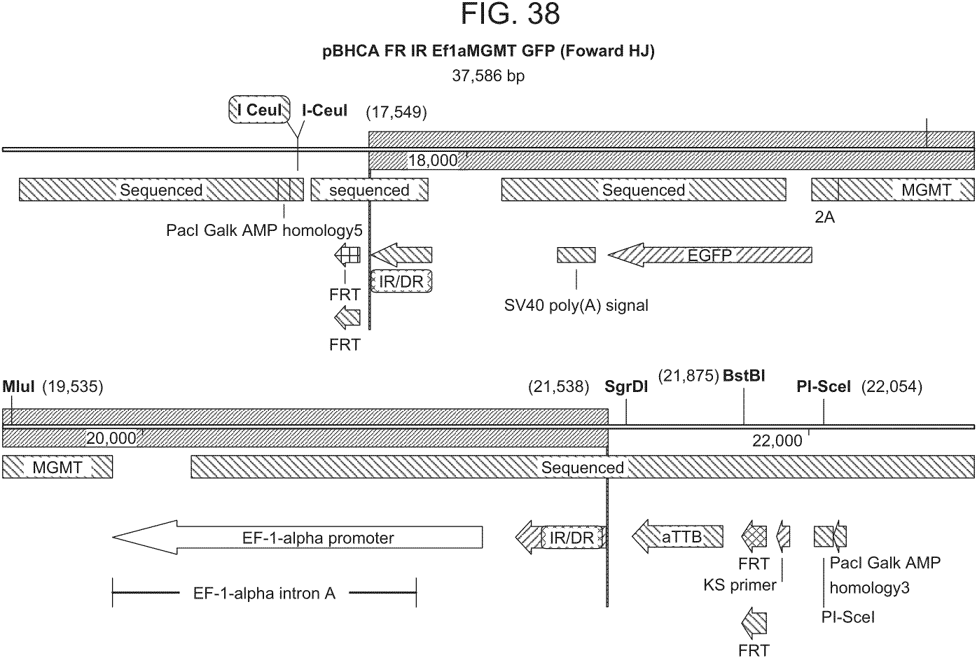

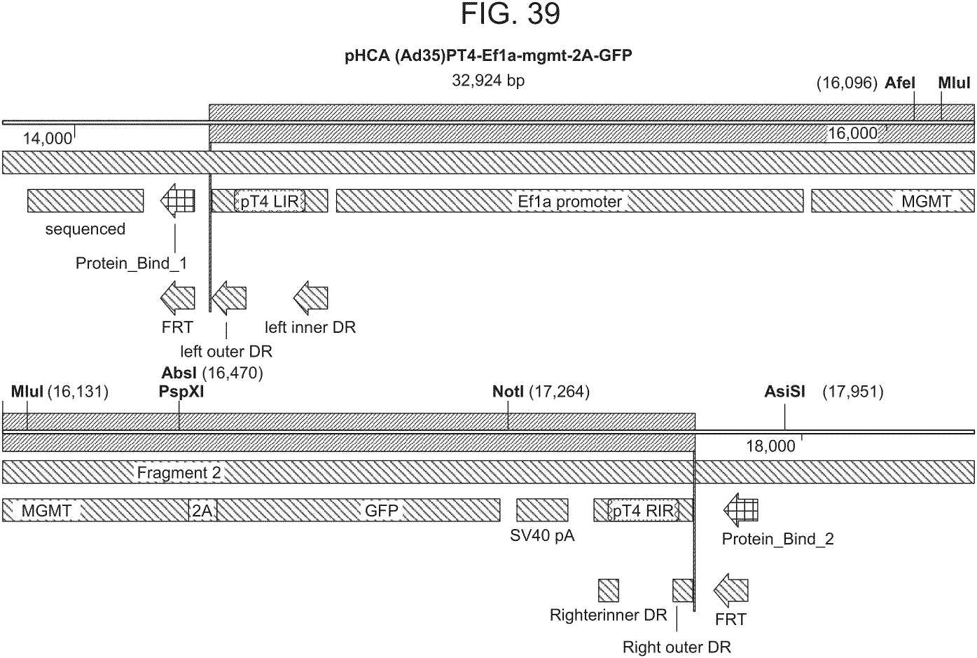

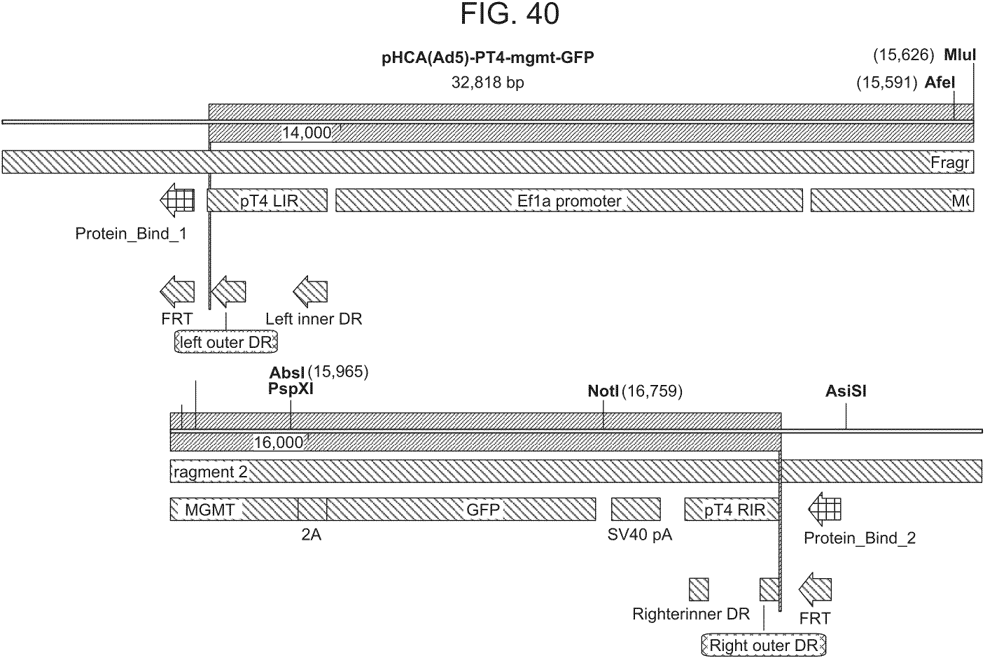

The present Example includes a nucleic acid referred to herein as pBHCA-PT0-EF1a-mgmt/GFP which includes a transposon having a length of 3.709 kb (FIG. 38). The transposon payload is flanked by transposon inverted repeats (IRs, in particular pT0 Sleeping Beauty IRs), which are in turn flanked by recombinase direct repeats (DRs, in particular FRT DRs). The transposon includes: (i) an eGFP coding sequence and (ii) an MGMT^{P140K} coding sequence, operably linked with an EF1a promoter.

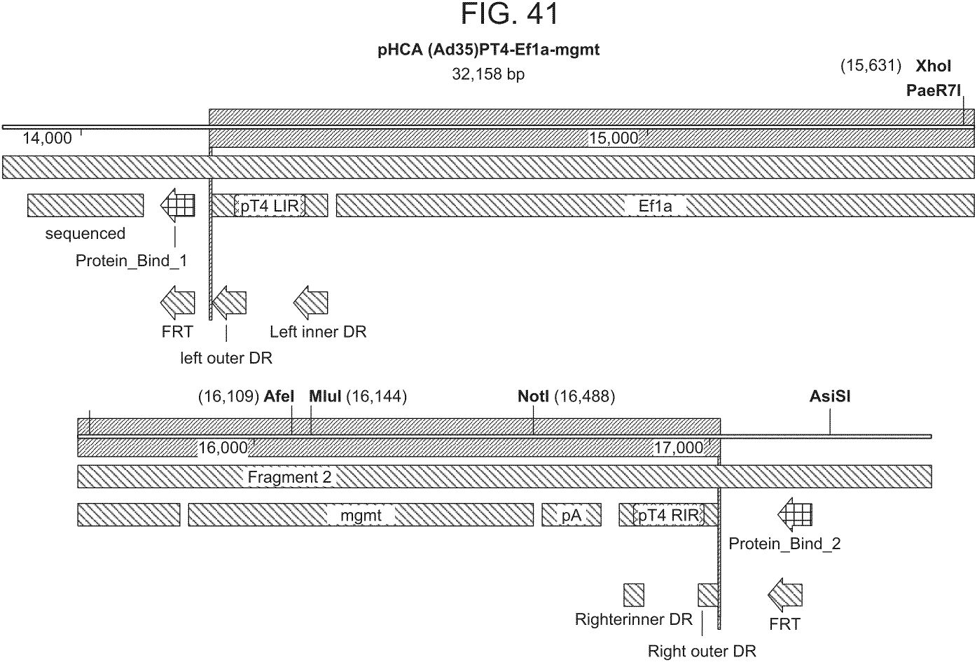

The present Example includes a nucleic acid referred to herein as pHCA(Ad35)-PT4-EF1a-mgmt/GFP which includes a transposon having a length of 3.547 kb (FIG. 39). The transposon payload is flanked by transposon inverted repeats (IRs, in particular pT4 Sleeping Beauty IRs), which are in turn flanked by recombinase direct repeats (DRs, in particular FRT DRs). The transposon includes: (i) a GFP coding sequence and (ii) an MGMT^{P140K} coding sequence, operably linked with an EF1α promoter.

The present Example includes a nucleic acid referred to herein as pHCA-Ad5-PT4-Ef1a-mgmt/GFP which includes a transposon having a length of 3.543 kb (FIG. 40). The transposon payload is flanked by transposon inverted repeats (IRs, in particular pT4 Sleeping Beauty IRs), which are in turn flanked by recombinase direct repeats (DRs, in particular FRT DRs). The transposon includes: (i) a GFP coding sequence and (ii) an MGMT^{P140K} coding sequence, operably linked with an EF1a promoter.

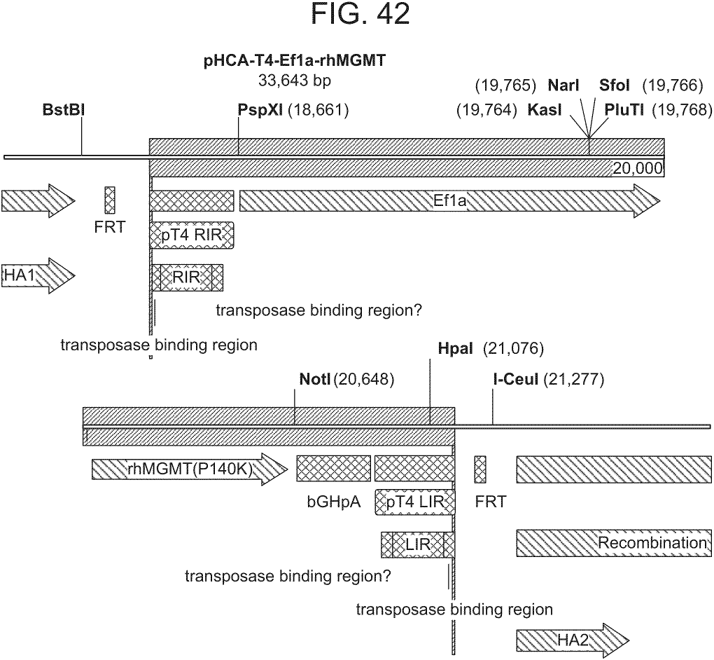

The present Example includes a nucleic acid referred to herein as pHCA(Ad35)-PT4-EF1a-mgmt which includes a transposon having a length of 2.781 kb (FIG. 41). The transposon payload is flanked by transposon inverted repeats (IRs, in particular pT4 Sleeping Beauty IRs), which are in turn flanked by recombinase direct repeats (DRs, in particular FRT DRs). The transposon includes: an MGMT^{P140K} selection cassette in which an MGMT^{P140K} coding sequence is operably linked with an EF1a promoter.

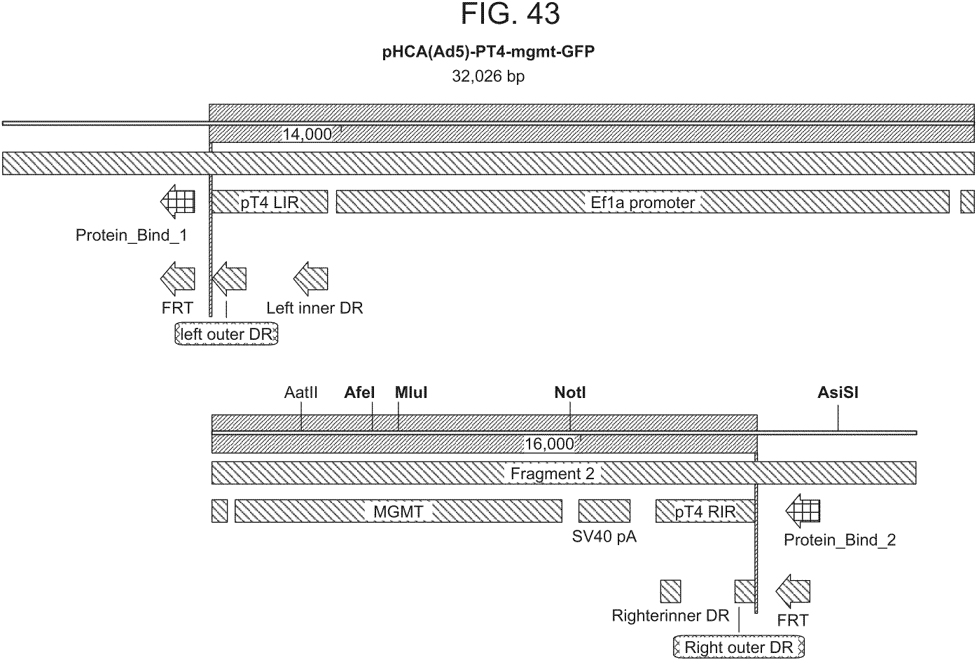

The present Example includes a nucleic acid referred to herein as pHCA-T4-Ef1a-rhMGMT which includes a transposon having a length of 2.777 kb (FIG. 42). The transposon payload is flanked by transposon inverted repeats (IRs, in particular pT4 Sleeping Beauty IRs), which are in turn flanked by recombinase direct repeats (DRs, in particular FRT DRs). The transposon includes: an MGMT^{P140K} selection cassette in which an MGMT^{P140K} coding sequence is operably linked with an EF1a promoter.

The present Example includes a nucleic acid referred to herein as pHCA-Ad5-PT4-Ef1a-mgmt which includes a transposon having a length of 2.751 kb (FIG. 43). The transposon payload is flanked by transposon inverted repeats (IRs, in particular pT4 Sleeping Beauty IRs), which are in turn flanked by recombinase direct repeats (DRs, in particular FRT DRs). The transposon includes: an MGMT^{P140K} selection cassette in which an MGMT^{P140K} coding sequence is operably linked with an EF1a promoter.

### (XII) Closing Paragraphs

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect, in this context, is any change in composition or method that reduces the ability of an adenoviral vector to carry a large transposon payload, and/or integrate a large payload into a target genome.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials is individually incorporated herein by reference in its entirety for their referenced teaching(s).

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster’s Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

### SUMMARY OF SEQUENCES

The nucleic acid and/or amino acid sequences described herein are shown using standard letter abbreviations, as defined in 37 C.F.R. §1.822. Only one strand of each nucleic acid sequence is shown, but the complementary strand is understood as included in embodiments where it would be appropriate. A computer readable text file, entitled “F053-0126US_SeqList.txt” created on or about May 22, 2023, with a file size of 136 KB, contains the sequence listing for this application and is hereby incorporated by reference in its entirety. In the accompanying Sequence Listing:

SEQ ID NO: 1 is the nucleotide sequence of a 5′ end vector sequence, Sleeping Beauty IR/DR sequence, integration junction (chr15, 6805206), shown in FIG. 2C.

CCCTGGGATTCCCCAAGGCAGGGGCGAGTCCTTTTGTATGAATTACTCAA
ATCGATAACTAGAAACTTAATTAACAACGAGATCTTATAATTTGCATACT
TCTGCCTGCTGGGGACTTTCCACACCCTAGCTGACACAAGAATTTGAAAT
ACATCCACAGGTACACCTCCAATTGACTCAAATGATGTCAATTAGTCTAT
CATAATCTTCTAAAGCCATGACATCATTTTAACTGGAATTTTCCAAGCTG TTTAAAGGCACAGTCAACTTAGTGTATGTAAACTTCTGACCCACTGGAAT TGTGATACAGTGAATTATAAGTGAAATAATCTGTCTGTAAACAATTGTTG GAAAAATGACTTGTGTCATGCACAAAGTAGATGTCCTAACTGACTTGCCA AAACTATTGTTTGTTAACAAGAAATTTGTGGAGTAGTTGAAAAACGAGTT TTAATGACTCCAACTTAAGTGTATGTAAACTTCCGACTTCAACTG[TA]A GAATGGCCCATTCATCTATAGTAGCACACAATATTTGCATTTGTGCGACA GTATAAGGGACAATTATGCTATCAGGCATTTTTCCAAAGTGAGTAATCGA AGTTTTTATACCTTTGTGTGCCATGTTTGCTACCATGGTGGGATAATCTT ACACGCGTTCTCGCGACCGGCCAGGAAAGACGCAACAAACCGGAATCTTC TGCGGCAAAAGCTTTATTGCTT

SEQ ID NO: 2 is the nucleotide sequence of a 5′ end vector sequence, Sleeping Beauty IR/DR sequence, integration junction (chrX, 16897322), shown in FIG. 2C.

TAGAAACTTAATTAACAACGAGATCTTATAATTTGCATACTTCTGCCTGC
TGGGGACTTTCCACACCCTAGCTGACACAAGAATTTGAAATACATCCACA
GGTACACCTCCAATTGACTCAAATGATGTCAATTAGTCTATCATAATCTT
CTAAAGCCATGACATCATTTTAACTGGAATTTTCCAAGCTGTTTAAAGGC
ACAGTCAACTTAGTGTATGTAAACTTCTGACCCACTGGAATTGTGATACA GTGAATTATAAGTGAAATAATCTGTCTGTAAACAATTGTTGGAAAAATGA CTTGTGTCATGCAAAGTAGATGTCCTAACTGACTTGCCAAAACTATTGTT TGTTAACAAGAAATTTGTGGAGTAGTTGAAAAACGAGTTTTAATGACTCC AACTTAAGTGTATGTAAACTTCCGACTTCAACTG[TA]CAAGTAGACCAA ATATCCATATACATAAAAGAAAAAAATAGAAAAAATTTCTAGTGACAGAA AAATGACAAAGAACATACTGTTTATTACTACTATTAAGATGTTTGCTTCC ATTACACTCATATGAGTCATGATATTTTTTCTTCATTTTTTTCTANTNNC ACTNGAAAT

SEQ ID NO: 3 is the nucleotide sequence of a 3′ end vector sequence, Sleeping Beauty IR/DR sequence, integration junction (chr4, 10207667), shown in FIG. 2C.

GTTGCTAGGAATGAGCCAAATTCATCTGTATTAAACAGTGGGAGCTTGTG
GAAGGCTACTCGAAATGTTTGACCCAAGTTAAACAATTTAAAGGCAATGC
TACCAAATACTAATTGAGTGTATGTTAACTTCTGACCCACTGGGAATGTG ATGAAAGAAATAAAAGCTGAAATGAATCATTCTCTCTACTATTATTCTGA TATTTCACATTCTTAAAATAAAGTGGTGATCCTAACTGACCTTAAGACAG GGAATCTTTACTCGGATTAAATGTCAGGAATTGTGAAAAAGTGAGTTTAA ATGTATTTGGCTAAGGTGTATGTAAACTTCCGACTTCAACTG[TA]TATC CTCCCCGTTGCACCCTCTTGATGATGCTGAGATGAACACAGATGCTCACT CCTTGAGGGCTCTAAGCTTATGCTGACACAGACACAGGTGCTCACTTCTA TGAATGGCCTAAGATTTGAGGACATCATGAGGACAAGTGTGATAAAATCT TGGAACAACCTCCCAGAGGTCT

SEQ ID NO: 4 is the nucleotide sequence of a Sleeping beauty IR/DR sequence, integration junction (chr7, 79796094), shown in FIG. 7B.

ACTTAAGTGTATGTAAACTTCCGACTTCAACTGTAGGGTACCTGATTCTC
TGGGCATCTCTGCCCACTACCATG

SEQ ID NO: 5 is the nucleotide sequence of a Sleeping beauty IR/DR sequence, Integration junction (repeat region), shown in FIG. 7B.

ACTTAAGTGTATGTAAACTTCCGACTTCAACTGTAAATTTTCCACCTTTT
TCAGTTTTCCTCGCCATATTTCATG

SEQ ID NO: 6 is the nucleotide sequence of Long β-globin LCR positions 5292319-5270789 (21,531 bp) of human chromosome 11:

GATCTCTATCCCCTCCTGTTTTCTCTACGTTATTTATATGGGTATCATCA
CCATCCTGGACAACATCAGGACAGATATCCCTCACCAAGCCAATGTTCCT
CTCTATGTTGGCTCAAATGTCCTTGAACTTTCCTTTCACCACCCTTTCCA
CAGTCAAAAGGATATTGTAGTTTAATGCCTCAGAGTTCAGCTTTTAAGCT
TCTGACAAATTATTCTTCCTCTTTAGGTTCTCCTTTATGGAATCTTCTGT
ACTGATGGCCATGTCCTTTAACTACTATGTAGATATCTGCTACTACCTGT
ATTATGCCTCTACCTTTATTAGCAGAGTTATCTGTACTGTTGGCATGACA
ATCATTTGTTAATATGACTTGCCTTTCCTTTTTCTGCTATTCTTGATCAA
ATGGCTCCTCTTTCTTGCTCCTCTCATTTCTCCTGCCTTCACTTGGACGT
GCTTCACGTAGTCTGTGCTTATGACTGGATTAAAAATTGATATGGACTTA
TCCTAATGTTGTTCGTCATAATATGGGTTTTATGGTCCATTATTATTTCC
TATGCATTGATCTGGAGAAGGCTTCAATCCTTTTACTCTTTGTGGAAAAT
ATCTGTAAACCTTCTGGTTCACTCTGCTATAGCAATTTCAGTTTAGGCTA
GTAAGCATGAGGATGCCTCCTTCTCTGATTTTTCCCACAGTCTGTTGGTC
ACAGAATAACCTGAGTGATTACTGATGAAAGAGTGAGAATGTTATTGATA
GTCACAATGACAAAAAACAAACAACTACAGTCAAAATGTTTCTCTTTTTA
TTAGTGGATTATATTTCCTGACCTATATCTGGCAGGACTCTTTAGAGAGG
TAGCTGAAGCTGCTGTTATGACCACTAGAGGGAAGAAGATACCTGTGGAG
CTAATGGTCCAAGATGGTGGAGCCCCAAGCAAGGAAGTTGTTAAGGAGCC
CTTTTGATTGAAGGTGGGTGCCCCCACCTTACAGGGACAGGACATCTGGA
TACTCCTCCCAGTTTCTCCAGTTTCCCTTTTTCCTAATATATCTCCTGAT
AAAATGTCTATACTCACTTCCCCATTTCTAATAATAAAGCAAAGGCTAGT
TAGTAAGACATCACCTTGCATTTTGAAAATGCCATAGACTTTCAAAATTA
TTTCATACATCGGTCTTTCTTTATTTCAAGAGTCCAGAAATGGCAACATT
ACCTTTGATTCAATGTAATGGAAAGAGCTCTTTCAAGAGACAGAGAAAAG
AATAATTTAATTTCTTTCCCCACACCTCCTTCCCTGTCTCTTACCCTATC
TTCCTTCCTTCTACCCTCCCCATTTCTCTCTCTCATTTCTCAGAAGTATA
TTTTGAAAGGATTCATAGCAGACAGCTAAGGCTGGTTTTTTCTAAGTGAA
GAAGTGATATTGAGAAGGTAGGGTTGCATGAGCCCTTTCAGTTTTTTAGT
TTATATACATCTGTATTGTTAGAATGTTTTATAATATAAATAAAATTATT
TCTCAGTTATATACTAGCTATGTAACCTGTGGATATTTCCTTAAGTATTA
CAAGCTATACTTAACTCACTTGGAAAACTCAAATAAATACCTGCTTCATA
GTTATTAATAAGGATTAAGTGAGATAATGCCCATAAGATTCCTATTAATA
ACAGATAAATACATACACACACACACACATTGAAAGGATTCTTACTTTGT
GCTAGGAACTATAATAAGTTCATTGATGCATTATATCATTAAGTTCTAAT
TTCAACACTAGAAGGCAGGTATTATCTAAATTTCATACTGGATACCTCCA
AACTCATAAAGATAATTAAATTGCCTTTTGTCATATATTTATTCAAAAGG
GTAAACTCAAACTATGGCTTGTCTAATTTTATATATCACCCTACTGAACA
TGACCCTATTGTGATATTTTATAAAATTATTCTCAAGTTATTATGAGGAT
GTTGAAAGACAGAGAGGATGGGGTGCTATGCCCCAAATCAGCCTCACAAT
TAAGCTAAGCAGCTAAGAGTCTTGCAGGGTAGTGTAGGGACCACAGGGTT
AAGGGGGCAGTAGAATTATACTCCCACTTTAGTTTCATTTCAAACAATCC
ATACACACACAGCCCTGAGCACTTACAAATTATACTACGCTCTATACTTT
TTGTTTAAATGTATAAATAAGTGGATGAAAGAATAGATAGATAGATAGAC
AGATAGATGATAGATAGAATAAATGCTTGCCTTCATAGCTGTCTCCCTAC
CTTGTTCAAAATGTTCCTGTCCAGACCAAAGTACCTTGCCTTCACTTAAG
TAATCAATTCCTAGGTTATATTCTGATGTCAAAGGAAGTCAAAAGATGTG
AAAAACAATTTCTGACCCACAACTCATGCTTTGTAGATGACTAGATCAAA
AAATTTCAGCCATATCTTAACAGTGAGTGAACAGGAAATCTCCTCTTTTC
CCTACATCTGAGATCCCAGCTTCTAAGACCTTCAATTCTCACTCTTGATG
CAACAGACCTTGGAAGCATACAGGAGAGCTGAACTTGGTCAACAAAGGAG
AAAAGTTTGTTGGCCTCCAAAGGCACAGCTCAAACTTTTCAAGCCTTCTC
TAATCTTAAAGGTAAACAAGGGTCTCATTTCTTTGAGAACTTCAGGGAAA
ATAGACAAGGACTTGCCTGGTGCTTTTGGTAGGGGAGCTTGCACTTTCCC
CCTTTCTGGAGGAAATATTTATCCCCAGGTAGTTCCCTTTTTGCACCAGT
GGTTCTTTGAAGAGACTTCCACCTGGGAACAGTTAAACAGCAACTACAGG
GCCTTGAACTGCACACTTTCAGTCCGGTCCTCACAGTTGAAAAGACCTAA
GCTTGTGCCTGATTTAAGCCTTTTTGGTCATAAAACATTGAATTCTAATC
TCCCTCTCAACCCTACAGTCACCCATTTGGTATATTAAAGATGTGTTGTC
TACTGTCTAGTATCCCTCAAGTAGTGTCAGGAATTAGTCATTTAAATAGT
CTGCAAGCCAGGAGTGGTGGCTCATGTCTGTAATTCCAGCACTTGAGAGG
TAGAAGTGGGAGGACTGCTTGAGCTCAAGAGTTTGATATTATCCTGGACA
ACATAGCAAGACCTCGTCTCTACTTAAAAAAAAAAAAAAAATTAGCCAGG
CATGTGATGTACACCTGTAGTCCCAGCTACTCAGGAGGCCGAAATGGGAG
GATCCCTTGAGCTCAGGAGGTCAAGGCTGCAGTGAGACATGATCTTGCCA
CTGCACTCCAGCCTGGACAGCAGAGTGAAACCTTGCCTCACGAAACAGAA
TACAAAAACAAACAAACAAAAAACTGCTCCGCAATGCGCTTCCTTGATGC
TCTACCACATAGGTCTGGGTACTTTGTACACATTATCTCATTGCTGTTCA
TAATTGTTAGATTAATTTTGTAATATTGATATTATTCCTAGAAAGCTGAG
GCCTCAAGATGATAACTTTTATTTTCTGGACTTGTAATAGCTTTCTCTTG
TATTCACCATGTTGTAACTTTCTTAGAGTAGTAACAATATAAAGTTATTG
TGAGTTTTTGCAAACACAGCAAACACAACGACCCATATAGACATTGATGT
GAAATTGTCTATTGTCAATTTATGGGAAAACAAGTATGTACTTTTTCTAC
TAAGCCATTGAAACAGGAATAACAGAACAAGATTGAAAGAATACATTTTC
CGAAATTACTTGAGTATTATACAAAGACAAGCACGTGGACCTGGGAGGAG
GGTTATTGTCCATGACTGGTGTGTGGAGACAAATGCAGGTTTATAATAGA
TGGGATGGCATCTAGCGCAATGACTTTGCCATCACTTTTAGAGAGCTCTT
GGGGACCCCAGTACACAAGAGGGGACGCAGGGTATATGTAGACATCTCAT
TCTTTTTCTTAGTGTGAGAATAAGAATAGCCATGACCTGAGTTTATAGAC
AATGAGCCCTTTTCTCTCTCCCACTCAGCAGCTATGAGATGGCTTGCCCT
GCCTCTCTACTAGGCTGACTCACTCCAAGGCCCAGCAATGGGCAGGGCTC
TGTCAGGGCTTTGATAGCACTATCTGCAGAGCCAGGGCCGAGAAGGGGTG
GACTCCAGAGACTCTCCCTCCCATTCCCGAGCAGGGTTTGCTTATTTATG
CATTTAAATGATATATTTATTTTAAAAGAAATAACAGGAGACTGCCCAGC
CCTGGCTGTGACATGGAAACTATGTAGAATATTTTGGGTTCCATTTTTTT
TTCCTTCTTTCAGTTAGAGGAAAAGGGGCTCACTGCACATACACTAGACA
GAAAGTCAGGAGCTTTGAATCCAAGCCTGATCATTTCCATGTCATACTGA
GAAAGTCCCCACCCTTCTCTGAGCCTCAGTTTCTCTTTTTATAAGTAGGA
GTCTGGAGTAAATGATTTCCAATGGCTCTCATTTCAATACAAAATTTCCG
TTTATTAAATGCATGAGCTTCTGTTACTCCAAGACTGAGAAGGAAATTGA
ACCTGAGACTCATTGACTGGCAAGATGTCCCCAGAGGCTCTCATTCAGCA
ATAAAATTCTCACCTTCACCCAGGCCCACTGAGTGTCAGATTTGCATGCA
CTAGTTCACGTGTGTAAAAAGGAGGATGCTTCTTTCCTTTGTATTCTCAC
ATACCTTTAGGAAAGAACTTAGCACCCTTCCCACACAGCCATCCCAATAA
CTCATTTCAGTGACTCAACCCTTGACTTTATAAAAGTCTTGGGCAGTATA
GAGCAGAGATTAAGAGTACAGATGCTGGAGCCAGACCACCTGAGTGATTA
GTGACTCAGTTTCTCTTAGTAGTTGTATGACTCAGTTTCTTCATCTGTAA
AATGGAGGGTTTTTTAATTAGTTTGTTTTTGAGAAAGGGTCTCACTCTGT
CACCCAAATGGGAGTGTAGTGGCAAAATCTCGGCTCACTGCAACTTGCAC
TTCCCAGGCTCAAGCGGTCCTCCCACCTCAACATCCTGAGTAGCTGGAAC
CACAGGTACACACCACCATACCTCGCTAATTTTTTGTATTTTTGGTAGAG
ATGGGGTTTCACATGTTACACAGGATGGTCTCAGACTCCGGAGCTCAAGC
AATCTGCCCACCTCAGCCTTCCAAAGTGCTGGGATTATAAGCATGATTAC
AGGAGTTTTAACAGGCTCATAAGATTGTTCTGCAGCCCGAGTGAGTTAAT
ACATGCAAAGAGTTTAAAGCAGTGACTTATAAATGCTAACTACTCTAGAA
ATGTTTGCTAGTATTTTTTGTTTAACTGCAATCATTCTTGCTGCAGGTGA
AAACTAGTGTTCTGTACTTTATGCCCATTCATCTTTAACTGTAATAATAA
AAATAACTGACATTTATTGAAGGCTATCAGAGACTGTAATTAGTGCTTTG
CATAATTAATCATATTTAATACTCTTGGATTCTTTCAGGTAGATACTATT
ATTATCCCCATTTTACTACAGTTAAAAAAACTACCTCTCAACTTGCTCAA
GCATACACTCTCACACACACAAACATAAACTACTAGCAAATAGTAGAATT
GAGATTTGGTCCTAATTATGTCTTTGCTCACTATCCAATAAATATTTATT
GACATGTACTTCTTGGCAGTCTGTATGCTGGATGCTGGGGATACAAAGAT
GTTTAAATTTAAGCTCCAGTCTCTGCTTCCAAAGGCCTCCCAGGCCAAGT
TATCCATTCAGAAAGCATTTTTTACTCTTTGCATTCCACTGTTTTTCCTA
AGTGACTAAAAAATTACACTTTATTCGTCTGTGTCCTGCTCTGGGATGAT
AGTCTGACTTTCCTAACCTGAGCCTAACATCCCTGACATCAGGAAAGACT
ACACCATGTGGAGAAGGGGTGGTGGTTTTGATTGCTGCTGTCTTCAGTTA
GATGGTTAACTTTGTGAAGTTGAAAACTGTGGCTCTCTGGTTGACTGTTA
GAGTTCTGGCACTTGTCACTATGCCTATTATTTAACAAATGCATGAATGC
TTCAGAATATGGGAATATTATCTTCTGGAATAGGGAATCAAGTTATATTA
TGTAACCCAGGATTAGAAGATTCTTCTGTGTGTAAGAATTTCATAAACAT
TAAGCTGTCTAGCAAAAGCAAGGGCTTGGAAAATCTGTGAGCTCCTCACC
ATATAGAAAGCTTTTAACCCATCATTGAATAAATCCCTATAGGGGATTTC
TACCCTGAGCAAAAGGCTGGTCTTGATTAATTCCCAAACTCATATAGCTC
TGAGAAAGTCTATGCTGTTAACGTTTTCTTGTCTGCTACCCCATCATATG
CACAACAATAAATGCAGGCCTAGGCATGACTGAAGGCTCTCTCATAATTC
TTGGTTGCATGAATCAGATTATCAACAGAAATGTTGAGACAAACTATGGG
GAAGCAGGGTATGAAAGAGCTCTGAATGAAATGGAAACCGCAATGCTTCC
TGCCCATTCAGGGCTCCAGCATGTAGAAATCTGGGGCTTTGTGAAGACTG
GCTTAAAATCAGAAGCCCCATTGGATAAGAGTAGGGAAGAACCTAGAGCC
TACGCTGAGCAGGTTTCCTTCATGTGACAGGGAGCCTCCTGCCCCGAACT
TCCAGGGATCCTCTCTTAAGTGTTTCCTGCTGGAATCTCCTCACTTCTAT
CTGGAAATGGTTTCTCCACAGTCCAGCCCCTGGCTAGTTGAAAGAGTTAC
CCATGCAGAGGCCCTCCTAGCATCCAGAGACTAGTGCTTAGATTCCTACT
TTCAGCGTTGGACAACCTGGATCCACTTGCCCAGTGTTCTTCCTTAGTTC
CTACCTTCGACCTTGATCCTCCTTTATCTTCCTGAACCCTGCTGAGATGA
TCTATGTGGGGAGAATGGCTTCTTTGAGAAACATCTTCTTCGTTAGTGGC
CTGCCCCTCATTCCCACTTTAATATCCAGAATCACTATAAGAAGAATATA
ATAAGAGGAATAACTCTTATTATAGGTAAGGGAAAATTAAGAGGCATACG
TGATGGGATGAGTAAGAGAGGAGAGGGAAGGATTAATGGACGATAAAATC
TACTACTATTTGTTGAGACCTTTTATAGTCTAATCAATTTTGCTATTGTT
TTCCATCCTCACGCTAACTCCATAAAAAAACACTATTATTATCTTTATTT
TGCCATGACAAGACTGAGCTCAGAAGAGTCAAGCATTTGCCTAAGGTCGG
ACATGTCAGAGGCAGTGCCAGACCTATGTGAGACTCTGCAGCTACTGCTC
ATGGGCCCTGTGCTGCACTGATGAGGAGGATCAGATGGATGGGGCAATGA
AGCAAAGGAATCATTCTGTGGATAAAGGAGACAGCCATGAAGAAGTCTAT
GACTGTAAATTTGGGAGCAGGAGTCTCTAAGGACTTGGATTTCAAGGAAT
TTTGACTCAGCAAACACAAGACCCTCACGGTGACTTTGCGAGCTGGTGTG
CCAGATGTGTCTATCAGAGGTTCCAGGGAGGGTGGGGTGGGGTCAGGGCT
GGCCACCAGCTATCAGGGCCCAGATGGGTTATAGGCTGGCAGGCTCAGAT
AGGTGGTTAGGTCAGGTTGGTGGTGCTGGGTGGAGTCCATGACTCCCAGG
AGCCAGGAGAGATAGACCATGAGTAGAGGGCAGACATGGGAAAGGTGGGG
GAGGCACAGCATAGCAGCATTTTTCATTCTACTACTACATGGGACTGCTC
CCCTATACCCCCAGCTAGGGGCAAGTGCCTTGACTCCTATGTTTTCAGGA
TCATCATCTATAAAGTAAGAGTAATAATTGTGTCTATCTCATAGGGTTAT
TATGAGGATCAAAGGAGATGCACACTCTCTGGACCAGTGGCCTAACAGTT
CAGGACAGAGCTATGGGCTTCCTATGTATGGGTCAGTGGTCTCAATGTAG
CAGGCAAGTTCCAGAAGATAGCATCAACCACTGTTAGAGATATACTGCCA
GTCTCAGAGCCTGATGTTAATTTAGCAATGGGCTGGGACCCTCCTCCAGT
AGAACCTTCTAACCAGCTGCTGCAGTCAAAGTCGAATGCAGCTGGTTAGA
CTTTTTTTAATGAAAGCTTAGCTTTCATTAAAGATTAAGCTCCTAAGCAG
GGCACAGATGAAATTGTCTAACAGCAACTTTGCCATCTAAAAAAATCTGA
CTTCACTGGAAACATGGAAGCCCAAGGTTCTGAACATGAGAAATTTTTAG
GAATCTGCACAGGAGTTGAGAGGGAAACAAGATGGTGAAGGGACTAGAAA
CCACATGAGAGACACGAGGAAATAGTGTAGATTTAGGCTGGAGGTAAATG
AAAGAGAAGTGGGAATTAATACTTACTGAAATCTTTCTATATGTCAGGTG
CCATTTTATGATATTTAATAATCTCATTACATATGGTAATTCTGTGAGAT
ATGTATTATTGAACATACTATAATTAATACTAATGATAAGTAACACCTCT
TGAGTACTTAGTATATGCTAGAATCAAATTTAAGTTTATCATATGAGGCC
GGGCACGGTGGCTCATATATGGGATTACATGCCTGTAATCCCAGCACTTT
GGGAGGCCAAGGCAATTGGATCACCTGAGGTCAGGAGTTCCAGACCAGCC
TGGCCAACATGGTGAAACCCCTTCTCTACTAAAAAATACAAAAAATCAGC
CAGGTGTGGTGGCACGCGTCTATAATCCCAGCTACTCAGGAGGCTGAGGC
AGGAGAATCACTTGAACCCAGGAGGTGGAGGTTGCAGTGAGCTAAGATTG
CACCACTGCACTCCAGCCTAGGCGACAGAGTGAGACTCCATCTCAAAAAA
AAAAAAAGAAGTTTATTATATGAATTAACTTAGTTTTACTCACACCAATA
CTCAGAAGTAGATTATTACCTCATTTATTGATGAGGAGCCCAATGTACTT
GTAGTGTAGATCAACTTATTGAAAGCACAAGCTAATAAGTAGACAATTAG
TAATTAGAAGTCAGATGGTCTGAGCTCTCCTACTGTCTACATTACATGAG
CTCTTATTAACTGGGGACTCGAAAATCAAAGACATGAAATAATTTGTCCA
AGCTTACAGAACCACCAAGTAGTAAGGCTAGGATGTAGACCCAGTTCTGC
TACCTCTGAAGACAGTGTTTTTTCCACAGCAAAACACAAACTCAGATATT
GTGGATGCGAGAAATTAGAAGTAGATATTCCTGCCCTGTGGCCCTTGCTT
CTTACTTTTACTTCTTGTCGATTGGAAGTTGTGGTCCAAGCCACAGTTGC
AGACCATACTTCCTCAACCATAATTGCATTTCTTCAGGAAAGTTTGAGGG
AGAAAAAGGTAAAGAAAAATTTAGAAACAACTTCAGAATAAAGAGATTTT
CTCTTGGGTTACAGAGATTGTCATATGACAAATTATAAGCAGACACTTGA
GAAAACTGAAGGCCCATGCCTGCCCAAATTACCCTTTGACCCCTTGGTCA
AGCTGCAACTTTGGTTAAAGGGAGTGTTTATGTGTTATAGTGTTCATTTA
CTCTTCTGGTCTAACCCATTGGCTCCGTCTTCATCCTGCAGTGACCTCAG
TGCCTCAGAAACATACATATGTTTGTCTAGTTTAAGTTTGTGTGAAATTC
TAACTAGCGTCAAGAACTGAGGGCCCTAAACTATGCTAGGAATAGTGCTG
TGGTGCTGTGATAGGTACACAAGAAATGAGAAGAAACTGCAGATTCTCTG
CATCTCCCTTTGCCGGGTCTGACAACAAAGTTTCCCCAAATTTTACCAAT
GCAAGCCATTTCTCCATATGCTAACTACTTTAAAATCATTTGGGGCTTCA
CATTGTCTTTCTCATCTGTAAAAAGAATGGAAGAACTCATTCCTACAGAA
CTCCCTATGTCTTCCCTGATGGGCTAGAGTTCCTCTTTCTCAAAAATTAG
CCATTATTGTATTTCCTTCTAAGCCAAAGCTCAGAGGTCTTGTATTGCCC
AGTGACATGCACACTGGTCAAAAGTAGGCTAAGTAGAAGGGTACTTTCAC
AGGAACAGAGAGCAAAAGAGGTGGGTGAATGAGAGGGTAAGTGAGAAAAG
ACAAATGAGAAGTTACAACATGATGGCTTGTTGTCTAAATATCTCCTAGG
GAATTATTGTGAGAGGTCTGAATAGTGTTGTAAAATAAGCTGAATCTGCT
GCCAACATTAACAGTCAAGAAATACCTCCGAATAACTGTACCTCCAATTA
TTCTTTAAGGTAGCATGCAACTGTAATAGTTGCATGTATATATTTATCAT
AATACTGTAACAGAAAACACTTACTGAATATATACTGTGTCCCTAGTTCT
TTACACAATAAACTAATCTCATCCTCATAATTCTATTAGCTAATACATAT
TATCATCCTATATTTCAGAGACTTCAAGAAGTTAAGCAACTTGCTCAAGA
TCATCTAAGAAGTAGGTGGTATTTCTGGGCTCATTTGGCCCCTCCTAATC
TCTCATGGCAACATGGCTGCCTAAAGTGTTGATTGCCTTAATTCATCAGG
GATGGGCTCATACTCACTGCAGACCTTAACTGGCATCCTCTTTTCTTATG
TGATCTGCCTGACCCTAGTAGACTTATGAAATTTCTGATGAGAAAGGAGA
GAGGAGAAAGGCAGAGCTGACTGTGATGAGTGATGAAGGTGCCTTCTCAT
CTGGGTACCAGTGGGGCCTCTAAGACTAAGTCACTCTGTCTCACTGTGTC
TTAGCCAGTTCCTTACAGCTTGCCCTGATGGGAGATAGAGAATGGGTATC
CTCCAACAAAAAAATAAATTTTCATTTCTCAAGGTCCAACTTATGTTTTC
TTAATTTTTAAAAAAATCTTGACCATTCTCCACTCTCTAAAATAATCCAC
AGTGAGAGAAACATTCTTTTCCCCCATCCCATAAATACCTCTATTAAATA
TGGAAAATCTGGGCATGGTGTCTCACACCTGTAATCCCAGCACTTTGGGA
GGCTGAGGTGGGTGGACTGCTTGGAGCTCAGGAGTTCAAGACCATCTTGG
ACAACATGGTGATACCCTGCCTCTACAAAAAGTACAAAAATTAGCCTGGC
ATGGTGGTGTGCACCTGTAATCCCAGCTATTAGGGTGGCTGAGGCAGGAG
AATTGCTTGAACCCGGGAGGCGGAGGTTGCAGTGAGCTGAGATCGTGCCA
CTGCACTCCAGCCTGGGGGACAGAGCACATTATAATTAACTGTTATTTTT
TACTTGGACTCTTGTGGGGAATAAGATACATGTTTTATTCTTATTTATGA
TTCAAGCACTGAAAATAGTGTTTAGCATCCAGCAGGTGCTTCAAAACCAT
TTGCTGAATGATTACTATACTTTTTACAAGCTCAGCTCCCTCTATCCCTT
CCAGCATCCTCATCTCTGATTAAATAAGCTTCAGTTTTTCCTTAGTTCCT
GTTACATTTCTGTGTGTCTCCATTAGTGACCTCCCATAGTCCAAGCATGA
GCAGTTCTGGCCAGGCCCCTGTCGGGGTCAGTGCCCCACCCCCGCCTTCT
GGTTCTGTGTAACCTTCTAAGCAAACCTTCTGGCTCAAGCACAGCAATGC
TGAGTCATGATGAGTCATGCTGAGGCTTAGGGTGTGTGCCCAGATGTTCT
CAGCCTAGAGTGATGACTCCTATCTGGGTCCCCAGCAGGATGCTTACAGG
GCAGATGGCAAAAAAAAGGAGAAGCTGACCACCTGACTAAAACTCCACCT
CAAACGGCATCATAAAGAAAATGGATGCCTGAGACAGAATGTGACATATT
CTAGAATATATTATTTCCTGAATATATATATATATATACACATATACGTA
TATATATATATATATATATATTTGTTGTTATCAATTGCCATAGAATGATT
AGTTATTGTGAATCAAATATTTATCTTGCAGGTGGCCTCTATACCTAGAA
GCGGCAGAATCAGGCTTTATTAATACATGTGTATAGATTTTTAGGATCTA
TACACATGTATTAATATGAAACAAGGATATGGAAGAGGAAGGCATGAAAA
CAGGAAAAGAAAACAAACCTTGTTTGCCATTTTAAGGCACCCCTGGACAG
CTAGGTGGCAAAAGGCCTGTGCTGTTAGAGGACACATGCTCACATACGGG
GTCAGATCTGACTTGGGGTGCTACTGGGAAGCTCTCATCTTAAGGATACA
TCTCAGGCCAGTCTTGGTGCATTAGGAAGATGTAGGCAACTCTGATCCTG
AGAGGAAAGAAACATTCCTCCAGGAGAGCTAAAAGGGTTCACCTGTGTGG
GTAACTGTGAAGGACTACAAGAGGATGAAAAACAATGACAGACAGACATA
ATGCTTGTGGGAGAAAAAACAGGAGGTCAAGGGGATAGAGAAGGCTTCCA
GAAGAATGGCTTTGAAGCTGGCTTCTGTAGGAGTTCACAGTGGCAAAGAT
GTTTCAGAAATGTGACATGACTTAAGGAACTATACAAAAAGGAACAAATT
TAAGGAGAGGCAGATAAATTAGTTCAACAGACATGCAAGGAATTTTCAGA
TGAATGTTATGTCTCCACTGAGCTTCTTGAGGTTAGCAGCTGTGAGGGTT
TTGCAGGCCCAGGACCCATTACAGGACCTCACGTATACTTGACACTGTTT
TTTGTATTCATTTGTGAATGAATGACCTCTTGTCAGTCTACTCGGTTTCG
CTGTGAATGAATGATGTCTTGTCAGCCTACTTGGTTTCGCTAAGAGCACA
GAGAGAAGATTTAGTGATGCTATGTAAAAACTTCCTTTTTGGTTCAAGTG
TATGTTTGTGATAGAAATGAAGACAGGCTACATGATGCATATCTAACATA
AACACAAACATTAAGAAAGGAAATCAACCTGAAGAGTATTTATACAGATA
ACAAAATACAGAGAGTGAGTTAAATGTGTAATAACTGTGGCACAGGCTGG
AATATGAGCCATTTAAATCACAAATTAATTAGAAAAAAAACAGTGGGGAA
AAAATTCCATGGATGGGTCTAGAAAGACTAGCATTGTTTTAGGTTGAGTG
GCAGTGTTTAAAGGGTGATATCAGACTAAACTTGAAATATGTGGCTAAAT
AACTAGAATACTCTTTATTTTTTCGTATCATGAATAGCAGATATAGCTTG
ATGGCCCCATGCTTGGTTTAACATCCTTGCTGTTCCTGACATGAAATCCT
TAATTTTTGACAAAGGGGCTATTCATTTTCATTTTATATTGGGCCTAGAA
ATTATGTAGATGGTCCTGAGGAAAAGTTTATAGCTTGTCTATTTCTCTCT
CTAACATAGTTGTCAGCACAATGCCTAGGCTATAGGAAGTACTCAAAGCT
TGTTAAATTGAATTCTATCCTTCTTATTCAATTCTACACATGGAGGAAAA
ACTCATCAGGGATGGAGGCACGCCTCTAAGGAAGGCAGGTGTGGCTCTGC
AGTGTGATTGGGTACTTGCAGGACGAAGGGTGGGGTGGGAGTGGCTAACC
TTCCATTCCTAGTGCAGAGGTCACAGCCTAAACATCAAATTCCTTGAGGT
GCGGTGGCTCACTCCTGTAATCACAGCAGTTTGGGACGCCAAGGTGGGCA
GATCACTTGAGGTCAGGAGTTGGACACCAGCCCAGCCAACATAGTGAAAC
CTGGTCTCTGCTTAAAAATATAAAAATTAGCTGGACGTGGTGACGGGAGC
CTGTAATCCAACTACTTGGGAGGCTGAGGCAGGAGAATCGCTTGAACCGG
GGAGGTGGAGTTTGCACTGAGCAGAGATCATGCCATTGCACTCCAGCCTC
CAGAGCGAGACTCTGTCTAAAGAAAAACGAAAACAAACAAACAAACAAAC
AAACAAAACCCATCAAATTCCCTGACCGAACAGAATTCTGTCTGATTGTT
CTCTGACTTATCTACCATTTTCCCTCCTTAAAGAAACTGTGAACTTCCTT
CAGCTAGAGGGGCCTGGCTCAGAAGCCTCTGGTCAGCATCCAAGAAATAC
TTGATGTCACTTTGGCTAAAGGTATGATGTGTAGACAAGCTCCAGAGATG
GTTTCTCATTTCCATATCCACCCACCCAGCTTTCCAATTTTAAAGCCAAT
TCTGAGGTAGAGACTGTGATGAACAAACACCTTGACAAAATTCAACCCAA
AGACTCACTTTGCCTAGCTTCAAAATCCTTACTCTGACATATACTCACAG
CCAGAAATTAGCATGCACTAGAGTGTGCATGAGTGCAACACACACACACA
CCAATTCCATATTCTCTGTCAGAAAATCCTGTTGGTTTTTCGTGAAAGGA
TGTTTTCAGAGGCTGACCCCTTGCCTTCACCTCCAATGCTACCACTCTGG
TCTAAGTCACTGTCACCACCACCTAAATTATAGCTGTTGACTCATAACAA
TCTTCCTGCTTCTACCACTGCCCCACTACAATTTCTTCCCAATATACTAT
CCAAATTAGTCTTTTCAAAATGTAAGTCATATATGGTCACCTCTTTGTTC
AAAGTCTTCTGATAGTTTCCTATATCATTTATAATAAAACCAAATCCTTA
CAATTCTCTACAATAGTTGTTCATGCATATATTATGTTTATTACAGATAC
ATATATATAGCTCTCATATAAATAAATATATATATTTATGTGTATGTGTG
TAGAGTGTTTTTTCTTACAACTCTATGATGTAGGTATTATTAGTGTCCCA
AATTTTATAATTTAGGACTTCTATGATCTCATCTTTTATTCTCCCCTTCA
CCGAATCTCATCCTACATTGGCCTTATTGATATTCCTTGAAAATTCTAAG
CATCTTACATCTTTAGGGTATTTACATTTGCCATTCCCTATGCCCTAAAT
ATTTAATCATAGTTTCATATAAATGGGTTCCTCATCATCTATGGGTACTC
TCTCAGGTGTTAACTTTATAGTGAGGACTTTCCTGCCATACTACTTAAAG
TAGCGATACCCTTTCACCCTGTCCTAATCACACTCTGGCCTTCATTTCAG
TTTTTTTTTTTTCTCCATAGCACCTAATCTCATTGGTATATAACATGTTT
CATTTGCTTATTTAATGTCAAGCTCTTTCCACTATCAAGTCCATGAAAAC
AGGAACTTTATTCCTCTATTCTGTTTTTGTGCTGTATTCTTAGCAATTTT
ACAATTTTGAATGAATGAATGAGCAGTCAAACACATATACAACTATAATT
AAAAGGATGTATGCTGACACATCCACTGCTATGCACACACAAAGAAATCA
GTGGAGTAGAGCTGGAAGTGCTAAGCCTGCATAGAGCTAGTTAGCCCTCC
GCAGGCAGAGCCTTGATGGGATTACTGAGTTCTAGAATTGGACTCATTTG
TTTTGTAGGCTGAGATTTGCTCTTGAAAACTTGTTCTGACCAAAATAAAA
GGCTCAAAAGATGAATATCGAAACCAGGGTGTTTTTTACACTGGAATTTA
TAACTAGAGCACTCATGTTTATGTAAGCAATTAATTGTTTCATCAGTCAG
GTAAAAGTAAAGAAAAACTGTGCCAAGGCAGGTAGCCTAATGCAATATGC
CACTAAAGTAAACATTATTTCATAGGTGTCAGATATGGCTTATTCATCCA
TCTTCATGGGAAGGATGGCCTTGGCCTGGACATCAGTGTTATGTGAGGTT
CAAAACACCTCTAGGCTATAAGGCAACAGAGCTCCTTTTTTTTTTTTCTG
TGCTTTCCTGGCTGTCCAAATCTCTAATGATAAGCATACTTCTATTCAAT
GAGAATATTCTGTAAGATTATAGTTAAGAATTGTGGGAGCCATTCCGTCT
CTTATAGTTAAATTTGAGCTTCTTTTATGATCACTGTTTTTTTAATATGC
TTTAAGTTCTGGGGTACATGTGCCATGGTGGTTTGCTGCACCCATCAACC
CGTCATCTACATTAGGTATTTCTCCTAATGCTATCCTTCCCCTAGCCCCC
CACCCCCAACAGGCCCCAGTGTGTGATGTTCCCCTCCCTGTGTCCATGGA
TCACTGGTTTTTTTTTGTTTTTTTTTTTTTTTTAAAGTCTCAGTTAAATT
TTTGGAATGTAATTTATTTTCCTGGTATCCTAGGACTTGCAAGTTATCTG
GTCACTTTAGCCCTCACGTTTTGATGATAATCACATATTTGTAAACACAA
CACACACACACACACACACACACATATATATATATATAAAACATATATAT
ACATAAACACACATAACATATTTATCGGGCATTTCTGAGCAACTAATCAT
GCAGGACTCTCAAACACTAACCTATAGCCTTTTCTATGTATCTACTTGTG
TAGAAACCAAGCGTGGGGACTGAGAAGGCAATAGCAGGAGCATTCTGACT
CTCACTGCCTTTAGCTAGGCCCCTCCCTCATCACAGCTCAGCATAGTCCT
GAGCTCTTATCTATATCCACACACAGTTTCTGACGCTGCCCAGCTATCAC
CATCCCAAGTCTAAAGAAAAAAATAATGGGTTTGCCCATCTCTGTTGATT
AGAAAACAAAACAAAATAAAATAAGCCCCTAAGCTCCCAGAAAACATGAC
TAAACCAGCAAGAAGAAGAAAATACAATAGGTATATGAGGAGACTGGTGA
CACTAGTGTCTGAATGAGGCTTGAGTACAGAAAAGAGGCTCTAGCAGCAT
AGTGGTTTAGAGGAGATGTTTCTTTCCTTCACAGATGCCTTAGCCTCAAT
AAGCTTGCGGTTGTGGAAGTTTACTTTCAGAACAAACTCCTGTGGGGCTA
GAATTATTGATGGCTAAAAGAAGCCCGGGGGAGGGAAAAATCATTCAGCA
TCCTCACCCTTAGTGACACAAAACAGAGGGGGCCTGGTTTTCCATATTTC
CTCATGATGGATGATCTCGTTAATGAAGGTGGTCTGACGAGATCATTGCT
TCTTCCATTTAAGCCTTGCTCACTTGCCAATCCTCAGTTTTAACCTTCTC
CAGAGAAATACACATTTTTTATTCAGGAAACATACTATGTTATAGTTTCA
ATACTAAATAATCAAAGTACTGAAGATAGCATGCATAGGCAAGAAAAAGT
CCTTAGCTTTATGTTGCTGTTGTTTCAGAATTTAAAAAAGATCACCAAGT
CAAGGACTTCTCAGTTCTAGCACTAGAGGTGGAATCTTAGCATATAATCA
GAGGTTTTTCAAAATTTCTAGACATAAGATTCAAAGCCCTGCACTTAAAA
TAGTCTCATTTGAATTAACTCTTTATATAAATTGAAAGCACATTCTGAAC
TACTTCAGAGTATTGTTTTATTTCTATGTTCTTAGTTCATAAATACATTA
GGCAATGCAATTTAATTAAAAAAACCCAAGAATTTCTTAGAATTTTAATC
ATGAAAATAAATGAAGGCATCTTTACTTACTCAAGGTCCCAAAAGGTCAA
AGAAACCAGGAAAGTAAAGCTATATTTCAGCGGAAAATGGGATATTTATG
AGTTTTCTAAGTTGACAGACTCAAGTTTTAACCTTCAGTGCCCATCATGT
AGGAAAGTGTGGCATAACTGGCTGATTCTGGCTTTCTACTCCTTTTTCCC
ATTAAAGATCCCTCCTGCTTAATTAACATTCACAAGTAACTCTGGTTGTA
CTTTAGGCACAGTGGCTCCCGAGGTCAGTCACACAATAGGATGTCTGTGC
TCCAAGTTGCCAGAGAGAGAGATTACTCTTGAGAATGAGCCTCAGCCCTG
GCTCAAACTCACCTGCAAACTTCGTGAGAGATGAGGCAGAGGTACACTAC
GAAAGCAACAGTTAGAAGCTAAATGATGAGAACACATGGACTCATAGAGG
GAAACAACGCATACTGGGGCCTATCAGAGGGTGGAGGGTGAGAGAAGGAG
AGGATCAGGAAAAATCACTAATGGATGCTAAGCGTAATACCTGAGTGATG
AGATCATCTATACAACAAACCCCCTTGACATTCATTTATCTATGTAACAA
ACCTGCACATCCTGTACATGTACCCCTGAACTTAAAATAAAAGTTGAAAA
CAAGAAAGCAACAGTTTGAACACTTGTTATGGTCTATTCTCTCATTCTTT
ACAATTACACTAGAAAATAGCCACAGGCTTCCTGCAAGGCAGCCACAGAA
TTTATGACTTGTGATATCCAAGTCATTCCTGGATAATGCAAAATCTAACA
CAAAATCTAGTAGAATCATTTGCTTACATCTATTTTTGTTCTGAGAATAT
AGATTTAGATACATAATGGAAGCAGAATAATTTAAAATCTGGCTAATTTA
GAATCCTAAGCAGCTCTTTTCCTATCAGTGGTTTACAAGCCTTGTTTATA
TTTTTCCTATTTTAAAAATAAAAATAAAGTAAGTTATTTGTGGTAAAGAA
TATTCATTAAAGTATTTATTTCTTAGATAATACCATGAAAAACATTCAGT
GAAGTGAAGGGCCTACTTTACTTAACAAGAATCTAATTTATATAATTTTT
CATACTAATAGCATCTAAGAACAGTACAATATTTGACTCTTCAGGTTAAA
CATATGTCATAAATTAGCCAGAAAGATTTAAGAAAATATTGGATGTTTCC
TTGTTTAAATTAGGCATCTTACAGTTTTTAGAATCCTGCATAGAACTTAA
GAAATTACAAATGCTAAAGCAAACCCAAACAGGCAGGAATTAATCTTCAT
CGAATTTGGGTGTTTCTTTCTAAAAGTCCTTTATACTTAAATGTCTTAAG
ACATACATAGATTTTATTTTACTAATTTTAATTATATAGACAATAAATGA
ATATTCTTACTGATTACTTTTTCTGACTGTCTAATCTTTCTGATCTATCC
TGGATGGCCATAACACTTATCTCTCTGAACTTTGGGCTTTTAATATAGGA
AAGAAAAGCAATAATCCATTTTTCATGGTATCTCATATGATAAACAAATA
AAATGCTTAAAAATGAGCAGGTGAAGCAATTTATCTTGAACCAACAAGCA
TCGAAGCAATAATGAGACTGCCCGCAGCCTACCTGACTTCTGAGTCAGGA
TTTATAAGCCTTGTTACTGAGACACAAACCTGGGCCTTTCAATGCTATAA
CCTTTCTTGAAGCTCCTCCCTACCACCTTTAGCCATAAGGAAACATGGAA
TGGGTCAGATCCCTGGATGCAAGCCAGGTCTGGAACCATAGGCAGTAAGG
AGAGAAGAAAATGTGGGCTCTGCAACTGGCTCCGAGGGAGCAGGAGAGGA
TCAACCCCATACTCTGAATCTAAGAGAAGACTGGTGTCCATACTCTGAAT
GGGAAGAATGATGGGATTACCCATAGGGCTTGTTTTAGGGAGAAACCTGT
TCTCCAAACTCTTGGCCTTGAGATACCTGGTCCTTATTCCTTGGACTTTG
GCAATGTCTGACCCTCACATTCAAGTTCTGAGGAAGGGCCACTGCCTTCA
TACTGTGGATCTGTAGCAAATTCCCCCTGAAAACCCAGAGCTGTATCTTA
ATTGGTTAAAAAAAATTATATTATCTCAACGACTGTTCTTCTCTGAGTAG
CCAAGCTCAGCTTGGTTCAAGCTACAAGCAGCTGAGCTGCTTTTTGTCTA
GTCATTGTTCTTTTATTTCAGTGGATCAAATACGTTCTTTCCAAACCTAG
GATCTTGTCTTCCTAGGCTATATATTTTGTCCCAGGAAGTCTTAATCTGG
GGTCCACAGAACACTAGGGGGCTGGTGAAGTTTATAGAAAAAAAATCTGT
ATTTTTACTTACATGTAACTGAAATTTAGCATTTTCTTCTACTTTGAATG
CAAAGGACAAACTAGAATGACATCATCAGTACCTATTGCATAGTTATAAA
GAGAAACCACAGATATTTTCATACTACACCATAGGTATTGCAGATCTTTT
TGTTTTTGTTTTTGTTTGAGATGGAGTTTCGCTCTTATTGCCCAGGCTGG
AGTGCAGTGGCATGATTTCGGCTCACTGCAACCTCCCCTTCCTGCATTCA
AGCAATTCTCCTGCCTTGGCCTCCTGAGTAGCTGGGGATTACAGGCACCT
GCCACCATGCCAGTCTAATTTTTGTATTTTTAGTAGAGATGGGGTTTCGC
CATGTTGGCCAGGCTGGTCTTGAACTCCTGACCTCAGATGATCTGCCCGC
CTTGGCCTCCTGAAGTGCTGGGATTATAGGTGTGAGCCACCACGCCTGGC
CCATTGCAGATATTTTTAATTCACATTTATCTGCATCACTACTTGGATCT
TAAGGTAGCTGTAGACCCAATCCTAGATCTAATGCTTTCATAAAGAAGCA
AATATAATAAATACTATACCACAAATGTAATGTTTGATGTCTGATAATGA
TATTTCAGTGTAATTAAACTTAGCACTCCTATGTATATTATTTGATGCAA
TAAAAACATATTTTTTTAGCACTTACAGTCTGCCAAACTGGCCTGTGACA
CAAAAAAAGTTTAGGAATTCCTGGTTTTGTCTGTGTTAGCCAATGGTTAG
AATATATGCTCAGAAAGATACCATTGGTTAATAGCTAAAAGAAAATGGAG
TAGAAATTCAGTGGCCTGGAATAATAACAATTTGGGCAGTCATTAAGTCA
GGTGAAGACTTCTGGAATCATGGGAGAAAAGCAAGGGAGACATTCTTACT
TGCCACAAGTGTTTTTTTTTTTTTTTTTTTTTATCACAAACATAAGAAAA
TATAATAAATAACAAAGTCAGGTTATAGAAGAGAGAAACGCTCTTAGTAA
ACTTGGAATATGGAATCCCCAAAGGCACTTGACTTGGGAGACAGGAGCCA
TACTGCTAAGTGAAAAAGACGAAGAACCTCTAGGGCCTGAACATACAGGA
AATTGTAGGAACAGAAATTCCTAGATCTGGTGGGGCAAGGGGAGCCATAG
GAGAAAGAAATGGTAGAAATGGATGGAGACGGAGGCAGAGGTGGGCAGAT
CATGAGGTCAAGAGATCGAGACCATCCTGGCAAACATGGTGAAATCCCGT
CTCTACTAAAAATAAAAAAATTAGCTGGGCATGGTGGCATGCGCCTGTAG
TCCCAGCTGCTCGGGAGGCTGAGGCAGGAGAATCGTTTGAACCCAGGAGG
CGAAGGTTGCAGTGAGCTGAGATAGTGCCATTGCACTCCAGTCTGGCAAC
AGAGTGAGACTCCGTCTCAAAAAAAAAAAAAAAAGAAAGAAAGAAAAGAA
AAAGAAAAAAGAAAAAATAAATGGATGTAGAACAAGCCAGAAGGAGGAAC
TGGGCTGGGGCAATGAGATTATGGTGATGTAAGGGACTTTTATAGAATTA
ACAATGCTGGAATTTGTGGAACTCTGCTTCTATTATTCCCCCAATCATTA
CTTCTGTCACATTGATAGTTAAATAATTTCTGTGAATTTATTCCTTGATT
CTAAAATATGAGGATAATGACAATGGTATTATAAGGGCAGATTAAGTGAT
ATAGCATGAGCAATATTCTTCAGGCACATGGATCGAATTGAATACACTGT
AAATCCCAACTTCCAGTTTCAGCTCTACCAAGTAAAGAGCTAGCAAGTCA
TCAAAATGGGGACATACAGAAAAAAAAAAGGACACTAGAGGAATAATATA
CCCTGACTCCTAGCCTGATTAATATATCGAT

SEQ ID NO: 7 is the nucleotide sequence of a Transposable transgene insert that includes positions 5228631-5227018 (1614 bp) of human chromosome 11:

GATCTCTATTTATTTAGCAATAATAGAGAAAGCATTTAAGAGAATAAAGC
AATGGAAATAAGAAATTTGTAAATTTCCTTCTGATAACTAGAAATAGAGG
ATCCAGTTTCTTTTGGTTAACCTAAATTTTATTTCATTTTATTGTTTTAT
TTTATTTTATTTTATTTTATTTTGTGTAATCGTAGTTTCAGAGTGTTAGA
GCTGAAAGGAAGAAGTAGGAGAAACATGCAAAGTAAAAGTATAACACTTT
CCTTACTAAACCGACATGGGTTTCCAGGTAGGGGCAGGATTCAGGATGAC
TGACAGGGCCCTTAGGGAACACTGAGACCCTACGCTGACCTCATAAATGC
TTGCTACCTTTGCTGTTTTAATTACATCTTTTAATAGCAGGAAGCAGAAC
TCTGCACTTCAAAAGTTTTTCCTCACCTGAGGAGTTAATTTAGTACAAGG
GGAAAAAGTACAGGGGGATGGGAGAAAGGCGATCACGTTGGGAAGCTATA
GAGAAAGAAGAGTAAATTTTAGTAAAGGAGGTTTAAACAAACAAAATATA
AAGAGAAATAGGAACTTGAATCAAGGAAATGATTTTAAAACGCAGTATTC
TTAGTGGACTAGAGGAAAAAAATAATCTGAGCCAAGTAGAAGACCTTTTC
CCCTCCTACCCCTACTTTCTAAGTCACAGAGGCTTTTTGTTCCCCCAGAC
ACTCTTGCAGATTAGTCCAGGCAGAAACAGTTAGATGTCCCCAGTTAACC
TCCTATTTGACACCACTGATTACCCCATTGATAGTCACACTTTGGGTTGT
AAGTGACTTTTTATTTATTTGTATTTTTGACTGCATTAAGAGGTCTCTAG
TTTTTTATCTCTTGTTTCCCAAAACCTAATAAGTAACTAATGCACAGAGC
ACATTGATTTGTATTTATTCTATTTTTAGACATAATTTATTAGCATGCAT
GAGCAAATTAAGAAAAACAACAACAAATGAATGCATATATATGTATATGT
ATGTGTGTATATATACACACATATATATATATATTTTTTCTTTTCTTACC
AGAAGGTTTTAATCCAAATAAGGAGAAGATATGCTTAGAACCGAGGTAGA
GTTTTCATCCATTCTGTCCTGTAAGTATTTTGCATATTCTGGAGACGCAG
GAAGAGATCCATCTACATATCCCAAAGCTGAATTATGGTAGACAAAACTC
TTCCACTTTTAGTGCATCAACTTCTTATTTGTGTAATAAGAAAATTGGGA
AAACGATCTTCAATATGCTTACCAAGCTGTGATTCCAAATATTACGTAAA
TACACTTGCAAAGGAGGATGTTTTTAGTAGCAATTTGTACTGATGGTATG
GGGCCAAGAGATATATCTTAGAGGGAGGGCTGAGGGTTTGAAGTCCAACT
CCTAAGCCAGTGCCAGAAGAGCCAAGGACAGGTACGGCTGTCATCACTTA
GACCTCACCCTGTGGAGCCACACCCTAGGGTTGGCCAATCTACTCCCAGG
AGCAGGGAGGGCAGGAGCCAGGGCTGGGCATAAAAGTCAGGGCAGAGCCA
TCTATTGCTTACATTTGCTTCTGACACAACTGTGTTCACTAGCAACCTCA
AACAGACACCATGG

SEQ ID NO: 8 is the amino acid sequence of a Her2-specific CDRL1: KASQDVSIGVA

SEQ ID NO: 9 is the amino acid sequence of a Her2-specific CDRL2: ASYRYT

SEQ ID NO: 10 is the amino acid sequence of a Her2-specific CDRL3: QQYYIYPYT

SEQ ID NO: 11 is the amino acid sequence of a Her2-specific CDRH1: GFTFTDYTMD

SEQ ID NO: 12 is the amino acid sequence of a Her2-specific CDRH2:

DVNPNSGGSIYNQRFK

SEQ ID NO: 13 is the amino acid sequence of a Her2-specific CDRH3: LGPSFYFDY

SEQ ID NO: 14 is the amino acid sequence of a PD-L1-specific CDRL1:

RASKGVSTSGYSYLH

SEQ ID NO: 15 is the amino acid sequence of a PD-L1-specific CDRL2: LASYLES

SEQ ID NO: 16 is the amino acid sequence of a PD-L1-specific CDRL3: QHSRDLPLT

SEQ ID NO: 17 is the amino acid sequence of a PD-L1-specific CDRH1: NYYMY

SEQ ID NO: 18 is the amino acid sequence of a PD-L1-specific CDRH2:

GINPSNGGTNFNEKFKN

SEQ ID NO: 19 is the amino acid sequence of a PD-L1 -specific CDRH3: RDYRFDMGFDY

SEQ ID NO: 20 is the amino acid sequence of an Avelumab-specific variable heavy chain:

EVQLLESGGGLVQPGGSLRLSCAASGFTFSSYIMMWVRQAPGKGLEWVSS
IYPSGGITFYADTVKGRFTISRDNSKNTLYLQMNSLRAEDTAVYYCARIK
LGTVTTVDYWGQGTLVTVSS

SEQ ID NO: 21 is the amino acid sequence of an Avelumab-specific variable light chain:

QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
FGTGTKVTVL

SEQ ID NO: 22 is the amino acid sequence of an Avelumab-specific CDRH1:

SGFTFSSYIMM

SEQ ID NO: 23 is the amino acid sequence of an Avelumab-specific CDRH2:

SIYPSGGITFYADTVKG

SEQ ID NO: 24 is the amino acid sequence of an Avelumab-specific CDRH3:

IKLGTVTTVDY

SEQ ID NO: 25 is the amino acid sequence of an Avelumab-specific CDRL1:

TGTSSDVGGYNYVS

SEQ ID NO: 26 is the amino acid sequence of an Avelumab-specific CDRL2: DVSNRPS

SEQ ID NO: 27 is the amino acid sequence of an Avelumab-specific CDRL3:

SSYTSSSTRV

SEQ ID NO: 28 is the amino acid sequence of an Atezolizumab-specific variable heavy chain includes:

EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS

SEQ ID NO: 29 is the amino acid sequence of an Atezolizumab-specific variable light chain:

DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIK

SEQ ID NO: 30 is the amino acid sequence of an Atezolizumab-specific CDRH1:

SGFTFSDSWIH

SEQ ID NO: 31 is the amino acid sequence of an Atezolizumab-specific CDRH2:

WISPYGGSTYYADSVKG

SEQ ID NO: 32 is the amino acid sequence of an Atezolizumab-specific CDRH3:

RHWPGGFDY

SEQ ID NO: 33 is the amino acid sequence of an Atezolizumab-specific CDRL1:

RASQDVSTAVA

SEQ ID NO: 34 is the amino acid sequence of an Atezolizumab-specific CDRL2:

SASFLYS

SEQ ID NO: 35 is the amino acid sequence of an Atezolizumab-specific CDRL3:

QQYLYHPAT

SEQ ID NO: 36 is the amino acid sequence of a PSMA-specific-specific CDRL1:

KASQDVGTAVD

SEQ ID NO: 37 is the amino acid sequence of a PSMA-specific CDRL2: WASTRHT

SEQ ID NO: 38 is the amino acid sequence of a PSMA-specific CDRL3: QQYNSYPLT

SEQ ID NO: 39 is the amino acid sequence of a PSMA-specific CDRH1: GYTFTEYTIH

SEQ ID NO: 40 is the amino acid sequence of a PSMA-specific CDRH2:

NINPNNGGTTYNQKFED

SEQ ID NO: 41 is the amino acid sequence of a PSMA-specific CDRH3: GWNFDY

SEQ ID NO: 42 is the amino acid sequence of a MUC16-specific CDRL1: SEDIYSG

SEQ ID NO: 43 is the amino acid sequence of a MUC16-specific CDRL3: GYSYSSTL

SEQ ID NO: 44 is the amino acid sequence of a MUC16-specific CDRH1: TLGMGVG

SEQ ID NO: 45 is the amino acid sequence of a MUC16-specific CDRH2:

HIWWDDDKYYNPALKS

SEQ ID NO: 46 is the amino acid sequence of a MUC16-specific CDRH3:

IGTAQATDALDY

SEQ ID NO: 47 is the amino acid sequence of a FOLR-specific CDRL1:

KASQSVSFAGTSLMH

SEQ ID NO: 48 is the amino acid sequence of a FOLR-specific CDRL2: RASNLEA

SEQ ID NO: 49 is the amino acid sequence of a FOLR-specific CDRL3: QQSREYPYT

SEQ ID NO: 50 is the amino acid sequence of a FOLR-specific CDRH1: GYFMN

SEQ ID NO: 51 is the amino acid sequence of a FOLR-specific CDRH2:

RIHPYDGDTFYNQKFQG

SEQ ID NO: 52 is the amino acid sequence of a FOLR-specific CDRH3: YDGSRAMDY

SEQ ID NO: 53 is the amino acid sequence of an Amatuximab-specific variable heavy chain:

QVQLQQSGPELEKPGASVKISCKASGYSFTGYTMNWVKQSHGKSLEWIGL
ITPYNGASSYNQKFRGKATLTVDKSSSTAYMDLLSLTSEDSAVYFCARGG
YDGRGFDYWGSGTPVTVSS.

SEQ ID NO: 54 is the amino acid sequence of an Amatuximab-specific variable light chain:

DIELTQSPAIMSASPGEKVTMTCSASSSVSYMHWYQQKSGTSPKRWIYDT
SKLASGVPGRFSGSGSGNSYSLTISSVEAEDDATYYCQQWSKHPLTFGSG
TKVEIK

SEQ ID NO: 55 is the amino acid sequence of an Amatuximab-specific CDRH1:

GYSFTGYTMN

SEQ ID NO: 56 is the amino acid sequence of an Amatuximab-specific CDRH2:

LITPYNGASSYNQ

SEQ ID NO: 57 is the amino acid sequence of an Amatuximab-specific CDRH3:

GGYDGRGFDY

SEQ ID NO: 58 is the amino acid sequence of an Amatuximab-specific CDRL1:

SASSSVSYMH

SEQ ID NO: 59 is the amino acid sequence of an Amatuximab-specific CDRL2: DTSKLAS

SEQ ID NO: 60 is the amino acid sequence of an Amatuximab-specific CDRL3:

QQWSKHPLT

SEQ ID NO: 61 is the amino acid sequence of Nef (66-97):

VGFPVTPQVPLRPMTYKAAVDLSHFLKEKGGL

SEQ ID NO: 62 is the amino acid sequence of Nef (116-145):

HTQGYFPDWQNYTPGPGVRYPLTFGWLYKL

SEQ ID NO: 63 is the amino acid sequence of Gag p17 (17-35):

EKIRLRPGGKKKYKLKHIV

SEQ ID NO: 64 is the amino acid sequence of Gag p17-p24 (253-284):

NPPIPVGEIYKRWIILGLNKIVRMYSPTSILD

SEQ ID NO: 65 is Pol 325-355 (RT 158-188):

AIFQSSMTKILEPFRKQNPDIVIYQYMDDLY

SEQ ID NO: 66 is the nucleotide sequence of a Sequence encoding the IR/DR and chromosomal sequence of Sleeping Beauty:

ACTTAAGTGTATGTAAACTTCCGACTTCAACTGTAGGGTACCTGATTCTC
TGGGCATCTCTGCCCACTACCATG

SEQ ID NO: 67 is the nucleotide sequence of a Sequence encoding the IR/DR and chromosomal sequence of Sleeping Beauty:

ACTTAAGTGTATGTAAACTTCCGACTTCAACTGTAAATTTTCCACCTTTT
TCAGTTTTCCTCGCCATATTTCATG

SEQ ID NO: 68 is the nucleotide sequence of an IR/DR encoding sequence of Sleeping Beauty: ACTTAAGTGTATGTAAACTTCCGACTTCAACTG

SEQ ID NO: 69 is the nucleotide sequence of a sequence encoding the IR/DR and chromosomal sequence of Sleeping Beauty:

CAGTCAACTTAGTGTATGTAAACTTCTGACCCACTGGAATTGTGATACAG
TGAATTATAAGTGAAATAATCTGTCTGTAAACAATTGTTGGAAAAATGAC
TTGTGTCATGCACAAAGTAGATGTCCTAACTGACTTGCCAAAACTATTGT
TTGTTAACAAGAAATTTGTGGAGTAGTTGAAAAACGAGTTTTAATGACTC
CAACTTAAGTGTATGTAAACTTCCGACTTCAACTGTA AGAATGGCCCATT
CATCTATAGTAGCACACAATATTTGCATTTGTGCGACAGTATAAGGGACA
ATTATGCTATCAGGCATTTTTCCAAAGTGAGTAATCGAAGTTTTTATACC
TTTGTGTGCCATGTTTGCTA

SEQ ID NO: 70 is the nucleotide sequence of a sequence encoding the IR/DR and chromosomal sequence of Sleeping Beauty:

CAGTCAACTTAGTGTATGTAAACTTCTGACCCACTGGAATTGTGATACAG
TGAATTATAAGTGAAATAATCTGTCTGTAAACAATTGTTGGAAAAATGAC
TTGTGTCATGCACAAAGTAGATGTCCTAACTGACTTGCCAAAACTATTGT
TTGTTAACAAGAAATTTGTGGAGTAGTTGAAAAACGAGTTTTAATGACTC
CAACTTAAGTGTATGTAAACTTCCGACTTCAACTGTA CAAGTAGACCAAA
TATCCATATACATAAAAGAAAAAAATAGAAAAAATTTCTAGTGACAGAAA
AATGACAAAGAACATACTGCTTTATTACTACTATTAAGATGTTTGCTTCC
ATTACACTCATATGAGTCA

SEQ ID NO: 71 is a Sequence encoding the IR/DR of Sleeping Beauty:

TTAGTGTATGTAAACTTCTGACCCACTGGAATTGTGATACAGTGAATTAT
AAGTGAAATAATCTGTCTGTAAACAATTGTTGGAAAAATGACTTGTGTCA
TGCACAAAGTAGATGTCCTAACTGACTTGCCAAAACTATTGTTTGTTAAC
AAGAAATTTGTGGAGTAGTTGAAAAACGAGTTTTAATGACTCCAACTTAA
GTGTATGTAAACTTCCGACTTCAACTG

SEQ ID NO: 72 is the nucleotide sequence of a sequence encoding the IR/DR and chromosomal sequence of Sleeping Beauty:

CAACTTGAGTGTATGTTAACTTCTGACCCACTGGGAATGTGATGAAAGAA
ATAAAAGCTGAAATGAATCATTCTCTCTACTATTATTCTGATATTTCACA
TTCTTAAAATAAAGTGGTGATCCTAACTGACCTTAAGACAGGGAATCTTT
ACTCGGATTAAATGTCAGGAATTGTGAAAAAGTGAGTTTAAATGTATTTG
GCTAAGGTGTATGTAAACTTCCGACTTCAACTGTA TATCCTCCCCGTTGC
ACCCTCTTGATGATGCTGAGATGAACACAGATGCTCACTCCTTGAGGGCT
CTAAGCTTATGCTGACACAGACACAGGTGCTCACTTCTATGAATGGCCTA
AGATTTGAGGACATCATGAGG

SEQ ID NO: 73 is the nucleotide sequence of a sequence encoding the IR/DR of Sleeping Beauty:

TTGAGTGTATGTTAACTTCTGACCCACTGGGAATGTGATGAAAGAAATAA
AAGCTGAAATGAATCATTCTCTCTACTATTATTCTGATATTTCACATTCT
TAAAATAAAGTGGTGATCCTAACTGACCTTAAGACAGGGAATCTTTACTC
GGATTAAATGTCAGGAATTGTGAAAAAGTGAGTTTAAATGTATTTGGCTA
AGGTGTATGTAAACTTCCGACTTCAACTG

SEQ ID NO: 74 is a Sleeping Beauty transposase enzyme:

MGKSKEISQDLRKKIVDLHKSGSSLGAISKRLKVPRSSVQTIVRKYKHHG
TTQPSYRSGRRRYLSPRDERTLVRKVQINPRTTAKDLVKMLEETGTKVSI
STVKRVLYRHNLKGRSARKKPLLQNRHKKARLRFATAHGDKDRTFWRNVL
WSDETKIELFGHNDHRYVWRKKGEACKPKNTIPTVKHGGGSIMLWGCFAA
GGTGALHKIDGIMRKENYVDILKQHLKTSVRKLKLGRKWVFQMDNDPKHT
SKVVAKWLKDNKVKVLEWPSQSPDLNPIENLWAELKKRVRARRPTNLTQL
HQLCQEEWAKIHPTYCGKLVEGYPKRLTQVKQFKGNATKY

SEQ ID NO: 75 is the amino acid sequence of a Hyperactive Sleeping Beauty is SB100X:

MGKSKEISQDLRKRIVDLHKSGSSLGAISKRLAVPRSSVQTIVRKYKHHG
TTQPSYRSGRRRYLSPRDERTLVRKVQINPRTTAKDLVKMLEETGTKVSI
STVKRVLYRHNLKGHSARKKPLLQNRHKKARLRFATAHGDKDRTFWRNVL
WSDETKIELFGHNDHRYVWRKKGEACKPKNTIPTVKHGGGSIMLWGCFAA
GGTGALHKIDGIMDAVQYVDILKQHLKTSVRKLKLGRKWVFQHDNDPKHT
SKVVAKWLKDNKVKVLEWPSQSPDLNPIENLWAELKKRVRARRPTNLTQL
HQLCQEEWAKIHPNYCGKLVEGYPKRLTQVKQFKGNATKY.

SEQ ID NO: 76 is the amino acid sequence of a piggyBac™ (PB) transposase:

MGSSLDDEHILSALLQSDDELVGEDSDSEISDHVSEDDVQSDTEEAFIDE
VHEVQPTSSGSEILDEQNVIEQPGSSLASNRILTLPQRTIRGKNKHCWST
SKSTRRSRVSALNIVRSQRGPTRMCRNIYDPLLCFKLFFTDEIISEIVKW
TNAEISLKRRESMTGATFRDTNEDEIYAFFGILVMTAVRKDNHMSTDDLF
DRSLSMVYVSVMSRDRFDFLIRCLRMDDKSIRPTLRENDVFTPVRKIWDL
FIHQCIQNYTPGAHLTIDEQLLGFRGRCPFRMYIPNKPSKYGIKILMMCD
SGTKYMINGMPYLGRGTQTNGVPLGEYYVKELSKPVHGSCRNITCDNWFT
SIPLAKNLLQEPYKLTIVGTVRSNKREIPEVLKNSRSRPVGTSMFCFDGP
LTLVSYKPKPAKMVYLLSSCDEDASINESTGKPQMVMYYNQTKGGVDTLD
QMCSVMTCSRKTNRWPMALLYGMINIACINSFIIYSHNVSSKGEKVQSRK
KFMRNLYMSLTSSFMRKRLEAPTLKRYLRDNISNILPNEVPGTSDDSTEE
PVMKKRTYCTYCPSKIRRKANASCKKCKKVICREHNIDMCQSCF

SEQ ID NO: 77 is the amino acid sequence of a Frog Prince transposase:

MPRPKEIQEQLRKKVIEIYQSGKGYKAISKALGIQRTTVRAIIHKWRRHG
TVVNLPRSGRPPKITPRAQRRLIQEVTKDPTTTSKELQASLASVKVSVHA
STIRKRLGKNGLHGRVPRRKPLLSKKNIKARLNFSTTHLDDPQDFWDNIL
WTDETKVELFGRCVSKYIWRRRNTAFHKKNIIPTVKYGGGSVMVWGCFAA
SGPGRLAVIKGTMNSAVYQEILKENVRPSVRVLKLKRTWVLQQDNDPKHT
SKSTTEWLKKNKMKTLEWPSQSPDLNPIEMLWYDLKKAVHARKPSNVTEL
GQFCKDEWAKIPPGRCKSLIARYRKRLVAVVAAKGGPTSY.

SEQ ID NO: 78 is the amino acid sequence of a TcBuster transposase:

MMLNWLKSGKLESQSQEQSSCYLENSNCLPPTLDSTDIIGEENKAGTTSR
KKRKYDEDYLNFGFTWTGDKDEPNGLCVICEQVVNNSSLNPAKLKRHLDT
KHPTLKGKSEYFKRKCNELNQKKHTFERYVRDDNKNLLKASYLVSLRIAK
QGEAYTIAEKLIKPCTKDLTTCVFGEKFASKVDLVPLSDTTISRRIEDMS
YFCEAVLVNRLKNAKCGFTLQMDESTDVAGLAILLVFVRYIHESSFEEDM
LFCKALPTQTTGEEIFNLLNAYFEKHSIPWNLCYHICTDGAKAMVGVIKG
VIARIKKLVPDIKASHCCLHRHALAVKRIPNALHEVLNDAVKMINFIKSR
PLNARVFALLCDDLGSLHKNLLLHTEVRWLSRGKVLTRFWELRDEIRIFF
NEREFAGKLNDTSWLQNLAYIADIFSYLNEVNLSLQGPNSTIFKVNSRIN
SIKSKLKLWEECITKNNTECFANLNDFLETSNTALDPNLKSNILEHLNGL
KNTFLEYFPPTCNNISWVENPFNECGNVDTLPIKEREQLIDIRTDTTLKS
SFVPDGIGPFWIKLMDEFPEISKRAVKELMPFVTTYLCEKSFSVYVATKT
KYRNRLDAEDDMRLQLTTIHPDIDNLCNNKQAQKSH

SEQ ID NO: 79 is the amino acid sequence of a Tol2 transposase:

MEEVCDSSAAASSTVQNQPQDQEHPWPYLREFFSLSGVNKDSFKMKCVLC
LPLNKEISAFKSSPSNLRKHIERMHPNYLKNYSKLTAQKRKIGTSTHASS
SKQLKVDSVFPVKHVSPVTVNKAILRYIIQGLHPFSTVDLPSFKELISTL
QPGISVITRPTLRSKIAEAALIMKQKVTAAMSEVEWIATTTDCWTARRKS
FIGVTAHWINPGSLERHSAALACKRLMGSHTFEVLASAMNDIHSEYEIRD
KVVCTTTDSGSNFMKAFRVFGVENNDIETEARRCESDDTDSEGCGEGSDG
VEFQDASRVLDQDDGFEFQLPKHQKCACHLLNLVSSVDAQKALSNEHYKK
LYRSVFGKCQALWNKSSRSALAAEAVESESRLQLLRPNQTRWNSTFMAVD
RILQICKEAGEGALRNICTSLEVPMFNPAEMLFLTEWANTMRPVAKVLDI
LQAETNTQLGWLLPSVHQLSLKLQRLHHSLRYCDPLVDALQQGIQTRFKH
MFEDPEIIAAAILLPKFRTSWTNDETIIKRGMDYIRVHLEPLDHKKELAN
SSSDDEDFFASLKPTTHEASKELDGYLACVSDTRESLLTFPAICSLSIKT
NTPLPASAACERLFSTAGLLFSPKRARLDTNNFENQLLLKLNLRFYNFE

SEQ ID NO: 80 is the nucleotide sequence of a SV40 promoter:

GGTGTGGAAAGTCCCCAGGCTCCCCAGCAGGCAGAAGTATGCAAAGCATG
CATCTCAATTAGTCAGCAACCAGGTGTGGAAAGTCCCCAGGCTCCCCAGC
AGGCAGAAGTATGCAAAGCATGCATCTCAATTAGTCAGCAACCATAGTCC
CGCCCCTAACTCCGCCCATCCCGCCCCTAACTCCGCCCAGTTCCGCCCAT
TCTCCGCCCCATGGCTGACTAATTTTTTTTATTTATGCAGAGGCCGAGGC
CGCCTCTGCCTCTGAGCTATTCCAGAAGTAGTGAGGAGGCTTTTTTGGAG
GCCTAGGCTTTTGCAAAAAGCTCCCGGGAGCTTGTATATCCATTTTCG.

SEQ ID NO: 81 is the nucleotide sequence of a dESV40 promoter:

GCATGCATCTCAATTAGTCAGCAACCATAGTCCCGCCCCTAACTCCGCCC
ATCCCGCCCCTAACTCCGCCCAGTTCCGCCCATTCTCCGCCCCATGGCTG
ACTAATTTTTTTTATTTATGCAGAGGCCGAGGCCGCCTCGGCCTCTGAGC
TATTCCAGAAGTAGTGAGGAGGCTTTTTTGGAGGCCTAGGCTTTTGCAAA
AAGCTT

SEQ ID NO: 82 is the nucleotide sequence of a Human telomerase catalytic subunit (hTERT) promoter:

TTGGCCCCTCCCTCGGGTTACCCCACAGCCTAGGCCGATTCGACCTCTCT
CCGCTGGGGCCCTCGCTGGCGTCCCTGCACCCTGGGAGCGCGAGCGGCGC
GCGGGCGGGGAAGCGCGGCCCAGACCCCCGGGTCCGCCCGGAGCAGCTGC
GCTGTCGGGGCCAGGCCGGGCTCCCAGTGGATTCGCGGGCACAGACGCCC
AGGACCGCGCTCCCCACGTGGCGGAGGGACTGGGGACCCGGGCACCCGTC
CTGCCCCTTCACCTTCCAGCTCCGCCTCCTCCGCGCGGACCCCGCCCCGT
CCCGACCCCTCCCGGGTCCCCGGCCCAGCCCCCTCCGGGCCCTCCCAGCC
CCTCCCCTTCCTTTACCGCGGCCCCGCCCTCTCCTCGCGGCGCGAGTTTC
AGGCAGCGCTGCGTCCTGCTGCGCACGTGGGAAGCCCTGGCCCCGGCCAC
CCCCGCCAGATCT

SEQ ID NO: 83 is the nucleotide sequence of a RSV promoter derived from the Schmidt-Ruppin A strain:

ACGCGTCATGTTTGACAGCTTATCATCGCAGATCCGTATGGTGCACTCTC
AGTACAATCTGCTCTGATGCCGCATAGTTAAGCCAGTATCTGCTCCCTGC
TTGTGTGTTGGAGGTCGCTGAGTAGTGCGCGAGCAAAATTTAAGCTACAA
CAAGGCAAGGCTTGACCGACAATTGCATGAAGAATCTGCTTAGGGTTAGG
CGTTTTGCGCTGCTTCGCGATGTACGGGCCAGATATTCGCGTATCTGAGG
GGACTAGGGTGTGTTTAGGCGAAAAGCGGGGCTTCGGTTGTACGCGGTTA
GGAGTCCCCTCAGGATATAGTAGTTTCGCTTTTGCATAGGGAGGGGGAAA
TGTAGTCTTATGCAATACTCTTGTAGTCTTGCAACATGGTAACGATGAGT
TAGCAACATGCCTTACAAGGAGAGAAAAAGCACCGTGCATGCCGATTGGT
GGAAGTAAGGTGGTACGATCGTGCCTTATTAGGAAGGCAACAGACGGGTC
TGACATGGATTGGACGAACCACTAAATTCCGCATTGCAGAGATATTGTAT
TTAAGTGCCTAGCTCGATACAATAAACGCCATTTGACCATTCACCACATT
GGTGTGCACCTCCAAGCTGGGTACCAGCTGCTAGCAAGCTTGAGATCT

SEQ ID NO: 84 is the nucleotide sequence of a hNIS promoter:

GAGTAGCTGGGATTACAGGCATGTGCCACCACGCCTCGCTAATATTAGTA
TTTTTCATACAGACAAGATCTCACTATGTTGCTCAGGGTAGTCTCGAATT
CTGGGACTCAAATGATCCTCCCACTTCAGCCTCCCAAAGTGCTGGGATTA
CAGGCATAAGCCATCATGCCCGGCCTCTGACGCTGTTTCTTTCAACCCCC
AGGATTTCAGATTCCACCAGCTTATGGAGAAGGGAACCAAGTTCGAGATG
CGTGATTGCCCAGAAAGTTGGAGGCTGAGCTGAGACTTGAACCCAGAGAC
CAGAACCTCCAGAGGTCAAAGTCCTCCTCCTGGGTCCCCCAGAGAAGGGC
CCTGAGATGACAGCTCGTTGGTCCTCATGGAAGCGTGACCCCCCCAGTAG
ACTTTCTCCCACACCCAACCTTGGTTTCCTCATCTATATGATAGGGACAA
GCCAGACTCTACCTCCCTGGTGGTCATGGTCTCCGCTTATTCGGGTTCAT
AACCTTAAAGGCCCCTCGCACCACCTCAGTGAGCCATTTATGCCTGGCAC
AGGGCCAACTCTCAGTGCATATCTGCAAAGGAACCAATGAATGAGTGAAT
GAAGTGACAAATGAATAAAGGAATAAATGAATGAGGCACTTATCATGTAC
CAGGCTTTCGTTACCACGTCCCATTTATTCCTCTGAGGCAGGGTCTATTT
TATCCTTGTTACAGATGGGGAAACTAAGGCCCAGGGAGGAGCAAAGTCTT
CCCCAAGTATGTACCCACTCAGAACTTGAGCTCTGAATGTCTCCCACCCA
GCTTAGCCCAAGAGCGGGGTTCAGTGATGCCCACCCCCTAAGGCTCTAGA
GAAAGGGGGTAGGCCCACATGCCAGTTTGGGGGTGGTAAAGCCAGGTAAG
TTTTCTTTATGGGTCCCCTGAAACCCTGAAAGTGAACCCCAGTCCTGCAT
GAAAGTGAGCTCCCCATAGCTCAAGGTATTCAAGCACAATACGGCTTTGA
GTGCTGAAGCAGGCTGTGCAGGCTTGGATAGTGACATGCCCTCTCTGAGC
CTCAATTTCCCCACCTGTCAACAGCAGACAGTGACAGCTGTGATCAGGGG
ATCACAGTGCATGGGGATGGGTGGGTGCATGGGGATGGAGGGGCATTTGG
GAGCCCTCCCCGATACCACCCCCTGCAGCCACCCAGATAGCCTGTCCTGG
CCTGTCTGTCCCAGTCCAGGGCTGAAAGGGTGCGGGTCCTGCCCGCCCCT
AGGTCTGGAGGCGGAGTCGCGGTGACCCGGGAGCCCAATAAATCTGCAAC
CCACAATCACGAGCTGCTCCCGTAAGCCCCAAGGCGACCTCCAGCTGTCA
GCGCTGAGCACAGCGCCCAGGGAGAGGGACAGACAGCCGGCTGCATGGGA
CAGCGGAACCCAGAGTGAGAGGGGAGGTGGCAGGACAGACAGACAGCAGG
GGCGGACGCAGAGACAGACAGCGGGGACAGGGAGGCCGACACGGACATCG
ACAGCCCATAGATTCCTAACCCAGGGAGCCCCGGCCCCTCTCGCCGCTTC
CCACCCCAGACGGAGCGGGGACAGGCTGCCGAGCATCCTCCCACCCGCCC
TCCCCGTCCTGCCTCCTCGGCCCCTGCCAGCTTCCCCCGCTTGAGCACGC
AGGGCGTCCGAGGACGCGCTGGGCCTCCGCACCCGCCCTCATGGAGGCCG
TGGAGACCGGGGAACGGCCCACCTTCGGAGCCTGGGACTA

SEQ ID NO: 85 is the nucleotide sequence of a Human glucocorticoid receptor 1A (hGR 1/Ap/e) promoter:

ATTAGAGATTGTAAATTGGGCTCTGAGCTTCCTACCAACAAAAGCACAAA
GGAAAATATGATCACTGGTATTAAAAAAAAACACCTATGGTTTCCAAAAG
ATTAAAACAAACCAGCAGTTTTATAGAAGCTAACACTAAAATCTAAAGGA
ACTACGTTCTATGGAGCCACTTAATATGGATAAACACTTTGACAATATTC
TTTCAACAACTACAGTAACAAGTTTCTTAGAGTCCATTTCTTTTTACATC
CATAATGAATTGTAAATCTTTTCTACTTCTTAAGTAAAACATCACCACTT
AATTCTGGTAACTTTTCCATATTAACTTTTTAGAACAATTGCAAACGTAC
CATAAATGATTGTTGTCACAGTGGTAACTATTTGACCCTGACTGTTATTT
TGTATATAGCAGCTTTTAAAATAAAAAGGCAACAAGTTTCTAGGCGTAAT
TTCCACAGATCTTTTATGTAAAACAATGACATCCTTTGCAACTTCTGCCA
TTTAATCTATCTCAAGCAAGCTCTCTGGAAACAAATCTATTTGAAAGATT
CTATTGTAATTAGAAATCAGGGTAACTGAATGCACTAGATGAAAACCTTC
TGACTGGGGCCAATGAAGTCAATAAAGTCAAAACTGCTGTGAATGCTCAA
CTGTCTGCAGATCAGATGTCTTGGGATGGAATCCGTTCTCGAGGCCACCA
TCATTAATATCAATTTGGCCATGTAATACAAGCCTCACTTGTTCCACTGT
TACAAATGTGCTTAAAACTGAGCTCATTTACAATCCAAATACATATGTAG
GATGGTAACCAAGGCATCACACTAATTTAGGTATTATGTTTTAGGGGGAA
CAAAAGGTATGTTAATATTTTATTCATCTCCAAATTAACTATAAATTGTG
CATTCTTGCATAGATCCTCCTTGGGAATGAGAAATTAGGAAAATCCAGTT
GTTAAAATGAATGCCTAAAATCAAAATAAAATTTGTTTTTCTGGCACCTG
CTTGATGACACAGACTAATAACCAATGACAAAATTCCCTTGAACCCAAGT
TTTCATTTCCTCCTATTGTGTGGTC

SEQ ID NO: 86 is the nucleotide sequence of a Human γ-globin forward primer:

5'- GTGCTTGAAGGGGAACAACTAC -3'

SEQ ID NO: 87 is the nucleotide sequence of a Human γ-globin reverse primer:

5'- CCTGGCCTCCAGATAACTACAC -3’

SEQ ID NO: 88 is the nucleotide sequence of an EF1α p1 forward primer:

CCCCCTCGAGGTCGACATGGCTAGAGACTTATCGAAAGCA

SEQ ID NO: 89 is the nucleotide sequence of an EF1α p1 reverse primer:

ATTCGATATCAAGCTCCAAGATCTGCACACTGGTATTT

SEQ ID NO: 90 is the nucleotide sequence of an EF1α p2 forward primer:

CCCCCTCGAGGTCGACGTACACGACATCACTTTCCCAGT

SEQ ID NO: 91 is the nucleotide sequence of an EF1α p2 reverse primer:

ATTCGATATCAAGCTCACACTGGTATTTCGGTTTTTG

SEQ ID NO: 92 is the nucleotide sequence of a 3′HS1 p1 forward primer:

CCCCCTCGAGGTCGACCTACACTCTCAGTCAGCCTATGGA

SEQ ID NO: 93 is the nucleotide sequence of a 3′HS1 p1 reverse primer:

ATTCGATATCAAGCTTAATCCCAAAAGGCTGATAGTCTC

SEQ ID NO: 94 is the nucleotide sequence of a 3′HS1 p2 forward: primer

CCCCCTCGAGGTCGACACATCTCTCACTTTCTCATCACCA

SEQ ID NO: 95 is the nucleotide sequence of a 3′HS1 p2 reverse primer:

ATTCGATATCAAGCTAAGTAACTGGGATTACAGGAGCAC

SEQ ID NO: 96 is the nucleotide sequence of a CD46F primer: 5′-AAAGGGCAAAT ACCTTAAGGGGTG-3′

SEQ ID NO: 97 is the nucleotide sequence of a CD46R primer: 5′-AGCACTTCGACCTAAAAATAGAGAT-3′

SEQ ID NO: 98 - long β-globin LCR with inserted Xhol site (positions 10655-10661):

GATCTCTATCCCCTCCTGTTTTCTCTACGTTATTTATATGGGTATCATCA
CCATCCTGGACAACATCAGGACAGATATCCCTCACCAAGCCAATGTTCCT
CTCTATGTTGGCTCAAATGTCCTTGAACTTTCCTTTCACCACCCTTTCCA
CAGTCAAAAGGATATTGTAGTTTAATGCCTCAGAGTTCAGCTTTTAAGCT
TCTGACAAATTATTCTTCCTCTTTAGGTTCTCCTTTATGGAATCTTCTGT
ACTGATGGCCATGTCCTTTAACTACTATGTAGATATCTGCTACTACCTGT
ATTATGCCTCTACCTTTATTAGCAGAGTTATCTGTACTGTTGGCATGACA
ATCATTTGTTAATATGACTTGCCTTTCCTTTTTCTGCTATTCTTGATCAA
ATGGCTCCTCTTTCTTGCTCCTCTCATTTCTCCTGCCTTCACTTGGACGT
GCTTCACGTAGTCTGTGCTTATGACTGGATTAAAAATTGATATGGACTTA
TCCTAATGTTGTTCGTCATAATATGGGTTTTATGGTCCATTATTATTTCC
TATGCATTGATCTGGAGAAGGCTTCAATCCTTTTACTCTTTGTGGAAAAT
ATCTGTAAACCTTCTGGTTCACTCTGCTATAGCAATTTCAGTTTAGGCTA
GTAAGCATGAGGATGCCTCCTTCTCTGATTTTTCCCACAGTCTGTTGGTC
ACAGAATAACCTGAGTGATTACTGATGAAAGAGTGAGAATGTTATTGATA
GTCACAATGACAAAAAACAAACAACTACAGTCAAAATGTTTCTCTTTTTA
TTAGTGGATTATATTTCCTGACCTATATCTGGCAGGACTCTTTAGAGAGG
TAGCTGAAGCTGCTGTTATGACCACTAGAGGGAAGAAGATACCTGTGGAG
CTAATGGTCCAAGATGGTGGAGCCCCAAGCAAGGAAGTTGTTAAGGAGCC
CTTTTGATTGAAGGTGGGTGCCCCCACCTTACAGGGACAGGACATCTGGA
TACTCCTCCCAGTTTCTCCAGTTTCCCTTTTTCCTAATATATCTCCTGAT
AAAATGTCTATACTCACTTCCCCATTTCTAATAATAAAGCAAAGGCTAGT
TAGTAAGACATCACCTTGCATTTTGAAAATGCCATAGACTTTCAAAATTA
TTTCATACATCGGTCTTTCTTTATTTCAAGAGTCCAGAAATGGCAACATT
ACCTTTGATTCAATGTAATGGAAAGAGCTCTTTCAAGAGACAGAGAAAAG
AATAATTTAATTTCTTTCCCCACACCTCCTTCCCTGTCTCTTACCCTATC
TTCCTTCCTTCTACCCTCCCCATTTCTCTCTCTCATTTCTCAGAAGTATA
TTTTGAAAGGATTCATAGCAGACAGCTAAGGCTGGTTTTTTCTAAGTGAA
GAAGTGATATTGAGAAGGTAGGGTTGCATGAGCCCTTTCAGTTTTTTAGT
TTATATACATCTGTATTGTTAGAATGTTTTATAATATAAATAAAATTATT
TCTCAGTTATATACTAGCTATGTAACCTGTGGATATTTCCTTAAGTATTA
CAAGCTATACTTAACTCACTTGGAAAACTCAAATAAATACCTGCTTCATA
GTTATTAATAAGGATTAAGTGAGATAATGCCCATAAGATTCCTATTAATA
ACAGATAAATACATACACACACACACACATTGAAAGGATTCTTACTTTGT
GCTAGGAACTATAATAAGTTCATTGATGCATTATATCATTAAGTTCTAAT
TTCAACACTAGAAGGCAGGTATTATCTAAATTTCATACTGGATACCTCCA
AACTCATAAAGATAATTAAATTGCCTTTTGTCATATATTTATTCAAAAGG
GTAAACTCAAACTATGGCTTGTCTAATTTTATATATCACCCTACTGAACA
TGACCCTATTGTGATATTTTATAAAATTATTCTCAAGTTATTATGAGGAT
GTTGAAAGACAGAGAGGATGGGGTGCTATGCCCCAAATCAGCCTCACAAT
TAAGCTAAGCAGCTAAGAGTCTTGCAGGGTAGTGTAGGGACCACAGGGTT
AAGGGGGCAGTAGAATTATACTCCCACTTTAGTTTCATTTCAAACAATCC
ATACACACACAGCCCTGAGCACTTACAAATTATACTACGCTCTATACTTT
TTGTTTAAATGTATAAATAAGTGGATGAAAGAATAGATAGATAGATAGAC
AGATAGATGATAGATAGAATAAATGCTTGCCTTCATAGCTGTCTCCCTAC
CTTGTTCAAAATGTTCCTGTCCAGACCAAAGTACCTTGCCTTCACTTAAG
TAATCAATTCCTAGGTTATATTCTGATGTCAAAGGAAGTCAAAAGATGTG
AAAAACAATTTCTGACCCACAACTCATGCTTTGTAGATGACTAGATCAAA
AAATTTCAGCCATATCTTAACAGTGAGTGAACAGGAAATCTCCTCTTTTC
CCTACATCTGAGATCCCAGCTTCTAAGACCTTCAATTCTCACTCTTGATG
CAACAGACCTTGGAAGCATACAGGAGAGCTGAACTTGGTCAACAAAGGAG
AAAAGTTTGTTGGCCTCCAAAGGCACAGCTCAAACTTTTCAAGCCTTCTC
TAATCTTAAAGGTAAACAAGGGTCTCATTTCTTTGAGAACTTCAGGGAAA
ATAGACAAGGACTTGCCTGGTGCTTTTGGTAGGGGAGCTTGCACTTTCCC
CCTTTCTGGAGGAAATATTTATCCCCAGGTAGTTCCCTTTTTGCACCAGT
GGTTCTTTGAAGAGACTTCCACCTGGGAACAGTTAAACAGCAACTACAGG
GCCTTGAACTGCACACTTTCAGTCCGGTCCTCACAGTTGAAAAGACCTAA
GCTTGTGCCTGATTTAAGCCTTTTTGGTCATAAAACATTGAATTCTAATC
TCCCTCTCAACCCTACAGTCACCCATTTGGTATATTAAAGATGTGTTGTC
TACTGTCTAGTATCCCTCAAGTAGTGTCAGGAATTAGTCATTTAAATAGT
CTGCAAGCCAGGAGTGGTGGCTCATGTCTGTAATTCCAGCACTTGAGAGG
TAGAAGTGGGAGGACTGCTTGAGCTCAAGAGTTTGATATTATCCTGGACA
ACATAGCAAGACCTCGTCTCTACTTAAAAAAAAAAAAAAAATTAGCCAGG
CATGTGATGTACACCTGTAGTCCCAGCTACTCAGGAGGCCGAAATGGGAG
GATCCCTTGAGCTCAGGAGGTCAAGGCTGCAGTGAGACATGATCTTGCCA
CTGCACTCCAGCCTGGACAGCAGAGTGAAACCTTGCCTCACGAAACAGAA
TACAAAAACAAACAAACAAAAAACTGCTCCGCAATGCGCTTCCTTGATGC
TCTACCACATAGGTCTGGGTACTTTGTACACATTATCTCATTGCTGTTCA
TAATTGTTAGATTAATTTTGTAATATTGATATTATTCCTAGAAAGCTGAG
GCCTCAAGATGATAACTTTTATTTTCTGGACTTGTAATAGCTTTCTCTTG
TATTCACCATGTTGTAACTTTCTTAGAGTAGTAACAATATAAAGTTATTG
TGAGTTTTTGCAAACACAGCAAACACAACGACCCATATAGACATTGATGT
GAAATTGTCTATTGTCAATTTATGGGAAAACAAGTATGTACTTTTTCTAC
TAAGCCATTGAAACAGGAATAACAGAACAAGATTGAAAGAATACATTTTC
CGAAATTACTTGAGTATTATACAAAGACAAGCACGTGGACCTGGGAGGAG
GGTTATTGTCCATGACTGGTGTGTGGAGACAAATGCAGGTTTATAATAGA
TGGGATGGCATCTAGCGCAATGACTTTGCCATCACTTTTAGAGAGCTCTT
GGGGACCCCAGTACACAAGAGGGGACGCAGGGTATATGTAGACATCTCAT
TCTTTTTCTTAGTGTGAGAATAAGAATAGCCATGACCTGAGTTTATAGAC
AATGAGCCCTTTTCTCTCTCCCACTCAGCAGCTATGAGATGGCTTGCCCT
GCCTCTCTACTAGGCTGACTCACTCCAAGGCCCAGCAATGGGCAGGGCTC
TGTCAGGGCTTTGATAGCACTATCTGCAGAGCCAGGGCCGAGAAGGGGTG
GACTCCAGAGACTCTCCCTCCCATTCCCGAGCAGGGTTTGCTTATTTATG
CATTTAAATGATATATTTATTTTAAAAGAAATAACAGGAGACTGCCCAGC
CCTGGCTGTGACATGGAAACTATGTAGAATATTTTGGGTTCCATTTTTTT
TTCCTTCTTTCAGTTAGAGGAAAAGGGGCTCACTGCACATACACTAGACA
GAAAGTCAGGAGCTTTGAATCCAAGCCTGATCATTTCCATGTCATACTGA
GAAAGTCCCCACCCTTCTCTGAGCCTCAGTTTCTCTTTTTATAAGTAGGA
GTCTGGAGTAAATGATTTCCAATGGCTCTCATTTCAATACAAAATTTCCG
TTTATTAAATGCATGAGCTTCTGTTACTCCAAGACTGAGAAGGAAATTGA
ACCTGAGACTCATTGACTGGCAAGATGTCCCCAGAGGCTCTCATTCAGCA
ATAAAATTCTCACCTTCACCCAGGCCCACTGAGTGTCAGATTTGCATGCA
CTAGTTCACGTGTGTAAAAAGGAGGATGCTTCTTTCCTTTGTATTCTCAC
ATACCTTTAGGAAAGAACTTAGCACCCTTCCCACACAGCCATCCCAATAA
CTCATTTCAGTGACTCAACCCTTGACTTTATAAAAGTCTTGGGCAGTATA
GAGCAGAGATTAAGAGTACAGATGCTGGAGCCAGACCACCTGAGTGATTA
GTGACTCAGTTTCTCTTAGTAGTTGTATGACTCAGTTTCTTCATCTGTAA
AATGGAGGGTTTTTTAATTAGTTTGTTTTTGAGAAAGGGTCTCACTCTGT
CACCCAAATGGGAGTGTAGTGGCAAAATCTCGGCTCACTGCAACTTGCAC
TTCCCAGGCTCAAGCGGTCCTCCCACCTCAACATCCTGAGTAGCTGGAAC
CACAGGTACACACCACCATACCTCGCTAATTTTTTGTATTTTTGGTAGAG
ATGGGGTTTCACATGTTACACAGGATGGTCTCAGACTCCGGAGCTCAAGC
AATCTGCCCACCTCAGCCTTCCAAAGTGCTGGGATTATAAGCATGATTAC
AGGAGTTTTAACAGGCTCATAAGATTGTTCTGCAGCCCGAGTGAGTTAAT
ACATGCAAAGAGTTTAAAGCAGTGACTTATAAATGCTAACTACTCTAGAA
ATGTTTGCTAGTATTTTTTGTTTAACTGCAATCATTCTTGCTGCAGGTGA
AAACTAGTGTTCTGTACTTTATGCCCATTCATCTTTAACTGTAATAATAA
AAATAACTGACATTTATTGAAGGCTATCAGAGACTGTAATTAGTGCTTTG
CATAATTAATCATATTTAATACTCTTGGATTCTTTCAGGTAGATACTATT
ATTATCCCCATTTTACTACAGTTAAAAAAACTACCTCTCAACTTGCTCAA
GCATACACTCTCACACACACAAACATAAACTACTAGCAAATAGTAGAATT
GAGATTTGGTCCTAATTATGTCTTTGCTCACTATCCAATAAATATTTATT
GACATGTACTTCTTGGCAGTCTGTATGCTGGATGCTGGGGATACAAAGAT
GTTTAAATTTAAGCTCCAGTCTCTGCTTCCAAAGGCCTCCCAGGCCAAGT
TATCCATTCAGAAAGCATTTTTTACTCTTTGCATTCCACTGTTTTTCCTA
AGTGACTAAAAAATTACACTTTATTCGTCTGTGTCCTGCTCTGGGATGAT
AGTCTGACTTTCCTAACCTGAGCCTAACATCCCTGACATCAGGAAAGACT
ACACCATGTGGAGAAGGGGTGGTGGTTTTGATTGCTGCTGTCTTCAGTTA
GATGGTTAACTTTGTGAAGTTGAAAACTGTGGCTCTCTGGTTGACTGTTA
GAGTTCTGGCACTTGTCACTATGCCTATTATTTAACAAATGCATGAATGC
TTCAGAATATGGGAATATTATCTTCTGGAATAGGGAATCAAGTTATATTA
TGTAACCCAGGATTAGAAGATTCTTCTGTGTGTAAGAATTTCATAAACAT
TAAGCTGTCTAGCAAAAGCAAGGGCTTGGAAAATCTGTGAGCTCCTCACC
ATATAGAAAGCTTTTAACCCATCATTGAATAAATCCCTATAGGGGATTTC
TACCCTGAGCAAAAGGCTGGTCTTGATTAATTCCCAAACTCATATAGCTC
TGAGAAAGTCTATGCTGTTAACGTTTTCTTGTCTGCTACCCCATCATATG
CACAACAATAAATGCAGGCCTAGGCATGACTGAAGGCTCTCTCATAATTC
TTGGTTGCATGAATCAGATTATCAACAGAAATGTTGAGACAAACTATGGG
GAAGCAGGGTATGAAAGAGCTCTGAATGAAATGGAAACCGCAATGCTTCC
TGCCCATTCAGGGCTCCAGCATGTAGAAATCTGGGGCTTTGTGAAGACTG
GCTTAAAATCAGAAGCCCCATTGGATAAGAGTAGGGAAGAACCTAGAGCC
TACGCTGAGCAGGTTTCCTTCATGTGACAGGGAGCCTCCTGCCCCGAACT
TCCAGGGATCCTCTCTTAAGTGTTTCCTGCTGGAATCTCCTCACTTCTAT
CTGGAAATGGTTTCTCCACAGTCCAGCCCCTGGCTAGTTGAAAGAGTTAC
CCATGCAGAGGCCCTCCTAGCATCCAGAGACTAGTGCTTAGATTCCTACT
TTCAGCGTTGGACAACCTGGATCCACTTGCCCAGTGTTCTTCCTTAGTTC
CTACCTTCGACCTTGATCCTCCTTTATCTTCCTGAACCCTGCTGAGATGA
TCTATGTGGGGAGAATGGCTTCTTTGAGAAACATCTTCTTCGTTAGTGGC
CTGCCCCTCATTCCCACTTTAATATCCAGAATCACTATAAGAAGAATATA
ATAAGAGGAATAACTCTTATTATAGGTAAGGGAAAATTAAGAGGCATACG
TGATGGGATGAGTAAGAGAGGAGAGGGAAGGATTAATGGACGATAAAATC
TACTACTATTTGTTGAGACCTTTTATAGTCTAATCAATTTTGCTATTGTT
TTCCATCCTCACGCTAACTCCATAAAAAAACACTATTATTATCTTTATTT
TGCCATGACAAGACTGAGCTCAGAAGAGTCAAGCATTTGCCTAAGGTCGG
ACATGTCAGAGGCAGTGCCAGACCTATGTGAGACTCTGCAGCTACTGCTC
ATGGGCCCTGTGCTGCACTGATGAGGAGGATCAGATGGATGGGGCAATGA
AGCAAAGGAATCATTCTGTGGATAAAGGAGACAGCCATGAAGAAGTCTAT
GACTGTAAATTTGGGAGCAGGAGTCTCTAAGGACTTGGATTTCAAGGAAT
TTTGACTCAGCAAACACAAGACCCTCACGGTGACTTTGCGAGCTGGTGTG
CCAGATGTGTCTATCAGAGGTTCCAGGGAGGGTGGGGTGGGGTCAGGGCT
GGCCACCAGCTATCAGGGCCCAGATGGGTTATAGGCTGGCAGGCTCAGAT
AGGTGGTTAGGTCAGGTTGGTGGTGCTGGGTGGAGTCCATGACTCCCAGG
AGCCAGGAGAGATAGACCATGAGTAGAGGGCAGACATGGGAAAGGTGGGG
GAGGCACAGCATAGCAGCATTTTTCATTCTACTACTACATGGGACTGCTC
CCCTATACCCCCAGCTAGGGGCAAGTGCCTTGACTCCTATGTTTTCAGGA
TCATCATCTATAAAGTAAGAGTAATAATTGTGTCTATCTCATAGGGTTAT
TATGAGGATCAAAGGAGATGCACACTCTCTGGACCAGTGGCCTAACAGTT
CAGGACAGAGCTATGGGCTTCCTATGTATGGGTCAGTGGTCTCAATGTAG
CAGGCAAGTTCCAGAAGATAGCATCAACCACTGTTAGAGATATACTGCCA
GTCTCAGAGCCTGATGTTAATTTAGCAATGGGCTGGGACCCTCCTCCAGT
AGAACCTTCTAACCAGCTGCTGCAGTCAAAGTCGAATGCAGCTGGTTAGA
CTTTTTTTAATGAAAGCTTAGCTTTCATTAAAGATTAAGCTCCTAAGCAG
GGCACAGATGAAATTGTCTAACAGCAACTTTGCCATCTAAAAAAATCTGA
CTTCACTGGAAACATGGAAGCCCAAGGTTCTGAACATGAGAAATTTTTAG
GAATCTGCACAGGAGTTGAGAGGGAAACAAGATGGTGAAGGGACTAGAAA
CCACATGAGAGACACGAGGAAATAGTGTAGATTTAGGCTGGAGGTAAATG
AAAGAGAAGTGGGAATTAATACTTACTGAAATCTTTCTATATGTCAGGTG
CCATTTTATGATATTTAATAATCTCATTACATATGGTAATTCTGTGAGAT
ATGTATTATTGAACATACTATAATTAATACTAATGATAAGTAACACCTCT
TGAGTACTTAGTATATGCTAGAATCAAATTTAAGTTTATCATATGAGGCC
GGGCACGGTGGCTCATATATGGGATTACATGCCTGTAATCCCAGCACTTT
GGGAGGCCAAGGCAATTGGATCACCTGAGGTCAGGAGTTCCAGACCAGCC
TGGCCAACATGGTGAAACCCCTTCTCTACTAAAAAATACAAAAAATCAGC
CAGGTGTGGTGGCACGCGTCTATAATCCCAGCTACTCAGGAGGCTGAGGC
AGGAGAATCACTTGAACCCAGGAGGTGGAGGTTGCAGTGAGCTAAGATTG
CACCACTGCACTCCAGCCTAGGCGACAGAGTGAGACTCCATCTCAAAAAA
AAAAAAAGAAGTTTATTATATGAATTAACTTAGTTTTACTCACACCAATA
CTCAGAAGTAGATTATTACCTCATTTATTGATGAGGAGCCCAATGTACTT
GTAGTGTAGATCAACTTATTGAAAGCACAAGCTAATAAGTAGACAATTAG
TAATTAGAAGTCAGATGGTCTGAGCTCTCCTACTGTCTACATTACATGAG
CTCTTATTAACTGGGGACTCGAAAATCAAAGACATGAAATAATTTGTCCA
AGCTTACAGAACCACCAAGTAGTAAGGCTAGGATGTAGACCCAGTTCTGC
TACCTCTGAAGACAGTGTTTTTTCCACAGCAAAACACAAACTCAGATATT
GTGGATGCGAGAAATTAGAAGTAGATATTCCTGCCCTGTGGCCCTTGCTT
CTTACTTTTACTTCTTGTCGATTGGAAGTTGTGGTCCAAGCCACAGTTGC
AGACCATACTTCCTCAACCATAATTGCATTTCTTCAGGAAAGTTTGAGGG
AGAAAAAGGTAAAGAAAAATTTAGAAACAACTTCAGAATAAAGAGATTTT
CTCTTGGGTTACAGAGATTGTCATATGACAAATTATAAGCAGACACTTGA
GAAAACTGAAGGCCCATGCCTGCCCAAATTACCCTTTGACCCCTTGGTCA
AGCTGCAACTTTGGTTAAAGGGAGTGTTTATGTGTTATAGTGTTCATTTA
CTCTTCTGGTCTAACCCATTGGCTCCGTCTTCATCCTGCAGTGACCTCAG
TGCCTCAGAAACATACATATGTTTGTCTAGTTTAAGTTTGTGTGAAATTC
TAACTAGCGTCAAGAACTGAGGGCCCTAAACTATGCTAGGAATAGTGCTG
TGGTGCTGTGATAGGTACACAAGAAATGAGAAGAAACTGCAGATTCTCTG
CATCTCCCTTTGCCGGGTCTGACAACAAAGTTTCCCCAAATTTTACCAAT
GCAAGCCATTTCTCCATATGCTAACTACTTTAAAATCATTTGGGGCTTCA
CATTGTCTTTCTCATCTGTAAAAAGAATGGAAGAACTCATTCCTACAGAA
CTCCCTATGTCTTCCCTGATGGGCTAGAGTTCCTCTTTCTCAAAAATTAG
CCATTATTGTATTTCCTTCTAAGCCAAAGCTCAGAGGTCTTGTATTGCCC
AGTGACATGCACACTGGTCAAAAGTAGGCTAAGTAGAAGGGTACTTTCAC
AGGAACAGAGAGCAAAAGAGGTGGGTGAATGAGAGGGTAAGTGAGAAAAG
ACAAATGAGAAGTTACAACATGATGGCTTGTTGTCTAAATATCTCCTAGG
GAATTATTGTGAGAGGTCTGAATAGTGTTGTAAAATAAGCTGAATCTGCT
GCCAACATTAACAGTCAAGAAATACCTCCGAATAACTGTACCTCCAATTA
TTCTTTAAGGTAGCATGCAACTGTAATAGTTGCATGTATATATTTATCAT
AATACTGTAACAGAAAACACTTACTGAATATATACTGTGTCCCTAGTTCT
TTACACAATAAACTAATCTCATCCTCATAATTCTATTAGCTAATACATAT
TATCATCCTATATTTCAGAGACTTCAAGAAGTTAAGCAACTTGCTCAAGA
TCATCTAAGAAGTAGGTGGTATTTCTGGGCTCATTTGGCCCCTCCTAATC
TCTCATGGCAACATGGCTGCCTAAAGTGTTGATTGCCTTAATTCATCAGG
GATGGGCTCATACTCACTGCAGACCTTAACTGGCATCCTCTTTTCTTATG
TGATCTGCCTGACCCTAGTAGACTTATGAAATTTCTGATGAGAAAGGAGA
GAGGAGAAAGGCAGAGCTGACTGTGATGAGTGATGAAGGTGCCTTCTCAT
CTGGCTCGAGGGTACCAGTGGGGCCTCTAAGACTAAGTCACTCTGTCTCA
CTGTGTCTTAGCCAGTTCCTTACAGCTTGCCCTGATGGGAGATAGAGAAT
GGGTATCCTCCAACAAAAAAATAAATTTTCATTTCTCAAGGTCCAACTTA
TGTTTTCTTAATTTTTAAAAAAATCTTGACCATTCTCCACTCTCTAAAAT
AATCCACAGTGAGAGAAACATTCTTTTCCCCCATCCCATAAATACCTCTA
TTAAATATGGAAAATCTGGGCATGGTGTCTCACACCTGTAATCCCAGCAC
TTTGGGAGGCTGAGGTGGGTGGACTGCTTGGAGCTCAGGAGTTCAAGACC
ATCTTGGACAACATGGTGATACCCTGCCTCTACAAAAAGTACAAAAATTA
GCCTGGCATGGTGGTGTGCACCTGTAATCCCAGCTATTAGGGTGGCTGAG
GCAGGAGAATTGCTTGAACCCGGGAGGCGGAGGTTGCAGTGAGCTGAGAT
CGTGCCACTGCACTCCAGCCTGGGGGACAGAGCACATTATAATTAACTGT
TATTTTTTACTTGGACTCTTGTGGGGAATAAGATACATGTTTTATTCTTA
TTTATGATTCAAGCACTGAAAATAGTGTTTAGCATCCAGCAGGTGCTTCA
AAACCATTTGCTGAATGATTACTATACTTTTTACAAGCTCAGCTCCCTCT
ATCCCTTCCAGCATCCTCATCTCTGATTAAATAAGCTTCAGTTTTTCCTT
AGTTCCTGTTACATTTCTGTGTGTCTCCATTAGTGACCTCCCATAGTCCA
AGCATGAGCAGTTCTGGCCAGGCCCCTGTCGGGGTCAGTGCCCCACCCCC
GCCTTCTGGTTCTGTGTAACCTTCTAAGCAAACCTTCTGGCTCAAGCACA
GCAATGCTGAGTCATGATGAGTCATGCTGAGGCTTAGGGTGTGTGCCCAG
ATGTTCTCAGCCTAGAGTGATGACTCCTATCTGGGTCCCCAGCAGGATGC
TTACAGGGCAGATGGCAAAAAAAAGGAGAAGCTGACCACCTGACTAAAAC
TCCACCTCAAACGGCATCATAAAGAAAATGGATGCCTGAGACAGAATGTG
ACATATTCTAGAATATATTATTTCCTGAATATATATATATATATACACAT
ATACGTATATATATATATATATATATATTTGTTGTTATCAATTGCCATAG
AATGATTAGTTATTGTGAATCAAATATTTATCTTGCAGGTGGCCTCTATA
CCTAGAAGCGGCAGAATCAGGCTTTATTAATACATGTGTATAGATTTTTA
GGATCTATACACATGTATTAATATGAAACAAGGATATGGAAGAGGAAGGC
ATGAAAACAGGAAAAGAAAACAAACCTTGTTTGCCATTTTAAGGCACCCC
TGGACAGCTAGGTGGCAAAAGGCCTGTGCTGTTAGAGGACACATGCTCAC
ATACGGGGTCAGATCTGACTTGGGGTGCTACTGGGAAGCTCTCATCTTAA
GGATACATCTCAGGCCAGTCTTGGTGCATTAGGAAGATGTAGGCAACTCT
GATCCTGAGAGGAAAGAAACATTCCTCCAGGAGAGCTAAAAGGGTTCACC
TGTGTGGGTAACTGTGAAGGACTACAAGAGGATGAAAAACAATGACAGAC
AGACATAATGCTTGTGGGAGAAAAAACAGGAGGTCAAGGGGATAGAGAAG
GCTTCCAGAAGAATGGCTTTGAAGCTGGCTTCTGTAGGAGTTCACAGTGG
CAAAGATGTTTCAGAAATGTGACATGACTTAAGGAACTATACAAAAAGGA
ACAAATTTAAGGAGAGGCAGATAAATTAGTTCAACAGACATGCAAGGAAT
TTTCAGATGAATGTTATGTCTCCACTGAGCTTCTTGAGGTTAGCAGCTGT
GAGGGTTTTGCAGGCCCAGGACCCATTACAGGACCTCACGTATACTTGAC
ACTGTTTTTTGTATTCATTTGTGAATGAATGACCTCTTGTCAGTCTACTC
GGTTTCGCTGTGAATGAATGATGTCTTGTCAGCCTACTTGGTTTCGCTAA
GAGCACAGAGAGAAGATTTAGTGATGCTATGTAAAAACTTCCTTTTTGGT
TCAAGTGTATGTTTGTGATAGAAATGAAGACAGGCTACATGATGCATATC
TAACATAAACACAAACATTAAGAAAGGAAATCAACCTGAAGAGTATTTAT
ACAGATAACAAAATACAGAGAGTGAGTTAAATGTGTAATAACTGTGGCAC
AGGCTGGAATATGAGCCATTTAAATCACAAATTAATTAGAAAAAAAACAG
TGGGGAAAAAATTCCATGGATGGGTCTAGAAAGACTAGCATTGTTTTAGG
TTGAGTGGCAGTGTTTAAAGGGTGATATCAGACTAAACTTGAAATATGTG
GCTAAATAACTAGAATACTCTTTATTTTTTCGTATCATGAATAGCAGATA
TAGCTTGATGGCCCCATGCTTGGTTTAACATCCTTGCTGTTCCTGACATG
AAATCCTTAATTTTTGACAAAGGGGCTATTCATTTTCATTTTATATTGGG
CCTAGAAATTATGTAGATGGTCCTGAGGAAAAGTTTATAGCTTGTCTATT
TCTCTCTCTAACATAGTTGTCAGCACAATGCCTAGGCTATAGGAAGTACT
CAAAGCTTGTTAAATTGAATTCTATCCTTCTTATTCAATTCTACACATGG
AGGAAAAACTCATCAGGGATGGAGGCACGCCTCTAAGGAAGGCAGGTGTG
GCTCTGCAGTGTGATTGGGTACTTGCAGGACGAAGGGTGGGGTGGGAGTG
GCTAACCTTCCATTCCTAGTGCAGAGGTCACAGCCTAAACATCAAATTCC
TTGAGGTGCGGTGGCTCACTCCTGTAATCACAGCAGTTTGGGACGCCAAG
GTGGGCAGATCACTTGAGGTCAGGAGTTGGACACCAGCCCAGCCAACATA
GTGAAACCTGGTCTCTGCTTAAAAATATAAAAATTAGCTGGACGTGGTGA
CGGGAGCCTGTAATCCAACTACTTGGGAGGCTGAGGCAGGAGAATCGCTT
GAACCGGGGAGGTGGAGTTTGCACTGAGCAGAGATCATGCCATTGCACTC
CAGCCTCCAGAGCGAGACTCTGTCTAAAGAAAAACGAAAACAAACAAACA
AACAAACAAACAAAACCCATCAAATTCCCTGACCGAACAGAATTCTGTCT
GATTGTTCTCTGACTTATCTACCATTTTCCCTCCTTAAAGAAACTGTGAA
CTTCCTTCAGCTAGAGGGGCCTGGCTCAGAAGCCTCTGGTCAGCATCCAA
GAAATACTTGATGTCACTTTGGCTAAAGGTATGATGTGTAGACAAGCTCC
AGAGATGGTTTCTCATTTCCATATCCACCCACCCAGCTTTCCAATTTTAA
AGCCAATTCTGAGGTAGAGACTGTGATGAACAAACACCTTGACAAAATTC
AACCCAAAGACTCACTTTGCCTAGCTTCAAAATCCTTACTCTGACATATA
CTCACAGCCAGAAATTAGCATGCACTAGAGTGTGCATGAGTGCAACACAC
ACACACACCAATTCCATATTCTCTGTCAGAAAATCCTGTTGGTTTTTCGT
GAAAGGATGTTTTCAGAGGCTGACCCCTTGCCTTCACCTCCAATGCTACC
ACTCTGGTCTAAGTCACTGTCACCACCACCTAAATTATAGCTGTTGACTC
ATAACAATCTTCCTGCTTCTACCACTGCCCCACTACAATTTCTTCCCAAT
ATACTATCCAAATTAGTCTTTTCAAAATGTAAGTCATATATGGTCACCTC
TTTGTTCAAAGTCTTCTGATAGTTTCCTATATCATTTATAATAAAACCAA
ATCCTTACAATTCTCTACAATAGTTGTTCATGCATATATTATGTTTATTA
CAGATACATATATATAGCTCTCATATAAATAAATATATATATTTATGTGT
ATGTGTGTAGAGTGTTTTTTCTTACAACTCTATGATGTAGGTATTATTAG
TGTCCCAAATTTTATAATTTAGGACTTCTATGATCTCATCTTTTATTCTC
CCCTTCACCGAATCTCATCCTACATTGGCCTTATTGATATTCCTTGAAAA
TTCTAAGCATCTTACATCTTTAGGGTATTTACATTTGCCATTCCCTATGC
CCTAAATATTTAATCATAGTTTCATATAAATGGGTTCCTCATCATCTATG
GGTACTCTCTCAGGTGTTAACTTTATAGTGAGGACTTTCCTGCCATACTA
CTTAAAGTAGCGATACCCTTTCACCCTGTCCTAATCACACTCTGGCCTTC
ATTTCAGTTTTTTTTTTTTCTCCATAGCACCTAATCTCATTGGTATATAA
CATGTTTCATTTGCTTATTTAATGTCAAGCTCTTTCCACTATCAAGTCCA
TGAAAACAGGAACTTTATTCCTCTATTCTGTTTTTGTGCTGTATTCTTAG
CAATTTTACAATTTTGAATGAATGAATGAGCAGTCAAACACATATACAAC
TATAATTAAAAGGATGTATGCTGACACATCCACTGCTATGCACACACAAA
GAAATCAGTGGAGTAGAGCTGGAAGTGCTAAGCCTGCATAGAGCTAGTTA
GCCCTCCGCAGGCAGAGCCTTGATGGGATTACTGAGTTCTAGAATTGGAC
TCATTTGTTTTGTAGGCTGAGATTTGCTCTTGAAAACTTGTTCTGACCAA
AATAAAAGGCTCAAAAGATGAATATCGAAACCAGGGTGTTTTTTACACTG
GAATTTATAACTAGAGCACTCATGTTTATGTAAGCAATTAATTGTTTCAT
CAGTCAGGTAAAAGTAAAGAAAAACTGTGCCAAGGCAGGTAGCCTAATGC
AATATGCCACTAAAGTAAACATTATTTCATAGGTGTCAGATATGGCTTAT
TCATCCATCTTCATGGGAAGGATGGCCTTGGCCTGGACATCAGTGTTATG
TGAGGTTCAAAACACCTCTAGGCTATAAGGCAACAGAGCTCCTTTTTTTT
TTTTCTGTGCTTTCCTGGCTGTCCAAATCTCTAATGATAAGCATACTTCT
ATTCAATGAGAATATTCTGTAAGATTATAGTTAAGAATTGTGGGAGCCAT
TCCGTCTCTTATAGTTAAATTTGAGCTTCTTTTATGATCACTGTTTTTTT
AATATGCTTTAAGTTCTGGGGTACATGTGCCATGGTGGTTTGCTGCACCC
ATCAACCCGTCATCTACATTAGGTATTTCTCCTAATGCTATCCTTCCCCT
AGCCCCCCACCCCCAACAGGCCCCAGTGTGTGATGTTCCCCTCCCTGTGT
CCATGGATCACTGGTTTTTTTTTGTTTTTTTTTTTTTTTTAAAGTCTCAG
TTAAATTTTTGGAATGTAATTTATTTTCCTGGTATCCTAGGACTTGCAAG
TTATCTGGTCACTTTAGCCCTCACGTTTTGATGATAATCACATATTTGTA
AACACAACACACACACACACACACACACACATATATATATATATAAAACA
TATATATACATAAACACACATAACATATTTATCGGGCATTTCTGAGCAAC
TAATCATGCAGGACTCTCAAACACTAACCTATAGCCTTTTCTATGTATCT
ACTTGTGTAGAAACCAAGCGTGGGGACTGAGAAGGCAATAGCAGGAGCAT
TCTGACTCTCACTGCCTTTAGCTAGGCCCCTCCCTCATCACAGCTCAGCA
TAGTCCTGAGCTCTTATCTATATCCACACACAGTTTCTGACGCTGCCCAG
CTATCACCATCCCAAGTCTAAAGAAAAAAATAATGGGTTTGCCCATCTCT
GTTGATTAGAAAACAAAACAAAATAAAATAAGCCCCTAAGCTCCCAGAAA
ACATGACTAAACCAGCAAGAAGAAGAAAATACAATAGGTATATGAGGAGA
CTGGTGACACTAGTGTCTGAATGAGGCTTGAGTACAGAAAAGAGGCTCTA
GCAGCATAGTGGTTTAGAGGAGATGTTTCTTTCCTTCACAGATGCCTTAG
CCTCAATAAGCTTGCGGTTGTGGAAGTTTACTTTCAGAACAAACTCCTGT
GGGGCTAGAATTATTGATGGCTAAAAGAAGCCCGGGGGAGGGAAAAATCA
TTCAGCATCCTCACCCTTAGTGACACAAAACAGAGGGGGCCTGGTTTTCC
ATATTTCCTCATGATGGATGATCTCGTTAATGAAGGTGGTCTGACGAGAT
CATTGCTTCTTCCATTTAAGCCTTGCTCACTTGCCAATCCTCAGTTTTAA
CCTTCTCCAGAGAAATACACATTTTTTATTCAGGAAACATACTATGTTAT
AGTTTCAATACTAAATAATCAAAGTACTGAAGATAGCATGCATAGGCAAG
AAAAAGTCCTTAGCTTTATGTTGCTGTTGTTTCAGAATTTAAAAAAGATC
ACCAAGTCAAGGACTTCTCAGTTCTAGCACTAGAGGTGGAATCTTAGCAT
ATAATCAGAGGTTTTTCAAAATTTCTAGACATAAGATTCAAAGCCCTGCA
CTTAAAATAGTCTCATTTGAATTAACTCTTTATATAAATTGAAAGCACAT
TCTGAACTACTTCAGAGTATTGTTTTATTTCTATGTTCTTAGTTCATAAA
TACATTAGGCAATGCAATTTAATTAAAAAAACCCAAGAATTTCTTAGAAT
TTTAATCATGAAAATAAATGAAGGCATCTTTACTTACTCAAGGTCCCAAA
AGGTCAAAGAAACCAGGAAAGTAAAGCTATATTTCAGCGGAAAATGGGAT
ATTTATGAGTTTTCTAAGTTGACAGACTCAAGTTTTAACCTTCAGTGCCC
ATCATGTAGGAAAGTGTGGCATAACTGGCTGATTCTGGCTTTCTACTCCT
TTTTCCCATTAAAGATCCCTCCTGCTTAATTAACATTCACAAGTAACTCT
GGTTGTACTTTAGGCACAGTGGCTCCCGAGGTCAGTCACACAATAGGATG
TCTGTGCTCCAAGTTGCCAGAGAGAGAGATTACTCTTGAGAATGAGCCTC
AGCCCTGGCTCAAACTCACCTGCAAACTTCGTGAGAGATGAGGCAGAGGT
ACACTACGAAAGCAACAGTTAGAAGCTAAATGATGAGAACACATGGACTC
ATAGAGGGAAACAACGCATACTGGGGCCTATCAGAGGGTGGAGGGTGAGA
GAAGGAGAGGATCAGGAAAAATCACTAATGGATGCTAAGCGTAATACCTG
AGTGATGAGATCATCTATACAACAAACCCCCTTGACATTCATTTATCTAT
GTAACAAACCTGCACATCCTGTACATGTACCCCTGAACTTAAAATAAAAG
TTGAAAACAAGAAAGCAACAGTTTGAACACTTGTTATGGTCTATTCTCTC
ATTCTTTACAATTACACTAGAAAATAGCCACAGGCTTCCTGCAAGGCAGC
CACAGAATTTATGACTTGTGATATCCAAGTCATTCCTGGATAATGCAAAA
TCTAACACAAAATCTAGTAGAATCATTTGCTTACATCTATTTTTGTTCTG
AGAATATAGATTTAGATACATAATGGAAGCAGAATAATTTAAAATCTGGC
TAATTTAGAATCCTAAGCAGCTCTTTTCCTATCAGTGGTTTACAAGCCTT
GTTTATATTTTTCCTATTTTAAAAATAAAAATAAAGTAAGTTATTTGTGG
TAAAGAATATTCATTAAAGTATTTATTTCTTAGATAATACCATGAAAAAC
ATTCAGTGAAGTGAAGGGCCTACTTTACTTAACAAGAATCTAATTTATAT
AATTTTTCATACTAATAGCATCTAAGAACAGTACAATATTTGACTCTTCA
GGTTAAACATATGTCATAAATTAGCCAGAAAGATTTAAGAAAATATTGGA
TGTTTCCTTGTTTAAATTAGGCATCTTACAGTTTTTAGAATCCTGCATAG
AACTTAAGAAATTACAAATGCTAAAGCAAACCCAAACAGGCAGGAATTAA
TCTTCATCGAATTTGGGTGTTTCTTTCTAAAAGTCCTTTATACTTAAATG
TCTTAAGACATACATAGATTTTATTTTACTAATTTTAATTATATAGACAA
TAAATGAATATTCTTACTGATTACTTTTTCTGACTGTCTAATCTTTCTGA
TCTATCCTGGATGGCCATAACACTTATCTCTCTGAACTTTGGGCTTTTAA
TATAGGAAAGAAAAGCAATAATCCATTTTTCATGGTATCTCATATGATAA
ACAAATAAAATGCTTAAAAATGAGCAGGTGAAGCAATTTATCTTGAACCA
ACAAGCATCGAAGCAATAATGAGACTGCCCGCAGCCTACCTGACTTCTGA
GTCAGGATTTATAAGCCTTGTTACTGAGACACAAACCTGGGCCTTTCAAT
GCTATAACCTTTCTTGAAGCTCCTCCCTACCACCTTTAGCCATAAGGAAA
CATGGAATGGGTCAGATCCCTGGATGCAAGCCAGGTCTGGAACCATAGGC
AGTAAGGAGAGAAGAAAATGTGGGCTCTGCAACTGGCTCCGAGGGAGCAG
GAGAGGATCAACCCCATACTCTGAATCTAAGAGAAGACTGGTGTCCATAC
TCTGAATGGGAAGAATGATGGGATTACCCATAGGGCTTGTTTTAGGGAGA
AACCTGTTCTCCAAACTCTTGGCCTTGAGATACCTGGTCCTTATTCCTTG
GACTTTGGCAATGTCTGACCCTCACATTCAAGTTCTGAGGAAGGGCCACT
GCCTTCATACTGTGGATCTGTAGCAAATTCCCCCTGAAAACCCAGAGCTG
TATCTTAATTGGTTAAAAAAAATTATATTATCTCAACGACTGTTCTTCTC
TGAGTAGCCAAGCTCAGCTTGGTTCAAGCTACAAGCAGCTGAGCTGCTTT
TTGTCTAGTCATTGTTCTTTTATTTCAGTGGATCAAATACGTTCTTTCCA
AACCTAGGATCTTGTCTTCCTAGGCTATATATTTTGTCCCAGGAAGTCTT
AATCTGGGGTCCACAGAACACTAGGGGGCTGGTGAAGTTTATAGAAAAAA
AATCTGTATTTTTACTTACATGTAACTGAAATTTAGCATTTTCTTCTACT
TTGAATGCAAAGGACAAACTAGAATGACATCATCAGTACCTATTGCATAG
TTATAAAGAGAAACCACAGATATTTTCATACTACACCATAGGTATTGCAG
ATCTTTTTGTTTTTGTTTTTGTTTGAGATGGAGTTTCGCTCTTATTGCCC
AGGCTGGAGTGCAGTGGCATGATTTCGGCTCACTGCAACCTCCCCTTCCT
GCATTCAAGCAATTCTCCTGCCTTGGCCTCCTGAGTAGCTGGGGATTACA
GGCACCTGCCACCATGCCAGTCTAATTTTTGTATTTTTAGTAGAGATGGG
GTTTCGCCATGTTGGCCAGGCTGGTCTTGAACTCCTGACCTCAGATGATC
TGCCCGCCTTGGCCTCCTGAAGTGCTGGGATTATAGGTGTGAGCCACCAC
GCCTGGCCCATTGCAGATATTTTTAATTCACATTTATCTGCATCACTACT
TGGATCTTAAGGTAGCTGTAGACCCAATCCTAGATCTAATGCTTTCATAA
AGAAGCAAATATAATAAATACTATACCACAAATGTAATGTTTGATGTCTG
ATAATGATATTTCAGTGTAATTAAACTTAGCACTCCTATGTATATTATTT
GATGCAATAAAAACATATTTTTTTAGCACTTACAGTCTGCCAAACTGGCC
TGTGACACAAAAAAAGTTTAGGAATTCCTGGTTTTGTCTGTGTTAGCCAA
TGGTTAGAATATATGCTCAGAAAGATACCATTGGTTAATAGCTAAAAGAA
AATGGAGTAGAAATTCAGTGGCCTGGAATAATAACAATTTGGGCAGTCAT
TAAGTCAGGTGAAGACTTCTGGAATCATGGGAGAAAAGCAAGGGAGACAT
TCTTACTTGCCACAAGTGTTTTTTTTTTTTTTTTTTTTTATCACAAACAT
AAGAAAATATAATAAATAACAAAGTCAGGTTATAGAAGAGAGAAACGCTC
TTAGTAAACTTGGAATATGGAATCCCCAAAGGCACTTGACTTGGGAGACA
GGAGCCATACTGCTAAGTGAAAAAGACGAAGAACCTCTAGGGCCTGAACA
TACAGGAAATTGTAGGAACAGAAATTCCTAGATCTGGTGGGGCAAGGGGA
GCCATAGGAGAAAGAAATGGTAGAAATGGATGGAGACGGAGGCAGAGGTG
GGCAGATCATGAGGTCAAGAGATCGAGACCATCCTGGCAAACATGGTGAA
ATCCCGTCTCTACTAAAAATAAAAAAATTAGCTGGGCATGGTGGCATGCG
CCTGTAGTCCCAGCTGCTCGGGAGGCTGAGGCAGGAGAATCGTTTGAACC
CAGGAGGCGAAGGTTGCAGTGAGCTGAGATAGTGCCATTGCACTCCAGTC
TGGCAACAGAGTGAGACTCCGTCTCAAAAAAAAAAAAAAAAGAAAGAAAG
AAAAGAAAAAGAAAAAAGAAAAAATAAATGGATGTAGAACAAGCCAGAAG
GAGGAACTGGGCTGGGGCAATGAGATTATGGTGATGTAAGGGACTTTTAT
AGAATTAACAATGCTGGAATTTGTGGAACTCTGCTTCTATTATTCCCCCA
ATCATTACTTCTGTCACATTGATAGTTAAATAATTTCTGTGAATTTATTC
CTTGATTCTAAAATATGAGGATAATGACAATGGTATTATAAGGGCAGATT
AAGTGATATAGCATGAGCAATATTCTTCAGGCACATGGATCGAATTGAAT
ACACTGTAAATCCCAACTTCCAGTTTCAGCTCTACCAAGTAAAGAGCTAG
CAAGTCATCAAAATGGGGACATACAGAAAAAAAAAAGGACACTAGAGGAA
TAATATACCCTGACTCCTAGCCTGATTAATATATCGAT

SEQ ID NO: 99 (exemplary ET3 sequence)

MQLELSTCVFLCLLPLGFSAIRRYYLGAVELSWDYRQSELLRELHVDTRF
PATAPGALPLGPSVLYKKTVFVEFTDQLFSVARPRPPWMGLLGPTIQAEV
YDTVVVTLKNMASHPVSLHAVGVSFWKSSEGAEYEDHTSQREKEDDKVLP
GKSQTYVWQVLKENGPTASDPPCLTYSYLSHVDLVKDLNSGLIGALLVCR
EGSLTRERTQNLHEFVLLFAVFDEGKSWHSARNDSWTRAMDPAPARAQPA
MHTVNGYVNRSLPGLIGCHKKSVYWHVIGMGTSPEVHSIFLEGHTFLVRH
HRQASLEISPLTFLTAQTFLMDLGQFLLFCHISSHHHGGMEAHVRVESCA
EEPQLRRKADEEEDYDDNLYDSDMDVVRLDGDDVSPFIQIRSVAKKHPKT
WVHYIAAEEEDWDYAPLVLAPDDRSYKSQYLNNGPQRIGRKYKKVRFMAY
TDETFKTREAIQHESGILGPLLYGEVGDTLLIIFKNQASRPYNIYPHGIT
DVRPLYSRRLPKGVKHLKDFPILPGEIFKYKWTVTVEDGPTKSDPRCLTR
YYSSFVNMERDLASGLIGPLLICYKESVDQRGNQIMSDKRNVILFSVFDE
NRSWYLTENIQRFLPNPAGVQLEDPEFQASNIMHSINGYVFDSLQLSVCL
HEVAYWYILSIGAQTDFLSVFFSGYTFKHKMVYEDTLTLFPFSGETVFMS
MENPGLWILGCHNSDFRNRGMTALLKVSSCDKNTGDYYEDSYEDISAYLL
SKNNAIEPRSFAQNSRPPSASAPKPPVLRRHQRDISLPTFQPEEDKMDYD
DIFSTETKGEDFDIYGEDENQDPRSFQKRTRHYFIAAVEQLWDYGMSESP
RALRNRAQNGEVPRFKKVVFREFADGSFTQPSYRGELNKHLGLLGPYIRA
EVEDNIMVTFKNQASRPYSFYSSLISYPDDQEQGAEPRHNFVQPNETRTY
FWKVQHHMAPTEDEFDCKAWAYFSDVDLEKDVHSGLIGPLLICRANTLNA
AHGRQVTVQEFALFFTIFDETKSWYFTENVERNCRAPCHLQMEDPTLKEN
YRFHAINGYVMDTLPGLVMAQNQRIRWYLLSMGSNENIHSIHFSGHVFSV
RKKEEYKMAVYNLYPGVFETVEMLPSKVGIWRIECLIGEHLQAGMSTTFL
VYSKKCQTPLGMASGHIRDFQITASGQYGQWAPKLARLHYSGSINAWSTK
EPFSWIKVDLLAPMIIHGIKTQGARQKFSSLYISQFIIMYSLDGKKWQTY
RGNSTGTLMVFFGNVDSSGIKHNIFNPPIIARYIRLHPTHYSIRSTLRME
LMGCDLNSCSMPLGMESKAISDAQITASSYFTNMFATWSPSKARLHLQGR
SNAWRPQVNNPKEWLQVDFQKTMKVTGVTTQGVKSLLTSMYVKEFLISSS
QDGHQWTLFFQNGKVKVFQGNQDSFTPVVNSLDPPLLTRYLRIHPQSWVH
QIALRMEVLGCEAQDLYV

SEQ ID NO: 100 (exemplary β-globin sequence)

MVHLTPEEKSAVTALWGKVNVDEVGGEALGRLLVVYPWTQRFFESFGDLS
TPDAVMGNPKVKAHGKKVLGAFSDGLAHLDNLKGTFATLSELHCDKLHVD
PENFRLLGNVLVCVLAHHFGKEFTPPVQAAYQKVVAGVANALAHKYH

SEQ ID NO: 101 (exemplary γ-globin sequence)

MGHFTEEDKATITSLWGKVNVEDAGGETLGRLLVVYPWTQRFFDSFGNLS
SASAIMGNPKVKAHGKKVLTSLGDATKHLDDLKGTFAQLSELHCDKLHVD
PENFKLLGNVLVTVLAIHFGKEFTPEVQASWQKMVTAVASALSSRYH

SEQ ID NO: 102 (exemplary 3′HS1 nucleic acid sequence)

CCAGGCTCCATTATTGATATAGTCATGATCTCCTCTGTTGGGGATGAAGT
AGGCAAATTTGAGGCACTAATTTACTTCTCACATTCTTTTCTTGAACAGA
AAGATAGAACTGGAAATTAATAGTAGTATATAAATTCAAAATTTTAGCTT
TAATAACATTTAATCAGACATAAATAATTATGGTAATGTGAATTTCAATA
AATAAATTTTAGTTCTAATATAAGTGTAACTGTGTAATATTCATACTTTT
TCTGAAGGCTTTACTAATTTGATATGGCATTACTTTTTTATTGCTGCCAA
AACTATTCTTATTCCACTGTGTGGTGATGAGAAAGTGAGAGATGTTCTGG
AGATGGTGATTATAGATAGCTTCCCTGAAGCCATAGTAACCCCCTGGAGA
AAAATTGGACCTGGAGTCTAGCAGCCTAGGTATGGGTACTCGATTTCTTA
GAAAGCCTTTACAATTTCCTTTATCTTAAAAATAAGGGTATTGAAGTAGA
ATTCTAGAATTTTCAGAGGACAACTTAAAATATGTGTAATAGTTTTAATT
ATTTATCCTCATAAATTTAACTGTTCATTTTAATATATTTAAGGATGAAT
TTTTTAAAAAGTTGATTTCATAAAAACGGGAATAGAAAGATGGTTCCATA
GGCTGACTGAGAGTGTAGAGGAGGGATGGGAAGGGAAAGAAGTTGATCTT
CAGTTAGACTAGAGGAATAAGTTTTAGTGATCTCTCACACTGCATAGTGA
ACACAGTTAATAATATATTATGTATTTAAATTAAAAATTGCTAAAAAATA
AATATTTTATGTTCTCACCACAAAAAAAGTTGGAAGGTGATTCATATGCT
AATTAGCTTGATAGACTCTCTCTACAATGTATATATAGATCAAACATCAC
ATTGTATCCCATAACATATTATATATATTATATATTTATATTATATATTA
TTATTGTATCCATTAATATATGCACTTATTATTTGCCAGGCAAATAAAAA
ATGTTTTTAAAATATAAATTTATTTGTAACCTCCTTTTACTTTTCTGCTT
GGTTTTCTTCTTTCATTCAGTGTTTACCAGTTTCTTATAGTTAATTTTAT
TTTAAGCTGTCTCACATTTTCTGAAGAAAAGGGAACATATTAAAGCCAAC
AAAACAAATACACTATCTTGCATGAGATGATTTATGTCATGGTACAATCA
AATGCTATAAATCTTATAAAAACTTCTCAAATGGTTAGATGGCTACAGTT
GAACAGATGGACCATGTCATATATTTTTTATAATGCTTCTAAGGTATGGC
TAATTTTTAAAAAATATTTTAGTAATGATGGGAATATTATTTATAGAAAT
CTTATAAAATATATAATGAAATATGTAATAAAGTCTAGATAAATGTGTAT
ATACATAATATATATTTATTACATAATATATAATATATAATGTATATTTA
TATATTACATGCATTATATATTAAATATAATACATTTTATATATTATATA
TTAAAATATGTAATAATATGTTATTAAATATATACAATAATCTATTACAT
TTTATGCTTATATAATATATAATAAATATATAGTATATAATAAATATACA
CTATATATTTGTATCTATATATGTTTATAAAGTCATTCCTCTAATTAGGT
CATAACCATTCAGGTAAACTGGAAATTTAAGCCTACTTCAGGTTTGTGGT
AAATAGATTCTCTCTGAACTAGCATATTCAGAATCATTAAACAGTCAGTT
CTTTGGACAAGTCTTATAGAATGTTCTTACCTCTTCAGCCATCCCAAGAC
TCTTGAGGGCCTGACCTCGCTTACACTAAAGCAGATCTGCCTTATGCATC
ACTGAAGTAGGGAGGGAAGAAAGTTTGATGAACTACTTCTGACCCCTAGT
GGTGTCCAGAAAAGACCATTAAAGGAATGACCTTTAAAGGATGGACATAC
AATTTTTTGTCCAAGGCAGGACATGTGTGGGTGTCTTTCAGTAATTATGT
TCTAAGAACAGCAAAAACTCCACTGCCTTGGCAAATAGGAATGTTTTAGT
TCTATAGAATTATAAAGAAGCTGTCTTTTAAACACAATATACTTTCTCTA
TGTCTTTGGAACAATGACTATTGGTCATTACCCTATTTTAAAGTAAGCAA
GTAATCACACAGGGAATTATTCTGAAAAGACAGAAAAAAAAAAAAAACCA
AGAGATTTCTGCATATGTAGGTCAGTTTTAATCAGAGGGCATCAGAAAAG
ACTCCTGAAAGAATGACCTGGTTATTATAATCACAGATTTGCTTTCCAAG
TCAACATTCCAGACAGTGCTCAGAGGGGATACGAAAACCCTTTTATTTCT
CCAGACTCAAATTCACTGCTATTTGTCTTCTCTATTTATTTTATTATAGG
CATTGTTCTGGTTGCTGGGAACTCAGACTGAGATACCATACACTGACTCT
CAGATAGCATAACACAACATGATGTCTTGGAAAACTGTAAATCTTTTTGT
TTTTTAAATACAGGTGGAGCATCTGGCACACCTGACATATTGATCTTGTT
TTTCTTTAAATCTTCATTTATTTACCTTATCAAAACTATGCTCTTTCATC
CTACCTTTCAAAACATATTTTAAAAAATCCTCCAACATGTATTTTGCTCT
GGTAATCCCAAAAGGCTGATAGTCTCTATGGTGGCAACATGGATAATACT
GTTCCCCATCTAGATGGTCTCATTTCTTCTGTATCTAGTCTGAAGAAGCC
TGAATGAAAGTAGATTTTTAAGCTTTGTAGCTAGTCTGAAGCCTTTGTAG
TCAGTCTGAAGAAACCTGCATGAAAATAGATTTTTTTTTTCCTTTGGGAC
AGAGTCTTGCTCTGTCGCCCAGACTGGAGTGCAATGGCGCGATCTCGGCT
CACTGCAACTTCCACCTCCCAGGATCAAGCAATTCTCCTGCCTCAGTCTC
CCAAGTAACTGGGATTACAGGAGCACACTGCCATGCCCAGCTAATTATTT
TTTGTGTTTTAGTAGAGACAGGGTTTCACC

What is claimed is:

1. An adenoviral donor vector comprising:
(a) an adenoviral capsid; and
(b) a linear, double-stranded DNA genome comprising:
(i) a transposon payload of at least 15 kb;
(ii) transposon inverted repeats (IRs) that flank the transposon payload; and
(iii) recombinase direct repeats (DRs) that flank the transposon inverted repeats.

2. An adenoviral donor genome comprising:
(a) a transposon payload of at least 15 kb;
(b) transposon inverted repeats (IRs) that flank the transposon payload; and
(c) recombinase direct repeats (DRs) that flank the transposon inverted repeats.

3. An adenoviral transposition system comprising:
(a) the adenoviral donor vector of claim 1; and
(b) an adenoviral support vector comprising
(i) the adenoviral capsid; and
(ii) an adenoviral support genome comprising a nucleic acid sequence encoding a transposase.

4. An adenoviral transposition system comprising:
(a) the adenoviral donor genome of claim 2; and
(b) an adenoviral support genome comprising a nucleic acid sequence encoding a transposase.

5. An adenoviral production system comprising:
(a) a nucleic acid comprising the adenoviral donor genome of claim 2; and
(b) a nucleic acid comprising an adenoviral helper genome comprising a conditional packaging element.

6. The vector of claim 1, wherein one or more of:
the transposon payload comprises a Long LCR, optionally wherein the Long LCR is a β-globin Long LCR comprising β-globin LCR HS1 to HS5;
the transposon payload comprises a Long LCR having a length of at least 27 kb, optionally wherein the Long LCR is a β-qlobin Long LCR comprising β-qlobin LCR HS1 to HS5; and
the transposon payload comprises an LCR set forth in Table 1.

7-8. (canceled)

9. The vectorof claim 1, wherein one or more of:
the transposon payload has a length of at least 16 kb, at least 17 kb, at least 18 kb, at least 19 kb, at least 20 kb, at least 21 kb, at least 22 kb, at least 23 kb, at least 24 kb, at least 25 kb, at least 30 kb, at least 35 kb, at least 38 kb, or at least 40 kb;
the transposon payload has a length of 15 kb-35 kb, 15 kb-30 kb, 20 kb-35 kb, or 20 kb-30 kb; and
the transposon payload has a length of 15 kb-32.4 kb, or 20 kb-32.4 kb.

10-11. (canceled)

12. The vector of claim 6, wherein one or more of:
the transposon payload comprises a nucleic acid sequence that encodes a protein, optionally wherein the protein is a therapeutic protein;
the transposon payload comprises a nucleic acid sequence that encodes a protein selected from the group consisting of a β globin replacement protein and a γ-globin replacement protein;
the transposon payload comprises a nucleic acid sequence that encodes a Factor VIII replacement protein; and
the transposon payload comprises a nucleic acid sequence that encodes a protein and the nucleic acid sequence that encodes the protein is operably linked with a promoter, optionally wherein the promoter is a β globin promoter.

13-15. (canceled)

16. The vectorof claim 1, wherein:
the transposon inverted repeats are Sleeping Beauty (SB) inverted repeats, optionally wherein the SB inverted repeats are pT4 inverted repeats; and/or
the recombinase direct repeats are FRT sites.

17. The vectorof claim 3, wherein one or more of:
the transposase is a Sleeping Beauty (SB) transposase;
the transposase is Sleeping Beauty 100x (SB100x;
the adenoviral support genome comprises a nucleic acid encoding a recombinase; and
the adenoviral support genome comprises a nucleic acid encoding a FLP recombinase.

18-20. (canceled)

21. The vector of claim 6, wherein one or more of:
the transposon payload comprises a β-globin long LCR, the transposon payload comprises a nucleic acid sequence that encodes β-globin operably linked with a β-globin promoter, the inverted repeats are SB inverted repeats, and the recombinase direct repeats are FRT sites;
the transposon payload comprises a selection cassette, optionally wherein the selection cassette comprises a nucleic acid sequence that encodes mgmt^{P140K}; and
the adenoviral capsid is modified for increased affinity to CD46, optionally wherein the adenoviral capsid is an Ad35++ capsid.

22-23. (canceled)

24. The adenoviral production system of claim 5, wherein the adenoviral helper genome conditional packaging element comprises a packaging sequence flanked by recombinase direct repeats, optionally wherein the recombinase direct repeats that flank the packaging sequence of the conditional packaging element are LoxP sites.

25. (canceled)

26. A cell comprising a vector according to claim 1, optionally where the cell is a hematopoietic cell.

27. A cell comprising in its genome the transposon payload of the vector of claim 1, wherein the transposon payload present in the genome of the cell is flanked by the transposon inverted repeats, optionally wherein the cell is a hematopoietic stem cell.

28. (canceled)

29. An adenovirus-producing cell comprising an adenoviral production system according to claim 5, optionally wherein the cell is a HEK293 cell.

30. A method of modifying a cell, the method comprising contacting the cell with a vector of claim 1.

31. A method of modifying a cell of a subject, the method comprising administering to the subject a vector of claim 1, optionally wherein the method does not involve isolations of the cell from the subject, further optionally wherein the adenoviral donor vector is administered to the subject intravenously.

32. (canceled)

33. A method of treating a disease or condition in a subject in need thereof, the method comprising administering to the subject a vector of claim 1, optionally wherein the adenoviral donor vector is administered to the subject intravenously.

34. (canceled)

35. The method of claim 31, wherein the method comprises administering to the subject a mobilization agent, optionally wherein the mobilization agent comprises one or more of granulocyte-colony stimulating factor (G-CSF), a CXCR4 antagonist, and a CXCR2 agonist.

36-37. (canceled)

38. The method of claim 31, wherein the transposon payload comprises a selection cassette and the method comprises administering a selection agent to the subject, optionally wherein the selection cassette encodes mgmt^{P140K} and the selection agent is O⁶BG/BCNU.

39. (canceled)

40. The method of claim 31, wherein one or more of:
the method causes integration and/or expression of at least one copy of the transposon payload in at least 20%, 30%, 40%, 50%, 60%, 70%, 80%, 90%, or 95% of cells expressing CD46;
the method causes integration and/or expression of at least one copy of the transposon payload in at least 20%, 30%, 40%, 50%, 60%, 70%, 80%, 90%, or 95% of hematopoietic stem cells and/or erythroid Ter119⁺ cells;
the method causes integration of an average of at least 2 copies of the transposon payload in the genomes of cells comprising at least 1 copy of the transposon payload;
the method causes integration of an average of at least 2.5 copies of the transposon payload in the genomes of cells comprising at least 1 copy of the transposon payload;
the method causes expression of a protein encoded by the transposon payload at a level that is at least about 20% of the level of reference, optionally wherein the reference is expression of an endogenous reference protein in the subject or in a reference population;
the method causes expression of a protein encoded by the transposon payload at a level that is at least about 25% of the level of reference, optionally wherein the reference is expression of an endogenous reference protein in the subject or in a reference population;
the subject is a subject suffering from thalassemia intermedia, wherein the transposase payload comprises a β-qlobin Long LCR comprising β-qlobin LCR HS1 to HS5 and a nucleic acid sequence encoding a β globin replacement protein and/or γ-globin replacement protein operably linked with a β globin promoter;
the subject is a subject suffering from hemophilia, wherein the transposase payload comprises a β-qlobin Long LCR comprising β-qlobin LCR HS1 to HS5 and a nucleic acid sequence encoding a Factor VIII replacement protein operably linked with a β globin promoter; and
the subject is a subject suffering from hemophilia, wherein the transposase payload comprises a β-qlobin Long LCR comprising β-qlobin LCR HS1 to HS5 and a nucleic acid sequence encoding a Factor VIII replacement protein operably linked with a β globin promoter and expression of the protein in the subject reduces at least one symptom of thalassemia intermedia and/or treats thalassemia intermedia.

41-48. (canceled)


SEQUENCE LISTING (102 sequences)

SEQ ID NO: 1 - DNA, 720 bp, Artificial Sequence
  5 end vector sequence, Sleeping Beauty IR/DR sequence, integration junction (chr15, 6805206)
ccctgggattccccaaggcaggggcgagtccttttgtatgaattactcaaatcgataactagaaacttaattaacaacgagatcttataatttgcatacttctgcctgctggggactttccacaccctagctgacacaagaatttgaaatacatccacaggtacacctccaattgactcaaatgatgtcaattagtctatcataatcttctaaagccatgacatcattttaactggaattttccaagctgtttaaaggcacagtcaacttagtgtatgtaaacttctgacccactggaattgtgatacagtgaattataagtgaaataatctgtctgtaaacaattgttggaaaaatgacttgtgtcatgcacaaagtagatgtcctaactgacttgccaaaactattgtttgttaacaagaaatttgtggagtagttgaaaaacgagttttaatgactccaacttaagtgtatgtaaacttccgacttcaactgtaagaatggcccattcatctatagtagcacacaatatttgcatttgtgcgacagtataagggacaattatgctatcaggcatttttccaaagtgagtaatcgaagtttttatacctttgtgtgccatgtttgctaccatggtgggataatcttacacgcgttctcgcgaccggccaggaaagacgcaacaaaccggaatcttctgcggcaaaagctttattgctt

SEQ ID NO: 2 - DNA, 607 bp, Artificial Sequence
  5 end vector sequence, Sleeping Beauty IR/DR sequence, integration junction (chrX, 16897322)
  misc_feature (594)..(594): n is a, c, g, or t
  misc_feature (596)..(597): n is a, c, g, or t
  misc_feature (602)..(602): n is a, c, g, or t
tagaaacttaattaacaacgagatcttataatttgcatacttctgcctgctggggactttccacaccctagctgacacaagaatttgaaatacatccacaggtacacctccaattgactcaaatgatgtcaattagtctatcataatcttctaaagccatgacatcattttaactggaattttccaagctgtttaaaggcacagtcaacttagtgtatgtaaacttctgacccactggaattgtgatacagtgaattataagtgaaataatctgtctgtaaacaattgttggaaaaatgacttgtgtcatgcaaagtagatgtcctaactgacttgccaaaactattgtttgttaacaagaaatttgtggagtagttgaaaaacgagttttaatgactccaacttaagtgtatgtaaacttccgacttcaactgtacaagtagaccaaatatccatatacataaaagaaaaaaatagaaaaaatttctagtgacagaaaaatgacaaagaacatactgtttattactactattaagatgtttgcttccattacactcatatgagtcatgatattttttcttcatttttttctantnncactngaaat

SEQ ID NO: 3 - DNA, 520 bp, Artificial Sequence
  3 end vector sequence, Sleeping Beauty IR/DR sequence, integration junction (chr4, 10207667)
gttgctaggaatgagccaaattcatctgtattaaacagtgggagcttgtggaaggctactcgaaatgtttgacccaagttaaacaatttaaaggcaatgctaccaaatactaattgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtatatcctccccgttgcaccctcttgatgatgctgagatgaacacagatgctcactccttgagggctctaagcttatgctgacacagacacaggtgctcacttctatgaatggcctaagatttgaggacatcatgaggacaagtgtgataaaatcttggaacaacctcccagaggtct

SEQ ID NO: 4 - DNA, 74 bp, Artificial Sequence
  Sleeping beauty IR/DR sequence, integration junction (chr7, 79796094)
acttaagtgtatgtaaacttccgacttcaactgtagggtacctgattctctgggcatctctgcccactaccatg

SEQ ID NO: 5 - DNA, 75 bp, Artificial Sequence
  Sleeping beauty IR/DR sequence, Integration junction (repeat region)
acttaagtgtatgtaaacttccgacttcaactgtaaattttccacctttttcagttttcctcgccatatttcatg

SEQ ID NO: 6 - DNA, 21531 bp, Homo sapiens
gatctctatcccctcctgttttctctacgttatttatatgggtatcatcaccatcctggacaacatcaggacagatatccctcaccaagccaatgttcctctctatgttggctcaaatgtccttgaactttcctttcaccaccctttccacagtcaaaaggatattgtagtttaatgcctcagagttcagcttttaagcttctgacaaattattcttcctctttaggttctcctttatggaatcttctgtactgatggccatgtcctttaactactatgtagatatctgctactacctgtattatgcctctacctttattagcagagttatctgtactgttggcatgacaatcatttgttaatatgacttgcctttcctttttctgctattcttgatcaaatggctcctctttcttgctcctctcatttctcctgccttcacttggacgtgcttcacgtagtctgtgcttatgactggattaaaaattgatatggacttatcctaatgttgttcgtcataatatgggttttatggtccattattatttcctatgcattgatctggagaaggcttcaatccttttactctttgtggaaaatatctgtaaaccttctggttcactctgctatagcaatttcagtttaggctagtaagcatgaggatgcctccttctctgatttttcccacagtctgttggtcacagaataacctgagtgattactgatgaaagagtgagaatgttattgatagtcacaatgacaaaaaacaaacaactacagtcaaaatgtttctctttttattagtggattatatttcctgacctatatctggcaggactctttagagaggtagctgaagctgctgttatgaccactagagggaagaagatacctgtggagctaatggtccaagatggtggagccccaagcaaggaagttgttaaggagcccttttgattgaaggtgggtgcccccaccttacagggacaggacatctggatactcctcccagtttctccagtttccctttttcctaatatatctcctgataaaatgtctatactcacttccccatttctaataataaagcaaaggctagttagtaagacatcaccttgcattttgaaaatgccatagactttcaaaattatttcatacatcggtctttctttatttcaagagtccagaaatggcaacattacctttgattcaatgtaatggaaagagctctttcaagagacagagaaaagaataatttaatttctttccccacacctccttccctgtctcttaccctatcttccttccttctaccctccccatttctctctctcatttctcagaagtatattttgaaaggattcatagcagacagctaaggctggttttttctaagtgaagaagtgatattgagaaggtagggttgcatgagccctttcagttttttagtttatatacatctgtattgttagaatgttttataatataaataaaattatttctcagttatatactagctatgtaacctgtggatatttccttaagtattacaagctatacttaactcacttggaaaactcaaataaatacctgcttcatagttattaataaggattaagtgagataatgcccataagattcctattaataacagataaatacatacacacacacacacattgaaaggattcttactttgtgctaggaactataataagttcattgatgcattatatcattaagttctaatttcaacactagaaggcaggtattatctaaatttcatactggatacctccaaactcataaagataattaaattgccttttgtcatatatttattcaaaagggtaaactcaaactatggcttgtctaattttatatatcaccctactgaacatgaccctattgtgatattttataaaattattctcaagttattatgaggatgttgaaagacagagaggatggggtgctatgccccaaatcagcctcacaattaagctaagcagctaagagtcttgcagggtagtgtagggaccacagggttaagggggcagtagaattatactcccactttagtttcatttcaaacaatccatacacacacagccctgagcacttacaaattatactacgctctatactttttgtttaaatgtataaataagtggatgaaagaatagatagatagatagacagatagatgatagatagaataaatgcttgccttcatagctgtctccctaccttgttcaaaatgttcctgtccagaccaaagtaccttgccttcacttaagtaatcaattcctaggttatattctgatgtcaaaggaagtcaaaagatgtgaaaaacaatttctgacccacaactcatgctttgtagatgactagatcaaaaaatttcagccatatcttaacagtgagtgaacaggaaatctcctcttttccctacatctgagatcccagcttctaagaccttcaattctcactcttgatgcaacagaccttggaagcatacaggagagctgaacttggtcaacaaaggagaaaagtttgttggcctccaaaggcacagctcaaacttttcaagccttctctaatcttaaaggtaaacaagggtctcatttctttgagaacttcagggaaaatagacaaggacttgcctggtgcttttggtaggggagcttgcactttccccctttctggaggaaatatttatccccaggtagttccctttttgcaccagtggttctttgaagagacttccacctgggaacagttaaacagcaactacagggccttgaactgcacactttcagtccggtcctcacagttgaaaagacctaagcttgtgcctgatttaagcctttttggtcataaaacattgaattctaatctccctctcaaccctacagtcacccatttggtatattaaagatgtgttgtctactgtctagtatccctcaagtagtgtcaggaattagtcatttaaatagtctgcaagccaggagtggtggctcatgtctgtaattccagcacttgagaggtagaagtgggaggactgcttgagctcaagagtttgatattatcctggacaacatagcaagacctcgtctctacttaaaaaaaaaaaaaaaattagccaggcatgtgatgtacacctgtagtcccagctactcaggaggccgaaatgggaggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttggggaccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttctgttactccaagactgagaaggaaattgaacctgagactcattgactggcaagatgtccccagaggctctcattcagcaataaaattctcaccttcacccaggcccactgagtgtcagatttgcatgcactagttcacgtgtgtaaaaaggaggatgcttctttcctttgtattctcacatacctttaggaaagaacttagcacccttcccacacagccatcccaataactcatttcagtgactcaacccttgactttataaaagtcttgggcagtatagagcagagattaagagtacagatgctggagccagaccacctgagtgattagtgactcagtttctcttagtagttgtatgactcagtttcttcatctgtaaaatggagggttttttaattagtttgtttttgagaaagggtctcactctgtcacccaaatgggagtgtagtggcaaaatctcggctcactgcaacttgcacttcccaggctcaagcggtcctcccacctcaacatcctgagtagctggaaccacaggtacacaccaccatacctcgctaattttttgtatttttggtagagatggggtttcacatgttacacaggatggtctcagactccggagctcaagcaatctgcccacctcagccttccaaagtgctgggattataagcatgattacaggagttttaacaggctcataagattgttctgcagcccgagtgagttaatacatgcaaagagtttaaagcagtgacttataaatgctaactactctagaaatgtttgctagtattttttgtttaactgcaatcattcttgctgcaggtgaaaactagtgttctgtactttatgcccattcatctttaactgtaataataaaaataactgacatttattgaaggctatcagagactgtaattagtgctttgcataattaatcatatttaatactcttggattctttcaggtagatactattattatccccattttactacagttaaaaaaactacctctcaacttgctcaagcatacactctcacacacacaaacataaactactagcaaatagtagaattgagatttggtcctaattatgtctttgctcactatccaataaatatttattgacatgtacttcttggcagtctgtatgctggatgctggggatacaaagatgtttaaatttaagctccagtctctgcttccaaaggcctcccaggccaagttatccattcagaaagcattttttactctttgcattccactgtttttcctaagtgactaaaaaattacactttattcgtctgtgtcctgctctgggatgatagtctgactttcctaacctgagcctaacatccctgacatcaggaaagactacaccatgtggagaaggggtggtggttttgattgctgctgtcttcagttagatggttaactttgtgaagttgaaaactgtggctctctggttgactgttagagttctggcacttgtcactatgcctattatttaacaaatgcatgaatgcttcagaatatgggaatattatcttctggaatagggaatcaagttatattatgtaacccaggattagaagattcttctgtgtgtaagaatttcataaacattaagctgtctagcaaaagcaagggcttggaaaatctgtgagctcctcaccatatagaaagcttttaacccatcattgaataaatccctataggggatttctaccctgagcaaaaggctggtcttgattaattcccaaactcatatagctctgagaaagtctatgctgttaacgttttcttgtctgctaccccatcatatgcacaacaataaatgcaggcctaggcatgactgaaggctctctcataattcttggttgcatgaatcagattatcaacagaaatgttgagacaaactatggggaagcagggtatgaaagagctctgaatgaaatggaaaccgcaatgcttcctgcccattcagggctccagcatgtagaaatctggggctttgtgaagactggcttaaaatcagaagccccattggataagagtagggaagaacctagagcctacgctgagcaggtttccttcatgtgacagggagcctcctgccccgaacttccagggatcctctcttaagtgtttcctgctggaatctcctcacttctatctggaaatggtttctccacagtccagcccctggctagttgaaagagttacccatgcagaggccctcctagcatccagagactagtgcttagattcctactttcagcgttggacaacctggatccacttgcccagtgttcttccttagttcctaccttcgaccttgatcctcctttatcttcctgaaccctgctgagatgatctatgtggggagaatggcttctttgagaaacatcttcttcgttagtggcctgcccctcattcccactttaatatccagaatcactataagaagaatataataagaggaataactcttattataggtaagggaaaattaagaggcatacgtgatgggatgagtaagagaggagagggaaggattaatggacgataaaatctactactatttgttgagaccttttatagtctaatcaattttgctattgttttccatcctcacgctaactccataaaaaaacactattattatctttattttgccatgacaagactgagctcagaagagtcaagcatttgcctaaggtcggacatgtcagaggcagtgccagacctatgtgagactctgcagctactgctcatgggccctgtgctgcactgatgaggaggatcagatggatggggcaatgaagcaaaggaatcattctgtggataaaggagacagccatgaagaagtctatgactgtaaatttgggagcaggagtctctaaggacttggatttcaaggaattttgactcagcaaacacaagaccctcacggtgactttgcgagctggtgtgccagatgtgtctatcagaggttccagggagggtggggtggggtcagggctggccaccagctatcagggcccagatgggttataggctggcaggctcagataggtggttaggtcaggttggtggtgctgggtggagtccatgactcccaggagccaggagagatagaccatgagtagagggcagacatgggaaaggtgggggaggcacagcatagcagcatttttcattctactactacatgggactgctcccctatacccccagctaggggcaagtgccttgactcctatgttttcaggatcatcatctataaagtaagagtaataattgtgtctatctcatagggttattatgaggatcaaaggagatgcacactctctggaccagtggcctaacagttcaggacagagctatgggcttcctatgtatgggtcagtggtctcaatgtagcaggcaagttccagaagatagcatcaaccactgttagagatatactgccagtctcagagcctgatgttaatttagcaatgggctgggaccctcctccagtagaaccttctaaccagctgctgcagtcaaagtcgaatgcagctggttagactttttttaatgaaagcttagctttcattaaagattaagctcctaagcagggcacagatgaaattgtctaacagcaactttgccatctaaaaaaatctgacttcactggaaacatggaagcccaaggttctgaacatgagaaatttttaggaatctgcacaggagttgagagggaaacaagatggtgaagggactagaaaccacatgagagacacgaggaaatagtgtagatttaggctggaggtaaatgaaagagaagtgggaattaatacttactgaaatctttctatatgtcaggtgccattttatgatatttaataatctcattacatatggtaattctgtgagatatgtattattgaacatactataattaatactaatgataagtaacacctcttgagtacttagtatatgctagaatcaaatttaagtttatcatatgaggccgggcacggtggctcatatatgggattacatgcctgtaatcccagcactttgggaggccaaggcaattggatcacctgaggtcaggagttccagaccagcctggccaacatggtgaaaccccttctctactaaaaaatacaaaaaatcagccaggtgtggtggcacgcgtctataatcccagctactcaggaggctgaggcaggagaatcacttgaacccaggaggtggaggttgcagtgagctaagattgcaccactgcactccagcctaggcgacagagtgagactccatctcaaaaaaaaaaaaagaagtttattatatgaattaacttagttttactcacaccaatactcagaagtagattattacctcatttattgatgaggagcccaatgtacttgtagtgtagatcaacttattgaaagcacaagctaataagtagacaattagtaattagaagtcagatggtctgagctctcctactgtctacattacatgagctcttattaactggggactcgaaaatcaaagacatgaaataatttgtccaagcttacagaaccaccaagtagtaaggctaggatgtagacccagttctgctacctctgaagacagtgttttttccacagcaaaacacaaactcagatattgtggatgcgagaaattagaagtagatattcctgccctgtggcccttgcttcttacttttacttcttgtcgattggaagttgtggtccaagccacagttgcagaccatacttcctcaaccataattgcatttcttcaggaaagtttgagggagaaaaaggtaaagaaaaatttagaaacaacttcagaataaagagattttctcttgggttacagagattgtcatatgacaaattataagcagacacttgagaaaactgaaggcccatgcctgcccaaattaccctttgaccccttggtcaagctgcaactttggttaaagggagtgtttatgtgttatagtgttcatttactcttctggtctaacccattggctccgtcttcatcctgcagtgacctcagtgcctcagaaacatacatatgtttgtctagtttaagtttgtgtgaaattctaactagcgtcaagaactgagggccctaaactatgctaggaatagtgctgtggtgctgtgataggtacacaagaaatgagaagaaactgcagattctctgcatctccctttgccgggtctgacaacaaagtttccccaaattttaccaatgcaagccatttctccatatgctaactactttaaaatcatttggggcttcacattgtctttctcatctgtaaaaagaatggaagaactcattcctacagaactccctatgtcttccctgatgggctagagttcctctttctcaaaaattagccattattgtatttccttctaagccaaagctcagaggtcttgtattgcccagtgacatgcacactggtcaaaagtaggctaagtagaagggtactttcacaggaacagagagcaaaagaggtgggtgaatgagagggtaagtgagaaaagacaaatgagaagttacaacatgatggcttgttgtctaaatatctcctagggaattattgtgagaggtctgaatagtgttgtaaaataagctgaatctgctgccaacattaacagtcaagaaatacctccgaataactgtacctccaattattctttaaggtagcatgcaactgtaatagttgcatgtatatatttatcataatactgtaacagaaaacacttactgaatatatactgtgtccctagttctttacacaataaactaatctcatcctcataattctattagctaatacatattatcatcctatatttcagagacttcaagaagttaagcaacttgctcaagatcatctaagaagtaggtggtatttctgggctcatttggcccctcctaatctctcatggcaacatggctgcctaaagtgttgattgccttaattcatcagggatgggctcatactcactgcagaccttaactggcatcctcttttcttatgtgatctgcctgaccctagtagacttatgaaatttctgatgagaaaggagagaggagaaaggcagagctgactgtgatgagtgatgaaggtgccttctcatctgggtaccagtggggcctctaagactaagtcactctgtctcactgtgtcttagccagttccttacagcttgccctgatgggagatagagaatgggtatcctccaacaaaaaaataaattttcatttctcaaggtccaacttatgttttcttaatttttaaaaaaatcttgaccattctccactctctaaaataatccacagtgagagaaacattcttttcccccatcccataaatacctctattaaatatggaaaatctgggcatggtgtctcacacctgtaatcccagcactttgggaggctgaggtgggtggactgcttggagctcaggagttcaagaccatcttggacaacatggtgataccctgcctctacaaaaagtacaaaaattagcctggcatggtggtgtgcacctgtaatcccagctattagggtggctgaggcaggagaattgcttgaacccgggaggcggaggttgcagtgagctgagatcgtgccactgcactccagcctgggggacagagcacattataattaactgttattttttacttggactcttgtggggaataagatacatgttttattcttatttatgattcaagcactgaaaatagtgtttagcatccagcaggtgcttcaaaaccatttgctgaatgattactatactttttacaagctcagctccctctatcccttccagcatcctcatctctgattaaataagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggcttagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctagaatatattatttcctgaatatatatatatatatacacatatacgtatatatatatatatatatatatttgttgttatcaattgccatagaatgattagttattgtgaatcaaatatttatcttgcaggtggcctctatacctagaagcggcagaatcaggctttattaatacatgtgtatagatttttaggatctatacacatgtattaatatgaaacaaggatatggaagaggaaggcatgaaaacaggaaaagaaaacaaaccttgtttgccattttaaggcacccctggacagctaggtggcaaaaggcctgtgctgttagaggacacatgctcacatacggggtcagatctgacttggggtgctactgggaagctctcatcttaaggatacatctcaggccagtcttggtgcattaggaagatgtaggcaactctgatcctgagaggaaagaaacattcctccaggagagctaaaagggttcacctgtgtgggtaactgtgaaggactacaagaggatgaaaaacaatgacagacagacataatgcttgtgggagaaaaaacaggaggtcaaggggatagagaaggcttccagaagaatggctttgaagctggcttctgtaggagttcacagtggcaaagatgtttcagaaatgtgacatgacttaaggaactatacaaaaaggaacaaatttaaggagaggcagataaattagttcaacagacatgcaaggaattttcagatgaatgttatgtctccactgagcttcttgaggttagcagctgtgagggttttgcaggcccaggacccattacaggacctcacgtatacttgacactgttttttgtattcatttgtgaatgaatgacctcttgtcagtctactcggtttcgctgtgaatgaatgatgtcttgtcagcctacttggtttcgctaagagcacagagagaagatttagtgatgctatgtaaaaacttcctttttggttcaagtgtatgtttgtgatagaaatgaagacaggctacatgatgcatatctaacataaacacaaacattaagaaaggaaatcaacctgaagagtatttatacagataacaaaatacagagagtgagttaaatgtgtaataactgtggcacaggctggaatatgagccatttaaatcacaaattaattagaaaaaaaacagtggggaaaaaattccatggatgggtctagaaagactagcattgttttaggttgagtggcagtgtttaaagggtgatatcagactaaacttgaaatatgtggctaaataactagaatactctttattttttcgtatcatgaatagcagatatagcttgatggccccatgcttggtttaacatccttgctgttcctgacatgaaatccttaatttttgacaaaggggctattcattttcattttatattgggcctagaaattatgtagatggtcctgaggaaaagtttatagcttgtctatttctctctctaacatagttgtcagcacaatgcctaggctataggaagtactcaaagcttgttaaattgaattctatccttcttattcaattctacacatggaggaaaaactcatcagggatggaggcacgcctctaaggaaggcaggtgtggctctgcagtgtgattgggtacttgcaggacgaagggtggggtgggagtggctaaccttccattcctagtgcagaggtcacagcctaaacatcaaattccttgaggtgcggtggctcactcctgtaatcacagcagtttgggacgccaaggtgggcagatcacttgaggtcaggagttggacaccagcccagccaacatagtgaaacctggtctctgcttaaaaatataaaaattagctggacgtggtgacgggagcctgtaatccaactacttgggaggctgaggcaggagaatcgcttgaaccggggaggtggagtttgcactgagcagagatcatgccattgcactccagcctccagagcgagactctgtctaaagaaaaacgaaaacaaacaaacaaacaaacaaacaaaacccatcaaattccctgaccgaacagaattctgtctgattgttctctgacttatctaccattttccctccttaaagaaactgtgaacttccttcagctagaggggcctggctcagaagcctctggtcagcatccaagaaatacttgatgtcactttggctaaaggtatgatgtgtagacaagctccagagatggtttctcatttccatatccacccacccagctttccaattttaaagccaattctgaggtagagactgtgatgaacaaacaccttgacaaaattcaacccaaagactcactttgcctagcttcaaaatccttactctgacatatactcacagccagaaattagcatgcactagagtgtgcatgagtgcaacacacacacacaccaattccatattctctgtcagaaaatcctgttggtttttcgtgaaaggatgttttcagaggctgaccccttgccttcacctccaatgctaccactctggtctaagtcactgtcaccaccacctaaattatagctgttgactcataacaatcttcctgcttctaccactgccccactacaatttcttcccaatatactatccaaattagtcttttcaaaatgtaagtcatatatggtcacctctttgttcaaagtcttctgatagtttcctatatcatttataataaaaccaaatccttacaattctctacaatagttgttcatgcatatattatgtttattacagatacatatatatagctctcatataaataaatatatatatttatgtgtatgtgtgtagagtgttttttcttacaactctatgatgtaggtattattagtgtcccaaattttataatttaggacttctatgatctcatcttttattctccccttcaccgaatctcatcctacattggccttattgatattccttgaaaattctaagcatcttacatctttagggtatttacatttgccattccctatgccctaaatatttaatcatagtttcatataaatgggttcctcatcatctatgggtactctctcaggtgttaactttatagtgaggactttcctgccatactacttaaagtagcgataccctttcaccctgtcctaatcacactctggccttcatttcagttttttttttttctccatagcacctaatctcattggtatataacatgtttcatttgcttatttaatgtcaagctctttccactatcaagtccatgaaaacaggaactttattcctctattctgtttttgtgctgtattcttagcaattttacaattttgaatgaatgaatgagcagtcaaacacatatacaactataattaaaaggatgtatgctgacacatccactgctatgcacacacaaagaaatcagtggagtagagctggaagtgctaagcctgcatagagctagttagccctccgcaggcagagccttgatgggattactgagttctagaattggactcatttgttttgtaggctgagatttgctcttgaaaacttgttctgaccaaaataaaaggctcaaaagatgaatatcgaaaccagggtgttttttacactggaatttataactagagcactcatgtttatgtaagcaattaattgtttcatcagtcaggtaaaagtaaagaaaaactgtgccaaggcaggtagcctaatgcaatatgccactaaagtaaacattatttcataggtgtcagatatggcttattcatccatcttcatgggaaggatggccttggcctggacatcagtgttatgtgaggttcaaaacacctctaggctataaggcaacagagctccttttttttttttctgtgctttcctggctgtccaaatctctaatgataagcatacttctattcaatgagaatattctgtaagattatagttaagaattgtgggagccattccgtctcttatagttaaatttgagcttcttttatgatcactgtttttttaatatgctttaagttctggggtacatgtgccatggtggtttgctgcacccatcaacccgtcatctacattaggtatttctcctaatgctatccttcccctagccccccacccccaacaggccccagtgtgtgatgttcccctccctgtgtccatggatcactggtttttttttgttttttttttttttttaaagtctcagttaaatttttggaatgtaatttattttcctggtatcctaggacttgcaagttatctggtcactttagccctcacgttttgatgataatcacatatttgtaaacacaacacacacacacacacacacacacatatatatatatataaaacatatatatacataaacacacataacatatttatcgggcatttctgagcaactaatcatgcaggactctcaaacactaacctatagccttttctatgtatctacttgtgtagaaaccaagcgtggggactgagaaggcaatagcaggagcattctgactctcactgcctttagctaggcccctccctcatcacagctcagcatagtcctgagctcttatctatatccacacacagtttctgacgctgcccagctatcaccatcccaagtctaaagaaaaaaataatgggtttgcccatctctgttgattagaaaacaaaacaaaataaaataagcccctaagctcccagaaaacatgactaaaccagcaagaagaagaaaatacaataggtatatgaggagactggtgacactagtgtctgaatgaggcttgagtacagaaaagaggctctagcagcatagtggtttagaggagatgtttctttccttcacagatgccttagcctcaataagcttgcggttgtggaagtttactttcagaacaaactcctgtggggctagaattattgatggctaaaagaagcccgggggagggaaaaatcattcagcatcctcacccttagtgacacaaaacagagggggcctggttttccatatttcctcatgatggatgatctcgttaatgaaggtggtctgacgagatcattgcttcttccatttaagccttgctcacttgccaatcctcagttttaaccttctccagagaaatacacattttttattcaggaaacatactatgttatagtttcaatactaaataatcaaagtactgaagatagcatgcataggcaagaaaaagtccttagctttatgttgctgttgtttcagaatttaaaaaagatcaccaagtcaaggacttctcagttctagcactagaggtggaatcttagcatataatcagaggtttttcaaaatttctagacataagattcaaagccctgcacttaaaatagtctcatttgaattaactctttatataaattgaaagcacattctgaactacttcagagtattgttttatttctatgttcttagttcataaatacattaggcaatgcaatttaattaaaaaaacccaagaatttcttagaattttaatcatgaaaataaatgaaggcatctttacttactcaaggtcccaaaaggtcaaagaaaccaggaaagtaaagctatatttcagcggaaaatgggatatttatgagttttctaagttgacagactcaagttttaaccttcagtgcccatcatgtaggaaagtgtggcataactggctgattctggctttctactcctttttcccattaaagatccctcctgcttaattaacattcacaagtaactctggttgtactttaggcacagtggctcccgaggtcagtcacacaataggatgtctgtgctccaagttgccagagagagagattactcttgagaatgagcctcagccctggctcaaactcacctgcaaacttcgtgagagatgaggcagaggtacactacgaaagcaacagttagaagctaaatgatgagaacacatggactcatagagggaaacaacgcatactggggcctatcagagggtggagggtgagagaaggagaggatcaggaaaaatcactaatggatgctaagcgtaatacctgagtgatgagatcatctatacaacaaacccccttgacattcatttatctatgtaacaaacctgcacatcctgtacatgtacccctgaacttaaaataaaagttgaaaacaagaaagcaacagtttgaacacttgttatggtctattctctcattctttacaattacactagaaaatagccacaggcttcctgcaaggcagccacagaatttatgacttgtgatatccaagtcattcctggataatgcaaaatctaacacaaaatctagtagaatcatttgcttacatctatttttgttctgagaatatagatttagatacataatggaagcagaataatttaaaatctggctaatttagaatcctaagcagctcttttcctatcagtggtttacaagccttgtttatatttttcctattttaaaaataaaaataaagtaagttatttgtggtaaagaatattcattaaagtatttatttcttagataataccatgaaaaacattcagtgaagtgaagggcctactttacttaacaagaatctaatttatataatttttcatactaatagcatctaagaacagtacaatatttgactcttcaggttaaacatatgtcataaattagccagaaagatttaagaaaatattggatgtttccttgtttaaattaggcatcttacagtttttagaatcctgcatagaacttaagaaattacaaatgctaaagcaaacccaaacaggcaggaattaatcttcatcgaatttgggtgtttctttctaaaagtcctttatacttaaatgtcttaagacatacatagattttattttactaattttaattatatagacaataaatgaatattcttactgattactttttctgactgtctaatctttctgatctatcctggatggccataacacttatctctctgaactttgggcttttaatataggaaagaaaagcaataatccatttttcatggtatctcatatgataaacaaataaaatgcttaaaaatgagcaggtgaagcaatttatcttgaaccaacaagcatcgaagcaataatgagactgcccgcagcctacctgacttctgagtcaggatttataagccttgttactgagacacaaacctgggcctttcaatgctataacctttcttgaagctcctccctaccacctttagccataaggaaacatggaatgggtcagatccctggatgcaagccaggtctggaaccataggcagtaaggagagaagaaaatgtgggctctgcaactggctccgagggagcaggagaggatcaaccccatactctgaatctaagagaagactggtgtccatactctgaatgggaagaatgatgggattacccatagggcttgttttagggagaaacctgttctccaaactcttggccttgagatacctggtccttattccttggactttggcaatgtctgaccctcacattcaagttctgaggaagggccactgccttcatactgtggatctgtagcaaattccccctgaaaacccagagctgtatcttaattggttaaaaaaaattatattatctcaacgactgttcttctctgagtagccaagctcagcttggttcaagctacaagcagctgagctgctttttgtctagtcattgttcttttatttcagtggatcaaatacgttctttccaaacctaggatcttgtcttcctaggctatatattttgtcccaggaagtcttaatctggggtccacagaacactagggggctggtgaagtttatagaaaaaaaatctgtatttttacttacatgtaactgaaatttagcattttcttctactttgaatgcaaaggacaaactagaatgacatcatcagtacctattgcatagttataaagagaaaccacagatattttcatactacaccataggtattgcagatctttttgtttttgtttttgtttgagatggagtttcgctcttattgcccaggctggagtgcagtggcatgatttcggctcactgcaacctccccttcctgcattcaagcaattctcctgccttggcctcctgagtagctggggattacaggcacctgccaccatgccagtctaatttttgtatttttagtagagatggggtttcgccatgttggccaggctggtcttgaactcctgacctcagatgatctgcccgccttggcctcctgaagtgctgggattataggtgtgagccaccacgcctggcccattgcagatatttttaattcacatttatctgcatcactacttggatcttaaggtagctgtagacccaatcctagatctaatgctttcataaagaagcaaatataataaatactataccacaaatgtaatgtttgatgtctgataatgatatttcagtgtaattaaacttagcactcctatgtatattatttgatgcaataaaaacatatttttttagcacttacagtctgccaaactggcctgtgacacaaaaaaagtttaggaattcctggttttgtctgtgttagccaatggttagaatatatgctcagaaagataccattggttaatagctaaaagaaaatggagtagaaattcagtggcctggaataataacaatttgggcagtcattaagtcaggtgaagacttctggaatcatgggagaaaagcaagggagacattcttacttgccacaagtgtttttttttttttttttttttatcacaaacataagaaaatataataaataacaaagtcaggttatagaagagagaaacgctcttagtaaacttggaatatggaatccccaaaggcacttgacttgggagacaggagccatactgctaagtgaaaaagacgaagaacctctagggcctgaacatacaggaaattgtaggaacagaaattcctagatctggtggggcaaggggagccataggagaaagaaatggtagaaatggatggagacggaggcagaggtgggcagatcatgaggtcaagagatcgagaccatcctggcaaacatggtgaaatcccgtctctactaaaaataaaaaaattagctgggcatggtggcatgcgcctgtagtcccagctgctcgggaggctgaggcaggagaatcgtttgaacccaggaggcgaaggttgcagtgagctgagatagtgccattgcactccagtctggcaacagagtgagactccgtctcaaaaaaaaaaaaaaaagaaagaaagaaaagaaaaagaaaaaagaaaaaataaatggatgtagaacaagccagaaggaggaactgggctggggcaatgagattatggtgatgtaagggacttttatagaattaacaatgctggaatttgtggaactctgcttctattattcccccaatcattacttctgtcacattgatagttaaataatttctgtgaatttattccttgattctaaaatatgaggataatgacaatggtattataagggcagattaagtgatatagcatgagcaatattcttcaggcacatggatcgaattgaatacactgtaaatcccaacttccagtttcagctctaccaagtaaagagctagcaagtcatcaaaatggggacatacagaaaaaaaaaaggacactagaggaataatataccctgactcctagcctgattaatatatcgat

SEQ ID NO: 7 - DNA, 1614 bp, Homo sapiens
gatctctatttatttagcaataatagagaaagcatttaagagaataaagcaatggaaataagaaatttgtaaatttccttctgataactagaaatagaggatccagtttcttttggttaacctaaattttatttcattttattgttttattttattttattttattttattttgtgtaatcgtagtttcagagtgttagagctgaaaggaagaagtaggagaaacatgcaaagtaaaagtataacactttccttactaaaccgacatgggtttccaggtaggggcaggattcaggatgactgacagggcccttagggaacactgagaccctacgctgacctcataaatgcttgctacctttgctgttttaattacatcttttaatagcaggaagcagaactctgcacttcaaaagtttttcctcacctgaggagttaatttagtacaaggggaaaaagtacagggggatgggagaaaggcgatcacgttgggaagctatagagaaagaagagtaaattttagtaaaggaggtttaaacaaacaaaatataaagagaaataggaacttgaatcaaggaaatgattttaaaacgcagtattcttagtggactagaggaaaaaaataatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacacatatatatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatgg

SEQ ID NO: 8 - protein, 11 aa, Artificial Sequence
  Her2-specific CDR
KASQDVSIGVA

SEQ ID NO: 9 - protein, 6 aa, Artificial Sequence
  Her2-specific CDR
ASYRYT

SEQ ID NO: 10 - protein, 9 aa, Artificial Sequence
  Her2-specific CDR
QQYYIYPYT

SEQ ID NO: 11 - protein, 10 aa, Artificial Sequence
  Her2-specific CDR
GFTFTDYTMD

SEQ ID NO: 12 - protein, 16 aa, Artificial Sequence
  Her2-specific CDR
DVNPNSGGSIYNQRFK

SEQ ID NO: 13 - protein, 9 aa, Artificial Sequence
  Her2-specific CDR
LGPSFYFDY

SEQ ID NO: 14 - protein, 15 aa, Artificial Sequence
  PD-L1-specific CDR
RASKGVSTSGYSYLH

SEQ ID NO: 15 - protein, 7 aa, Artificial Sequence
  PD-L1-specific CDR
LASYLES

SEQ ID NO: 16 - protein, 9 aa, Artificial Sequence
  PD-L1-specific CDR
QHSRDLPLT

SEQ ID NO: 17 - protein, 5 aa, Artificial Sequence
  PD-L1-specific CDR
NYYMY

SEQ ID NO: 18 - protein, 17 aa, Artificial Sequence
  PD-L1-specific CDR
GINPSNGGTNFNEKFKN

SEQ ID NO: 19 - protein, 11 aa, Artificial Sequence
  PD-L1-specific CDR
RDYRFDMGFDY

SEQ ID NO: 20 - protein, 120 aa, Artificial Sequence
  Avelumab-specific variable heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 21 - protein, 110 aa, Artificial Sequence
  Avelumab-specific variable light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 22 - protein, 11 aa, Artificial Sequence
  Avelumab-specific CDR
SGFTFSSYIMM

SEQ ID NO: 23 - protein, 17 aa, Artificial Sequence
  Avelumab-specific CDR
SIYPSGGITFYADTVKG

SEQ ID NO: 24 - protein, 11 aa, Artificial Sequence
  Avelumab-specific CDR
IKLGTVTTVDY

SEQ ID NO: 25 - protein, 14 aa, Artificial Sequence
  Avelumab-specific CDR
TGTSSDVGGYNYVS

SEQ ID NO: 26 - protein, 7 aa, Artificial Sequence
  Avelumab-specific CDR
DVSNRPS

SEQ ID NO: 27 - protein, 10 aa, Artificial Sequence
  Avelumab-specific CDR
SSYTSSSTRV

SEQ ID NO: 28 - protein, 118 aa, Artificial Sequence [sequence omitted]

SEQ ID NO: 29 - protein, 107 aa, Artificial Sequence [sequence omitted]

SEQ ID NO: 30 - protein, 11 aa, Artificial Sequence
  Atezolizumab-specific CDR
SGFTFSDSWIH

SEQ ID NO: 31 - protein, 17 aa, Artificial Sequence
  Atezolizumab-specific CDR
WISPYGGSTYYADSVKG

SEQ ID NO: 32 - protein, 9 aa, Artificial Sequence
  Atezolizumab-specific CDR
RHWPGGFDY

SEQ ID NO: 33 - protein, 11 aa, Artificial Sequence
  Atezolizumab-specific CDR
RASQDVSTAVA

SEQ ID NO: 34 - protein, 7 aa, Artificial Sequence
  Atezolizumab-specific CDR
SASFLYS

SEQ ID NO: 35 - protein, 9 aa, Artificial Sequence
  Atezolizumab-specific CDR
QQYLYHPAT

SEQ ID NO: 36 - protein, 11 aa, Artificial Sequence
  PSMA-specific-CDR
KASQDVGTAVD

SEQ ID NO: 37 - protein, 7 aa, Artificial Sequence
  PSMA-specific-CDR
WASTRHT

SEQ ID NO: 38 - protein, 9 aa, Artificial Sequence
  PSMA-specific-CDR
QQYNSYPLT

SEQ ID NO: 39 - protein, 10 aa, Artificial Sequence
  PSMA-specific-CDR
GYTFTEYTIH

SEQ ID NO: 40 - protein, 17 aa, Artificial Sequence
  PSMA-specific-CDR
NINPNNGGTTYNQKFED

SEQ ID NO: 41 - protein, 6 aa, Artificial Sequence
  PSMA-specific-CDR
GWNFDY

SEQ ID NO: 42 - protein, 7 aa, Artificial Sequence
  MUC16-specific-CDR
SEDIYSG

SEQ ID NO: 43 - protein, 8 aa, Artificial Sequence
  MUC16-specific-CDR
GYSYSSTL

SEQ ID NO: 44 - protein, 7 aa, Artificial Sequence
  MUC16-specific-CDR
TLGMGVG

SEQ ID NO: 45 - protein, 16 aa, Artificial Sequence
  MUC16-specific-CDR
HIWWDDDKYYNPALKS

SEQ ID NO: 46 - protein, 12 aa, Artificial Sequence
  MUC16-specific-CDR
IGTAQATDALDY

SEQ ID NO: 47 - protein, 15 aa, Artificial Sequence
  FOLR-specific-CDR
KASQSVSFAGTSLMH

SEQ ID NO: 48 - protein, 7 aa, Artificial Sequence
  FOLR-specific-CDR
RASNLEA

SEQ ID NO: 49 - protein, 9 aa, Artificial Sequence
  FOLR-specific-CDR
QQSREYPYT

SEQ ID NO: 50 - protein, 5 aa, Artificial Sequence
  FOLR-specific-CDR
GYFMN

SEQ ID NO: 51 - protein, 17 aa, Artificial Sequence
  FOLR-specific-CDR
RIHPYDGDTFYNQKFQG

SEQ ID NO: 52 - protein, 9 aa, Artificial Sequence
  FOLR-specific-CDR
YDGSRAMDY

SEQ ID NO: 53 - protein, 119 aa, Artificial Sequence
  Amatuximab-specific variable heavy chain
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 54 - protein, 106 aa, Artificial Sequence
  Amatuximab-specific variable light chain
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 55 - protein, 10 aa, Artificial Sequence
  Amatuximab-specific CDR
GYSFTGYTMN

SEQ ID NO: 56 - protein, 13 aa, Artificial Sequence
  Amatuximab-specific CDR
LITPYNGASSYNQ

SEQ ID NO: 57 - protein, 10 aa, Artificial Sequence
  Amatuximab-specific CDR
GGYDGRGFDY

SEQ ID NO: 58 - protein, 10 aa, Artificial Sequence
  Amatuximab-specific CDR
SASSSVSYMH

SEQ ID NO: 59 - protein, 7 aa, Artificial Sequence
  Amatuximab-specific CDR
DTSKLAS

SEQ ID NO: 60 - protein, 9 aa, Artificial Sequence
  Amatuximab-specific CDR
QQWSKHPLT

SEQ ID NO: 61 - protein, 32 aa, Artificial Sequence
  Nef
VGFPVTPQVPLRPMTYKAAVDLSHFLKEKGGL

SEQ ID NO: 62 - protein, 30 aa, Artificial Sequence
  Nef
HTQGYFPDWQNYTPGPGVRYPLTFGWLYKL

SEQ ID NO: 63 - protein, 19 aa, Artificial Sequence
  Gag p17
EKIRLRPGGKKKYKLKHIV

SEQ ID NO: 64 - protein, 32 aa, Artificial Sequence
  Gag p17-p24
NPPIPVGEIYKRWIILGLNKIVRMYSPTSILD

SEQ ID NO: 65 - protein, 31 aa, Artificial Sequence
  Pol
AIFQSSMTKILEPFRKQNPDIVIYQYMDDLY

SEQ ID NO: 66 - DNA, 74 bp, Artificial Sequence; identical to SEQ ID NO: 4
  IR/DR and chromosomal sequence of Sleeping Beauty

SEQ ID NO: 67 - DNA, 75 bp, Artificial Sequence; identical to SEQ ID NO: 5
  IR/DR and chromosomal sequence of Sleeping Beauty

SEQ ID NO: 68 - DNA, 33 bp, Artificial Sequence
  IR/DR and chromosomal sequence of Sleeping Beauty
acttaagtgtatgtaaacttccgacttcaactg

SEQ ID NO: 69 - DNA, 370 bp, Artificial Sequence
  IR/DR and chromosomal sequence of Sleeping Beauty
cagtcaacttagtgtatgtaaacttctgacccactggaattgtgatacagtgaattataagtgaaataatctgtctgtaaacaattgttggaaaaatgacttgtgtcatgcacaaagtagatgtcctaactgacttgccaaaactattgtttgttaacaagaaatttgtggagtagttgaaaaacgagttttaatgactccaacttaagtgtatgtaaacttccgacttcaactgtaagaatggcccattcatctatagtagcacacaatatttgcatttgtgcgacagtataagggacaattatgctatcaggcatttttccaaagtgagtaatcgaagtttttatacctttgtgtgccatgtttgcta

SEQ ID NO: 70 - DNA, 369 bp, Artificial Sequence
  IR/DR and chromosomal sequence of Sleeping Beauty
cagtcaacttagtgtatgtaaacttctgacccactggaattgtgatacagtgaattataagtgaaataatctgtctgtaaacaattgttggaaaaatgacttgtgtcatgcacaaagtagatgtcctaactgacttgccaaaactattgtttgttaacaagaaatttgtggagtagttgaaaaacgagttttaatgactccaacttaagtgtatgtaaacttccgacttcaactgtacaagtagaccaaatatccatatacataaaagaaaaaaatagaaaaaatttctagtgacagaaaaatgacaaagaacatactgctttattactactattaagatgtttgcttccattacactcatatgagtca

SEQ ID NO: 71 - DNA, 227 bp, Artificial Sequence
  IR/DR of Sleeping Beauty
ttagtgtatgtaaacttctgacccactggaattgtgatacagtgaattataagtgaaataatctgtctgtaaacaattgttggaaaaatgacttgtgtcatgcacaaagtagatgtcctaactgacttgccaaaactattgtttgttaacaagaaatttgtggagtagttgaaaaacgagttttaatgactccaacttaagtgtatgtaaacttccgacttcaactg

SEQ ID NO: 72 - DNA, 371 bp, Artificial Sequence
  IR/DR and chromosomal sequence of Sleeping Beauty
caacttgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtatatcctccccgttgcaccctcttgatgatgctgagatgaacacagatgctcactccttgagggctctaagcttatgctgacacagacacaggtgctcacttctatgaatggcctaagatttgaggacatcatgagg

SEQ ID NO: 73 - DNA, 229 bp, Artificial Sequence
  IR/DR of Sleeping Beauty
ttgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactg

SEQ ID NO: 74 - protein, 340 aa, Artificial Sequence
  Sleeping Beauty transposase enzyme
MGKSKEISQDLRKKIVDLHKSGSSLGAISKRLKVPRSSVQTIVRKYKHHGTTQPSYRSGRRRYLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 75 - protein, 340 aa, Artificial Sequence
  Hyperactive Sleeping Beauty is SB100X
MGKSKEISQDLRKRIVDLHKSGSSLGAISKRLAVPRSSVQTIVRKYKHHGTTQPSYRSGRRRYLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 76 - protein, 594 aa, Trichoplusia ni
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 77 - protein, 340 aa, Rana pipiens
MPRPKEIQEQLRKKVIEIYQSGKGYKAISKALGIQRTTVRAIIHKWRRHGTVVNLPRSGRPPKITPRAQRRLIQEVTKDPTTTSKELQASLASVKVSVHASTIRKRLGKNGLHGRVPRRKPLLSKKNIKARLNFSTTHLDDPQDFWDNILWTDETKVELFGRCVSKYIWRRRNTAFHKKNIIPTVKYGGGSVMVWGCFAASGPGRLAVIKGTMNSAVYQEILKENVRPSVRVLKLKRTWVLQQDNDPKHTSKSTTEWLKKNKMKTLEWPSQSPDLNPIEMLWYDLKKAVHARKPSNVTELGQFCKDEWAKIPPGRCKSLIARYRKRLVAVVAAKGGPTSY

SEQ ID NO: 78 - protein, 636 aa, Tribolium castaneum
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLKNAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 79 - protein, 649 aa, Oryzias latipes
MEEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 80 - DNA, 348 bp, Artificial Sequence
  SV40 promoter
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcg

SEQ ID NO: 81 - DNA, 206 bp, Artificial Sequence
  dESV40 promoter
gcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 82 - DNA, 463 bp, Homo sapiens
ttggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttaccgcggccccgccctctcctcgcggcgcgagtttcaggcagcgctgcgtcctgctgcgcacgtgggaagccctggccccggccacccccgccagatct

SEQ ID NO: 83 - DNA, 648 bp, Rous sarcoma virus
acgcgtcatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagctgctagcaagcttgagatct

SEQ ID NO: 84 - DNA, 1740 bp, Homo sapiens
gagtagctgggattacaggcatgtgccaccacgcctcgctaatattagtatttttcatacagacaagatctcactatgttgctcagggtagtctcgaattctgggactcaaatgatcctcccacttcagcctcccaaagtgctgggattacaggcataagccatcatgcccggcctctgacgctgtttctttcaacccccaggatttcagattccaccagcttatggagaagggaaccaagttcgagatgcgtgattgcccagaaagttggaggctgagctgagacttgaacccagagaccagaacctccagaggtcaaagtcctcctcctgggtcccccagagaagggccctgagatgacagctcgttggtcctcatggaagcgtgacccccccagtagactttctcccacacccaaccttggtttcctcatctatatgatagggacaagccagactctacctccctggtggtcatggtctccgcttattcgggttcataaccttaaaggcccctcgcaccacctcagtgagccatttatgcctggcacagggccaactctcagtgcatatctgcaaaggaaccaatgaatgagtgaatgaagtgacaaatgaataaaggaataaatgaatgaggcacttatcatgtaccaggctttcgttaccacgtcccatttattcctctgaggcagggtctattttatccttgttacagatggggaaactaaggcccagggaggagcaaagtcttccccaagtatgtacccactcagaacttgagctctgaatgtctcccacccagcttagcccaagagcggggttcagtgatgcccaccccctaaggctctagagaaagggggtaggcccacatgccagtttgggggtggtaaagccaggtaagttttctttatgggtcccctgaaaccctgaaagtgaaccccagtcctgcatgaaagtgagctccccatagctcaaggtattcaagcacaatacggctttgagtgctgaagcaggctgtgcaggcttggatagtgacatgccctctctgagcctcaatttccccacctgtcaacagcagacagtgacagctgtgatcaggggatcacagtgcatggggatgggtgggtgcatggggatggaggggcatttgggagccctccccgataccaccccctgcagccacccagatagcctgtcctggcctgtctgtcccagtccagggctgaaagggtgcgggtcctgcccgcccctaggtctggaggcggagtcgcggtgacccgggagcccaataaatctgcaacccacaatcacgagctgctcccgtaagccccaaggcgacctccagctgtcagcgctgagcacagcgcccagggagagggacagacagccggctgcatgggacagcggaacccagagtgagaggggaggtggcaggacagacagacagcaggggcggacgcagagacagacagcggggacagggaggccgacacggacatcgacagcccatagattcctaacccagggagccccggcccctctcgccgcttcccaccccagacggagcggggacaggctgccgagcatcctcccacccgccctccccgtcctgcctcctcggcccctgccagcttcccccgcttgagcacgcagggcgtccgaggacgcgctgggcctccgcacccgccctcatggaggccgtggagaccggggaacggcccaccttcggagcctgggacta

SEQ ID NO: 85 - DNA, 1075 bp, Homo sapiens
attagagattgtaaattgggctctgagcttcctaccaacaaaagcacaaaggaaaatatgatcactggtattaaaaaaaaacacctatggtttccaaaagattaaaacaaaccagcagttttatagaagctaacactaaaatctaaaggaactacgttctatggagccacttaatatggataaacactttgacaatattctttcaacaactacagtaacaagtttcttagagtccatttctttttacatccataatgaattgtaaatcttttctacttcttaagtaaaacatcaccacttaattctggtaacttttccatattaactttttagaacaattgcaaacgtaccataaatgattgttgtcacagtggtaactatttgaccctgactgttattttgtatatagcagcttttaaaataaaaaggcaacaagtttctaggcgtaatttccacagatcttttatgtaaaacaatgacatcctttgcaacttctgccatttaatctatctcaagcaagctctctggaaacaaatctatttgaaagattctattgtaattagaaatcagggtaactgaatgcactagatgaaaaccttctgactggggccaatgaagtcaataaagtcaaaactgctgtgaatgctcaactgtctgcagatcagatgtcttgggatggaatccgttctcgaggccaccatcattaatatcaatttggccatgtaatacaagcctcacttgttccactgttacaaatgtgcttaaaactgagctcatttacaatccaaatacatatgtaggatggtaaccaaggcatcacactaatttaggtattatgttttagggggaacaaaaggtatgttaatattttattcatctccaaattaactataaattgtgcattcttgcatagatcctccttgggaatgagaaattaggaaaatccagttgttaaaatgaatgcctaaaatcaaaataaaatttgtttttctggcacctgcttgatgacacagactaataaccaatgacaaaattcccttgaacccaagttttcatttcctcctattgtgtggtc

SEQ ID NO: 86 - DNA, 22 bp, Artificial Sequence
  primer
gtgcttgaaggggaacaactac

SEQ ID NO: 87 - DNA, 22 bp, Artificial Sequence
  primer
cctggcctccagataactacac

SEQ ID NO: 88 - DNA, 40 bp, Artificial Sequence
  primer
ccccctcgaggtcgacatggctagagacttatcgaaagca

SEQ ID NO: 89 - DNA, 38 bp, Artificial Sequence
  primer
attcgatatcaagctccaagatctgcacactggtattt

SEQ ID NO: 90 - DNA, 39 bp, Artificial Sequence
  primer
ccccctcgaggtcgacgtacacgacatcactttcccagt

SEQ ID NO: 91 - DNA, 37 bp, Artificial Sequence
  primer
attcgatatcaagctcacactggtatttcggtttttg

SEQ ID NO: 92 - DNA, 40 bp, Artificial Sequence
  primer
ccccctcgaggtcgacctacactctcagtcagcctatgga

SEQ ID NO: 93 - DNA, 39 bp, Artificial Sequence
  primer
attcgatatcaagcttaatcccaaaaggctgatagtctc

SEQ ID NO: 94 - DNA, 40 bp, Artificial Sequence
  primer
ccccctcgaggtcgacacatctctcactttctcatcacca

SEQ ID NO: 95 - DNA, 39 bp, Artificial Sequence
  primer
attcgatatcaagctaagtaactgggattacaggagcac

SEQ ID NO: 96 - DNA, 24 bp, Artificial Sequence
  primer
aaagggcaaataccttaaggggtg

SEQ ID NO: 97 - DNA, 25 bp, Artificial Sequence
  primer
agcacttcgacctaaaaatagagat

SEQ ID NO: 98 - DNA, 21538 bp, Artificial Sequence
  long beta-globin LCR with inserted XhoI site
gatctctatcccctcctgttttctctacgttatttatatgggtatcatcaccatcctggacaacatcaggacagatatccctcaccaagccaatgttcctctctatgttggctcaaatgtccttgaactttcctttcaccaccctttccacagtcaaaaggatattgtagtttaatgcctcagagttcagcttttaagcttctgacaaattattcttcctctttaggttctcctttatggaatcttctgtactgatggccatgtcctttaactactatgtagatatctgctactacctgtattatgcctctacctttattagcagagttatctgtactgttggcatgacaatcatttgttaatatgacttgcctttcctttttctgctattcttgatcaaatggctcctctttcttgctcctctcatttctcctgccttcacttggacgtgcttcacgtagtctgtgcttatgactggattaaaaattgatatggacttatcctaatgttgttcgtcataatatgggttttatggtccattattatttcctatgcattgatctggagaaggcttcaatccttttactctttgtggaaaatatctgtaaaccttctggttcactctgctatagcaatttcagtttaggctagtaagcatgaggatgcctccttctctgatttttcccacagtctgttggtcacagaataacctgagtgattactgatgaaagagtgagaatgttattgatagtcacaatgacaaaaaacaaacaactacagtcaaaatgtttctctttttattagtggattatatttcctgacctatatctggcaggactctttagagaggtagctgaagctgctgttatgaccactagagggaagaagatacctgtggagctaatggtccaagatggtggagccccaagcaaggaagttgttaaggagcccttttgattgaaggtgggtgcccccaccttacagggacaggacatctggatactcctcccagtttctccagtttccctttttcctaatatatctcctgataaaatgtctatactcacttccccatttctaataataaagcaaaggctagttagtaagacatcaccttgcattttgaaaatgccatagactttcaaaattatttcatacatcggtctttctttatttcaagagtccagaaatggcaacattacctttgattcaatgtaatggaaagagctctttcaagagacagagaaaagaataatttaatttctttccccacacctccttccctgtctcttaccctatcttccttccttctaccctccccatttctctctctcatttctcagaagtatattttgaaaggattcatagcagacagctaaggctggttttttctaagtgaagaagtgatattgagaaggtagggttgcatgagccctttcagttttttagtttatatacatctgtattgttagaatgttttataatataaataaaattatttctcagttatatactagctatgtaacctgtggatatttccttaagtattacaagctatacttaactcacttggaaaactcaaataaatacctgcttcatagttattaataaggattaagtgagataatgcccataagattcctattaataacagataaatacatacacacacacacacattgaaaggattcttactttgtgctaggaactataataagttcattgatgcattatatcattaagttctaatttcaacactagaaggcaggtattatctaaatttcatactggatacctccaaactcataaagataattaaattgccttttgtcatatatttattcaaaagggtaaactcaaactatggcttgtctaattttatatatcaccctactgaacatgaccctattgtgatattttataaaattattctcaagttattatgaggatgttgaaagacagagaggatggggtgctatgccccaaatcagcctcacaattaagctaagcagctaagagtcttgcagggtagtgtagggaccacagggttaagggggcagtagaattatactcccactttagtttcatttcaaacaatccatacacacacagccctgagcacttacaaattatactacgctctatactttttgtttaaatgtataaataagtggatgaaagaatagatagatagatagacagatagatgatagatagaataaatgcttgccttcatagctgtctccctaccttgttcaaaatgttcctgtccagaccaaagtaccttgccttcacttaagtaatcaattcctaggttatattctgatgtcaaaggaagtcaaaagatgtgaaaaacaatttctgacccacaactcatgctttgtagatgactagatcaaaaaatttcagccatatcttaacagtgagtgaacaggaaatctcctcttttccctacatctgagatcccagcttctaagaccttcaattctcactcttgatgcaacagaccttggaagcatacaggagagctgaacttggtcaacaaaggagaaaagtttgttggcctccaaaggcacagctcaaacttttcaagccttctctaatcttaaaggtaaacaagggtctcatttctttgagaacttcagggaaaatagacaaggacttgcctggtgcttttggtaggggagcttgcactttccccctttctggaggaaatatttatccccaggtagttccctttttgcaccagtggttctttgaagagacttccacctgggaacagttaaacagcaactacagggccttgaactgcacactttcagtccggtcctcacagttgaaaagacctaagcttgtgcctgatttaagcctttttggtcataaaacattgaattctaatctccctctcaaccctacagtcacccatttggtatattaaagatgtgttgtctactgtctagtatccctcaagtagtgtcaggaattagtcatttaaatagtctgcaagccaggagtggtggctcatgtctgtaattccagcacttgagaggtagaagtgggaggactgcttgagctcaagagtttgatattatcctggacaacatagcaagacctcgtctctacttaaaaaaaaaaaaaaaattagccaggcatgtgatgtacacctgtagtcccagctactcaggaggccgaaatgggaggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttggggaccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttctgttactccaagactgagaaggaaattgaacctgagactcattgactggcaagatgtccccagaggctctcattcagcaataaaattctcaccttcacccaggcccactgagtgtcagatttgcatgcactagttcacgtgtgtaaaaaggaggatgcttctttcctttgtattctcacatacctttaggaaagaacttagcacccttcccacacagccatcccaataactcatttcagtgactcaacccttgactttataaaagtcttgggcagtatagagcagagattaagagtacagatgctggagccagaccacctgagtgattagtgactcagtttctcttagtagttgtatgactcagtttcttcatctgtaaaatggagggttttttaattagtttgtttttgagaaagggtctcactctgtcacccaaatgggagtgtagtggcaaaatctcggctcactgcaacttgcacttcccaggctcaagcggtcctcccacctcaacatcctgagtagctggaaccacaggtacacaccaccatacctcgctaattttttgtatttttggtagagatggggtttcacatgttacacaggatggtctcagactccggagctcaagcaatctgcccacctcagccttccaaagtgctgggattataagcatgattacaggagttttaacaggctcataagattgttctgcagcccgagtgagttaatacatgcaaagagtttaaagcagtgacttataaatgctaactactctagaaatgtttgctagtattttttgtttaactgcaatcattcttgctgcaggtgaaaactagtgttctgtactttatgcccattcatctttaactgtaataataaaaataactgacatttattgaaggctatcagagactgtaattagtgctttgcataattaatcatatttaatactcttggattctttcaggtagatactattattatccccattttactacagttaaaaaaactacctctcaacttgctcaagcatacactctcacacacacaaacataaactactagcaaatagtagaattgagatttggtcctaattatgtctttgctcactatccaataaatatttattgacatgtacttcttggcagtctgtatgctggatgctggggatacaaagatgtttaaatttaagctccagtctctgcttccaaaggcctcccaggccaagttatccattcagaaagcattttttactctttgcattccactgtttttcctaagtgactaaaaaattacactttattcgtctgtgtcctgctctgggatgatagtctgactttcctaacctgagcctaacatccctgacatcaggaaagactacaccatgtggagaaggggtggtggttttgattgctgctgtcttcagttagatggttaactttgtgaagttgaaaactgtggctctctggttgactgttagagttctggcacttgtcactatgcctattatttaacaaatgcatgaatgcttcagaatatgggaatattatcttctggaatagggaatcaagttatattatgtaacccaggattagaagattcttctgtgtgtaagaatttcataaacattaagctgtctagcaaaagcaagggcttggaaaatctgtgagctcctcaccatatagaaagcttttaacccatcattgaataaatccctataggggatttctaccctgagcaaaaggctggtcttgattaattcccaaactcatatagctctgagaaagtctatgctgttaacgttttcttgtctgctaccccatcatatgcacaacaataaatgcaggcctaggcatgactgaaggctctctcataattcttggttgcatgaatcagattatcaacagaaatgttgagacaaactatggggaagcagggtatgaaagagctctgaatgaaatggaaaccgcaatgcttcctgcccattcagggctccagcatgtagaaatctggggctttgtgaagactggcttaaaatcagaagccccattggataagagtagggaagaacctagagcctacgctgagcaggtttccttcatgtgacagggagcctcctgccccgaacttccagggatcctctcttaagtgtttcctgctggaatctcctcacttctatctggaaatggtttctccacagtccagcccctggctagttgaaagagttacccatgcagaggccctcctagcatccagagactagtgcttagattcctactttcagcgttggacaacctggatccacttgcccagtgttcttccttagttcctaccttcgaccttgatcctcctttatcttcctgaaccctgctgagatgatctatgtggggagaatggcttctttgagaaacatcttcttcgttagtggcctgcccctcattcccactttaatatccagaatcactataagaagaatataataagaggaataactcttattataggtaagggaaaattaagaggcatacgtgatgggatgagtaagagaggagagggaaggattaatggacgataaaatctactactatttgttgagaccttttatagtctaatcaattttgctattgttttccatcctcacgctaactccataaaaaaacactattattatctttattttgccatgacaagactgagctcagaagagtcaagcatttgcctaaggtcggacatgtcagaggcagtgccagacctatgtgagactctgcagctactgctcatgggccctgtgctgcactgatgaggaggatcagatggatggggcaatgaagcaaaggaatcattctgtggataaaggagacagccatgaagaagtctatgactgtaaatttgggagcaggagtctctaaggacttggatttcaaggaattttgactcagcaaacacaagaccctcacggtgactttgcgagctggtgtgccagatgtgtctatcagaggttccagggagggtggggtggggtcagggctggccaccagctatcagggcccagatgggttataggctggcaggctcagataggtggttaggtcaggttggtggtgctgggtggagtccatgactcccaggagccaggagagatagaccatgagtagagggcagacatgggaaaggtgggggaggcacagcatagcagcatttttcattctactactacatgggactgctcccctatacccccagctaggggcaagtgccttgactcctatgttttcaggatcatcatctataaagtaagagtaataattgtgtctatctcatagggttattatgaggatcaaaggagatgcacactctctggaccagtggcctaacagttcaggacagagctatgggcttcctatgtatgggtcagtggtctcaatgtagcaggcaagttccagaagatagcatcaaccactgttagagatatactgccagtctcagagcctgatgttaatttagcaatgggctgggaccctcctccagtagaaccttctaaccagctgctgcagtcaaagtcgaatgcagctggttagactttttttaatgaaagcttagctttcattaaagattaagctcctaagcagggcacagatgaaattgtctaacagcaactttgccatctaaaaaaatctgacttcactggaaacatggaagcccaaggttctgaacatgagaaatttttaggaatctgcacaggagttgagagggaaacaagatggtgaagggactagaaaccacatgagagacacgaggaaatagtgtagatttaggctggaggtaaatgaaagagaagtgggaattaatacttactgaaatctttctatatgtcaggtgccattttatgatatttaataatctcattacatatggtaattctgtgagatatgtattattgaacatactataattaatactaatgataagtaacacctcttgagtacttagtatatgctagaatcaaatttaagtttatcatatgaggccgggcacggtggctcatatatgggattacatgcctgtaatcccagcactttgggaggccaaggcaattggatcacctgaggtcaggagttccagaccagcctggccaacatggtgaaaccccttctctactaaaaaatacaaaaaatcagccaggtgtggtggcacgcgtctataatcccagctactcaggaggctgaggcaggagaatcacttgaacccaggaggtggaggttgcagtgagctaagattgcaccactgcactccagcctaggcgacagagtgagactccatctcaaaaaaaaaaaaagaagtttattatatgaattaacttagttttactcacaccaatactcagaagtagattattacctcatttattgatgaggagcccaatgtacttgtagtgtagatcaacttattgaaagcacaagctaataagtagacaattagtaattagaagtcagatggtctgagctctcctactgtctacattacatgagctcttattaactggggactcgaaaatcaaagacatgaaataatttgtccaagcttacagaaccaccaagtagtaaggctaggatgtagacccagttctgctacctctgaagacagtgttttttccacagcaaaacacaaactcagatattgtggatgcgagaaattagaagtagatattcctgccctgtggcccttgcttcttacttttacttcttgtcgattggaagttgtggtccaagccacagttgcagaccatacttcctcaaccataattgcatttcttcaggaaagtttgagggagaaaaaggtaaagaaaaatttagaaacaacttcagaataaagagattttctcttgggttacagagattgtcatatgacaaattataagcagacacttgagaaaactgaaggcccatgcctgcccaaattaccctttgaccccttggtcaagctgcaactttggttaaagggagtgtttatgtgttatagtgttcatttactcttctggtctaacccattggctccgtcttcatcctgcagtgacctcagtgcctcagaaacatacatatgtttgtctagtttaagtttgtgtgaaattctaactagcgtcaagaactgagggccctaaactatgctaggaatagtgctgtggtgctgtgataggtacacaagaaatgagaagaaactgcagattctctgcatctccctttgccgggtctgacaacaaagtttccccaaattttaccaatgcaagccatttctccatatgctaactactttaaaatcatttggggcttcacattgtctttctcatctgtaaaaagaatggaagaactcattcctacagaactccctatgtcttccctgatgggctagagttcctctttctcaaaaattagccattattgtatttccttctaagccaaagctcagaggtcttgtattgcccagtgacatgcacactggtcaaaagtaggctaagtagaagggtactttcacaggaacagagagcaaaagaggtgggtgaatgagagggtaagtgagaaaagacaaatgagaagttacaacatgatggcttgttgtctaaatatctcctagggaattattgtgagaggtctgaatagtgttgtaaaataagctgaatctgctgccaacattaacagtcaagaaatacctccgaataactgtacctccaattattctttaaggtagcatgcaactgtaatagttgcatgtatatatttatcataatactgtaacagaaaacacttactgaatatatactgtgtccctagttctttacacaataaactaatctcatcctcataattctattagctaatacatattatcatcctatatttcagagacttcaagaagttaagcaacttgctcaagatcatctaagaagtaggtggtatttctgggctcatttggcccctcctaatctctcatggcaacatggctgcctaaagtgttgattgccttaattcatcagggatgggctcatactcactgcagaccttaactggcatcctcttttcttatgtgatctgcctgaccctagtagacttatgaaatttctgatgagaaaggagagaggagaaaggcagagctgactgtgatgagtgatgaaggtgccttctcatctggctcgagggtaccagtggggcctctaagactaagtcactctgtctcactgtgtcttagccagttccttacagcttgccctgatgggagatagagaatgggtatcctccaacaaaaaaataaattttcatttctcaaggtccaacttatgttttcttaatttttaaaaaaatcttgaccattctccactctctaaaataatccacagtgagagaaacattcttttcccccatcccataaatacctctattaaatatggaaaatctgggcatggtgtctcacacctgtaatcccagcactttgggaggctgaggtgggtggactgcttggagctcaggagttcaagaccatcttggacaacatggtgataccctgcctctacaaaaagtacaaaaattagcctggcatggtggtgtgcacctgtaatcccagctattagggtggctgaggcaggagaattgcttgaacccgggaggcggaggttgcagtgagctgagatcgtgccactgcactccagcctgggggacagagcacattataattaactgttattttttacttggactcttgtggggaataagatacatgttttattcttatttatgattcaagcactgaaaatagtgtttagcatccagcaggtgcttcaaaaccatttgctgaatgattactatactttttacaagctcagctccctctatcccttccagcatcctcatctctgattaaataagcttcagtttttccttagttcctgttacatttctgtgtgtctccattagtgacctcccatagtccaagcatgagcagttctggccaggcccctgtcggggtcagtgccccacccccgccttctggttctgtgtaaccttctaagcaaaccttctggctcaagcacagcaatgctgagtcatgatgagtcatgctgaggcttagggtgtgtgcccagatgttctcagcctagagtgatgactcctatctgggtccccagcaggatgcttacagggcagatggcaaaaaaaaggagaagctgaccacctgactaaaactccacctcaaacggcatcataaagaaaatggatgcctgagacagaatgtgacatattctagaatatattatttcctgaatatatatatatatatacacatatacgtatatatatatatatatatatatttgttgttatcaattgccatagaatgattagttattgtgaatcaaatatttatcttgcaggtggcctctatacctagaagcggcagaatcaggctttattaatacatgtgtatagatttttaggatctatacacatgtattaatatgaaacaaggatatggaagaggaaggcatgaaaacaggaaaagaaaacaaaccttgtttgccattttaaggcacccctggacagctaggtggcaaaaggcctgtgctgttagaggacacatgctcacatacggggtcagatctgacttggggtgctactgggaagctctcatcttaaggatacatctcaggccagtcttggtgcattaggaagatgtaggcaactctgatcctgagaggaaagaaacattcctccaggagagctaaaagggttcacctgtgtgggtaactgtgaaggactacaagaggatgaaaaacaatgacagacagacataatgcttgtgggagaaaaaacaggaggtcaaggggatagagaaggcttccagaagaatggctttgaagctggcttctgtaggagttcacagtggcaaagatgtttcagaaatgtgacatgacttaaggaactatacaaaaaggaacaaatttaaggagaggcagataaattagttcaacagacatgcaaggaattttcagatgaatgttatgtctccactgagcttcttgaggttagcagctgtgagggttttgcaggcccaggacccattacaggacctcacgtatacttgacactgttttttgtattcatttgtgaatgaatgacctcttgtcagtctactcggtttcgctgtgaatgaatgatgtcttgtcagcctacttggtttcgctaagagcacagagagaagatttagtgatgctatgtaaaaacttcctttttggttcaagtgtatgtttgtgatagaaatgaagacaggctacatgatgcatatctaacataaacacaaacattaagaaaggaaatcaacctgaagagtatttatacagataacaaaatacagagagtgagttaaatgtgtaataactgtggcacaggctggaatatgagccatttaaatcacaaattaattagaaaaaaaacagtggggaaaaaattccatggatgggtctagaaagactagcattgttttaggttgagtggcagtgtttaaagggtgatatcagactaaacttgaaatatgtggctaaataactagaatactctttattttttcgtatcatgaatagcagatatagcttgatggccccatgcttggtttaacatccttgctgttcctgacatgaaatccttaatttttgacaaaggggctattcattttcattttatattgggcctagaaattatgtagatggtcctgaggaaaagtttatagcttgtctatttctctctctaacatagttgtcagcacaatgcctaggctataggaagtactcaaagcttgttaaattgaattctatccttcttattcaattctacacatggaggaaaaactcatcagggatggaggcacgcctctaaggaaggcaggtgtggctctgcagtgtgattgggtacttgcaggacgaagggtggggtgggagtggctaaccttccattcctagtgcagaggtcacagcctaaacatcaaattccttgaggtgcggtggctcactcctgtaatcacagcagtttgggacgccaaggtgggcagatcacttgaggtcaggagttggacaccagcccagccaacatagtgaaacctggtctctgcttaaaaatataaaaattagctggacgtggtgacgggagcctgtaatccaactacttgggaggctgaggcaggagaatcgcttgaaccggggaggtggagtttgcactgagcagagatcatgccattgcactccagcctccagagcgagactctgtctaaagaaaaacgaaaacaaacaaacaaacaaacaaacaaaacccatcaaattccctgaccgaacagaattctgtctgattgttctctgacttatctaccattttccctccttaaagaaactgtgaacttccttcagctagaggggcctggctcagaagcctctggtcagcatccaagaaatacttgatgtcactttggctaaaggtatgatgtgtagacaagctccagagatggtttctcatttccatatccacccacccagctttccaattttaaagccaattctgaggtagagactgtgatgaacaaacaccttgacaaaattcaacccaaagactcactttgcctagcttcaaaatccttactctgacatatactcacagccagaaattagcatgcactagagtgtgcatgagtgcaacacacacacacaccaattccatattctctgtcagaaaatcctgttggtttttcgtgaaaggatgttttcagaggctgaccccttgccttcacctccaatgctaccactctggtctaagtcactgtcaccaccacctaaattatagctgttgactcataacaatcttcctgcttctaccactgccccactacaatttcttcccaatatactatccaaattagtcttttcaaaatgtaagtcatatatggtcacctctttgttcaaagtcttctgatagtttcctatatcatttataataaaaccaaatccttacaattctctacaatagttgttcatgcatatattatgtttattacagatacatatatatagctctcatataaataaatatatatatttatgtgtatgtgtgtagagtgttttttcttacaactctatgatgtaggtattattagtgtcccaaattttataatttaggacttctatgatctcatcttttattctccccttcaccgaatctcatcctacattggccttattgatattccttgaaaattctaagcatcttacatctttagggtatttacatttgccattccctatgccctaaatatttaatcatagtttcatataaatgggttcctcatcatctatgggtactctctcaggtgttaactttatagtgaggactttcctgccatactacttaaagtagcgataccctttcaccctgtcctaatcacactctggccttcatttcagttttttttttttctccatagcacctaatctcattggtatataacatgtttcatttgcttatttaatgtcaagctctttccactatcaagtccatgaaaacaggaactttattcctctattctgtttttgtgctgtattcttagcaattttacaattttgaatgaatgaatgagcagtcaaacacatatacaactataattaaaaggatgtatgctgacacatccactgctatgcacacacaaagaaatcagtggagtagagctggaagtgctaagcctgcatagagctagttagccctccgcaggcagagccttgatgggattactgagttctagaattggactcatttgttttgtaggctgagatttgctcttgaaaacttgttctgaccaaaataaaaggctcaaaagatgaatatcgaaaccagggtgttttttacactggaatttataactagagcactcatgtttatgtaagcaattaattgtttcatcagtcaggtaaaagtaaagaaaaactgtgccaaggcaggtagcctaatgcaatatgccactaaagtaaacattatttcataggtgtcagatatggcttattcatccatcttcatgggaaggatggccttggcctggacatcagtgttatgtgaggttcaaaacacctctaggctataaggcaacagagctccttttttttttttctgtgctttcctggctgtccaaatctctaatgataagcatacttctattcaatgagaatattctgtaagattatagttaagaattgtgggagccattccgtctcttatagttaaatttgagcttcttttatgatcactgtttttttaatatgctttaagttctggggtacatgtgccatggtggtttgctgcacccatcaacccgtcatctacattaggtatttctcctaatgctatccttcccctagccccccacccccaacaggccccagtgtgtgatgttcccctccctgtgtccatggatcactggtttttttttgttttttttttttttttaaagtctcagttaaatttttggaatgtaatttattttcctggtatcctaggacttgcaagttatctggtcactttagccctcacgttttgatgataatcacatatttgtaaacacaacacacacacacacacacacacacatatatatatatataaaacatatatatacataaacacacataacatatttatcgggcatttctgagcaactaatcatgcaggactctcaaacactaacctatagccttttctatgtatctacttgtgtagaaaccaagcgtggggactgagaaggcaatagcaggagcattctgactctcactgcctttagctaggcccctccctcatcacagctcagcatagtcctgagctcttatctatatccacacacagtttctgacgctgcccagctatcaccatcccaagtctaaagaaaaaaataatgggtttgcccatctctgttgattagaaaacaaaacaaaataaaataagcccctaagctcccagaaaacatgactaaaccagcaagaagaagaaaatacaataggtatatgaggagactggtgacactagtgtctgaatgaggcttgagtacagaaaagaggctctagcagcatagtggtttagaggagatgtttctttccttcacagatgccttagcctcaataagcttgcggttgtggaagtttactttcagaacaaactcctgtggggctagaattattgatggctaaaagaagcccgggggagggaaaaatcattcagcatcctcacccttagtgacacaaaacagagggggcctggttttccatatttcctcatgatggatgatctcgttaatgaaggtggtctgacgagatcattgcttcttccatttaagccttgctcacttgccaatcctcagttttaaccttctccagagaaatacacattttttattcaggaaacatactatgttatagtttcaatactaaataatcaaagtactgaagatagcatgcataggcaagaaaaagtccttagctttatgttgctgttgtttcagaatttaaaaaagatcaccaagtcaaggacttctcagttctagcactagaggtggaatcttagcatataatcagaggtttttcaaaatttctagacataagattcaaagccctgcacttaaaatagtctcatttgaattaactctttatataaattgaaagcacattctgaactacttcagagtattgttttatttctatgttcttagttcataaatacattaggcaatgcaatttaattaaaaaaacccaagaatttcttagaattttaatcatgaaaataaatgaaggcatctttacttactcaaggtcccaaaaggtcaaagaaaccaggaaagtaaagctatatttcagcggaaaatgggatatttatgagttttctaagttgacagactcaagttttaaccttcagtgcccatcatgtaggaaagtgtggcataactggctgattctggctttctactcctttttcccattaaagatccctcctgcttaattaacattcacaagtaactctggttgtactttaggcacagtggctcccgaggtcagtcacacaataggatgtctgtgctccaagttgccagagagagagattactcttgagaatgagcctcagccctggctcaaactcacctgcaaacttcgtgagagatgaggcagaggtacactacgaaagcaacagttagaagctaaatgatgagaacacatggactcatagagggaaacaacgcatactggggcctatcagagggtggagggtgagagaaggagaggatcaggaaaaatcactaatggatgctaagcgtaatacctgagtgatgagatcatctatacaacaaacccccttgacattcatttatctatgtaacaaacctgcacatcctgtacatgtacccctgaacttaaaataaaagttgaaaacaagaaagcaacagtttgaacacttgttatggtctattctctcattctttacaattacactagaaaatagccacaggcttcctgcaaggcagccacagaatttatgacttgtgatatccaagtcattcctggataatgcaaaatctaacacaaaatctagtagaatcatttgcttacatctatttttgttctgagaatatagatttagatacataatggaagcagaataatttaaaatctggctaatttagaatcctaagcagctcttttcctatcagtggtttacaagccttgtttatatttttcctattttaaaaataaaaataaagtaagttatttgtggtaaagaatattcattaaagtatttatttcttagataataccatgaaaaacattcagtgaagtgaagggcctactttacttaacaagaatctaatttatataatttttcatactaatagcatctaagaacagtacaatatttgactcttcaggttaaacatatgtcataaattagccagaaagatttaagaaaatattggatgtttccttgtttaaattaggcatcttacagtttttagaatcctgcatagaacttaagaaattacaaatgctaaagcaaacccaaacaggcaggaattaatcttcatcgaatttgggtgtttctttctaaaagtcctttatacttaaatgtcttaagacatacatagattttattttactaattttaattatatagacaataaatgaatattcttactgattactttttctgactgtctaatctttctgatctatcctggatggccataacacttatctctctgaactttgggcttttaatataggaaagaaaagcaataatccatttttcatggtatctcatatgataaacaaataaaatgcttaaaaatgagcaggtgaagcaatttatcttgaaccaacaagcatcgaagcaataatgagactgcccgcagcctacctgacttctgagtcaggatttataagccttgttactgagacacaaacctgggcctttcaatgctataacctttcttgaagctcctccctaccacctttagccataaggaaacatggaatgggtcagatccctggatgcaagccaggtctggaaccataggcagtaaggagagaagaaaatgtgggctctgcaactggctccgagggagcaggagaggatcaaccccatactctgaatctaagagaagactggtgtccatactctgaatgggaagaatgatgggattacccatagggcttgttttagggagaaacctgttctccaaactcttggccttgagatacctggtccttattccttggactttggcaatgtctgaccctcacattcaagttctgaggaagggccactgccttcatactgtggatctgtagcaaattccccctgaaaacccagagctgtatcttaattggttaaaaaaaattatattatctcaacgactgttcttctctgagtagccaagctcagcttggttcaagctacaagcagctgagctgctttttgtctagtcattgttcttttatttcagtggatcaaatacgttctttccaaacctaggatcttgtcttcctaggctatatattttgtcccaggaagtcttaatctggggtccacagaacactagggggctggtgaagtttatagaaaaaaaatctgtatttttacttacatgtaactgaaatttagcattttcttctactttgaatgcaaaggacaaactagaatgacatcatcagtacctattgcatagttataaagagaaaccacagatattttcatactacaccataggtattgcagatctttttgtttttgtttttgtttgagatggagtttcgctcttattgcccaggctggagtgcagtggcatgatttcggctcactgcaacctccccttcctgcattcaagcaattctcctgccttggcctcctgagtagctggggattacaggcacctgccaccatgccagtctaatttttgtatttttagtagagatggggtttcgccatgttggccaggctggtcttgaactcctgacctcagatgatctgcccgccttggcctcctgaagtgctgggattataggtgtgagccaccacgcctggcccattgcagatatttttaattcacatttatctgcatcactacttggatcttaaggtagctgtagacccaatcctagatctaatgctttcataaagaagcaaatataataaatactataccacaaatgtaatgtttgatgtctgataatgatatttcagtgtaattaaacttagcactcctatgtatattatttgatgcaataaaaacatatttttttagcacttacagtctgccaaactggcctgtgacacaaaaaaagtttaggaattcctggttttgtctgtgttagccaatggttagaatatatgctcagaaagataccattggttaatagctaaaagaaaatggagtagaaattcagtggcctggaataataacaatttgggcagtcattaagtcaggtgaagacttctggaatcatgggagaaaagcaagggagacattcttacttgccacaagtgtttttttttttttttttttttatcacaaacataagaaaatataataaataacaaagtcaggttatagaagagagaaacgctcttagtaaacttggaatatggaatccccaaaggcacttgacttgggagacaggagccatactgctaagtgaaaaagacgaagaacctctagggcctgaacatacaggaaattgtaggaacagaaattcctagatctggtggggcaaggggagccataggagaaagaaatggtagaaatggatggagacggaggcagaggtgggcagatcatgaggtcaagagatcgagaccatcctggcaaacatggtgaaatcccgtctctactaaaaataaaaaaattagctgggcatggtggcatgcgcctgtagtcccagctgctcgggaggctgaggcaggagaatcgtttgaacccaggaggcgaaggttgcagtgagctgagatagtgccattgcactccagtctggcaacagagtgagactccgtctcaaaaaaaaaaaaaaaagaaagaaagaaaagaaaaagaaaaaagaaaaaataaatggatgtagaacaagccagaaggaggaactgggctggggcaatgagattatggtgatgtaagggacttttatagaattaacaatgctggaatttgtggaactctgcttctattattcccccaatcattacttctgtcacattgatagttaaataatttctgtgaatttattccttgattctaaaatatgaggataatgacaatggtattataagggcagattaagtgatatagcatgagcaatattcttcaggcacatggatcgaattgaatacactgtaaatcccaacttccagtttcagctctaccaagtaaagagctagcaagtcatcaaaatggggacatacagaaaaaaaaaaggacactagaggaataatataccctgactcctagcctgattaatatatcgat

SEQ ID NO: 99 - protein, 1468 aa, Artificial Sequence
  ET3SEQuence
MQLELSTCVFLCLLPLGFSAIRRYYLGAVELSWDYRQSELLRELHVDTRFPATAPGALPLGPSVLYKKTVFVEFTDQLFSVARPRPPWMGLLGPTIQAEVYDTVVVTLKNMASHPVSLHAVGVSFWKSSEGAEYEDHTSQREKEDDKVLPGKSQTYVWQVLKENGPTASDPPCLTYSYLSHVDLVKDLNSGLIGALLVCREGSLTRERTQNLHEFVLLFAVFDEGKSWHSARNDSWTRAMDPAPARAQPAMHTVNGYVNRSLPGLIGCHKKSVYWHVIGMGTSPEVHSIFLEGHTFLVRHHRQASLEISPLTFLTAQTFLMDLGQFLLFCHISSHHHGGMEAHVRVESCAEEPQLRRKADEEEDYDDNLYDSDMDVVRLDGDDVSPFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFAQNSRPPSASAPKPPVLRRHQRDISLPTFQPEEDKMDYDDIFSTETKGEDFDIYGEDENQDPRSFQKRTRHYFIAAVEQLWDYGMSESPRALRNRAQNGEVPRFKKVVFREFADGSFTQPSYRGELNKHLGLLGPYIRAEVEDNIMVTFKNQASRPYSFYSSLISYPDDQEQGAEPRHNFVQPNETRTYFWKVQHHMAPTEDEFDCKAWAYFSDVDLEKDVHSGLIGPLLICRANTLNAAHGRQVTVQEFALFFTIFDETKSWYFTENVERNCRAPCHLQMEDPTLKENYRFHAINGYVMDTLPGLVMAQNQRIRWYLLSMGSNENIHSIHFSGHVFSVRKKEEYKMAVYNLYPGVFETVEMLPSKVGIWRIECLIGEHLQAGMSTTFLVYSKKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYV

SEQ ID NO: 100 - protein, 147 aa, Artificial Sequence
  beta-globin
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 101 - protein, 147 aa, Artificial Sequence
  gamma globin
MGHFTEEDKATITSLWGKVNVEDAGGETLGRLLVVYPWTQRFFDSFGNLSSASAIMGNPKVKAHGKKVLTSLGDATKHLDDLKGTFAQLSELHCDKLHVDPENFKLLGNVLVTVLAIHFGKEFTPEVQASWQKMVTAVASALSSRYH

SEQ ID NO: 102 - DNA, 3030 bp, Artificial Sequence
  3'HS1
ccaggctccattattgatatagtcatgatctcctctgttggggatgaagtaggcaaatttgaggcactaatttacttctcacattcttttcttgaacagaaagatagaactggaaattaatagtagtatataaattcaaaattttagctttaataacatttaatcagacataaataattatggtaatgtgaatttcaataaataaattttagttctaatataagtgtaactgtgtaatattcatactttttctgaaggctttactaatttgatatggcattacttttttattgctgccaaaactattcttattccactgtgtggtgatgagaaagtgagagatgttctggagatggtgattatagatagcttccctgaagccatagtaaccccctggagaaaaattggacctggagtctagcagcctaggtatgggtactcgatttcttagaaagcctttacaatttcctttatcttaaaaataagggtattgaagtagaattctagaattttcagaggacaacttaaaatatgtgtaatagttttaattatttatcctcataaatttaactgttcattttaatatatttaaggatgaattttttaaaaagttgatttcataaaaacgggaatagaaagatggttccataggctgactgagagtgtagaggagggatgggaagggaaagaagttgatcttcagttagactagaggaataagttttagtgatctctcacactgcatagtgaacacagttaataatatattatgtatttaaattaaaaattgctaaaaaataaatattttatgttctcaccacaaaaaaagttggaaggtgattcatatgctaattagcttgatagactctctctacaatgtatatatagatcaaacatcacattgtatcccataacatattatatatattatatatttatattatatattattattgtatccattaatatatgcacttattatttgccaggcaaataaaaaatgtttttaaaatataaatttatttgtaacctccttttacttttctgcttggttttcttctttcattcagtgtttaccagtttcttatagttaattttattttaagctgtctcacattttctgaagaaaagggaacatattaaagccaacaaaacaaatacactatcttgcatgagatgatttatgtcatggtacaatcaaatgctataaatcttataaaaacttctcaaatggttagatggctacagttgaacagatggaccatgtcatatattttttataatgcttctaaggtatggctaatttttaaaaaatattttagtaatgatgggaatattatttatagaaatcttataaaatatataatgaaatatgtaataaagtctagataaatgtgtatatacataatatatatttattacataatatataatatataatgtatatttatatattacatgcattatatattaaatataatacattttatatattatatattaaaatatgtaataatatgttattaaatatatacaataatctattacattttatgcttatataatatataataaatatatagtatataataaatatacactatatatttgtatctatatatgtttataaagtcattcctctaattaggtcataaccattcaggtaaactggaaatttaagcctacttcaggtttgtggtaaatagattctctctgaactagcatattcagaatcattaaacagtcagttctttggacaagtcttatagaatgttcttacctcttcagccatcccaagactcttgagggcctgacctcgcttacactaaagcagatctgccttatgcatcactgaagtagggagggaagaaagtttgatgaactacttctgacccctagtggtgtccagaaaagaccattaaaggaatgacctttaaaggatggacatacaattttttgtccaaggcaggacatgtgtgggtgtctttcagtaattatgttctaagaacagcaaaaactccactgccttggcaaataggaatgttttagttctatagaattataaagaagctgtcttttaaacacaatatactttctctatgtctttggaacaatgactattggtcattaccctattttaaagtaagcaagtaatcacacagggaattattctgaaaagacagaaaaaaaaaaaaaaccaagagatttctgcatatgtaggtcagttttaatcagagggcatcagaaaagactcctgaaagaatgacctggttattataatcacagatttgctttccaagtcaacattccagacagtgctcagaggggatacgaaaacccttttatttctccagactcaaattcactgctatttgtcttctctatttattttattataggcattgttctggttgctgggaactcagactgagataccatacactgactctcagatagcataacacaacatgatgtcttggaaaactgtaaatctttttgttttttaaatacaggtggagcatctggcacacctgacatattgatcttgtttttctttaaatcttcatttatttaccttatcaaaactatgctctttcatcctacctttcaaaacatattttaaaaaatcctccaacatgtattttgctctggtaatcccaaaaggctgatagtctctatggtggcaacatggataatactgttccccatctagatggtctcatttcttctgtatctagtctgaagaagcctgaatgaaagtagatttttaagctttgtagctagtctgaagcctttgtagtcagtctgaagaaacctgcatgaaaatagattttttttttcctttgggacagagtcttgctctgtcgcccagactggagtgcaatggcgcgatctcggctcactgcaacttccacctcccaggatcaagcaattctcctgcctcagtctcccaagtaactgggattacaggagcacactgccatgcccagctaattattttttgtgttttagtagagacagggtttcacc